(19) United States
(12) Patent Application Publication
(10) Pub. No.: US 2023/0296626 A1
(43) Pub. Date: Sep. 21, 2023

(54) COMPOSITIONS AND METHODS FOR TREATING INFLAMMASOME RELATED DISEASES OR CONDITIONS

(71) Applicant: University of Miami, Miami, FL (US)

(72) Inventors: Robert W. Keane, Miami, FL (US); Dalton W. Deitrich, Miami, FL (US); Juan Pablo de Rivero Vaccari, Miami, FL (US); Helen M. Bramlett, Miami, FL (US)

(73) Assignee: University of Miami, Miami, FL (US)

(21) Appl. No.: 17/921,600

(22) PCT Filed: Apr. 27, 2021
(86) PCT No.: PCT/US2021/029419

§ 371 Date: Oct. 26, 2022

Related U.S. Application Data
(60) Provisional application No. 63/062,622, filed on Aug. 7, 2020.
(60) Provisional application No. 63/016,033, filed on Apr. 27, 2020.

(51) Int. Cl. G01N 33/68; C07K 16/18; A61P 29/00

(52) U.S. Cl. CPC G01N 33/6896; C07K 16/18; A61P 29/00; G01N 33/6893; C07K 2317/565; C07K 2317/24; A61K 2039/505

(57) ABSTRACT

Compositions and methods for detecting components of the inflammasome in a sample from a subject as markers for inflammasome-related diseases or disorders such as multiple sclerosis, stroke, mild cognitive impairment, Alzheimer's disease, age-related macular degeneration, NASH, inflammaging or traumatic brain injury. Methods of using such inflammasome markers to determine prognosis, direct treatment and monitor response to treatment for the subject with an inflammasome-related disease or disorder such as multiple sclerosis, stroke, mild cognitive impairment, Alzheimer's disease, age-related macular degeneration, NASH, inflammaging or traumatic brain injury are also described.

### CROSS REFERENCE TO RELATED APPLICATIONS

This application claims priority to International Patent Application No. PCT/US2021/029419, which claims the benefit of U.S. Provisional Application No. 63/062,622, filed Aug. 7, 2020 and U.S. Provisional Application No. 63/016,033, filed Apr. 27, 2020, each of which is herein incorporated by reference in its entirety for all purposes.

### STATEMENT AS TO FEDERALLY SPONSORED RESEARCH

This invention was made with U.S. government support under grant number 4R42NS086274-02 awarded by the National Institute of Neurological Disorders and Stroke (NINDS) as well as grant number 5R42NS086274-03 awarded by the National Institute of Health. The U.S. government has certain rights in the invention.

### FIELD

The invention relates generally to the fields of immunology and medicine. More particularly, the invention relates to compositions and methods for detecting ASC (Apoptosis-associated Speck-like protein containing a Caspase Activating Recruitment Domain (CARD)) activity, caspase-1, IL-18, IL-1β, NOD-like receptors (NLR), Absent in Melanoma 2 (AIM2)-like receptors (ALR) and other inflammasome proteins alone or in combination with control biomarker proteins in samples obtained from a mammal as biomarkers for diseases, conditions or disorders such as multiple sclerosis (MS), stroke, mild cognitive impairment (MCI), Alzheimer's Disease (AD), age-related macular degeneration (AMD), age-related inflammation or traumatic brain injury (TBI). Finally, the invention relates to methods of treating neurological diseases, disorders and/or conditions alone or in combination with assessing expression levels of said inflammasome proteins using agents directed to said inflammasome proteins.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is UNMI_015_03WO_SeqList_ST25.txt. The text file is −43 KB, and was created on Apr. 27, 2021, and is being submitted electronically via EFS-Web.

### BACKGROUND

Multiple sclerosis (MS) is a progressive autoimmune disorder that affects the central nervous system (CNS). Pathologically, it is characterized by demyelination in the spinal cord and brain as well as the presence of inflammatory lesions (Compston A. The pathogenesis and basis for treatment in multiple sclerosis. Clin Neurol Neurosurg. 2004; 106:246-8). Clinically, patients with MS present blurred vision, muscle weakness, fatigue, dizziness, as well as balance and gate problems (Compston A. The pathogenesis and basis for treatment in multiple sclerosis. Clin Neurol Neurosurg. 2004; 106:246-8). In the United States, alone, there are 400,000 patients with MS and about 2 million patients worldwide (Compston A. The pathogenesis and basis for treatment in multiple sclerosis. Clin Neurol Neurosurg. 2004; 106:246-8).

Since the 1960s immunoglobulin (Ig) G oligoclonal bands (OCB) have been used as a classic biomarker in the diagnosis of MS (Stangel M, Fredrikson S, Meinl E, Petzold A, Stuve O and Tumani H. The utility of cerebrospinal fluid analysis in patients with multiple sclerosis. Nat Rev Neurol. 2013; 9:267-76). However, the specificity of IgG-OCB is only 61%, as a result, other diagnostic criteria is needed to clinically determine the diagnosis of MS (Teunissen C E, Malekzadeh A, Leurs C, Bridel C and Killestein J. Body fluid biomarkers for multiple sclerosis—the long road to clinical application. Nat Rev Neurol. 2015; 11:585-96), yet CSF-restricted IgG-OCB is a good predictor for conversion from CIS to CDMS, independently of MRI (Tintore M, Rovira A, Rio J, Tur C, Pelayo R, Nos C, Tellez N, Perkal H, Comabella M, Sastre-Garriga J and Montalban X. Do oligoclonal bands add information to MRI in first attacks of multiple sclerosis? Neurology. 2008; 70:1079-83). Similar results have been obtained when analyzing IgM-OCB (Villar L M, Masjuan J, Gonzalez-Porque P, Plaza J, Sadaba M C, Roldan E, Bootello A and Alvarez-Cermeno J C. Intrathecal IgM synthesis predicts the onset of new relapses and a worse disease course in MS. Neurology. 2002; 59:555-9). An important area of research in the field of MS is the identification of suitable biomarkers to predict who is at risk of developing MS, biomarkers of disease progression or exacerbation, as well as biomarkers of treatment response and prognosis.

There are 17.5 million deaths related to cardiovascular disease every year, of which 6.7 million occur as a result of stroke (Mendis S, Davis S and Norrving B. Organizational update: the world health organization global status report on noncommunicable diseases 2014; one more landmark step in the combat against stroke and vascular disease. Stroke. 2015; 46:e121-2). Even though there have been some large studies of stroke biomarkers, there is yet to be a gold standard biomarker that is used in the care of stroke patients. There is still a need for a biomarker that offers high sensitivity and high specificity for stroke.

The US Center for Disease Control (CDC) defines a traumatic brain injury (TBI) “as a disruption in the normal function of the brain that can be caused by a bump, blow, or jolt to the head, or penetrating head injury.” As of 2010, the CDC recorded 823.7 TBI-related emergency room visits, hospitalizations and deaths per 100,000 individuals in the US. (US Centers for Disease Control “Traumatic Brain Injury and Concussion Website. www.cdc.gov/traumaticbraininjury/index.html (as of 21 Jun. 2018)). An important area of research in the field of TBI is the identification of suitable biomarkers to at risk of developing TBI, biomarkers of disease diagnosis, progression or exacerbation, as well as biomarkers of treatment response and prognosis. Previous work on the inflammasome has indicated that inflammasome proteins can be used as biomarkers after traumatic brain injury. The inflammasome is a multiprotein complex of the innate immune response involved in the activation of caspase-1 and the processing of the inflammatory cytokines IL-1beta and IL18. The inflammasome contributes to the inflammatory response after injury to the brain and the spinal cord, among others.

During ageing, chronic, sterile, low-grade inflammation—called inflammaging—develops, which contributes to the pathogenesis of age-related diseases. From an evolutionary perspective, a variety of stimuli sustain inflammaging, including pathogens (non-self), endogenous cell debris and misplaced molecules (self) and nutrients and gut microbiota (quasi-self). A limited number of receptors, whose degeneracy allows them to recognize many signals and to activate the innate immune responses, sense these stimuli. However, the presence of biomarkers that can aid in the diagnosis of inflammaging as well as therapeutic targets and/or agents that can be used to treat inflammaging and/or age-related diseases are lacking.

A great deal of interest has been generated concerning the topic of a boundary or transitional state between normal aging and dementia, or Alzheimer disease (AD). This condition has received several descriptors including mild cognitive impairment (MCI), incipient dementia, and isolated memory impairment. Subjects with a mild cognitive impairment (MCI) have a memory impairment beyond that expected for age and education in the absence of dementia. These subjects are becoming the focus of many prediction studies and early intervention trials. However, the diagnostic criteria for MCI has not generally been elucidated and the presence of biomarkers is lacking. Moreover, the diagnosis of subjects at an early stage compared to a more advanced stage of AD is imperative to improve treatment outcomes.

Age-related macular degeneration (AMD) is a leading cause of blindness in the older population and affects over 11 million people in the United States alone and over 170 million people worldwide. AMD is a progressive degenerative disease that can result in irreversible vision loss. Patients in the early stages of AMD often experience no symptoms, and the disease is typically not detected until later, when vision loss begins to occur. As there is currently no cure for AMD, it is imperative that observable biomarkers be found to help screen for the disease in order to diagnose the early stages of AMD and slow its progression. (Zarbin M A. Current concepts in the pathogenesis of age-related macular degeneration. Arch Ophthalmol 2004; 122:598-614.; Ozaki E, Campbell M, Kiang A S, Humphries M, Doyle S L, Humphries P Inflammation in age-related macular degeneration. Adv Exp Med Biol 2014; 801:229-235.)

Thus, presented herein for addressing the above identified needs are inflammasome components useful as biomarkers with high sensitivity and specificity for various conditions associated with inflammation and methods of treating said conditions by targeting said inflammasome components.

### SUMMARY

In one aspect, provided herein is a method of evaluating a patient suspected of having multiple sclerosis (MS), the method comprising: measuring the level of at least one inflammasome protein in a biological sample obtained from the patient; determining the presence or absence of a protein signature associated with MS, wherein the protein signature comprises an elevated level of the at least one inflammasome protein; and selecting the patient as having MS if the patient exhibits the presence of the protein signature. In some cases, the patient is presenting with clinical symptoms consistent with MS. In some cases, the MS is relapsing-remitting MS (RRMS), secondary-progressive MS (SPMS), primary-progressive MS (PPMS), or progressive-relapsing MS (PRMS). In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1beta, apoptosis-associated speck-like protein containing a caspase recruitment domain (ASC), caspase-1, or combinations thereof. In some cases, the at least one inflammasome protein comprises each of caspase-1, IL-18, IL-1beta and ASC. In some cases, the at least one inflammasome protein comprises ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the level of the at least one inflammasome protein in the protein signature is enhanced relative to the level of the at least one inflammasome protein in a biological sample obtained from a control. In some cases, the biological sample obtained from the control is cerebrospinal fluid (CSF), CNS microdialysate, saliva, serum, plasma, urine or serum-derived extracellular vesicles (EVs). In some cases, the control is a healthy individual, wherein the healthy individual is an individual not presenting with clinical symptoms consistent with MS. In some cases, the at least one inflammasome protein comprises ASC, wherein the level of ASC is at least about 50% higher than the level of ASC in the biological sample obtained from a control. In some cases, the level of the at least one inflammasome protein in the protein signature is enhanced relative to a pre-determined reference value or range of reference values. In some cases, the biological sample obtained from patient is serum and the patient is selected as having MS with a sensitivity of at least about 80%, about 85%, about 90%, about 95%, about 99% or about 100% and a specificity of at least about 90%. In some cases, the biological sample is serum and the patient is selected as having MS with a specificity of at least about 80%, about 85%, about 90%, about 95%, about 99% or about 100%. In some cases, the biological sample is serum and the patient is selected as having MS with a sensitivity of at least 90% and a specificity of at least 80%. In some cases, the at least one inflammasome protein comprises ASC. In some cases, a cut-off value for determining the sensitivity, specificity or both is selected from Table 7. In some cases, the sensitivity and/or sensitivity is determined using the area under curve (AUC) from receiver operator characteristic (ROC) curves with confidence intervals of 95%.

In another aspect, provided herein is a method of evaluating a patient suspected of having suffered a stroke, the method comprising: measuring the level of at least one inflammasome protein in a biological sample obtained from the patient; determining the presence or absence of a protein signature associated with stroke or a stroke-related injury, wherein the protein signature comprises an elevated level of the at least one inflammasome protein; and selecting the patient as having suffered from a stroke if the patient exhibits the presence of the protein signature. In some cases, the patient is presenting with clinical symptoms consistent with stroke, wherein the stroke is ischemic stroke, transient ischemic stroke or hemorrhagic stroke. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1beta, apoptosis-associated speck-like protein containing a caspase recruitment domain (ASC), caspase-1, or combinations thereof. In some cases, the at least one inflammasome protein comprises each of caspase-1, IL-18, IL-1beta and ASC. In some cases, the at least one inflammasome protein comprises ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the level of the at least one inflammasome protein in the protein signature is enhanced relative to the level of the at least one inflammasome protein in a biological sample obtained from a control. In some cases, the biological sample obtained from the control is cerebrospinal fluid (CSF), CNS microdialysate, saliva, serum, plasma, urine or serum-derived extracellular vesicles (EVs). In some cases, the control is a healthy individual, wherein the healthy individual is an individual not presenting with clinical symptoms consistent with MS. In some cases, the at least one inflammasome protein comprises ASC, wherein the level of ASC in a serum sample obtained from the subject is at least 70% higher than the level of ASC in a serum sample obtained from a control. In some cases, the at least one inflammasome protein comprises ASC, wherein the level of ASC in a serum-derived EV sample obtained from the subject is at least 110% higher than the level of ASC in a serum-derived EV sample obtained from a control. In some cases, the level of the at least one inflammasome protein in the protein signature is enhanced relative to a pre-determined reference value or range of reference values. In some cases, the biological sample obtained from patient is serum and the patient is selected as having suffered a stroke with a sensitivity of at least about 80%, about 85%, about 90%, about 95%, about 99% or about 100% and a specificity of at least about 90%. In some cases, the biological sample is serum and the patient is selected as having suffered a stroke with a specificity of at least about 80%, about 85%, about 90%, about 95%, about 99% or about 100%. In some cases, the biological sample is serum and the patient is selected as having suffered a stroke with a sensitivity of at least 100% and a specificity of at least 95%. In some cases, the at least one inflammasome protein comprises ASC. In some cases, a cut-off value for determining the sensitivity, specificity or both is selected from Table 8. In some cases, the biological sample obtained from patient is serum-derived EVs and the patient is selected as having suffered a stroke with a sensitivity of at least about 80%, about 85%, about 90%, about 95%, about 99% or about 100% and a specificity of at least about 90%. In some cases, the biological sample is serum-derived EVs and the patient is selected as having suffered a stroke with a specificity of at least about 80%, about 85%, about 90%, about 95%, about 99% or about 100%. In some cases, the biological sample is serum-derived EVs and the patient is selected as having suffered a stroke with a sensitivity of at least 100% and a specificity of at least 100%. In some cases, the at least one inflammasome protein comprises ASC. In some cases, a cut-off value for determining the sensitivity, specificity or both is selected from Table 9. In some cases, the sensitivity and/or sensitivity is determined using the area under curve (AUC) from receiver operator characteristic (ROC) curves with confidence intervals of 95%.

In yet another aspect, provided herein is a method of treating a patient diagnosed with multiple sclerosis (MS), the method comprising administering a standard of care treatment for MS to the patient, wherein the diagnosis of MS was made by detecting an elevated level of at least one inflammasome protein in a biological sample obtained from the patient. In some cases, the MS is relapsing-remitting MS (RRMS), secondary-progressive MS (SPMS), primary-progressive MS (PPMS), or progressive-relapsing MS (PRMS). In some cases, the standard of care treatment is selected from therapies directed towards modifying disease outcome, managing relapses, managing symptoms or any combination thereof. In some cases, the therapies directed toward modifying disease outcome are selected from beta-interferons, glatiramer acetate, fingolimod, teriflunomide, dimethyl fumarate, mitoxanthrone, ocrelizumab, alemtuzumab, daclizumab and natalizumab.

In still another aspect, provided herein is a method of treating a patient diagnosed with stroke or a stroke related injury, the method comprising administering a standard of care treatment for stroke or stroke-related injury to the patient, wherein the diagnosis of stroke or stroke-related injury was made by detecting an elevated level of at least one inflammasome protein in a biological sample obtained from the patient. In some cases, the stroke is ischemic stroke, transient ischemic stroke or hemorrhagic stroke. In some cases, the stroke is ischemic stroke or transient ischemic stroke and the standard of care treatment is selected from tissue plasminogen activator (tPA), antiplatelet medicine, anticoagulants, a carotid artery angioplasty, carotid endarterectomy, intra-arterial thrombolysis and mechanical clot removal in cerebral ischemia (MERCI) or a combination thereof. In some cases, the stroke is hemorrhagic stroke and the standard of care treatment is an aneurysm clipping, coil embolization or arteriovenous malformation (AVM) repair. In some cases, the elevated level of the at least one inflammasome protein is measured by an immunoassay utilizing one or more antibodies directed against the at least one inflammasome protein. In some cases, the level of the at least one inflammasome protein is enhanced relative to the level of the at least one inflammasome protein in a control sample. In some cases, the level of the at least one inflammasome protein is enhanced relative to a pre-determined reference value or range of reference values. In some cases, the at least one inflammasome protein is interleukin 18 (IL-18), apoptosis-associated speck-like protein containing a caspase recruitment domain (ASC), caspase-1, or combinations thereof. In some cases, the at least one inflammasome protein is caspase-1, IL-18, and ASC. In some cases, the at least one inflammasome protein is ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the biological sample is cerebrospinal fluid (CSF), CNS microdialysate, saliva, serum, plasma, urine or serum-derived extracellular vesicles (EVs).

In a still further aspect, provided herein is a method of evaluating a patient suspected of having traumatic brain injury (TBI), the method comprising: measuring the level of at least one inflammasome protein in a biological sample obtained from the patient; determining the presence or absence of a protein signature associated with TBI, wherein the protein signature comprises an elevated level of the at least one inflammasome protein; and selecting the patient as having TBI if the patient exhibits the presence of the protein signature. In some cases, the patient is presenting with clinical symptoms consistent with TBI. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises caspase-1. In some cases, the at least one inflammasome protein comprises ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the level of the at least one inflammasome protein in the protein signature is enhanced relative to the level of the at least one inflammasome protein in a biological sample obtained from a control. In some cases, the at least one inflammasome protein comprises caspase-1, wherein the level of caspase-1 is at least 50% higher than the level of caspase-1 in the biological sample obtained from the control. In some cases, the at least one inflammasome protein comprises ASC, wherein the level of ASC is at least 50% higher than the level of ASC in the biological sample obtained from the control. In some cases, the biological sample obtained from the control is cerebrospinal fluid (CSF), CNS microdialysate, saliva, serum, plasma, urine or serum-derived extracellular vesicles (EVs). In some cases, the control is a healthy individual, wherein the healthy individual is an individual not presenting with clinical symptoms consistent with TBI. In some cases, the level of the at least one inflammasome protein in the protein signature is enhanced relative to a pre-determined reference value or range of reference values. In some cases, the biological sample obtained from patient is serum and the patient is selected as having TBI with a sensitivity of at least about 80%, about 85%, about 90%, about 95%, about 99% or about 100% and a specificity of at least about 90%. In some cases, the biological sample is serum and the patient is selected as having TBI with a specificity of at least about 80%, about 85%, about 90%, about 95%, about 99% or about 100%. In some cases, the biological sample is serum and the patient is selected as having TBI with a sensitivity of at least 90% and a specificity of at least 80%. In some cases, the sensitivity and/or sensitivity is determined using the area under curve (AUC) from receiver operator characteristic (ROC) curves with confidence intervals of 95%. In some cases, the at least one inflammasome protein comprises ASC. In some cases, a cut-off value for determining the sensitivity, specificity or both is selected from Tables 11B, 12B, 14A, 16, 17 or 19. In some cases, the at least one inflammasome protein comprises caspase-1. In some cases, a cut-off value for determining the sensitivity, specificity or both is selected from Tables 11A or 15.

In yet another aspect, provided herein is a method of evaluating a patient suspected of having a brain injury, the method comprising: measuring the level of at least one inflammasome protein in a biological sample obtained from the patient; determining the presence or absence of a protein signature associated with brain injury, wherein the protein signature comprises an elevated level of the at least one inflammasome protein; and selecting the patient as having brain injury if the patient exhibits the presence of the protein signature. In some cases, the patient is presenting with clinical symptoms consistent with brain injury. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the at least one inflammasome protein comprises caspase-1. In some cases, the level of the at least one inflammasome protein in the protein signature is enhanced relative to the level of the at least one inflammasome protein in a biological sample obtained from a control. In some cases, the at least one inflammasome protein comprises ASC, wherein the level of ASC is at least 50% higher than the level of ASC in the biological sample obtained from the control. In some cases, the at least one inflammasome protein comprises caspase-1, wherein the level of caspase-1 is at least 50% higher than the level of caspase-1 in the biological sample obtained from the control. In some cases, the biological sample obtained from the control is cerebrospinal fluid (CSF), CNS microdialysate, saliva, serum, plasma, urine or serum-derived extracellular vesicles (EVs). In some cases, the control is a healthy individual, wherein the healthy individual is an individual not presenting with clinical symptoms consistent with brain injury. In some cases, the brain injury is selected from a traumatic brain injury, stroke, mild cognitive impairment or multiple sclerosis. In some cases, the level of the at least one inflammasome protein in the protein signature is enhanced relative to a pre-determined reference value or range of reference values. In some cases, the brain injury is traumatic brain injury (TBI). In some cases, the biological sample obtained from patient is serum and the patient is selected as having TBI with a sensitivity of at least about 80%, about 85%, about 90%, about 95%, about 99% or about 100% and a specificity of at least about 90%. In some cases, the biological sample is serum and the patient is selected as having TBI with a specificity of at least about 80%, about 85%, about 90%, about 95%, about 99% or about 100%. In some cases, the biological sample is serum and the patient is selected as having TBI with a sensitivity of at least 90% and a specificity of at least 80%. In some cases, the sensitivity and/or sensitivity is determined using the area under curve (AUC) from receiver operator characteristic (ROC) curves with confidence intervals of 95%. In some cases, the at least one inflammasome protein comprises ASC. In some cases, a cut-off value for determining the sensitivity, specificity or both is selected from Tables 11B, 12B, 14A, 16, 17 or 19. In some cases, the at least one inflammasome protein comprises caspase-1. In some cases, a cut-off value for determining the sensitivity, specificity or both is selected from Tables 11A or 15. In some cases, the brain injury is mid cognitive impairment (MCI). In some cases, the biological sample obtained from patient is serum and the patient is selected as having MCI with a sensitivity of at least 75%, 80%, 85%, 90%, 95%, 99% or 100%. In some cases, the biological sample is serum and the patient is selected as having MCI with a specificity of at least about 55%, about 60%, about 65%, about 70%, about 75%, about 80%, about 85%, about 90%, about 95%, about 99% or about 100%. In some cases, the biological sample is serum and the patient is selected as having MCI with a sensitivity of at least 90% and a specificity of at least 70%. In some cases, the sensitivity and/or sensitivity is determined using the area under curve (AUC) from receiver operator characteristic (ROC) curves with confidence intervals of 95%. In some cases, the at least one inflammasome protein comprises ASC. In some cases, a cut-off value for determining the sensitivity, specificity or both is selected from Tables 22 or 23. In some cases, the at least one inflammasome protein comprises IL-18. In some cases, a cut-off value for determining the sensitivity, specificity or both is selected from Tables 22 or 25. In some cases, the brain injury is multiple sclerosis (MS). In some cases, the biological sample obtained from patient is serum and the patient is selected as having MS with a sensitivity of at least about 80%, about 85%, about 90%, about 95%, about 99% or about 100% and a specificity of at least about 90%. In some cases, the biological sample is serum and the patient is selected as having MS with a specificity of at least about 80%, about 85%, about 90%, about 95%, about 99% or about 100%. In some cases, the biological sample is serum and the patient is selected as having MS with a sensitivity of at least 90% and a specificity of at least 80%. In some cases, the at least one inflammasome protein comprises ASC. In some cases, a cut-off value for determining the sensitivity, specificity or both is selected from Table 7. In some cases, the sensitivity and/or sensitivity is determined using the area under curve (AUC) from receiver operator characteristic (ROC) curves with confidence intervals of 95%. In some cases, the brain injury is stroke. In some cases, the biological sample obtained from patient is serum and the patient is selected as having suffered a stroke with a sensitivity of at least about 80%, about 85%, about 90%, about 95%, about 99% or about 100% and a specificity of at least 90%. In some cases, the biological sample is serum and the patient is selected as having suffered a stroke with a specificity of at least about 80%, about 85%, about 90%, about 95%, about 99% or about 100%. In some cases, the biological sample is serum and the patient is selected as having suffered a stroke with a sensitivity of at least 100% and a specificity of at least 95%. In some cases, the at least one inflammasome protein comprises ASC. In some cases, a cut-off value for determining the sensitivity, specificity or both is selected from Table 8. In some cases, the biological sample obtained from patient is serum-derived EVs and the patient is selected as having suffered a stroke with a sensitivity of at least about 80%, about 85%, about 90%, about 95%, about 99% or about 100% and a specificity of at least 90%. In some cases, the biological sample is serum-derived EVs and the patient is selected as having suffered a stroke with a specificity of at least about 80%, about 85%, about 90%, about 95%, about 99% or about 100%. In some cases, the biological sample is serum-derived EVs and the patient is selected as having suffered a stroke with a sensitivity of at least 100% and a specificity of at least 100%. In some cases, the at least one inflammasome protein comprises ASC. In some cases, a cut-off value for determining the sensitivity, specificity or both is selected from Table 9. In some cases, the sensitivity and/or sensitivity is determined using the area under curve (AUC) from receiver operator characteristic (ROC) curves with confidence intervals of 95%.

In a still further aspect, provided herein is a method of evaluating a patient suspected of having mild cognitive impairment (MCI) the method comprising: measuring the level of at least one inflammasome protein in a biological sample obtained from the patient; determining the presence or absence of a protein signature associated with MCI, wherein the protein signature comprises an elevated level of the at least one inflammasome protein; and selecting the patient as having MCI if the patient exhibits the presence of the protein signature. In some cases, the patient is presenting with clinical symptoms consistent with MCI. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises ASC. In some cases, the at least one inflammasome protein comprises IL-18. In some cases, the antibody binds to the PYRIN-PAAD-DAPIN domain (PYD), C-terminal caspase-recruitment domain (CARD) domain or a portion of the PYD or CARD domain of the ASC protein. In some cases, the level of the at least one inflammasome protein in the protein signature is enhanced relative to the level of the at least one inflammasome protein in a biological sample obtained from a control. In some cases, the at least one inflammasome protein comprises ASC, wherein the level of ASC is at least 50% higher than the level of ASC in the biological sample obtained from the control. In some cases, the at least one inflammasome protein comprises IL-18, wherein the level of IL-18 is at least 25% higher than the level of IL-18 in the biological sample obtained from the control.

In one aspect, provided herein is a method of evaluating a patient suspected of having mild cognitive impairment (MCI), the method comprising: measuring an expression level of at least one inflammasome protein in a biological sample obtained from the patient; comparing the expression level of the at least one inflammasome protein in the biological sample to an expression level of one or more control MCI biomarkers; and selecting the patient as having MCI if the expression level of the at least one inflammasome protein in the biological sample is similar to the expression level of the one or more control MCI biomarkers. In some cases, the expression level of the at least one inflammasome protein is similar to the expression level of the one or more control MCI biomarkers if the expression level or a parameter representative of the expression level of the at least one inflammasome protein is within 10%, 9%, 8%, 7%, 6%, 5%, 4%, 3%, 2% or 1% of the expression level or a parameter representative of the expression level of the one or more control MCI biomarkers. In some cases, the expression level of the one or more control MCI biomarkers is measured in the biological sample obtained from the patient. In some cases, the expression level of the one or more control MCI biomarkers is measured in a biological sample obtained from an individual previously diagnosed with MCI. In some cases, the biological sample obtained from the individual previously diagnosed with MCI is a same type of biological sample obtained from the patient suspected of suffering from MCI. In some cases, the expression level of the at least one inflammasome protein and the expression level of the one or more control MCI biomarkers are enhanced relative to the expression level of the at least one inflammasome protein and the expression level of the one or more control MCI biomarkers in a biological sample obtained from a control. In some cases, the biological sample obtained from the control is a same type of biological sample obtained from the patient suspected of suffering from MCI. In some cases, the control is a healthy individual, wherein the healthy individual is an individual not presenting with clinical symptoms consistent with MCI. In some cases, the expression level of the at least one inflammasome protein and the expression level of the one or more control MCI biomarkers are enhanced relative to a pre-determined reference value or range of reference values for the at least one inflammasome protein and the one or more control MCI biomarkers. In some cases, the parameter representative of the expression level of the at least one inflammasome protein and the parameter representative of the expression level of the one or more control MCI biomarkers is an area under curve (AUC). In some cases, the patient is presenting with clinical symptoms consistent with MCI. In some cases, the biological sample obtained from the patient suspected of suffering from MCI is cerebrospinal fluid (CSF), CNS microdialysate, saliva, serum, plasma, urine or serum-derived extracellular vesicles (EVs). In some cases, the expression level of the at least one inflammasome protein and/or the one or more control MCI biomarkers is measured by an immunoassay utilizing one or more antibodies directed against the at least one inflammasome protein and/or the one or more control MCI biomarkers. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises ASC. In some cases, the at least one inflammasome protein comprises IL-18. In some cases, the one or more control MCI biomarkers are neurofilament light polypeptide (NFL), soluble APP-alpha (sAPPα) and/or soluble APP-beta (sAPPβ). In some cases, the at least one inflammasome protein is ASC and the one or more control MCI biomarkers is soluble APP-alpha (sAPPα), wherein the AUC for ASC is 0.974 and the AUC for sAPP-alpha is 0.9687. In some cases, the at least one inflammasome protein is ASC and the one or more control MCI biomarkers is soluble APP-beta (sAPPβ), wherein the AUC for ASC is 0.974 and the AUC for sAPP-beta is 0.9068. In some cases, the at least one inflammasome protein is ASC and the one or more control MCI biomarkers is neurofilament light polypeptide (NFL) wherein the AUC for ASC is 0.974 and the AUC for NFL is 0.7734. In some cases, the biological sample obtained from the patient is serum and the patient is selected as having MCI with a sensitivity of at least 70%, 75%, 80%, 85%, 90%, 95%, 99% or 100% and a specificity of at least 55%. In some cases, the biological sample obtained from the patient is serum and the patient is selected as having MCI with a sensitivity of at least 70%, 75%, 80%, 85%, 90%, 95%, 99% or 100%. In some cases, the biological sample obtained from the patient is serum and the patient is selected as having MCI with a sensitivity of at least 70% and a specificity of at least 55%. In some cases, the specificity and/or sensitivity is determined using receiver operator characteristic (ROC) curves with confidence intervals of 95%. In some cases, said method further comprises assessing the presence of one or more symptoms associated with MCI in order to select the patient as having MCI. In some cases, the one or more symptoms associated with MCI are forgetfulness, lack of focus, anxiety, difficulty making decisions, difficulty understanding instructions, difficulty planning, trouble navigating familiar environments, impulsivity, or questionable judgment as well as judging the time or sequence of steps needed to complete a complex task or visual perception.

In another aspect, provided herein is a method of evaluating a patient suspected of having Alzheimer's Disease (AD), the method comprising: measuring an expression level of at least one inflammasome protein in a biological sample obtained from the patient; comparing the expression level of the at least one inflammasome protein in the biological sample to an expression level of one or more control AD biomarkers; and selecting the patient as having AD if the expression level of the at least one inflammasome protein in the biological sample is similar to the expression level of the one or more control AD biomarkers. In some cases, the expression level of the at least one inflammasome protein is similar to the expression level of the one or more control AD biomarkers if the expression level or a parameter representative of the expression level of the at least one inflammasome protein is within 10%, 9%, 8%, 7%, 6%, 5%, 4%, 3%, 2% or 1% of the expression level or a parameter representative of the expression level of the one or more control AD biomarkers. In some cases, the expression level of the one or more control AD biomarkers is measured in the biological sample obtained from the patient. In some cases, the expression level of the one or more control AD biomarkers is measured in a biological sample obtained from an individual previously diagnosed with AD. In some cases, the biological sample obtained from the individual previously diagnosed with AD is a same type of biological sample obtained from the patient suspected of suffering from AD. In some cases, the expression level of the at least one inflammasome protein and the expression level of the one or more control AD biomarkers are enhanced relative to the expression level of the at least one inflammasome protein and the expression level of the one or more control AD biomarkers in a biological sample obtained from a control. In some cases, the biological sample obtained from the control is a same type of biological sample obtained from the patient suspected of suffering from AD. In some cases, the control is a healthy individual, wherein the healthy individual is an individual not presenting with clinical symptoms consistent with AD. In some cases, the expression level of the at least one inflammasome protein and the expression level of the one or more control AD biomarkers are enhanced relative to a pre-determined reference value or range of reference values for the at least one inflammasome protein and the one or more control AD biomarkers. In some cases, the parameter representative of the expression level of the at least one inflammasome protein and the parameter representative of the expression level of the one or more control AD biomarkers is an area under curve (AUC). In some cases, the patient is presenting with clinical symptoms consistent with AD. In some cases, the biological sample obtained from the patient suspected of suffering from AD is cerebrospinal fluid (CSF), CNS microdialysate, saliva, serum, plasma, urine or serum-derived extracellular vesicles (EVs). In some cases, the expression level of the at least one inflammasome protein and/or the one or more control AD biomarkers is measured by an immunoassay utilizing one or more antibodies directed against the at least one inflammasome protein and/or the one or more control AD biomarkers. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises ASC. In some cases, the at least one inflammasome protein comprises IL-18. In some cases, the one or more control AD biomarkers are neurofilament light polypeptide (NFL), soluble APP-alpha (sAPPα) and/or soluble APP-beta (sAPPβ). In some cases, the at least one inflammasome protein is ASC and the one or more control AD biomarkers is soluble APP-alpha (sAPPα), wherein the AUC for ASC is 0.833 and the AUC for sAPPα is 0.956. In some cases, the at least one inflammasome protein is ASC and the one or more control AD biomarkers is soluble APPβ (sAPPβ), wherein the AUC for ASC is 0.833 and the AUC for sAPPβ is 0.919. In some cases, the at least one inflammasome protein is ASC and the one or more control AD biomarkers is neurofilament light polypeptide (NFL), wherein the AUC for ASC is 0.833 and the AUC for NFL is 0.717. In some cases, the biological sample obtained from the patient is serum and the patient is selected as having AD with a sensitivity of at least 70%, 75%, 80%, 85%, 90%, 95%, 99% or 100% and a specificity of at least 55%. In some cases, the biological sample obtained from the patient is serum and the patient is selected as having AD with a sensitivity of at least 70%, 75%, 80%, 85%, 90%, 95%, 99% or 100%. In some cases, the biological sample obtained from the patient is serum and the patient is selected as having AD with a sensitivity of at least 70% and a specificity of at least 55%. In some cases, the specificity and/or sensitivity is determined using receiver operator characteristic (ROC) curves with confidence intervals of 95%. In some cases, said method further comprises assessing the presence of one or more symptoms associated with AD in order to select the patient as having AD. In some cases, the one or more symptoms associated with AD are forgetfulness, lack of focus, anxiety, feeling anxious or overwhelmed when making decisions, difficulty understanding instructions or planning things, trouble navigating familiar environments, difficulty performing tasks, forgetting material that was just read, losing or misplacing a valuable object, difficulty with organization, confusion with time or place, trouble controlling bladder or bowels, personality or behavioral changes such as changes in mood or personality; changes in sleep patterns, difficulty communicating such as problems with words in speaking or writing, vulnerability to infections, impulsivity, or questionable judgment, trouble understanding visual images and spatial relationships, misplacing things and losing the ability to retrace steps, decreased or poor judgement, withdrawal from work or social activities. In some cases, the parameter representative of the expression level of the at least one inflammasome protein and the parameter representative of the expression level of the one or more control MCI biomarkers is a cut-off value. In some cases, at least one inflammasome protein is ASC and the cut-off value is above 264.9 pg/ml and below 560 pg/ml. In some cases, the parameter representative of the expression level of the at least one inflammasome protein and the parameter representative of the expression level of the one or more control MCI biomarkers is a cut-off value. In some cases, the at least one inflammasome protein is ASC and the cut-off value is above 560 pg/ml.

In one aspect, provided herein is a method of determining whether a patient is suffering from mild cognitive impairment (MCI) or Alzheimer's Disease (AD), the method comprising: measuring an expression level of at least one inflammasome protein in a biological sample obtained from the patient; comparing the expression level of the at least one inflammasome protein in the biological sample to a pre-determined reference value or range of reference values for the at least one inflammasome protein; and selecting the patient as having AD if the expression level of the at least one inflammasome protein is within the predetermined range of reference values or MCI if the expression level is above a pre-determined reference value. In some cases, the at least one inflammasome protein is ASC. In some cases, the predetermined range of reference values is between 264.9 pg/ml and 560 pg/ml. In some cases, the pre-determined reference value is above 560 pg/ml.

In another aspect, provided herein is a method of evaluating a patient suspected of age-related macular degeneration (AMD), the method comprising: measuring an expression level of at least one inflammasome protein in a biological sample obtained from the patient; determining the presence or absence of a protein signature associated with AMD, wherein the protein signature comprises an elevated expression level of the at least one inflammasome protein; and selecting the patient as having AMD if the patient exhibits the presence of the protein signature. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the level of the at least one inflammasome protein in the protein signature is enhanced relative to the level of the at least one inflammasome protein in a biological sample obtained from a control. In some cases, the biological sample obtained from the control is cerebrospinal fluid (CSF), CNS microdialysate, saliva, serum, plasma, urine or serum-derived extracellular vesicles (EVs). In some cases, the control is a healthy individual not exhibiting the clinical symptoms of AMD. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises ASC, and wherein the AUC for ASC is 0.9823. In some cases, the at least one inflammasome protein comprises IL-18, and wherein the AUC for IL-18 is 0.7286. In some cases, the biological sample obtained from the patient is serum and the patient is selected as having AMD with a sensitivity of at least 70%, 75%, 80%, 85%, 90%, 95%, 99%, or 100%. In some cases, the biological sample obtained from the patient is serum and the patient is selected as having AMD with a sensitivity of at least 70%, 75%, 80%, 85%, 90%, 95%, 99%, or 100% and a specificity of at least 55%. In some cases, the specificity and/or sensitivity is determined using receiver operator characteristic (ROC) curves with confidence intervals of 95%. In some cases, said method further comprises assessing the presence of one or more symptoms associated with AMD in order to select the patient having AMD. In some cases, the one or more symptoms associated with AMD are blurred vision, fuzzy vision, seeing straight lines as wavy or distorted, seeing blurry areas on a printed page, difficulty reading or seeing details in low light levels, extra sensitivity to glare, dark or blurry areas in the center of vision, whiteout in the center of vision, or a change in the perception of color. In some cases, the parameter representative of the expression level of the at least one inflammasome protein is a cut-off value. In some cases, the at least one inflammasome protein is ASC, and the cut-off value is above 365.6 pg/mL. In some cases, the at least one inflammasome protein is IL-18, and the cut-off value is above 242.4 pg/mL.

In one aspect, provided herein is a method of treating inflammaging in a subject, the method comprises administering to the subject a therapeutically effective amount of a monoclonal antibody or an antibody fragment thereof of that binds specifically to ASC, wherein the antibody or the antibody fragment comprises a heavy chain variable (VH) region and a light chain variable (VL) region, wherein the VH region amino acid sequence comprises HCDR1 of SEQ ID NO: 6, HCDR2 of SEQ ID NO: 7 and HCDR3 of SEQ ID NO: 8, or a variant thereof having at least one amino acid substitution in HCDR1, HCDR2, and/or HCDR3; and wherein the VL region amino acid sequence comprises LCDR1 of SEQ ID NO: 12, LCDR2 of SEQ ID NO: 13 and LCDR3 of SEQ ID NO: 14, or a variant thereof having at least one amino acid substitution in LCDR1, LCDR2, and/or LCDR3, thereby treating inflammaging in the subject. In some cases, the VH region amino acid sequence of the monoclonal antibody or the antibody fragment thereof comprises SEQ ID NO: 18, 19, 20, 21, 22, or an amino acid sequence that is at least 95%, 96%, 97%, 98% or 99% identical to the amino acid sequence of SEQ ID NO: 18, 19, 20, 21 or 22; and wherein the VL region amino acid sequence of the monoclonal antibody or the antibody fragment thereof comprises SEQ ID NO: 28, 29, 30, 31, or an amino acid sequence that is at least 95%, 96%, 97%, 98% or 99% identical to the amino acid sequence of SEQ ID NO: 28, 29, 30 or 31. In some cases, the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28. In some cases, the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and wherein the VL region amino acid sequence of the monoclonal antibody or the antibody fragment thereof comprises SEQ ID NO: 29 or an amino acid sequence that is at least 95%, 96%, 97%, 98% or 99% identical to the amino acid sequence of SEQ ID NO: 29. In some cases, the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30. In some cases, the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31. In some cases, the VH region amino acid sequence of the monoclonal antibody or the antibody fragment thereof comprises SEQ ID NO: 19, or an amino acid sequence that is at least 95%, 96%, 97%, 98% or 99% identical to the amino acid sequence of SEQ ID NO: 19; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28. In some cases, the VH region amino acid sequence of the monoclonal antibody or the antibody fragment thereof comprises SEQ ID NO: 19, or an amino acid sequence that is at least 95%, 96%, 97%, 98% or 99% identical to the amino acid sequence of SEQ ID NO: 19; and wherein the VL region amino acid sequence comprises SEQ ID NO: 29 or an amino acid sequence that is at least 95%, 96%, 97%, 98% or 99% identical to the amino acid sequence of SEQ ID NO: 29. In some cases, the VH region amino acid sequence of the monoclonal antibody or the antibody fragment thereof comprises SEQ ID NO: 19, or an amino acid sequence that is at least 95%, 96%, 97%, 98% or 99% identical to the amino acid sequence of SEQ ID NO: 19; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30. In some cases, the VH region amino acid sequence of the monoclonal antibody or the antibody fragment thereof comprises SEQ ID NO: 19, or an amino acid sequence that is at least 95%, 96%, 97%, 98% or 99% identical to the amino acid sequence of SEQ ID NO: 19; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31. In some cases, the VH region amino acid sequence of the monoclonal antibody or the antibody fragment thereof comprises SEQ ID NO: 20, or an amino acid sequence that is at least 95%, 96%, 97%, 98% or 99% identical to the amino acid sequence of SEQ ID NO: 20; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28. In some cases, the VH region amino acid sequence comprises SEQ ID NO: 20, or an amino acid sequence that is at least 95%, 96%, 97%, 98% or 99% identical to the amino acid sequence of SEQ ID NO: 20; and wherein the VL region amino acid sequence comprises SEQ ID NO: 29 or an amino acid sequence that is at least 95%, 96%, 97%, 98% or 99% identical to the amino acid sequence of SEQ ID NO: 29. In some cases, the VH region amino acid sequence of the monoclonal antibody or the antibody fragment thereof comprises SEQ ID NO: 20, or an amino acid sequence that is at least 95%, 96%, 97%, 98% or 99% identical to the amino acid sequence of SEQ ID NO: 20; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30. In some cases, the VH region amino acid sequence of the monoclonal antibody or the antibody fragment thereof comprises SEQ ID NO: 20, or an amino acid sequence that is at least 95%, 96%, 97%, 98% or 99% identical to the amino acid sequence of SEQ ID NO: 20; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31. In some cases, the VH region amino acid sequence of the monoclonal antibody or the antibody fragment thereof comprises SEQ ID NO: 21, or an amino acid sequence that is at least 95%, 96%, 97%, 98% or 99% identical to the amino acid sequence of SEQ ID NO: 21; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28. In some cases, the VH region amino acid sequence comprises SEQ ID NO: 21, or an amino acid sequence that is at least 95%, 96%, 97%, 98% or 99% identical to the amino acid sequence of SEQ ID NO: 21; and wherein the VL region amino acid sequence comprises SEQ ID NO: 29 or an amino acid sequence that is at least 95%, 96%, 97%, 98% or 99% identical to the amino acid sequence of SEQ ID NO: 29. In some cases, the VH region amino acid sequence of the monoclonal antibody or the antibody fragment thereof comprises SEQ ID NO: 21, or an amino acid sequence that is at least 95%, 96%, 97%, 98% or 99% identical to the amino acid sequence of SEQ ID NO: 21; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30. In some cases, the VH region amino acid sequence of the monoclonal antibody or the antibody fragment thereof comprises SEQ ID NO: 21, or an amino acid sequence that is at least 95%, 96%, 97%, 98% or 99% identical to the amino acid sequence of SEQ ID NO: 21; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31. In some cases, the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28. In some cases, the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and wherein the VL region amino acid sequence of the monoclonal antibody or the antibody fragment thereof comprises SEQ ID NO: 29 or an amino acid sequence that is at least 95%, 96%, 97%, 98% or 99% identical to the amino acid sequence of SEQ ID NO: 29. In some cases, the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30. In some cases, the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31. In some cases, the ASC is human ASC protein. In some cases, the antibody fragment is a Fab, an F(ab′)2, a Fab′, a scFv, a single domain antibody, a diabody or a single chain camelid antibody. In some cases, the monoclonal antibody or the antibody fragment thereof is human, humanized or chimeric. In some cases, the administering the monoclonal antibody or the antibody fragment thereof reduces levels of at least inflammatory cytokine. In some cases, the administration of the monoclonal antibody or the antibody fragment thereof results in inhibition of inflammasome activation in the subject. In some cases, the administration of the monoclonal antibody or the antibody fragment thereof results in a reduction in the activity of ASC as compared to a control. In some cases, the control is an untreated subject. In some cases, the administration is intracerebroventricularly, intraperitoneally, intravenously or by inhalation.

### BRIEF DESCRIPTION OF THE DRAWINGS

FIG. 1A-1D illustrates that inflammasome proteins are elevated in the serum of MS patients. Protein levels in pg/ml of caspase-1 (FIG. 1A), ASC (FIG. 1B), IL-1β (FIG. 1C) and IL-18 (FIG. 1D) in serum samples from patients with MS and healthy donors. p-value of significance is shown above each box plot. Box and whiskers are shown for the 5th and 95th percentile. Caspase-1: N=9 control and 19 MS; ASC: N=115 control and 32 MS; IL-1β: N=21 control and 8 MS; and IL-18: N=119 control and 32 MS.

FIG. 2A-2D illustrates ROC curves for caspase-1 (FIG. 2A), ASC (FIG. 2B), IL-1β (FIG. 2C) and IL-18 (FIG. 2D) from serum samples of MS and healthy donors.

FIG. 3 illustrates inflammasome proteins in serum as biomarkers of MS. ROC curves for caspase-1, ASC, IL-1beta and IL-18. Caspase-1: N=9 control and 19 MS; ASC: N=115 control and 32 MS; IL-1beta: N=21 control and 8 MS; and IL-18: N=119 control and 32 MS.

FIG. 4 illustrates a table containing the characteristics of the subjects with Multiple Sclerosis (MS) from Example 1.

FIG. 5A-5D illustrates inflammasome proteins are elevated in the serum of stroke patients. Protein levels in pg/ml of caspase-1 (FIG. 5A), ASC (FIG. 5B), IL-1beta (FIG. 5C) and IL-18 (FIG. 5D) in serum samples from patients with stroke and healthy donors. p-value of significance is shown above each box plot. Box and whiskers are shown for the 5th and 95th percentile. N.S.=Not Significant. Caspase-1: N=8 control and 13 stroke; ASC: N=75 control and 16 stroke; IL-1beta: N=9 control and 8 stroke; and IL-18: N=79 control and 15 stroke.

FIG. 6 illustrates inflammasome proteins in serum as biomarkers of stroke. ROC curves for caspase-1, ASC, IL-1beta and IL-18. Caspase-1: N=8 control and 13 stroke; ASC: N=75 control and 16 stroke; IL-1beta: N=9 control and 8 stroke; and IL-18: N=79 control and 15 stroke.

FIG. 7A illustrates a comparison of total protein levels from serum-derived extracellular vesicle (EV). A Bradford Assay was carried following EV isolation from serum to determine total protein concentration in isolates with the Invitrogen kit (INVTR) and the ExoQuick kit (EQ). Data presented as mean+/−SEM. N=6 per group. FIG. 7B depicts a representative image of total protein loaded. Stain-free image of serum-derived EV proteins. Equal amounts of protein lysates (10 ml) were loaded in each lane of a Criterion gel. FIG. 7C depicts a bar graph shows quantification of the entire lane corresponding to loaded EV isolated with the Invitrogen kit (INV) and the ExoQuick kit (EQ).

FIG. 8A-8F illustrates EV characterization in serum from stroke patients. FIG. 8A depicts a representative immunoblot of CD81 and NCAM positive EV isolated with the Invitrogen Kit (IN) and the ExoQuick Kit (EQ). +Contr: Positive control of isolated EV. Quantification of CD81- (FIG. 8B) and NCAM- (FIG. 8C) positive EV isolated from serum with the Invitrogen kit (INV) and the ExoQuick kit (EQ). FIG. 8D depicts an electron microscopy image of EV isolated by two different techniques. Bar=100 nm. Nanoparticle tracking analysis/particle size distribution of isolated serum-derived EV. Nanoparticle tracking analysis predicts size distribution and concentration of particles in serum-derived EV samples isolated with the Invitrogen kit (FIG. 8E) and the ExoQuick kit (FIG. 8F).

FIG. 9A-9C illustrates that ASC is elevated in serum-derived EV of stroke patients. Protein levels in pg/ml of ASC (FIG. 9A), IL-1beta (FIG. 9B) and IL-18 (FIG. 9C) in serum-derived EV from patients with stroke and healthy donors. p-value of significance is shown above each box plot. Box and whiskers are shown for the 5th and 95th percentile. N.S.=Not Significant. ASC: N=16 control and 16 stroke; IL-1beta: N=10 control and 9 stroke; and IL-18: N=16 control and 13 stroke.

FIG. 10 illustrates Inflammasome proteins in serum-derived EV as biomarkers of stroke. ROC curves for ASC, IL-1beta and IL-18. ASC: N=16 control and 16 stroke; IL-1beta: N=10 control and 9 stroke; and IL-18: N=16 control and 13 stroke.

FIG. 11 illustrates a table containing the characteristics of the subjects with stroke from Example 2.

FIG. 12A-12D illustrates ROC curves for caspase-1 (FIG. 12A), ASC (FIG. 12B), IL-1beta (FIG. 12C) and IL-18 (FIG. 12D) from serum samples of stroke and healthy donors.

FIG. 13A-13F illustrates the characterization of inflammasome proteins in serum-derived EV. FIG. 13A depicts a representative image of immunoblot analyses of inflammasome proteins in EV from serum. Quantification of immunoblot analysis of (FIG. 13B) NLRP3, (FIG. 13C) caspase-1, (FIG. 13D) ASC, (FIG. 13E) IL-1beta, and (FIG. 13F) IL-18 in EV derived from serum using the Invitrogen kit (IN) and the ExoQuick kit (EQ). Data presented as mean+/−SEM. N=6 per group. * p<0.05.

FIG. 14A-14C illustrates ROC curves for ASC (FIG. 14A), IL-1beta (FIG. 14B) and IL-18 (FIG. 14C) from serum-derived extracellular vesicles of stroke and healthy donors.

FIG. 15A-15D illustrates how inflammasome proteins are elevated in the serum of TBI patients. Protein levels in pg/ml of ASC (FIG. 15A), caspase-1 (FIG. 15B), IL-18 (FIG. 15C) and IL-1β (FIG. 15D) in serum samples from patients with TBI and healthy donors (controls). ASC: N=120 control, 20 TBI. Caspase-1: N=11 control 19, TBI. IL-18: N=120 control, 21 TBI. IL-1β: N=25 control, 10 TBI. Box and whiskers are shown for the 5th and 95th percentile. * p<0.05.

FIG. 16A-16D illustrates ROC curves for caspase-1 (FIG. 16A), ASC (FIG. 16B), IL-1β (FIG. 16C) and IL-18 (FIG. 16D) from serum samples of TBI patients and healthy donors.

FIG. 17A-17B illustrates how inflammasome proteins are elevated in the CSF of TBI patients. Protein levels in pg/ml of ASC (FIG. 17A) and IL-18 (FIG. 17B) in CSF samples from patients with TBI and healthy donors (controls). ASC: N=21 control, 15 TBI. IL-18: N=24 control, 16 TBI. Box and whiskers are shown for the 5th and 95th percentile. * p<0.05.

FIG. 18A-18B illustrates ROC curves for ASC (FIG. 18A) and IL-18 (FIG. 18B) from CSF samples of TBI patients and healthy donors.

FIG. 19A-19C illustrates inflammasome proteins as prognostic biomarkers of TBI. Protein levels in pg/ml of caspase-1 (FIG. 19A), ASC (FIG. 19B), and IL-18 (FIG. 19C) in serum samples from patients with TBI. Groups were divided into favorable and unfavorable outcomes based on the GOSE. p-value of significance is shown above each box plot. Box and whiskers are shown for the 5th and 95th percentile. Caspase-1: N=4 favorable and 16 unfavorable ASC: N=5 favorable and 16 unfavorable; and IL-18: N=5 favorable and 16 unfavorable.

FIG. 20A-20B illustrates ROC curves for ASC outcomes (Favorable vs. Unfavorable) for the 2^{nd} (FIG. 20A) and 4th (FIG. 20B) collection.

FIG. 21A-21D illustrates inflammasome proteins are elevated in the serum of MCI and AD patients. Protein levels in pg/ml of ASC (FIG. 21A), caspase-1 (FIG. 21B), IL-18 (FIG. 21C) and IL-1beta (FIG. 21D) in serum samples from patients with MCI, AD, and age-matched healthy donors (control). * denotes p-value of significance compared to control, and ** denotes p-value of significance between MCI and AD. ASC: N=66 control, 32 MCI, 31 AD. Caspase-1: N=7 control, 23 MCI, 15 AD. IL-18: N=69 control, 31 MCI, 32 AD. IL-1beta: N=9 control, 9 MCI, 8 AD. Box and whiskers are shown for the 5th and 95th percentile. *** p<0.05.

FIG. 22A-22D illustrates ROC curves for ASC (FIG. 22A), caspase-1 (FIG. 22B), IL-18 (FIG. 22C) and IL-1beta (FIG. 22D) from serum samples of MCI and age-matched healthy donors.

FIG. 23A illustrates inflammasome proteins in serum as biomarkers of MCI. The ROC curves for caspase-1, ASC, IL-1beta and IL-18 from FIGS. 22A-22D are superimposed onto a single graph.

FIG. 23B illustrates inflammasome proteins in serum as biomarkers of AD. ROC curves for caspase-1, ASC, IL-1beta and IL-18 from serum samples of AD and aged-matched healthy donors are superimposed onto a single graph.

FIG. 23C illustrates inflammasome proteins in serum as biomarkers of MCI. ROC curves for caspase-1, ASC, IL-1beta and IL-18 from serum samples of AD and serum samples from MCI are superimposed onto a single graph.

FIG. 24A-24C illustrates proteins that are elevated in the serum of MCI and AD patients. Protein levels in pg/ml of sAPPα (FIG. 24A), sAPPβ (FIG. 24B), and NFL (FIG. 24C) in serum samples from patients with MCI, AD, and age-matched healthy donors (control).

FIG. 25A illustrates inflammasome proteins in serum as biomarkers of MCI. ROC curves for NFL, sAPPα, sAPPβ, and ASC from serum samples of MCI and aged-matched healthy donors are superimposed onto a single graph.

FIG. 25B illustrates inflammasome proteins in serum as biomarkers of AD. ROC curves for NFL, sAPPα, sAPPβ, and ASC from serum samples of MCI and aged-matched healthy donors are superimposed onto a single graph.

FIG. 25C illustrates inflammasome proteins in serum as biomarkers of MCI. ROC curves for NFL, sAPPα, sAPPβ, and ASC from serum samples of MCI and AD are superimposed onto a single graph.

FIG. 26A illustrates a linear regression analysis between IL-18 and ASC protein levels.

FIG. 26B illustrates a logarithmic transformation of linear regression analysis between IL-18 and ASC protein levels.

FIG. 26C illustrates a linear regression analysis between sAPPα and sAPPβ levels.

FIG. 26D illustrates a logarithmic transformation of linear regression analysis between sAPPα and sAPPβ protein levels.

FIG. 26E illustrates a fit of the linear regression analysis between IL-18 and ASC protein levels.

FIG. 26F illustrates a fit of the logarithmic transformation of the linear regression analysis between IL-18 and ASC protein levels.

FIG. 26G illustrates residual analysis results of the linear regression analysis between IL-18 and ASC protein levels.

FIG. 26H illustrates residual analysis results of the logarithmic transformation of the linear regression analysis between IL-18 and ASC protein levels.

FIG. 26I illustrates a fit of the linear regression analysis between sAPPα and sAPPβ protein levels.

FIG. 26J illustrates a fit of the logarithmic transformation of the linear regression analysis between sAPPα and sAPPβ protein levels.

FIG. 26K illustrates residual analysis results of the linear regression analysis between sAPPα and sAPPβ protein levels.

FIG. 26L illustrates residual analysis results of the logarithmic transformation of the linear regression analysis between sAPPα and sAPPβ protein levels.

FIG. 27A illustrates cluster analysis using ASC protein levels in control, MCI, and AD patients. FIG. 27A shows clustering using a Gaussian Mixture Modelling method.

FIG. 27B illustrates cluster analysis using ASC protein levels in control, MCI, and AD patients. FIG. 27B shows a cluster dendrogram.

FIG. 27C illustrates cluster analysis using ASC protein levels in control, MCI, and AD patients. FIG. 27C shows a coordinate plot.

FIG. 28A-28D illustrates inflammasome proteins are elevated in the serum of AMD patients. Protein levels in pg/ml of ASC (FIG. 28A), caspase-1 (FIG. 28B), IL-18 (FIG. 28C) and IL-1beta (FIG. 28D) in serum samples from patients with AMD are shown.

FIG. 29A-29D illustrates ROC curves for ASC (FIG. 29A), caspase-1 (FIG. 29B), IL-18 (FIG. 29C) and IL-1beta (FIG. 29D) from serum samples of AMD donors.

FIG. 30A-30D illustrates the expression of the inflammasome proteins ASC (FIG. 30A), caspase-1 (FIG. 30B), IL-18 (FIG. 30C) and IL-1beta (FIG. 30D) in patients with wet AMD and patients with dry AMD.

FIG. 31 illustrates residual analysis results of the linear regression analysis between ASC and IL-18 protein levels in patients with AMD.

FIG. 32 illustrates a binomial logistic regression for the protein levels of ASC in serum or patients with and without an AMD diagnosis.

FIG. 33 illustrates a binomial logistic regression for the protein levels of IL-18 in serum or patients with and without an AMD diagnosis.

FIG. 34 illustrates that a monoclonal antibody directed against ASC (i.e., IC-100 (mAb)) inhibits IL-1beta activation in the cortex of aged mice. Mice were treated with IC-100 (5 mg/kg) and saline control (i.p.) and sacrificed 3 days later. Immunoblot of cortical protein lysates of young (3 months) and aged (18 months) mice blotted for IL-1beta. Data presented as mean+/−SEM. 3 m: 3 months, 18 m: 18 months. Sal: Saline. N=6 per group. * p<0.05.

FIG. 35A-35D illustrates that a monoclonal antibody directed against ASC (i.e., IC-100 (MAb)) inhibits NLRP1 inflammasome activation in the cortex of aged mice. Mice were treated with IC-100 (5 mg/kg) and saline control (i.p.) and sacrificed 3 days later. FIG. 35A shows a representative immunoblot of cortical protein lysates of young (3 months) and aged (18 months) mice blotted for NLRP1, caspase-1 and ASC, while FIGS. 35B-35D depict the relative density units for NLRP1 (FIG. 35B), caspase-1 (FIG. 35C) and ASC (FIG. 35D) as determined from representative immunoblots such as the immunoblot depicted in FIG. 35A. Data presented as mean+/−SEM. 3 m: 3 months, 18 m: 18 months. Sal: Saline. N=6 per group. * p<0.05.

FIG. 36A-36C illustrates that a monoclonal antibody directed against ASC (i.e., IC-100 (MAb)) inhibits non-canonical inflammasome activation in the cortex of aged mice. Mice were treated with IC-100 (5 mg/kg) and saline control (i.p.) and sacrificed 3 days later. FIG. 36A shows a representative immunoblot of cortical protein lysates of young (3 months) and aged (18 months) mice blotted for caspase-8 and caspase-11, while FIGS. 36B-36C depict the relative density units for caspase-8 (FIG. 36B) and caspase-11 (FIG. 35C) as determined from representative immunoblots such as the immunoblot depicted in FIG. 36A. Data presented as mean+/−SEM. 3 m: 3 months, 18 m: 18 months. Sal: Saline. N=6 per group. * p<0.05.

FIG. 37 illustrates formation of the non-canonical NLRP1-ASC-caspase-8 inflammasome in the cortex of aged mice. Cortical protein lysates of aged (18 months saline and IC-100 treated) and young mice (3 months) were co-immunoprecipitated (IP) with IC-100 (Anti-ASC) and blotted for ASC, caspase-8, NLRP1 and caspase-1 indicating protein-protein interactions among these proteins. 3 m: 3 months, 18 m: 18 months. Sal: Saline.

FIG. 38 shows the results of a linear regression analysis between ASC and the pro-inflammatory cytokine IL-18.

FIG. 39 shows results of the analysis of the residuals in order to evaluate the fit of the linear model.

FIG. 40 shows the estimate coefficient of ASC following a binomial logistic regression for the proteins levels of ASC in serum of patients with and without an AMD diagnosis.

FIG. 41 shows the estimate coefficient of IL-18 following a binomial logistic regression for the proteins levels of ASC in serum of patients with and without an AMD diagnosis.

FIG. 42A-42D illustrates the expression of the inflammasome proteins ASC (FIG. 42A) and IL-18 (FIG. 42B) as well as known NASH biomarkers Gal-3 (FIG. 42C) and C-Reactive protein (CRP; FIG. 42D) from serum samples of patients with NASH.

FIG. 43A-43D illustrates ROC curves for ASC (FIG. 43A), IL-18 (FIG. 43B), Gal-3 (FIG. 43C) and C-Reactive Protein (FIG. 43D) from serum samples of NASH donors.

FIG. 44 illustrates inflammasome proteins in serum as biomarkers of NASH. The ROC curves for IL-18, ASC and Gal-3 from FIGS. 43A-43C are superimposed onto a single graph.

### DETAILED DESCRIPTION

### Definitions

Unless otherwise defined, all technical terms used herein have the same meaning as commonly understood by one of ordinary skill in the art to which this invention belongs.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world. Although compositions and methods similar or equivalent to those described herein can be used in the practice or testing of the present invention, suitable compositions and methods are described below.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”. The use of the alternative (e.g., “or”) should be understood to mean either one, both, or any combination thereof of the alternatives. As used herein, the terms “about” and “consisting essentially of” mean+/−20% of the indicated range, value, or structure, unless otherwise indicated.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. The particular embodiments discussed below are illustrative only and not intended to be limiting. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Throughout this disclosure, various aspects of the methods and compositions provided herein can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, “protein” and “polypeptide” are used synonymously to mean any peptide-linked chain of amino acids, regardless of length or post-translational modification, e.g., glycosylation or phosphorylation.

As used herein, the term “antibody” refers generally and broadly to immunoglobulins (Ig) molecules and immunologically active portions or fragments of immunoglobulin molecules, i.e., molecules that contain an antigen binding site that specifically binds (immunoreacts with) an antigen (e.g., ASC, NLRP1, AIM2, etc.). The antibodies provided herein can be polyclonal antibodies, monoclonal antibodies (mAbs), chimeric antibodies, humanized antibodies, anti-idiotypic (anti-Id) antibodies to antibodies that can be labeled in soluble or bound form, as well as active fragments, regions or derivatives thereof. The antibodies for use herein may be chimeric, humanized, or human.

By “specifically binds” or “immunoreacts with” is meant that the antibody reacts with one or more antigenic determinants of the desired antigen and does not react with other polypeptides. In certain embodiments, an antibody is said to specifically bind an antigen when it preferentially recognizes its target antigen in a complex mixture of proteins and/or macromolecules. The term “antibody” broadly refers to an immunoglobulin (Ig) molecule, generally comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art. Such anti-ASC and anti-NLRP1 antibodies of the present invention are capable of binding portions of ASC and NLRP1, respectively, which interfere with caspase-1 activation.

As used herein, the term “humanized antibody” refers to an antibody in which minimal portions of a non-human antibody are introduced into an otherwise human antibody.

As used herein, the term “human antibody” refers to an antibody in which substantially every part of the protein is substantially non-immunogenic in humans, with only minor sequence changes or variations.

In a full-length antibody, each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY) and class (e.g., IgG1, IgG2, IgG3, IgG4, IgA1 and IgA2) or subclass. IgG, IgD, and IgE antibodies generally contain two identical heavy chains and two identical light chains and two antigen combining domains, each composed of a heavy chain variable region (VH) and a light chain variable region (VL). Generally IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies); in this way the IgM molecule has ten antigen binding domains, each again composed of a VH and a VL. A cell surface form of IgM also exists and this has two heavy chain/two light chain structure similar to IgG, IgD, and IgE antibodies.

The term “antigen binding fragment” or “antigen binding portion” or “antigen binding site” or “binding domain” or “binding region”, as used herein, can refer to the domain, region, portion, or site of a protein, polypeptide, oligopeptide, or peptide or antibody or binding domain derived from an antibody that retains the ability to specifically bind to an antigen (e.g., ASC protein). Exemplary binding domains include single-chain antibody variable regions (e.g., domain antibodies, sFv, scFv, scFab), fusion proteins comprising an antibody portion (e.g., a domain antibody), receptor ectodomains, and ligands (e.g., cytokines, chemokines). In one embodiment, the fusion protein comprises one or more CDR(s). In another embodiment, the fusion protein comprises CDR H3 (VH CDR3) and/or CDR L3 (VL CDR3). For purposes of this invention, a fusion protein contains one or more antibodies and additional amino acid sequence such as for example, a heterologous sequence or a homologous sequence from another region, attached to the N- or C-terminus of the antibody or antibody fragment thereof. Exemplary heterologous sequences include, but are not limited to a “tag” such as a FLAG tag or a 6His tag or an enzyme or a polypeptide which increases the half-life of the antibody in the blood. Tags are well known in the art. The additional amino acid sequence, which can include amino- and/or carboxyl-terminal fusions can range in length from one residue to polypeptides containing a hundred or more residues, as well as intra-sequence insertions of single or multiple amino acid residues.

An antigen binding site can be generally formed by the heavy chain variable region (VH) and the light chain variable region (VL) immunoglobulin domains, with the antigen-binding interface formed by six surface polypeptide loops, termed complementarity determining regions (CDRs). There are three CDRs each in VH (HCDR1, HCDR2, HCDR3) and VL (LCDR1, LCDR2, LCDR3), together with framework regions (FRs). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions).

The term “CDR region” or “CDR” can be mean the hypervariable regions of the heavy or light chains of the immunoglobulin as defined by Kabat et al., 1991 (Kabat, E. A. et al., (1991) Sequences of Proteins of Immunological Interest, 5th Edition. US Department of Health and Human Services, Public Service, NIH, Washington), and later editions. An antibody typically contains 3 heavy chain CDRs and 3 light chain CDRs.

It has been shown that the antigen binding function of an antibody can be performed by fragments of a full-length antibody. Antibody and antibody fragment embodiments may also be bispecific, trispecific, dual specific, or multi-specific formats; specifically binding to two or more different antigens. Examples of binding fragments encompassed within the term “antigen binding fragment” of an antibody include: (i) an Fab fragment consisting of VL, VH, CL and CH1 domains (Ward, E. S. et al., (1989) Nature 341, 544-546); (ii) an Fd fragment consisting of the VH and CH1 domains (McCafferty et al., (1990) Nature, 348, 552-554); (iii) an Fv fragment consisting of the VL and VH domains of a single antibody (Holt et al., (2003) Trends in Biotechnology 21, 484-490); (iv) a dAb fragment (Ward, E. S. et al., Nature 341, 544-546 (1989), McCafferty et al., (1990) Nature, 348, 552-554, Holt et al., (2003) Trends in Biotechnology 21, 484-490], which consists of a VH or a VL domain; (v) isolated CDR regions; (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., (1988) Science, 242, 423-426, Huston et al., (1988) PNAS USA, 85, 5879-5883). The invention also encompasses a Fab′ fragment. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen binding fragment” of an antibody. In certain embodiments of the invention, scFv molecules may be incorporated into a fusion protein. In some embodiments, the invention includes a single chain camelid antibody; (viii) bispecific single chain Fv dimers (PCT/U.S. Pat. No. 92,109,965) and (ix) “diabodies”, multivalent or multispecific fragments constructed by gene fusion (WO94/13804; Holliger, P. (1993) et al., Proc. Natl. Acad. Sci. USA 90 6444-6448). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding fragments are known in the art (Kontermann and Dubel eds., Antibody Engineering (2001) Springer-Verlag. New York. 790 pp.). In some aspects, the invention includes a single domain antibody. In general, the term “antibody” when used herein encompasses an “antibody fragment”. An antibody fragment generally retains the antigen-binding properties of a full length antibody.

Fv, scFv or diabody molecules may be stabilized by incorporation of disulfide bridges linking the VH and VL domains (Reiter, Y. et al., Nature Biotech, 14, 1239-1245, 1996). Minibodies comprising a scFv joined to a CH3 domain may also be made (Hu, S. et al., (1996) Cancer Res., 56, 3055-3061). Other examples of binding fragments can be Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

“Fv” when used herein can refer to the minimum fragment of an antibody that retains both antigen-recognition and antigen-binding sites. “Fab” when used herein can refer to a fragment of an antibody that comprises the constant domain of the light chain and the CH1 domain of the heavy chain. The term “mAb” refers to monoclonal antibody.

“Fc region” or “Fc domain” refers to a polypeptide sequence corresponding to or derived from the portion of a source antibody that is responsible for binding to antibody receptors on cells and the C1q component of complement. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, CH2, and CH3 domains. However, more recently the term has been applied to a single chain consisting of CH3, CH2, and at least a portion of the hinge sufficient to form a disulfide-linked dimer with a second such chain. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences. In one embodiment, the antibodies or antibody fragments derived therefrom provided herein (e.g., the anti-ASC monoclonal antibodies or antibody fragments thereof) have a modified Fc region or domain. In some cases, the modified Fc region or domain can confer increased thermal stability to the resultant antibody or antibody fragment derived therefrom. The increased thermal stability can result in increased serum half-life. The Fc region or domain can be modified as described in US20160193295, the contents of which are herein incorporated by reference. As described in US20160193295, the Fc region or domain can be modified to possess a deletion of one or more cysteine residues in the hinge region and substitution with a sulfhydryl-containing residue of one or more CH3-interface amino acids. In another embodiment, the Fc region or domain of the antibodies or antibody fragments derived therefrom provided herein (e.g., the anti-ASC monoclonal antibodies or antibody fragments thereof) can be stabilized by engineering the Fc region to possess intradomain disulfide bonds as described in Wozniak-Knopp G, Stadlmann J, Rüker F (2012) Stabilization of the Fc Fragment of Human IgG1 by Engineered Intradomain Disulfide Bonds. PLoS ONE 7(1): e30083, the contents of which are herein incorporated by reference. In yet another embodiment, the antibodies have Fc regions modified as described in WO 99/58572, which is herein incorporated by reference. In still other embodiments, the Fc region or domain can be modified as described in U.S. Pat. No. 9,574,010, the contents of which are herein incorporated by reference.

As used herein, the term “epitope” includes any protein determinant capable of specific binding to an immunoglobulin or an immunoglobulin fragment. Epitopic determinants usually consist of chemically active surface groupings of molecules such as amino acids or sugar side chains and usually have specific three dimensional structural characteristics, as well as specific charge characteristics. The term “epitope” also refers to a unit of structure conventionally bound by an immunoglobulin heavy chain variable (VH) region and a light chain variable (VL) region pair. An epitope may define the minimum binding site for an antibody, and thus represent the target of specificity of an antibody.

By the terms “Apoptosis-associated Speck-like protein containing a Caspase Activating Recruitment Domain (CARD)” and “ASC” is meant an expression product of an ASC gene or isoforms thereof, or a protein that shares at least 65%, 75%, 80%, 85%, 90%, 95%, 96%, 97%, 98%, or 99% amino acid sequence identity with ASC (e.g., NP_037390 (Q9ULZ3-1), NP_660183 (Q9ULZ3-2) or Q9ULZ3-3 in human or NP_758825 (BAC43754) in rat) and displays a functional activity of ASC. A “functional activity” of a protein is any activity associated with the physiological function of the protein. Functional activities of ASC include, for example, recruitment of proteins for activation of caspase-1 and initiation of cell death.

By the term “ASC gene,” or “ASC nucleic acid” is meant a native ASC-encoding nucleic acid sequence, genomic sequences from which ASC cDNA can be transcribed, and/or allelic variants and homologues of the foregoing. The terms encompass double-stranded DNA, single-stranded DNA, and RNA.

As used herein, the term “inflammasome” or “canonical inflammasome” means a multi-protein (e.g., at least two proteins) complex that activates caspase-1. Further, the term “inflammasome” can refer to a multi-protein complex that activates caspase-1 activity, which in turn regulates IL-1β, IL-18 and IL-33 processing and activation. See Arend et al. 2008; Li et al. 2008; and Martinon et al. 2002, each of which is incorporated by reference in their entireties. The terms “NLRP1 inflammasome”, “NALP1 inflammasome”, “NLRP2 inflammasome”, “NALP2 inflammasome”, “NLRP3 inflammasome”, “NALP3 inflammasome”, “NLRC4 inflammasome”, “IPAF inflammasome” or “AIM2 inflammasome” mean a protein complex of at least caspase-1 and one adaptor protein, e.g., ASC. For example, the terms “NLRP1 inflammasome” and “NALP1 inflammasome” can mean a multiprotein complex containing NLRP1, ASC, caspase-1, caspase-11, XIAP, and pannexin-1 for activation of caspase-1 and processing of interleukin-1β, interleukin-18 and interleukin-33. The terms “NLRP2 inflammasome” and “NALP2 inflammasome” can mean a multiprotein complex containing NLRP2 (aka NALP2), ASC and caspase-1, while the terms “NLRP3 inflammasome” and “NALP3 inflammasome” can mean a multiprotein complex containing NLRP3 (aka NALP3), ASC and the terms “NLRC4 inflammasome” and “IPAF inflammasome” can mean a multiprotein complex containing NLRC4 (aka IPAF), ASC and caspase-1. Additionally, the term “AIM2 Inflammasome” can mean a multiprotein complex comprising AIM2, ASC and caspase-1.

As used herein, the term “non-canonical inflammasome” means a multi-protein (e.g., at least two proteins) complex that activates a caspase other than caspase-1. The non-canonical inflammasome can be comprised of an NLR such as NLRP1 or NLRP3 that interacts with a caspase other than caspase-1. For example, the non-canonical NLRP1-caspase-8 inflammasome is comprised of NLRP-1, caspase-8 and ASC.

As interchangeably used herein, “amyloid precursor protein” and “APP” can mean an expression product of an APP gene or isoforms, a cleavage product of APP, or a protein that shares at least 65%, 75%, 80%, 85%, 90%, 95%, 96%, 97%, 98%, or 99% amino acid sequence identity with APP (e.g., accession number(s) NP001129603.1, NP_001129601.1, P05067). Non-limiting examples of cleavage products of APP (SEQ ID NO: 36) include soluble amyloid precursor protein α (sAPPα) (SEQ ID NO: 37), soluble amyloid precursor protein β (sAPPβ) (SEQ ID NO: 38), amyloid-β 1-42 (Aβ₍₁₋₄₂₎) (SEQ ID NO: 39), or amyloid-β 1-40 (Aβ₍₁₋₄₀₎) (SEQ ID NO: 40).

As interchangeably used herein, “neurofilament light chain,” “NfL,” and “NFL” can mean an expression product of an NFL gene or isoforms, a cleavage product of NFL, or a protein that shares at least 65%, 75%, 80%, 85%, 90%, 95%, 96%, 97%, 98%, or 99% amino acid sequence identity with NFL (e.g., accession number(s) P07196) (SEQ ID NO: 41).

As used herein, a “control biomarker” or “control biomarker protein” can mean any gene, expression product of a gene, or protein that is utilized in the compositions and methods of the disclosure that is known in the art to be associated with or indicative or diagnostic of a brain injury. For example, the brain injury can be MCI and/or AD and the control biomarker or control biomarker protein can be NFL, amyloid-β (Aβ₍₁₋₄₂₎), T-Tau, sAPPα, or sAPPβ. In some cases, the control biomarkers for a specific brain injury can be referred to as a control biomarker for that specific brain injury. For example, a control biomarker for MCI or AD can be referred to as a control MCI biomarker or control AD biomarker, respectively.

As used herein, the phrase “sequence identity” means the percentage of identical subunits at corresponding positions in two sequences (e.g., nucleic acid sequences, amino acid sequences) when the two sequences are aligned to maximize subunit matching, i.e., taking into account gaps and insertions. Sequence identity can be measured using sequence analysis software (e.g., Sequence Analysis Software Package from Accelrys CGC, San Diego, Calif.).

By the phrases “therapeutically effective amount” and “effective dosage” is meant an amount sufficient to produce a therapeutically (e.g., clinically) desirable result; the exact nature of the result will vary depending on the nature of the disorder being treated. For example, where the disorder to be treated is SCI, the result can be an improvement in motor skills and locomotor function, a decreased spinal cord lesion, etc. The compositions described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean a mammalian subject to be treated, such as, for example, human patients. In some cases, the methods of the invention find use in experimental animals, in veterinary applications, and in the development of animal models for disease, including, but not limited to, rodents including mice, rats, and hamsters, as well as primates.

As interchangeably used herein, “Absent in Melanoma 2” and “AIM2” can mean an expression product of an AIM2 gene or isoforms; or a protein that shares at least 65%, 75%, 80%, 85%, 90%, 95%, 96%, 97%, 98%, or 99% amino acid sequence identity with AIM2 (e.g., accession number(s) NX_014862, NP004824, XP016858337, XP005245673, AAB81613, BAF84731, AAH10940) and displays a functional activity of AIM2.

As interchangeably used herein, “NALP1” and “NLRP1” mean an expression product of an NALP1 or NLRP1 gene or isoforms; or a protein that shares at least 65%, 75%, 80%, 85%, 90%, 95%, 96%, 97%, 98%, or 99% amino acid sequence identity with NALP1 (e.g., accession number(s) AAH51787, NP_001028225, NP_127500, NP_127499, NP_127497, NP055737) and displays a functional activity of NALP1.

As interchangeably used herein, “NALP2” and “NLRP2” mean an expression product of an NALP2 or NLRP2 gene or isoforms; or a protein that shares at least 65%, 75%, 80%, 85%, 90%, 95%, 96%, 97%, 98%, or 99% amino acid sequence identity with NALP2 (e.g., accession number(s) NP_001167552, NP_001167553, NP_001167554 or NP_060322) and displays a functional activity of NALP2.

As interchangeably used herein, “NALP3” and “NLRP3” mean an expression product of an NALP3 or NLRP3 gene or isoforms; or a protein that shares at least 65%, 75%, 80%, 85%, 90%, 95%, 96%, 97%, 98%, or 99% amino acid sequence identity with NALP3 (e.g., accession number(s) NP_001073289, NP_001120933, NP_001120934, NP_001230062, NP_004886, NP_899632, XP_011542350, XP_016855670, XP_016855671, XP_016855672 or XP_016855673) and displays a functional activity of NALP3.

As interchangeably used herein, “NLRC4” and “IPAF” mean an expression product of an NLRC4 or IPAF gene or isoforms; or a protein that shares at least 65%, 75%, 80%, 85%, 90%, 95%, 96%, 97%, 98%, or 99% amino acid sequence identity with NLRC4 (e.g., accession number(s) NP_001186067, NP001186068, NP_001289433 or NP_067032) and displays a functional activity of NLRC4.

By the term “stroke” and “ischemic stroke” is meant when blood flow is interrupted to part of the brain or spinal cord. By the term “ischemic stroke” and “transient ischemic stroke” is meant when blood flow is interrupted to part of the brain or spinal cord by blockage of an artery that supplies oxygen-rich blood to the brain or spinal cord. By the term “hemorrhagic stroke” is meant when blood flow is interrupted to part of the brain or spinal cord when an artery in the brain or spinal cord leaks blood or ruptures.

By “traumatic injury to the CNS” is meant any insult to the CNS from an external mechanical force, possibly leading to permanent or temporary impairments of CNS function.

The term ‘inflammaging’ as used herein can refer to a chronic, low-grade inflammation that can occur as an organism ages. Inflammaging can be macrophage centered, involve several tissues and organs, including the gut microbiota, and can be characterized by a complex balance between pro- and anti-inflammatory responses. In some cases, inflammaging can refer to a chronic, pro-inflammatory state. The major source of inflammatory stimuli that can characterize or be associated with inflammaging can be represented by endogenous/self, misplaced, or altered molecules resulting from damaged and/or dead cells and organelles (cell debris), recognized by receptors of the innate immune system. While their production is physiological and increases with age, their disposal by the proteasome via autophagy and/or mitophagy progressively declines. This ‘autoreactive/autoimmune’ process can fuel the onset or progression of chronic diseases that can accelerate and propagate the aging process locally and systemically.

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Immunology techniques are generally known in the art and are described in detail in methodology treatises such as Advances in Immunology, volume 93, ed. Frederick W. Alt, Academic Press, Burlington, Mass., 2007; Making and Using Antibodies: A Practical Handbook, eds. Gary C. Howard and Matthew R. Kaser, CRC Press, Boca Raton, Fla., 2006; Medical Immunology, 6^{th} ed., edited by Gabriel Virella, Informa Healthcare Press, London, England, 2007; and Harlow and Lane ANTIBODIES: A Laboratory Manual, Cold Spring Harbor Laboratory Press, Cold Spring Harbor, N.Y., 1988.

Overview

Provided herein are compositions and methods for diagnosing or evaluating a patient suspected of having inflammation or a disease, disorder or condition caused by or associated with inflammation. The method can comprise measuring the level of at least one inflammasome protein in a biological sample obtained from the patient; determining the presence or absence of a protein signature associated with inflammation or the disease, disorder or condition caused by or associated with inflammation, wherein the protein signature comprises an elevated level of the at least one inflammasome protein; and selecting the patient as having inflammation or the disease, disorder or condition caused by or associated with inflammation if the patient exhibits the presence of the protein signature. In some cases, the method further comprises measuring an expression level of at least one control biomarker protein and wherein the protein signature further comprises an elevated expression level of at least one control biomarker protein. The at least one control biomarker protein is any protein whose expression level has been previously shown to be associated with inflammation or the disease, disorder or condition caused by or associated with inflammation. The inflammation can be an innate immune inflammation. The inflammation can be an inflammasome-related inflammation. The disease, disorder or condition can be selected from the group consisting of a brain injury, an age-related disease, inflammaging, an autoimmune, autoinflammatory, metabolic or neurodegenerative disease. In some cases, the disease, disorder or condition is inflammaging. In some cases, the age-related disease is age-related macular degeneration (AMD). In some cases, the disease, disorder or condition is a brain injury. The brain injury can be selected from the group consisting of traumatic brain injury (TBI), stroke and spinal cord injury (SCI). The autoimmune or neurodegenerative disease can be selected from amyotrophic lateral sclerosis (ALS), Alzheimer's disease (AD), Parkinson's disease (PD), muscular dystrophy (MD), immune dysfunction muscular CNS breakdown, systemic lupus erythematosus, lupus nephritis, rheumatoid arthritis, inflammatory bowel disease (e.g., Crohn's Disease and ulcerative colitis) and multiple sclerosis (MS). The metabolic disease can be selected from metabolic syndrome, obesity, diabetes mellitus, diabetic nephropathy or diabetic kidney disease (DKD), insulin resistance, atherosclerosis, a lipid storage disorder, a glycogen storage disease, medium-chain acyl-coenzyme A dehydrogenase deficiency, non-alcoholic fatty liver disease (e.g., Nonalcoholic steatohepatitis (NASH)) and gout. The autoinflammatory disease can be cryopyrin-associated periodic syndrome (CAPS). CAPS can encompass familial cold autoinflammatory syndrome (FCAS), Muckle-Wells syndrome (MWS) and neonatal-onset multisystem inflammatory disease (NOMID). In one embodiment, the brain injury is MS. In another embodiment, the brain injury is stroke. In yet another embodiment, the brain injury is TBI. In still another embodiment, the brain injury is MCI. In still another embodiment, the brain injury is AD. In embodiments where the brain injury is MCI or AD, the control biomarker proteins can be NFL, amyloid-β (Aβ₍₁₋₄₂₎), T-Tau, sAPPα, sAPPβ or any combination thereof. The disease, disorder or condition can be inflammaging or an age-related disease. In another embodiment, the age-related disease is age-related macular degeneration (AMD).

Also provided herein are methods treating patients suffering from or suspected of suffering from inflammation or a disease, disorder or condition caused by or associated with inflammation. The inflammation can be an innate immune inflammation. The inflammation can be an inflammasome-related inflammation. The disease, disorder or condition can be selected from the group consisting of a brain injury, an age-related disease, inflammaging, an autoimmune, autoinflammatory, metabolic or neurodegenerative disease. In some cases, the disease, disorder or condition is inflammaging. In some cases, the age-related disease is age-related macular degeneration (AMD). In some cases, the disease, disorder or condition is a brain injury. The brain injury can be selected from the group consisting of traumatic brain injury (TBI), stroke and spinal cord injury (SCI). The autoimmune or neurodegenerative disease can be selected from amyotrophic lateral sclerosis (ALS), Alzheimer's disease (AD), Parkinson's disease (PD), muscular dystrophy (MD), immune dysfunction muscular CNS breakdown, systemic lupus erythematosus, lupus nephritis, rheumatoid arthritis, inflammatory bowel disease (e.g., Crohn's Disease and ulcerative colitis) and multiple sclerosis (MS). The metabolic disease can be selected from metabolic syndrome, obesity, diabetes mellitus, diabetic nephropathy or diabetic kidney disease (DKD), insulin resistance, atherosclerosis, a lipid storage disorder, a glycogen storage disease, medium-chain acyl-coenzyme A dehydrogenase deficiency, non-alcoholic fatty liver disease (e.g., Nonalcoholic steatohepatitis (NASH)) and gout. The autoinflammatory disease can be cryopyrin-associated periodic syndrome (CAPS). CAPS can encompass familial cold autoinflammatory syndrome (FCAS), Muckle-Wells syndrome (MWS) and neonatal-onset multisystem inflammatory disease (NOMID). Any method of treating provided herein can entail administering a treatment to the patients suffering from or suspected of suffering from the disease, disorder or condition associated with inflammation. Administration of the treatment in a method for treating a disease, disorder or condition associated with inflammation as provided herein can reduce inflammation in the patient. The reduction can be as compared to a control (e.g., untreated patient and/or patient prior to treatment). In some cases, the treatment is a standard of care treatment. In some cases, the treatment is a neuroprotective treatment. Such neuroprotective treatments can include drugs that reduce excitotoxicity, oxidative stress, and inflammation. Thus, suitable neuroprotective treatments include, but are not limited to, methylprednisolone, 17alpha-estradiol, 17beta-estradiol, ginsenoside, progesterone, simvastatin, deprenyl, minocycline, resveratrol, and other glutamate receptor antagonists (e.g. NMDA receptor antagonists) and antioxidants. In some embodiments, the treatments are antibodies against an inflammasome protein or binding fragments thereof, such as the antibodies directed against inflammasome proteins provided herein.

Also provided herein are monoclonal antibodies or an antibody fragments thereof that bind specifically to Apoptosis-associated Spec-like protein containing a Caspase Activating Recruitment Domain (ASC). The monoclonal antibodies or fragments thereof can bind specifically to an antigenic fragment of ASC that comprises, consists of or consists essentially of an amino acid sequence of KKFKLKLLSVPLREGYGRIPR (SEQ ID NO. 5). Further to this embodiment, the invention contemplates use of the monoclonal antibodies or antibody fragments thereof in a method for treating inflammation in a subject. The inflammation can be caused by the patient suffering from disease, disorder or condition associated with inflammation. The inflammation can be an innate immune inflammation. The inflammation can be an inflammasome-related inflammation. The disease, disorder or condition can be selected from the group consisting of a brain injury, an age-related disease, inflammaging, an autoimmune, autoinflammatory, metabolic or neurodegenerative disease. In some cases, the disease, disorder or condition is inflammaging. In some cases, the age-related disease is age-related macular degeneration (AMD). In some cases, the disease, disorder or condition is a brain injury. The brain injury can be selected from the group consisting of traumatic brain injury (TBI), stroke and spinal cord injury (SCI). The autoimmune or neurodegenerative disease can be selected from amyotrophic lateral sclerosis (ALS), Alzheimer's disease (AD), Parkinson's disease (PD), muscular dystrophy (MD), immune dysfunction muscular CNS breakdown, systemic lupus erythematosus, lupus nephritis, rheumatoid arthritis, inflammatory bowel disease (e.g., Crohn's Disease and ulcerative colitis) and multiple sclerosis (MS). The metabolic disease can be selected from metabolic syndrome, obesity, diabetes mellitus, diabetic nephropathy or diabetic kidney disease (DKD), insulin resistance, atherosclerosis, a lipid storage disorder, a glycogen storage disease, medium-chain acyl-coenzyme A dehydrogenase deficiency, non-alcoholic fatty liver disease (e.g., Nonalcoholic steatohepatitis (NASH)) and gout. The autoinflammatory disease can be cryopyrin-associated periodic syndrome (CAPS). CAPS can encompass familial cold autoinflammatory syndrome (FCAS), Muckle-Wells syndrome (MWS) and neonatal-onset multisystem inflammatory disease (NOMID). In one embodiment, the monoclonal antibodies or antibody fragments thereof provided herein can be used in a method for reducing inflammation in a mammal as described in U.S. Pat. No. 8,685,400, the contents of which are herein incorporated by reference in their entirety. The monoclonal antibody or antibody fragment thereof of this embodiment can be present in a composition such as, for example, a pharmaceutical composition as provided herein. In some cases, the monoclonal antibody or fragment thereof is used in combination with one or more other agents in the methods of treatment provided herein. The other agents can be any agent provided herein (e.g., EV uptake inhibitors) and/or antibodies or antibody fragments directed against other inflammasome components (e.g., IL-18, caspase-1, NALP1, AIM2, etc.).

Diagnostic Methods

In some cases, provided herein are methods for diagnosing or evaluating a patient suspected of having inflammation or a disease, disorder or condition caused by or associated with inflammation that can comprise detecting an expression level of at least one inflammasome protein in a biological sample obtained from a patient suspected of suffering from inflammation or a disease, disorder or condition caused by or associated with inflammation, detecting an expression level of at least one control protein in a control biological sample; comparing the expression level of the at least one inflammasome protein in the biological sample obtained from the patient suspected of suffering from inflammation or the disease, disorder or condition caused by or associated with inflammation and the expression level of the at least control protein in the control biological sample; and selecting the patient as having inflammation or the disease, disorder or condition caused by or associated with inflammation based on the comparison. In some cases, an increased expression level of the detected expression level of the at least one inflammasome protein in the biological sample obtained from the patient suspected of suffering from inflammation or the disease, disorder or condition caused by or associated with inflammation as compared to the expression level of the at least one control protein in the control biological sample selects the patient as having inflammation or the disease, disorder or condition caused by or associated with inflammation. In some cases, a decreased expression level of the detected expression level of the at least one inflammasome protein in the biological sample obtained from the patient suspected of suffering from inflammation or the disease, disorder or condition caused by or associated with inflammation as compared to the expression level of the at least one control protein in the control biological sample selects the patient as having inflammation or the disease, disorder or condition caused by or associated with inflammation. In some cases, the control biological sample can be a biological sample obtained from a subject not suspected of suffering from inflammation or the disease, disorder or condition caused by or associated with inflammation and the at least one control protein can be the at least one inflammasome protein detected in the biological sample obtained from the patient suspected of suffering from inflammation or the disease, disorder or condition caused by or associated with inflammation. In some cases, the control biological sample can be a biological sample obtained from the patient suspected of suffering from inflammation or the disease, disorder or condition caused by or associated with inflammation and the at least one control protein can be a control biomarker protein. The control biomarker protein can be any protein whose expression level has been previously shown to be associated with inflammation or the disease, disorder or condition caused by or associated with inflammation. In one embodiment, an elevated expression level of the control biomarker protein has been previously shown to be associated with or diagnostic of inflammation or the disease, disorder or condition caused by or associated with inflammation. In one embodiment, the disease, disorder or condition caused by or associated with inflammation is MCI or AD and the at least one control protein is a control biomarker protein selected from NFL, amyloid-β (Aβ₍₁₋₄₂₎), T-Tau, sAPPα, and sAPPβ. In one embodiment, the disease, disorder or condition caused by or associated with inflammation is NASH and the at least one control protein is a control biomarker protein selected from Gal-3 and CRP (hs-CRP). In one embodiment, any method provided herein for diagnosing or evaluating a disease, disorder or condition caused by or associated with inflammation in a patient suspected of suffering from the disease, disorder or condition caused by or associated with inflammation by measuring an expression level of at least one inflammasome protein in a biological sample obtained from the patient can be performed in combination with determining the expression level of biomarkers whose altered expression levels are known or suspected to be associated with the disease, disorder or condition caused by or associated with inflammation. In one embodiment, any method provided herein for diagnosing or evaluating a disease, disorder or condition caused by or associated with inflammation in a patient suspected of suffering from the disease, disorder or condition caused by or associated with inflammation by the measuring the expression level of at least one inflammasome protein in a biological sample obtained from the patient can be performed in combination with one or more additional diagnostic assessments. Detection of an altered expression level of the at least inflammasome protein in the biological sample obtained from the patient can be used to confirm a diagnosis of a particular disease, disorder or condition caused by or associated with inflammation determined using one or more additional diagnostic assessments. Detection of an altered expression level of the at least inflammasome protein in the biological sample obtained from the patient can be used to increase the accuracy or strengthen a diagnosis of a particular disease, disorder or condition caused by or associated with inflammation determined using one or more additional diagnostic assessments. The one or more additional diagnostic assessments can be selected from the group consisting of assessment of clinical parameters, examination of morphological indicators in tissue biopsies, and assessment or evaluation of symptoms associated with a particular disease, disorder or condition caused by or associated with inflammation. Any of the diagnostic methods provided herein with respect to determining levels of inflammasome proteins in a biological samples obtained from patients can be used as an adjunct to known diagnostic methods for a particular disease, disorder or condition caused by or associated with inflammation.

In other cases, provided herein are methods for diagnosing or evaluating a patient suspected of having inflammation or a disease, disorder or condition caused by or associated with inflammation that can comprise detecting an expression level of at least one inflammasome protein and at least one control biomarker protein in a biological sample obtained from a patient suspected of suffering from inflammation or a disease, disorder or condition caused by or associated with inflammation, detecting an expression level of the at least one inflammasome protein and the at least one control biomarker protein in a control biological sample, comparing the expression level of the at least one inflammasome protein and the at least one control biomarker protein in the biological sample obtained from the patient suspected of suffering from inflammation or the disease, disorder or condition caused by or associated with inflammation and the control biological sample, and selecting the patient as having inflammation or the disease, disorder or condition caused by or associated with inflammation based on the comparison. In some cases, an increased expression level of the detected expression level of the at least one inflammasome protein and the at least one control biomarker protein in the biological sample obtained from the patient suspected of suffering from inflammation or the disease, disorder or condition caused by or associated with inflammation as compared to the expression levels in the control biological sample selects the patient as having inflammation or the disease, disorder or condition caused by or associated with inflammation. In some cases, a decreased expression level of the detected expression level of the at least one inflammasome protein and the at least one control biomarker protein in the biological sample obtained from the patient suspected of suffering from inflammation or the disease, disorder or condition caused by or associated with inflammation as compared to the expression levels in the control biological sample selects the patient as having inflammation or the disease, disorder or condition caused by or associated with inflammation. In some cases, the control biological sample can be a biological sample obtained from a subject not suspected of suffering from inflammation or the disease, disorder or condition caused by or associated with inflammation and the at least one control protein can be the at least one inflammasome protein detected in the biological sample obtained from the patient suspected of suffering from inflammation or the disease, disorder or condition caused by or associated with inflammation. The control biomarker protein can be any protein whose expression level has been previously shown to be associated with inflammation or the disease, disorder or condition caused by or associated with inflammation. In one embodiment, an elevated expression level of the control biomarker protein has been previously shown to be associated with or diagnostic of inflammation or the disease, disorder or condition caused by or associated with inflammation. In one embodiment, the disease, disorder or condition caused by or associated with inflammation is MCI or AD and the at least one control protein is a control biomarker protein selected from NFL, amyloid-β (Aβ₍₁₋₄₂₎), T-Tau, sAPPα, and sAPPβ. In one embodiment, the disease, disorder or condition caused by or associated with inflammation is NASH and the at least one control protein is a control biomarker protein selected from Gal-3 and CRP (hs-CRP).

In one embodiment, provided herein is a method for diagnosing or evaluating a patient of having multiple sclerosis (MS) comprising measuring the level of at least one inflammasome protein in a biological sample obtained from the patient; determining the presence or absence of a protein signature associated with MS, wherein the protein signature comprises an elevated level of the at least one inflammasome protein; and selecting the patient as having the MS if the patient exhibits the presence of the protein signature. The patient can present with clinical symptoms consistent with MS. Through use of the methods and compositions provided herein, the patient can be diagnosed with any type of MS known in the art. The MS can be relapsing-remitting MS (RRMS), secondary-progressive MS (SPMS), primary-progressive MS (PPMS), or progressive-relapsing MS (PRMS). In some cases the method further comprises measuring in a sample obtained from a patient the expression level of a control biomarker(s) whose altered levels of expression have been shown to be associated with MS such as, for example, NFL and using detection of an altered expression level of said control biomarker(s) in combination with a detected altered expression level of one or more inflammasome proteins in order to positively diagnose MS in the patient. In some cases, the method further comprises assessing a patient's clinical features/symptoms with respect to MS and using detection of an altered expression level of one or more inflammasome proteins in a sample obtained from the patient in order to positively diagnose MS in the patient.

In another embodiment, provided herein is a method for diagnosing or evaluating a patient suspected of having suffered a stroke, the method comprising: measuring the level of at least one inflammasome protein in a biological sample obtained from the patient; determining the presence or absence of a protein signature associated with stroke or a stroke-related injury, wherein the protein signature comprises an elevated level of the at least one inflammasome protein; and selecting the patient as having suffered from a stroke if the patient exhibits the presence of the protein signature. The patient can present with any clinical symptoms known in the art consistent with stroke. The stroke can be ischemic stroke, transient ischemic stroke or hemorrhagic stroke. In some cases the method further comprises measuring in a sample obtained from a patient the expression level of a control biomarker(s) whose altered levels of expression have been shown to be associated with stroke and using detection of an altered expression level of said control biomarker(s) in combination with a detected altered expression level of one or more inflammasome proteins in order to positively diagnose stroke in the patient. In some cases, the method further comprises assessing a patient's clinical features/symptoms with respect to stroke and using detection of an altered expression level of one or more inflammasome proteins in a sample obtained from the patient in order to positively diagnose stroke in the patient.

In one embodiment, provided herein is a method for diagnosing or evaluating a patient of having traumatic brain injury (TBI) comprising measuring the level of at least one inflammasome protein in a biological sample obtained from the patient; determining the presence or absence of a protein signature associated with TBI, wherein the protein signature comprises an elevated level of the at least one inflammasome protein; and selecting the patient as having a TBI if the patient exhibits the presence of the protein signature. The patient can present with clinical symptoms consistent with TBI. Through use of the methods and compositions provided herein, the patient can be diagnosed with any type of TBI known in the art. In some cases the method further comprises measuring in a sample obtained from a patient the expression level of a control biomarker(s) whose altered levels of expression have been shown to be associated with TBI and using detection of an altered expression level of said control biomarker(s) in combination with a detected altered expression level of one or more inflammasome proteins in order to positively diagnose TBI in the patient. In some cases, the method further comprises assessing a patient's clinical features/symptoms with respect to TBI and using detection of an altered expression level of one or more inflammasome proteins in a sample obtained from the patient in order to positively diagnose TBI in the patient.

In one embodiment, provided herein is a method for diagnosing or evaluating a patient of having cognitive impairment. The cognitive impairment can be mild or severe. In one embodiment, the cognitive impairment is mild cognitive impairment (MCI). The method comprises measuring the level of at least one inflammasome protein in a biological sample obtained from the patient; determining the presence or absence of a protein signature associated with cognitive impairment (e.g., MCI), wherein the protein signature comprises an elevated level of the at least one inflammasome protein; and selecting the patient as having a cognitive impairment (e.g., MCI) if the patient exhibits the presence of the protein signature. In some cases, the method further comprises measuring an expression level of at least one control biomarker protein and wherein the protein signature further comprises an elevated expression level of at least one control biomarker protein. The at least one control biomarker protein is any protein whose expression level has been previously shown to be associated with the brain injury. The at least one control biomarker protein can be selected from NFL, amyloid-β (Aβ₍₁₋₄₂₎), T-Tau, sAPPα, or sAPPβ. The patient can present with clinical symptoms consistent with cognitive impairment (e.g., MCI). Through use of the methods and compositions provided herein, the patient can be diagnosed with any type of cognitive impairment known in the art such as, for example, MCI. Examples of symptoms often displayed by subject's affected with MCI can include forgetfulness (forget things more frequently and/or forget important events), lack of focus (lose train of thought), feel anxious or overwhelmed when making decisions, understanding instructions or planning things, trouble navigating familiar environments, and/or impulsivity and questionable judgment. Subjects with MCI may also experience depression, irritability, anxiety or apathy. In some cases the method further comprises measuring in a sample obtained from a patient the expression level of a control biomarker(s) whose altered levels of expression have been shown to be associated with MCI such as, for example, NFL, amyloid-β (Aβ₍₁₋₄₂₎), T-Tau, sAPPα, or sAPPβ and using detection of an altered expression level of said control biomarker(s) in combination with a detected altered expression level of one or more inflammasome proteins in order to positively diagnose MCI in the patient. In some cases, the method further comprises assessing a patient's clinical features/symptoms with respect to MCI and using detection of an altered expression level of one or more inflammasome proteins in a sample obtained from the patient in order to positively diagnose MCI in the patient.

In one embodiment, provided herein is a method for diagnosing or evaluating a patient with Alzheimer's disease (AD). In some embodiments, Alzheimer's disease causes dementia. In some embodiments, the patient has AD that is classified as early-stage (mild), middle-stage (moderate), or late-stage (severe). In one embodiment, the AD is early-stage. In some embodiments, the AD is middle-stage. In some embodiments, the AD is late-stage. The method comprises measuring an expression level of at least one inflammasome protein in a biological sample obtained from the patient; determining the presence or absence of a protein signature associated with cognitive impairment (e.g., AD), wherein the protein signature comprises an elevated level of the at least one inflammasome protein; and selecting the patient as having a cognitive impairment (e.g., AD) if the patient exhibits the presence of the protein signature. In some cases, the method further comprises measuring an expression level of at least one control biomarker protein and wherein the protein signature further comprises an elevated expression level of at least one control biomarker protein. The at least one control biomarker protein is any protein whose expression level has been previously shown to be associated with the brain injury. The at least one control biomarker protein can be selected from NFL, amyloid-β (Aβ₍₁₋₄₂₎), T-Tau, sAPPα, or sAPPβ. The patient can present with clinical symptoms consistent with AD. Through use of the methods and compositions provided herein, the patient can be diagnosed with any type of AD known in the art such as, for example, mild-stage, moderate-stage, or late-stage. Examples of symptoms often displayed by subject's affected with AD can include forgetfulness (forget things more frequently and/or forget important events), lack of focus (lose train of thought), feel anxious or overwhelmed when making decisions, understanding instructions or planning things, trouble navigating familiar environments, difficulty performing tasks, forgetting material that was just read, losing or misplacing a valuable object, experiencing increased trouble with planning or organizing, confusion, trouble controlling bladder or bowels, personality and behavioral changes, changes in sleep patterns, difficulty communicating, vulnerability to infections, and/or impulsivity and questionable judgment. Subjects with AD may also experience depression, irritability, anxiety or apathy. In some cases the method further comprises measuring in a sample obtained from a patient the expression level of a control biomarker(s) whose altered levels of expression have been shown to be associated with AD such as, for example, NFL, amyloid-β (Aβ₍₁₋₄₂₎), T-Tau, sAPPα, or sAPPβ and using detection of an altered expression level of said control biomarker(s) in combination with a detected altered expression level of one or more inflammasome proteins in order to positively diagnose AD in the patient. In some cases, the method further comprises assessing a patient's clinical features/symptoms with respect to AD and using detection of an altered expression level of one or more inflammasome proteins in a sample obtained from the patient in order to positively diagnose AD in the patient.

In one embodiment, provided herein is a method for diagnosing or evaluating a patient with age-related inflammation or inflammaging. The method comprises measuring an expression level of at least one inflammasome protein in a biological sample obtained from the patient; determining the presence or absence of a protein signature associated with inflammaging, wherein the protein signature comprises an elevated level of the at least one inflammasome protein; and selecting the patient as having inflammaging if the patient exhibits the presence of the protein signature. In some cases, the method further comprises measuring an expression level of at least one control biomarker protein and wherein the protein signature further comprises an elevated expression level of at least one control biomarker protein. The at least one control biomarker protein is any protein whose expression level has been previously shown to be associated with inflammaging. The patient can present with clinical symptoms consistent with inflammaging.

In one embodiment, provided herein is a method for diagnosing or evaluating a patient with age-related macular degeneration (AMD). In some embodiments, a patient with AMD has a damaged macula. The macula is a part of the retina. In some embodiments, patients with AMD experience loss of central vision and fine details, but retain peripheral vision. There are two types of AMD: dry AMD and wet AMD. Dry AMD is characterized by the presence of insoluble extracellular aggregates or drusen in the macula. Drusen affect the retinal pigmented epithelium (RPE) and the photoreceptor layer, and when advanced, it eventually may progress to RPE atrophy and severe vision loss. The less common form of AMD is wet AMD, which is characterized by choroidal neovascularization (CNV) and if left untreated may rapidly progress to blindness. In some embodiments, the methods herein are used to diagnose patients wet AMD. In some embodiments, the methods herein are used to diagnose patients with dry AMD. In some embodiments, the methods described herein are used to diagnose patients with wet AMD and dry AMD. In some embodiments, the methods described herein are used to distinguish between a patient that has wet AMD and dry AMD. This distinction is important, because treatments effective for wet AMD, such as anti-vascular endothelial growth factory therapy (anti-VEGF) therapy, are not effective for dry AMD. The method comprises measuring an expression level of at least one inflammasome protein in a biological sample obtained from the patient; determining the presence or absence of a protein signature associated with AMD, wherein the protein signature comprises an elevated level of the at least one inflammasome protein; and selecting the patient as having AMD if the patient exhibits the presence of the protein signature. In some cases, the method further comprises measuring an expression level of at least one control biomarker protein and wherein the protein signature further comprises an elevated expression level of at least one control biomarker protein. The at least one control biomarker protein is any protein whose expression level has been previously shown to be associated with AMD. The patient can present with clinical symptoms consistent with AMD. The patient can present with abnormal changes in the macular area such as the presence of drusen or fluid in the macula, pigment epithelial detachment as revealed by a comprehensive eye exam that includes an optical coherent tomography (OCT) of the macula. Through use of the methods and compositions provided herein, the patient can be diagnosed with any type of AMD known in the art such as, for example, wet AMD or dry AMD. Examples of symptoms often displayed by subject's affected with AMD can include blurred or “fuzzy” vision, straight lines, such as sentences on a page, appearing wavy or distorted, blurry areas on a printed page, difficulty reading or seeing details in low light levels, extra sensitivity to glare, dark, blurry areas, or whiteout that appears in the center of vision, or a change in the perception of color. In some cases the method further comprises measuring in a sample obtained from a patient the expression level of a control biomarker(s) whose altered levels of expression have been shown to be associated with AMD and using detection of an altered expression level of said control biomarker(s) in combination with a detected altered expression level of one or more inflammasome proteins in order to positively diagnose AMD in the patient. In some cases, the method further comprises assessing a patient's clinical features/symptoms with respect to AMD and using detection of an altered expression level of one or more inflammasome proteins in a sample obtained from the patient in order to positively diagnose AMD in the patient.

In one embodiment, provided herein is a method for diagnosing or evaluating a patient with Nonalcoholic fatty liver disease (NAFLD). The method comprises measuring an expression level of at least one inflammasome protein in a biological sample obtained from the patient; determining the presence or absence of a protein signature associated with NAFLD, wherein the protein signature comprises an elevated level of the at least one inflammasome protein; and selecting the patient as having NAFLD if the patient exhibits the presence of the protein signature. In some cases, the method further comprises measuring an expression level of at least one control biomarker protein and wherein the protein signature further comprises an elevated expression level of at least one control biomarker protein. The at least one control biomarker protein is any protein whose expression level has been previously shown to be associated with NAFLD. The patient can present with clinical symptoms consistent with NAFLD. Through use of the methods and compositions provided herein, the patient can be diagnosed with any type of NAFLD known in the art such as, for example, fatty liver or Nonalcoholic steatohepatitis (NASH).

In one embodiment, provided herein is a method for diagnosing or evaluating a patient suspected of suffering from NASH by measuring an expression level of at least one inflammasome protein in a biological sample obtained from a patient suffering from or suspected of suffering from NASH in combination with determining the expression level of biomarkers whose altered expression levels are known or suspected to be associated with NASH. In one embodiment, provided herein is a method for diagnosing or evaluating a patient suspected of suffering from NASH by the measuring the expression level of at least one inflammasome protein in a biological sample obtained from a patient suspected of suffering from NASH in combination with one or more additional diagnostic assessments. Detection of an altered expression level of the at least inflammasome protein in the biological sample obtained from the patient can be used to confirm a NASH diagnosis determined using one or more additional diagnostic assessments. Detection of an altered expression level of the at least inflammasome protein in the biological sample obtained from the patient can be used to increase the accuracy or strengthen a NASH diagnosis determined using one or more additional diagnostic assessments. The one or more additional diagnostic assessments can be selected from the group consisting of assessment of clinical parameters, examination of morphological indicators in liver biopsies, determining levels of inflammatory cytokines and chemokines, assessment of adipokines, assessment of hepatic fibrosis biomarkers, assessment of oxidative stress, assessment of mitochondrial dysfunction and assessment of apoptosis biomarkers. Examples of inflammatory cytokines and chemokines used as biomarkers for NASH include TNF-alpha, IL-6, the chemokine CC-chemokine ligand-2 (chemo-attractant protein-1), and high-sensitivity C-reactive protein (hs-CRP). Examples of apoptosis biomarkers include CK-18, sFas and hyaluronic acid. Examples of adipokines include leptin, adiponectin, resistin, retinol binding protein 4 and ghrelin. Examples of oxidative stress biomarkers include 13-hydroxy-octadecadienoic acid, SOD2 and cytochrome p450 2E1 (CYP2E1). Examples of mitochondrial dysfunction biomarkers include CK-7 and CK-18. Hepatic fibrosis markers can include Galectin-3 (Gal-3), hyaluronic acid, procollagen III N-terminal peptide, TGF-β and TIMP1. Examples of clinical parameters can be selected from body mass index, waist circumference, blood or serum levels of alanine aminotransferase (ALT), aspartate aminotransferase (AST), total cholesterol, low-density lipoprotein, triglycerides, glucose, insulin resistance and metabolic and proteomic profile analyses.

In one aspect of the invention, the method of diagnosing or evaluating a patient suspected of having inflammation or a disease, disorder or condition associated with inflammation (e.g., NASH, MCI, TBI, AD, AMD, inflammaging, stroke or MS) comprises determining the presence or absence of a protein signature associated with inflammation or the disease, disorder or condition associated with inflammation based on the measured level, abundance, or concentration of one or more inflammasome proteins alone or in combination with one or more control biomarker proteins in a biological sample obtained from the patient. In certain embodiments, the protein signature comprises an elevated level of at least one inflammasome protein and/or an elevated level of at least one control biomarker protein. The level of the at least one inflammasome protein and/or control biomarker protein in the protein signature may be enhanced relative to the level or percentage of the at least one inflammasome protein and/or the at least one control biomarker protein in a biological sample obtained from a control subject or relative to a pre-determined reference value or range of reference values as further described herein. The control subject can be a healthy individual. The healthy individual can be an individual who does not exhibit symptoms associated with inflammation or the disease, disorder or condition associated with inflammation (e.g., NASH, MCI, AMD, TBI, AD, inflammaging, stroke or MS). The protein signature may, in certain embodiments, comprise an elevated level at least one inflammasome proteins. The at least one control biomarker protein is any protein whose expression level has been previously shown to be associated with inflammation or the disease, disorder or condition associated with inflammation. In some embodiments, the control biomarker proteins is Gal-3, CRP (hs-CRP), NFL, amyloid-β (Aβ₍₁₋₄₂₎), T-Tau, sAPPα, or sAPPβ. Patients who exhibit the protein signature may be selected or identified as having inflammation or the disease, disorder or condition associated with inflammation (e.g., NASH, MCI, AD, TBI, AMD, inflammaging, stroke or MS).

In some embodiments, the measured level, concentration, or abundance of one or more inflammasome proteins alone or in combination with one or more control biomarker proteins in the biological sample is used to prepare a protein profile or signature that is indicative of the severity of inflammation or the disease, disorder or condition associated with inflammation (e.g., NASH, MCI, TBI, AD, AMD, inflammaging, stroke or MS). In some cases, the protein profile may comprise the level, abundance, percentage or concentration of one or more inflammasome proteins measured in the patient's biological sample in relation to the level, abundance, percentage or concentration of the one or more inflammasome proteins in a biological sample obtained from a control subject or in relation to a pre-determined value or range of reference values as described herein. In some cases, the protein profile may comprise the level, abundance, percentage or concentration of one or more inflammasome proteins and one or more control biomarker proteins measured in the patient's biological sample in relation to the level, abundance, percentage or concentration of the one or more inflammasome proteins and the one or more control biomarker proteins in a biological sample obtained from a control subject or in relation to a pre-determined value or range of reference values as described herein. The control subject can be a healthy individual. The healthy individual can be an individual who does not exhibit symptoms associated with inflammation or the disease, disorder or condition associated with inflammation (e.g., NASH, MCI, TBI, AD, AMD, inflammaging, stroke or MS). The one or more control biomarker protein(s) can be any protein whose expression level has been previously shown to be associated with inflammation or the disease, disorder or condition associated with inflammation. In some embodiments, the control biomarker protein is Gal-3, CRP (hs-CRP), NFL, amyloid-β (Aβ₍₁₋₄₂₎), T-Tau, sAPPα, or sAPPβ.

The level, percentage or concentration of at least one inflammasome protein and/or the control biomarker proteins can be assessed at a single time point and compared to a pre-determined reference value or range of reference values or can be assessed at multiple time points and compared to a pre-determined reference value or to previously assessed values.

As used herein, “pre-determined reference value” or range of reference values can refer to a pre-determined value or range of reference values of the level or concentration of an inflammasome protein and/or control biomarker protein ascertained from a known sample. For instance, the pre-determined reference value or range of reference values can reflect the level or concentration of an inflammasome protein and/or control biomarker protein in a biological sample obtained from a control subject (i.e., healthy subject). The control subject may, in some embodiments, be age-matched to the patients being evaluated. The biological sample obtained from the patient and the control subject can both be the same type of sample (e.g., serum or serum-derived extracellular vesicles (EVs). Thus, in particular embodiments, the measured level, percentage or concentration of at least one inflammasome protein and/or control biomarker protein is compared or determined relative to the level, percentage or concentration of said at least one inflammasome protein and/or control biomarker protein in a control sample (i.e. obtained from a healthy subject). The control or healthy subject can be a subject that does not exhibit symptoms associated with inflammation or the disease, disorder or condition associated with inflammation brain injury (e.g., NASH, MCI, TBI, AD, stroke, inflammaging, AMD, or MS). The control biomarker protein can be any protein whose expression level has been previously shown to be associated with the brain injury. In some embodiments, the control biomarker protein is GAL-3, CRP (hs-CRP), NFL, amyloid-β (Aβ₍₁₋₄₂₎), T-Tau, sAPPα, or sAPPβ.

In other embodiments, the pre-determined reference value or range of reference values can reflect the level or concentration of an inflammasome protein and/or control biomarker protein in a sample obtained from a patient with a known severity of inflammation or a disease, disorder or condition associated with inflammation (e.g., NASH, MCI, TBI, AD, AMD, inflammaging, stroke or MS) as assessed by clinical measures or post mortem analysis. A pre-determined reference value can also be a known amount or concentration of an inflammasome protein and/or control biomarker protein. Such a known amount or concentration of an inflammasome and/or control biomarker protein may correlate with an average level or concentration of the inflammasome and/or control biomarker protein from a population of control subjects or a population of patients with known levels of inflammation or said disease, disorder or condition associated with inflammation. In another embodiment, the pre-determined reference value can be a range of values, which, for instance, can represent a mean plus or minus a standard deviation or confidence interval. A range of reference values can also refer to individual reference values for a particular inflammasome and/or control biomarker protein across various levels of inflammation or a disease, disorder or condition associated with inflammation (e.g., NASH, AD, MCI, TBI, AMD, inflammaging, stroke or MS) severity. The control biomarker protein can be any protein whose expression level has been previously shown to be associated with the brain injury. In some embodiments, the control biomarker proteins is Gal-3, CRP (hs-CRP), NFL, amyloid-β (Aβ₍₁₋₄₂₎), T-Tau, sAPPα, or sAPPβ. In certain embodiments, an increase in the level of one or more inflammasome proteins (e.g., ASC, caspase-1 or IL-18) and/or control biomarker proteins (e.g. Gal-3, CRP (hs-CRP), NFL, sAPPα, sAPPβ, T-Tau or AB₍₁₋₄₂₎) relative to a pre-determined reference value or range of reference values is indicative of a more severe form of inflammation or the disease, disorder or condition associated with inflammation (e.g., brain injury).

The at least one inflammasome protein detected or measured in any of the methods provided herein can be one or a plurality of inflammasome proteins. In one embodiment, the at least one inflammasome protein is a plurality of inflammasome proteins. The plurality can be at least or at most 2, 3, 4 or 5 inflammasome proteins. The at least one inflammasome protein or plurality of inflammasome proteins can be a component of any inflammasome known in the art, such as, for example, the NAPL1/NLRP1, NALP2/NLRP2, NALP3/NLRP3, IPAF/NLRC4 or AIM2 inflammasome. In some cases, the at least one inflammasome protein or plurality of inflammasome proteins can be a component of a canonical inflammasome or non-canonical inflammasome. In one embodiment, the at least one inflammasome protein is apoptosis-associated speck-like protein containing a caspase recruitment domain (ASC), caspase-1, interleukin-18 (IL-18) or interleukin-1beta (IL-1beta). In one embodiment, the at least one inflammasome protein is apoptosis-associated speck-like protein containing a caspase recruitment domain (ASC). In one embodiment, the at least one inflammasome protein is caspase-1. In one embodiment, the at least one inflammasome protein is IL-18. The at least one control biomarker protein detected or measured in any of the methods provided herein can be any protein whose expression level has been previously shown to be associated with a brain injury. In one embodiment, the at least one control biomarker protein is Gal-3. In one embodiment, the at least one control biomarker protein is CRP (hs-CRP). In one embodiment, the at least one control biomarker protein is NFL. In some embodiments, the at least one control biomarker protein is sAPPα. In some embodiments, the at least one control biomarker protein is sAPPβ. In some embodiments, the at least one control biomarker protein is Aβ₍₁₋₄₂₎. In some embodiments, the at least one control biomarker protein is Aβ₍₁₋₄₀₎. In some embodiments, the at least one control biomarker protein is APP. In some embodiments, the at least one control biomarker protein is T-Tau.

The inflammasome proteins of the methods provided herein and/or control biomarker proteins (e.g., control biomarker proteins such as Gal-3, CRP (hs-CRP), NFL, sAPPα, sAPPβ, or AB₍₁₋₄₂₎) can be measured in a biological sample by various methods known to those skilled in the art. For instance, proteins can be measured by methods including, but not limited to, liquid chromatography, gas chromatography, mass spectrometry, immunoassays, radioimmunoassays, immunofluorescent assays, FRET-based assays, immunoblot, ELISAs, or liquid chromatography followed by mass spectrometry (e.g., MALDI MS). One of skill in the art can ascertain other suitable methods for measuring and quantitating any particular biomarker protein of the invention.

In one embodiment, the at least one inflammasome protein or plurality of inflammasome proteins detected or measured in any of the methods provided herein can be detected or measured through the use of an immunoassay. In one embodiment, the at least one control biomarker protein is detected or measured in any of the methods provided herein can be detected or measured through the use of an immunoassay. The immunoassay can be any immunoassay known in the art. For example, the immunoassay can be an immunoblot, enzyme-linked immunosorbent assay (ELISA) or a microfluidic immunoassay. An example of a microfluidic immunoassay for use in the methods provided herein is the Simple Plex™ Platform (Protein Simple, San Jose, Calif.).

Any immunoassay for use in the methods provided herein can utilize an antibody directed against an inflammasome protein. The inflammasome component can be a component of any canonical or non-canonical inflammasome known in the art, such as, for example, the NAPL1, NALP2, NALP3, NLRC4 or AIM2 inflammasome. In one embodiment, the inflammasome protein is apoptosis-associated speck-like protein containing a caspase recruitment domain (ASC), caspase-1, interleukin-18 (IL-18) or interleukin-1beta (IL-1beta). In one embodiment, the inflammasome protein is apoptosis-associated speck-like protein containing a caspase recruitment domain (ASC). In one embodiment, the inflammasome protein is caspase-1. In one embodiment, the inflammasome protein is IL-18. In one embodiment, the inflammasome protein is IL-1beta.

Any immunoassay for use in the methods provided herein can utilize an antibody directed against a control biomarker protein. The control biomarker protein can be Gal-3, CRP (hs-CRP), NFL, sAPPα, sAPPβ, or Aβ₍₁₋₄₂₎.

Any suitable antibody that specifically binds ASC can be used, e.g., a custom or commercially available ASC antibody can be used in the methods provided herein. The anti-ASC antibody can be an antibody that specifically binds to a domain or portion thereof of a mammalian ASC protein such as, for example a human or rat ASC protein. Examples of anti-ASC antibodies for use in the methods herein can be those found in U.S. Pat. No. 8,685,400, the contents of which are herein incorporated by reference in its entirety. Examples of commercially available anti-ASC antibodies for use in the methods provided herein include, but are not limited to 04-147 Anti-ASC, clone 2E1-7 mouse monoclonal antibody from Millipore Sigma, AB3607—Anti-ASC Antibody from Millipore Sigma, orb194021 Anti-ASC from Biorbyt, LS-C331318-50 Anti-ASC from LifeSpan Biosciences, AF3805 Anti-ASC from R & D Systems, NBP1-78977 Anti-ASC from Novus Biologicals, 600-401-Y67 Anti-ASC from Rockland Immunochemicals, D086-3 Anti-ASC from MBL International, AL177 anti-ASC from Adipogen, monoclonal anti-ASC (clone o93E9) antibody, anti-ASC antibody (F-9) from Santa Cruz Biotechnology, anti-ASC antibody (B-3) from Santa Cruz Biotechnology, ASC polyclonal antibody—ADI-905-173 from Enzo Life Sciences, or A161 Anti-Human ASC-Leinco Technologies. The human ASC protein can be accession number NP_037390.2 (Q9ULZ3-1), NP_660183 (Q9ULZ3-2) or Q9ULZ3-3. The rat ASC protein can be accession number NP_758825 (BAC43754). The mouse ASC protein can be accession number NP_075747.3. In one embodiment, the antibody binds to a PYRIN-PAAD-DAPIN domain (PYD) or a portion or fragment thereof of a mammalian ASC protein (e.g. human or rat ASC). In this embodiment, an antibody as described herein specifically binds to an amino acid sequence having at least 65% (e.g., 65, 70, 75, 80, 85%) sequence identity with a PYD domain or fragment thereof of human or rat ASC. In one embodiment, the antibody binds to a C-terminal caspase-recruitment domain (CARD) or a portion or fragment thereof of a mammalian ASC protein (e.g. human or rat ASC). In this embodiment, an antibody as described herein specifically binds to an amino acid sequence having at least 65% (e.g., 65, 70, 75, 80, 85%) sequence identity with a CARD domain or fragment thereof of human or rat ASC. In another embodiment, the antibody is an antibody that specifically binds to a region of rat ASC, e.g., amino acid sequence ALRQTQPYLVTDLEQS (SEQ ID NO: 1) (i.e., residues 178-193 of rat ASC, accession number BAC43754). In this embodiment, an antibody as described herein specifically binds to an amino acid sequence having at least 65% (e.g., 65, 70, 75, 80, 85%) sequence identity with amino acid sequence ALRQTQPYLVTDLEQS (SEQ ID NO: 1) of rat ASC. In another embodiment, the antibody is an antibody that specifically binds to a region of human ASC, e.g., amino acid sequence RESQSYLVEDLERS (SEQ ID NO: 2). In this embodiment, an antibody as described herein specifically binds to an amino acid sequence having at least 65% (e.g., 65, 70, 75, 80, 85%) sequence identity with amino acid sequence RESQSYLVEDLERS (SEQ ID NO: 2) of human ASC.

Any suitable anti-NLRP1 antibody (e.g., commercially available or custom) can be used in the methods provided herein. Examples of anti-NLRP1 antibodies for use in the methods herein can be those found in U.S. Pat. No. 8,685,400, the contents of which are herein incorporated by reference in its entirety. Examples of commercially available anti-NLRP1 antibodies for use in the methods provided herein include, but are not limited to human NLRP1 polyclonal antibody AF6788 from R&D Systems, EMD Millipore rabbit polyclonal anti-NLRP1 ABF22, Novus Biologicals rabbit polyclonal anti-NLRP1 NB100-56148, Sigma-Aldrich mouse polyclonal anti-NLRP1 SAB1407151, Abcam rabbit polyclonal anti-NLRP1 ab3683, Biorbyt rabbit polyclonal anti-NLRP1 orb325922, my BioSource rabbit polyclonal anti-NLRP1 MBS7001225, R&D systems sheep polyclonal AF6788, Aviva Systems mouse monoclonal anti-NLRP1 oaed00344, Aviva Systems rabbit polyclonal anti-NLRP1 ARO54478_P050, Origene rabbit polyclonal anti-NLRP1 APO7775PU-N, Antibodies online rabbit polyclonal anti-NLRP1 ABIN768983, Prosci rabbit polyclonal anti-NLRP1 3037, Proteintech rabbit polyclonal anti-NLRP1 12256-1-AP, Enzo mouse monoclonal anti-NLRP1 ALX-804-803-C100, Invitrogen mouse monoclonal anti-NLRP1 MA1-25842, GeneTex mouse monoclonal anti-NLRP1 GTX16091, Rockland rabbit polyclonal anti-NLRP1 200-401-CX5, or Cell Signaling Technology rabbit polyclonal anti-NLRP1 4990. The human NLRP1 protein can be accession number AAH51787, NP_001028225, NP_055737, NP_127497, NP_127499, or NP_127500. In one embodiment, the antibody binds to a Pyrin, NACHT, LRR1-6, FIIND or CARD domain or a portion or fragment thereof of a mammalian NLRP1 protein (e.g. human NLRP1). In this embodiment, an antibody as described herein specifically binds to an amino acid sequence having at least 65% (e.g., 65%, 70%, 75%, 80%, 85%) sequence identity with a specific domain (e.g., Pyrin, NACHT, LRR1-6, FIIND or CARD) or fragment thereof of human NLRP1. In one embodiment, a chicken anti-NLRP1 polyclonal that was custom-designed and produced by Ayes Laboratories can be used. This antibody can be directed against the following amino acid sequence in human NLRP1: CEYYTEIREREREKSEKGR (SEQ ID NO: 3). In one embodiment, the antibody specifically binds to an amino acid sequence having at least 85% sequence identity with amino acid sequence SEQ ID NO: 3 or MEE SQS KEE SNT EG-cys (SEQ ID NO: 4).

Any suitable antibody that specifically binds caspase-1 can be used, e.g., a custom or commercially available, in the methods provided herein. Examples of commercially available anti-caspase-1 antibodies for use in the methods provided herein include: R&D Systems: Cat #MAB6215, or Cat #AF6215; Cell Signaling: Cat #3866, #225, or #4199; Novus Biologicals: Cat #NB100-56565, #NBP1-45433, #NB100-56564, #MAB6215, #AF6215, #NBP2-67487, #NBP2-15713, #NBP2-15712, #NBP1-87680, #NB120-1872, #NBP1-76605, or #H00000834-M01.

Any suitable antibody that specifically binds caspase-8 can be used, e.g., a custom or commercially available, in the methods provided herein. Examples of commercially available anti-caspase-8 antibodies for use in the methods provided herein include: Abcam: Cat #ab25901, ab227430, ab108333, ab220171, ab4052, ab231948, ab32397, ab61755, ab138485, ab 208774, ab32125, ab231475, ab247233, ab2553, ab232046, ab194145 or ab119809; Novus Biologicals: Cat #NB100-56116, #NB100-56527, #NBP1-05123, #AF705, #AF1650, #MAB704, #NBP2-15722, #NBP1-76610, #NBP2-22183, #NBP2-67803, #NB500-208 or #NBP2-67355; Santa Cruz Biotechnology Cat #8CSP03; Cell Signaling Technology: Cat. #4790 or #9746.

Any suitable antibody that specifically binds caspase-11 can be used, e.g., a custom or commercially available, in the methods provided herein. Examples of commercially available anti-caspase-11 antibodies for use in the methods provided herein include: Abcam: Cat #ab180673, ab240991, ab22684 or ab69540; Novus Biological Cat #NB120-10454; Cell Signaling Technology Cat #14340, or ThermoFisher Cat #14-9935-82.

Any suitable antibody that specifically binds IL-18 can be used, e.g., a custom or commercially available, in the methods provided herein. Examples of commercially available anti-IL-18 antibodies for use in the methods provided herein include: R&D Systems: Cat #D044-3, Cat #D045-3, #MAB646, #AF2548, #D043-3, #MAB2548, MAB9124, #MAB91241, #MAB91243, MAB91244, or #MAB91242; Novus Biologicals: Cat #AF2548, #D043-3, #MAB2548, #MAB9124, #MAB91243, #MAB91244, #MAB91241, #D045-3, #MAB91242, or #D044-3.

Any suitable antibody that specifically binds IL-1beta can be used, e.g., a custom or commercially available, in the methods provided herein. Examples of commercially available anti-IL-18 antibodies for use in the methods provided herein include: R&D Systems: Cat #MAB601, Cat #MAB201, #MAB6964, #MAB601R, #MAB8406, or #MAB6215; Cell Signaling: Cat #31202, #63124, #12426, or #12507; Novus Biologicals: Cat #AF-201-NA, #NB600-633, #MAB201, #MAB601, #NBP1-19775, #NBP2-27345, #AB-201-NA, #NBP2-27342, #NBP2-67865, #NBP2-27343, #NBP2-27340, #NBP2-27340, #NB120-8319, #23600002, #MAB8406, #NB100-73053, #NB120-10749, or #MAB601R.

Any suitable antibody that specifically binds NFL can be used, e.g., a custom or commercially available, in the methods provided herein. Examples of commercially available anti-NFL antibodies for use in the methods provided herein include: Boster Bio: Cat #MA1070; BioLegend: Cat #837801; R&D Systems: Cat #MAB2216, #MAB22162, Novus Biologicals: #NB300-131 or #NBP2-31201. Other examples of anti-Nfl antibodies for use in the methods provided herein include the anti-Nfl antibodies prepared by Uman Diagnostics.

Any suitable antibody that specifically binds APP can be used, e.g., a custom or commercially available, in the methods provided herein. Examples of commercially available anti-APP antibodies for use in the methods provided herein include: United States Biological: Cat #303112; St. John's Laboratory: Cat #STJ113456; Biorbyt: Cat #orb223652, Cat #orb223651, United States Biological: Cat #253944, Cat #253943.

Any suitable antibody that specifically binds Gal-3 can be used, e.g., a custom or commercially available, in the methods provided herein. Examples of commercially available anti-Gal-3 antibodies for use in the methods provided herein include: Abcam Cat #ab209344, ab76466, ab76245, ab2785 and ab31707; Santa Cruz Biotechnology: Cat #sc-23938; Novus Biological: Cat #AF1197, Cat #AF1154, Cat #NB300-538, Cat #NBP1-92690, Cat #MAB1197, Cat #NBP2-16589 and Cat #MAB11541.

Any suitable antibody that specifically binds CRP can be used, e.g., a custom or commercially available, in the methods provided herein. Examples of commercially available anti-CRP antibodies for use in the methods provided herein include: Abcam Cat #ab32412, ab256492, ab256525, ab207756 and ab51016; HyTest Ltd cat #4C28-C6; Genescript cat #hsCRP (11C2).

Methods for determining monoclonal antibody specificity and affinity by competitive inhibition can be found in Harlow, et al., Antibodies: A Laboratory Manual, Cold Spring Harbor Laboratory Press, Cold Spring Harbor, N.Y., 1988, Colligan et al., eds., Current Protocols in Immunology, Greene Publishing Assoc. and Wiley Interscience, N.Y., (1992, 1993), and Muller, Meth. Enzymol. 92:589-601, 1983, which references are entirely incorporated herein by reference.

Anti-inflammasome (e.g., Anti-ASC and anti-NLRP1) and/or anti-control biomarker protein antibodies of the present invention can be routinely made according to methods such as, but not limited to inoculation of an appropriate animal with the polypeptide or an antigenic fragment, in vitro stimulation of lymphocyte populations, synthetic methods, hybridomas, and/or recombinant cells expressing nucleic acid encoding such anti-ASC, anti-NFL, anti-sAPPα/β, anti-NLRP1 antibodies. Immunization of an animal using purified recombinant ASC or peptide fragments thereof, e.g., residues 178-193 (SEQ ID NO: 1) of rat ASC (e.g., accession number BAC43754) or SEQ ID NO: 2 of human ASC, is an example of a method of preparing anti-ASC antibodies. Similarly, immunization of an animal using purified recombinant NLRP1 or peptide fragments thereof, e.g., residues MEE SQS KEE SNT EG-cys (SEQ ID NO: 4) of rat NALP1 or SEQ ID NO: 3 of human NALP1, is an example of a method of preparing anti-NLRP1 antibodies.

Monoclonal antibodies that specifically bind ASC, NLRP1, sAPPα, sAPPβ, or NFL may be obtained by methods known to those skilled in the art. See, for example Kohler and Milstein, Nature 256:495-497, 1975; U.S. Pat. No. 4,376,110; Ausubel et al., eds., Current Protocols in Molecular Biology, Greene Publishing Assoc. and Wiley Interscience, N.Y., (1987, 1992); Harlow and Lane ANTIBODIES: A Laboratory Manual Cold Spring Harbor Laboratory Press, Cold Spring Harbor, N.Y., 1988; Colligan et al., eds., Current Protocols in Immunology, Greene Publishing Assoc. and Wiley Interscience, N.Y., (1992, 1993), the contents of which are incorporated entirely herein by reference. Such antibodies may be of any immunoglobulin class including IgG, IgM, IgE, IgA, GILD and any subclass thereof. A hybridoma producing a monoclonal antibody of the present invention may be cultivated in vitro, in situ or in vivo.

In some instances, the methods provided herein can be capable of diagnosing or detecting inflammation or a disease, disorder or condition caused by or associated with inflammation (e.g., N A S H, A D, MCI, AMD, inflammaging, stroke, MS or TB I) with a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%.

In some instances, the methods provided herein c an be capable of diagnosing or detecting inflammation or a disease, disorder or condition caused by or associated with inflammation (e.g., NASH, MCI, stroke, MS, AMD, inflammaging, AD, or TBI) with a sensitivity and/or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%.

In one embodiment, the disease, disorder or condition caused by or associated with inflammation is a brain injury. In one embodiment, the brain injury is MS such that detection of an elevated level of ASC in serum obtained from the patient as compared to a control (e.g., a pre-determined reference value or range of reference values) as provided herein determines that the patient has MS with a sensitivity of at least 75, 80, 90%, 95%, 99% or 100%. In another embodiment, the brain injury is MS such that detection of an elevated level of ASC in serum obtained from the patient as compared to a control (e.g., a pre-determined reference value or range of reference values) as provided herein determines that the patient has MS with a specificity of at least 75%, 80%, 85%, 90%, 95%, 99% or 100%. The pre-determined reference value for these embodiments can be the cut-off values shown in Table 7. In yet another embodiment, the brain injury is MS such that detection of an elevated level of ASC in serum obtained from the patient as compared to a control (e.g., a pre-determined reference value or range of reference values) as provided herein determines that the patient has MS with a sensitivity of at least 90%, and a specificity of at least 80%. The pre-determined reference value for this embodiment can be the cut-off values shown in Table 7. In some cases, the range of reference values can be from about 300 pg/ml to about 340 pg/ml to attain a sensitivity of at least 90% and a specificity of at least 80%.

In one embodiment, the brain injury is stroke such that detection of an elevated level of ASC in serum obtained from the patient as compared to a control (e.g., a pre-determined reference value or range of reference values) as provided herein determines that the patient has suffered a stroke with a sensitivity of at least 75, 80, 90%, 95%, 99% or 100%. In another embodiment, the brain injury is stroke such that detection of an elevated level of ASC in serum obtained from the patient as compared to a control (e.g., a pre-determined reference value or range of reference values) as provided herein determines that the patient has MS with a specificity of at least 75%, 80%, 85%, 90%, 95%, 99% or 100%. The pre-determined reference value for these embodiments can be the cut-off values shown in Table 8. In another embodiment, the brain injury is stroke such that detection of an elevated level of ASC in serum obtained from the patient as compared to a control (e.g., a pre-determined reference value or range of reference values) as provided herein determines that the patient suffered a stroke with a sensitivity of at least 100% and a specificity of at least 90%. The pre-determined reference value for this embodiment can be the cut-off values shown in Table 8. In some cases, the range of reference values can be from about 380 pg/ml to about 405 pg/ml to attain a sensitivity of at least 100% and a specificity of at least 90%. The stroke can be ischemic or hemorrhagic as provided herein.

In one embodiment, the brain injury is stroke such that detection of an elevated level of ASC in serum-derived EVs obtained from the patient as compared to a control (e.g., a pre-determined reference value or range of reference values) as provided herein determines that the patient has suffered a stroke with a sensitivity of at least 75%, 80%, 85%, 90%, 95%, 99% or 100%. In another embodiment, the brain injury is stroke such that detection of an elevated level of ASC in serum-derived EVs obtained from the patient as compared to a control (e.g., a pre-determined reference value or range of reference values) as provided herein determines that the patient has MS with a specificity of at least 75, 80, 90%, 95%, 99% or 100%. The pre-determined reference value for these embodiments can be the cut-off values shown in Table 9. In another embodiment, the brain injury is stroke such that detection of an elevated level of ASC in serum-derived EVs obtained from the patient as compared to a control (e.g., a pre-determined reference value or range of reference values) as provided herein determines that the patient suffered a stroke with a sensitivity of at least 100% and a specificity of at least 90%. The pre-determined reference value for this embodiment can be the cut-off values shown in Table 9. In some cases, the range of reference values can be from about 70 pg/ml to about 90 pg/ml to attain a sensitivity of at least 100% and a specificity of at least 90%. The stroke can be ischemic or hemorrhagic as provided herein.

In one embodiment, the brain injury is TBI such that detection of an elevated level of ASC in serum obtained from the patient as compared to a control (e.g., a pre-determined reference value or range of reference values) as provided herein determines that the patient has TBI with a sensitivity of at least 75, 80, 90%, 95%, 99% or 100%. In another embodiment, the brain injury is TBI such that detection of an elevated level of ASC in serum obtained from the patient as compared to a control (e.g., a pre-determined reference value or range of reference values) as provided herein determines that the patient has TBI with a specificity of at least 75%, 80%, 85%, 90%, 95%, 99% or 100%. The pre-determined reference value for these embodiments can be the cut-off values shown in Table 16. In yet another embodiment, the brain injury is TBI such that detection of an elevated level of ASC in serum obtained from the patient as compared to a control (e.g., a pre-determined reference value or range of reference values) as provided herein determines that the patient has TBI with a sensitivity of at least 90%, and a specificity of at least 80%. The pre-determined reference value for this embodiment can be the cut-off values shown in Table 16. In some cases, the range of reference values can be from about 275 pg/ml to about 450 pg/ml to attain a sensitivity of at least 80% and a specificity of at least 70%.

In one embodiment, the brain injury is TBI such that detection of an elevated level of caspase-1 in serum obtained from the patient as compared to a control (e.g., a pre-determined reference value or range of reference values) as provided herein determines that the patient has TBI with a sensitivity of at least 75%, 80%, 90%, 95%, 99% or 100%. In another embodiment, the brain injury is TBI such that detection of an elevated level of caspase-1 in serum obtained from the patient as compared to a control (e.g., a pre-determined reference value or range of reference values) as provided herein determines that the patient has TBI with a specificity of at least 75%, 80%, 85%, 90%, 95%, 99% or 100%. The pre-determined reference value for these embodiments can be the cut-off values shown in Table 15. In yet another embodiment, the brain injury is TBI such that detection of an elevated level of caspase-1 in serum obtained from the patient as compared to a control (e.g., a pre-determined reference value or range of reference values) as provided herein determines that the patient has TBI with a sensitivity of at least 90%, and a specificity of at least 80%. The pre-determined reference value for this embodiment can be the cut-off values shown in Table 15. In some cases, the range of reference values can be from about 2.812 pg/ml to about 1.853 pg/ml to attain a sensitivity of at least 70% and a specificity of at least 75%.

In one embodiment, the brain injury is MCI such that detection of an elevated level of ASC in serum obtained from the patient as compared to a control (e.g., a pre-determined reference value or range of reference values) as provided herein determines that the patient has MCI with a sensitivity of at least 75%, 80%, 85%, 90%, 95%, 99% or 100%. In another embodiment, the brain injury is MCI such that detection of an elevated level of ASC in serum obtained from the patient as compared to a control (e.g., a pre-determined reference value or range of reference values) as provided herein determines that the patient has MCI with a specificity of at least 50%, 55%, 60% 65%, 70%, 75%, 80%, 85%, 90%, 95%, 99% or 100%. The pre-determined reference value for these embodiments can be the cut-off values shown in Tables 22A and 23. In yet another embodiment, the brain injury is MCI such that detection of an elevated level of ASC in serum obtained from the patient as compared to a control (e.g., a pre-determined reference value or range of reference values) as provided herein determines that the patient has MCI with a sensitivity of at least 90%, and a specificity of at least 70%. The pre-determined reference value(s) for this embodiment can be the cut-off values shown in Tables 22A and 23. In some cases, the range of reference values can be about 257 pg/ml to about 342 pg/ml to attain a sensitivity of at least 90% and a specificity of at least 70%. In some cases, the cut-off value is above 560 pg/ml.

In one embodiment, the brain injury is MCI such that detection of an elevated level of IL-18 in serum obtained from the patient as compared to a control (e.g., a pre-determined reference value or range of reference values) as provided herein determines that the patient has MCI with a sensitivity of at least 75%, 80%, 85%, 90%, 95%, 99% or 100%. In another embodiment, the brain injury is MCI such that detection of an elevated level of IL-18 in serum obtained from the patient as compared to a control (e.g., a pre-determined reference value or range of reference values) as provided herein determines that the patient has MCI with a specificity of at least 50%, 55%, 60%, 65%, 75%, 80%, 85%, 90%, 95%, 99% or 100%. The pre-determined reference value for these embodiments can be the cut-off values shown in Tables 22A and 25. In yet another embodiment, the brain injury is MCI such that detection of an elevated level of IL-18 in serum obtained from the patient as compared to a control (e.g., a pre-determined reference value or range of reference values) as provided herein determines that the patient has MCI with a sensitivity of at least 70%, and a specificity of at least 55%. The pre-determined reference value for this embodiment can be the cut-off values shown in Tables 22A and 25. In some cases, the range of reference values from about 200 pg/ml to about 214 pg/ml to attain a sensitivity of at least 70% and a specificity of at least 50%.

In one embodiment, the brain injury is MCI such that detection of an elevated level of caspase-1 in serum obtained from the patient as compared to a control (e.g., a pre-determined reference value or range of reference values) as provided herein determines that the patient has MCI with a sensitivity of at least 55%, 60%, 65%, 70%, 75%, 80%, 85%, 90%, 95%, 99% or 100%. In another embodiment, the brain injury is MCI such that detection of an elevated level of caspase-1 in serum obtained from the patient as compared to a control (e.g., a pre-determined reference value or range of reference values) as provided herein determines that the patient has MCI with a specificity of at least 40%, 45%, 50%, 55%, 60%, 65%, 75%, 80%, 85%, 90%, 95%, 99% or 100%. The pre-determined reference value for these embodiments can be the cut-off values shown in Table 22A. In yet another embodiment, the brain injury is MCI such that detection of an elevated level of caspase-1 in serum obtained from the patient as compared to a control (e.g., a pre-determined reference value or range of reference values) as provided herein determines that the patient has MCI with a sensitivity of at least 65%, and a specificity of at least 40%. The pre-determined reference value for this embodiment can be the cut-off values shown in Table 22A. In some cases, a reference values of about 1.75 pg/ml is used to attain a sensitivity of at least 65% and a specificity of at least 40%.

In one embodiment, the brain injury is MCI such that detection of an elevated level of IL-1β in serum obtained from the patient as compared to a control (e.g., a pre-determined reference value or range of reference values) as provided herein determines that the patient has MCI with a sensitivity of at least 55%, 60%, 65%, 70%, 75%, 80%, 85%, 90%, 95%, 99% or 100%. In another embodiment, the brain injury is MCI such that detection of an elevated level of IL-1β in serum obtained from the patient as compared to a control (e.g., a pre-determined reference value or range of reference values) as provided herein determines that the patient has MCI with a specificity of at least 40%, 45%, 50%, 55%, 60%, 65%, 75%, 80%, 85%, 90%, 95%, 99% or 100%. The pre-determined reference value for these embodiments can be the cut-off values shown in Table 22A. In yet another embodiment, the brain injury is MCI such that detection of an elevated level of IL-1β in serum obtained from the patient as compared to a control (e.g., a pre-determined reference value or range of reference values) as provided herein determines that the patient has MCI with a sensitivity of at least 65%, and a specificity of at least 55%. The pre-determined reference value for this embodiment can be the cut-off values shown in Table 22A. In some cases, a reference values of about 0.684 pg/ml is used to attain a sensitivity of at least 65% and a specificity of at least 50%.

In one embodiment, the brain injury is MCI such that detection of an elevated level of sAPPα in serum obtained from the patient as compared to a control (e.g., a pre-determined reference value or range of reference values) as provided herein determines that the patient has MCI with a sensitivity of at least 75%, 80%, 85%, 90%, 95%, 96%, 97%, 98%, 99% or 100%. In another embodiment, the brain injury is MCI such that detection of an elevated level of sAPPα in serum obtained from the patient as compared to a control (e.g., a pre-determined reference value or range of reference values) as provided herein determines that the patient has MCI with a specificity of at least 70%, 75%, 80%, 85%, 90%, 95%, 99% or 100%. The pre-determined reference value for these embodiments can be the cut-off values shown in Table 22A. In yet another embodiment, the brain injury is MCI such that detection of an elevated level of sAPPα in serum obtained from the patient as compared to a control (e.g., a pre-determined reference value or range of reference values) as provided herein determines that the patient has MCI with a sensitivity of at least 95%, and a specificity of at least 70%. The pre-determined reference value for this embodiment can be the cut-off values shown in Table 22A. In some cases, a reference values of about 1.39 ng/mL is used to attain a sensitivity of at least 95% and a specificity of at least 70%.

In one embodiment, the brain injury is MCI such that detection of an elevated level of sAPPβ in serum obtained from the patient as compared to a control (e.g., a pre-determined reference value or range of reference values) as provided herein determines that the patient has MCI with a sensitivity of at least 75%, 80%, 85%, 90%, 95%, 99% or 100%. In another embodiment, the brain injury is MCI such that detection of an elevated level of sAPPβ in serum obtained from the patient as compared to a control (e.g., a pre-determined reference value or range of reference values) as provided herein determines that the patient has MCI with a specificity of at least 75%, 80%, 85%, 90%, 95%, 99% or 100%. The pre-determined reference value for these embodiments can be the cut-off values shown in Table 22A. In yet another embodiment, the brain injury is MCI such that detection of an elevated level of sAPPβ in serum obtained from the patient as compared to a control (e.g., a pre-determined reference value or range of reference values) as provided herein determines that the patient has MCI with a sensitivity of at least 90%, and a specificity of at least 75%. The pre-determined reference value for this embodiment can be the cut-off values shown in Table 22A. In some cases, a reference values of about 0.26 ng/mL is used to attain a sensitivity of at least 90% and a specificity of at least 75%.

In one embodiment, the brain injury is MCI such that detection of an elevated level of NFL in serum obtained from the patient as compared to a control (e.g., a pre-determined reference value or range of reference values) as provided herein determines that the patient has MCI with a sensitivity of at least 55%, 60%, 65%, 70%, 75%, 80%, 85%, 90%, 95%, 99% or 100%. In another embodiment, the brain injury is MCI such that detection of an elevated level of NFL in serum obtained from the patient as compared to a control (e.g., a pre-determined reference value or range of reference values) as provided herein determines that the patient has MCI with a specificity of at least 50%, 55%, 60%, 65%, 70%, 75%, 80%, 85%, 90%, 95%, 99% or 100%. The pre-determined reference value for these embodiments can be the cut-off values shown in Table 22A. In yet another embodiment, the brain injury is MCI such that detection of an elevated level of NFL in serum obtained from the patient as compared to a control (e.g., a pre-determined reference value or range of reference values) as provided herein determines that the patient has MCI with a sensitivity of at least 70%, and a specificity of at least 75%. The pre-determined reference value for this embodiment can be the cut-off values shown in Table 22A. In some cases, a reference values of about 24 pg/mL is used to attain a sensitivity of at least 70% and a specificity of at least 75%.

In one embodiment, the brain injury is AD such that detection of an elevated level of ASC in serum obtained from the patient as compared to a control (e.g., a pre-determined reference value or range of reference values) as provided herein determines that the patient has AD with a sensitivity of at least 75%, 80%, 85%, 90%, 95%, 99% or 100%. In another embodiment, the brain injury is AD such that detection of an elevated level of ASC in serum obtained from the patient as compared to a control (e.g., a pre-determined reference value or range of reference values) as provided herein determines that the patient has AD with a specificity of at least 50%, 55%, 60% 65%, 70%, 75%, 80%, 85%, 90%, 95%, 99% or 100%. The pre-determined reference value for these embodiments can be the cut-off values shown in Table 22B. In yet another embodiment, the brain injury is AD such that detection of an elevated level of ASC in serum obtained from the patient as compared to a control (e.g., a pre-determined reference value or range of reference values) as provided herein determines that the patient has AD with a sensitivity of at least 80%, and a specificity of at least 70%. The pre-determined reference value(s) for this embodiment can be the cut-off values shown in Tables 22B. In some cases, a reference value of about 259 pg/mL can attain a sensitivity of at least 80% and a specificity of at least 70%. In some cases, the cut-off values for diagnosing AD vs. MCI is above 264.9 pg/ml and below 560 pg/ml.

In one embodiment, the brain injury is AD such that detection of an elevated level of IL-18 in serum obtained from the patient as compared to a control (e.g., a pre-determined reference value or range of reference values) as provided herein determines that the patient has AD with a sensitivity of at least 65%, 70%, 75%, 80%, 85%, 90%, 95%, 99% or 100%. In another embodiment, the brain injury is AD such that detection of an elevated level of IL-18 in serum obtained from the patient as compared to a control (e.g., a pre-determined reference value or range of reference values) as provided herein determines that the patient has AD with a specificity of at least 40%, 45%, 50%, 55%, 60%, 65%, 75%, 80%, 85%, 90%, 95%, 99% or 100%. The pre-determined reference value for these embodiments can be the cut-off values shown in Table 22B. In yet another embodiment, the brain injury is AD such that detection of an elevated level of IL-18 in serum obtained from the patient as compared to a control (e.g., a pre-determined reference value or range of reference values) as provided herein determines that the patient has AD with a sensitivity of at least 70%, and a specificity of at least 40%. The pre-determined reference value for this embodiment can be the cut-off values shown in Table 22B. In some cases, a reference values of about 196 pg/ml is used to attain a sensitivity of at least 70% and a specificity of at least 40%.

In one embodiment, the brain injury is AD such that detection of an elevated level of caspase-1 in serum obtained from the patient as compared to a control (e.g., a pre-determined reference value or range of reference values) as provided herein determines that the patient has AD with a sensitivity of at least 55%, 60%, 65%, 70%, 75%, 80%, 85%, 90%, 95%, 99% or 100%. In another embodiment, the brain injury is AD such that detection of an elevated level of caspase-1 in serum obtained from the patient as compared to a control (e.g., a pre-determined reference value or range of reference values) as provided herein determines that the patient has AD with a specificity of at least 40%, 45%, 50%, 55%, 60%, 65%, 75%, 80%, 85%, 90%, 95%, 99% or 100%. The pre-determined reference value for these embodiments can be the cut-off values shown in Table 22B. In yet another embodiment, the brain injury is AD such that detection of an elevated level of caspase-1 in serum obtained from the patient as compared to a control (e.g., a pre-determined reference value or range of reference values) as provided herein determines that the patient has AD with a sensitivity of at least 65%, and a specificity of at least 55%. The pre-determined reference value for this embodiment can be the cut-off values shown in Table 22B. In some cases, a reference values of about 1.78 pg/ml is used to attain a sensitivity of at least 65% and a specificity of at least 55%.

In one embodiment, the brain injury is AD such that detection of an elevated level of IL-1β in serum obtained from the patient as compared to a control (e.g., a pre-determined reference value or range of reference values) as provided herein determines that the patient has AD with a sensitivity of at least 55%, 60%, 65%, 70%, 75%, 80%, 85%, 90%, 95%, 99% or 100%. In another embodiment, the brain injury is AD such that detection of an elevated level of IL-1β in serum obtained from the patient as compared to a control (e.g., a pre-determined reference value or range of reference values) as provided herein determines that the patient has AD with a specificity of at least 40%, 45%, 50%, 55%, 60%, 65%, 75%, 80%, 85%, 90%, 95%, 99% or 100%. The pre-determined reference value for these embodiments can be the cut-off values shown in Table 22B. In yet another embodiment, the brain injury is AD such that detection of an elevated level of IL-1β in serum obtained from the patient as compared to a control (e.g., a pre-determined reference value or range of reference values) as provided herein determines that the patient has AD with a sensitivity of at least 65%, and a specificity of at least 55%. The pre-determined reference value for this embodiment can be the cut-off values shown in Table 22B. In some cases, a reference values of about 0.693 pg/ml is used to attain a sensitivity of at least 75% and a specificity of at least 40%.

In one embodiment, the brain injury is AD such that detection of an elevated level of sAPPα in serum obtained from the patient as compared to a control (e.g., a pre-determined reference value or range of reference values) as provided herein determines that the patient has AD with a sensitivity of at least 75%, 80%, 85%, 90%, 95%, 99% or 100%. In another embodiment, the brain injury is AD such that detection of an elevated level of sAPPα in serum obtained from the patient as compared to a control (e.g., a pre-determined reference value or range of reference values) as provided herein determines that the patient has AD with a specificity of at least 75%, 80%, 85%, 90%, 95%, 99% or 100%. The pre-determined reference value for these embodiments can be the cut-off values shown in Table 22B. In yet another embodiment, the brain injury is AD such that detection of an elevated level of sAPPα in serum obtained from the patient as compared to a control (e.g., a pre-determined reference value or range of reference values) as provided herein determines that the patient has AD with a sensitivity of at least 90%, and a specificity of at least 90%. The pre-determined reference value for this embodiment can be the cut-off values shown in Table 22B. In some cases, a reference values of about 2.5 ng/mL is used to attain a sensitivity of at least 90% and a specificity of at least 90%.

In one embodiment, the brain injury is AD such that detection of an elevated level of sAPPβ in serum obtained from the patient as compared to a control (e.g., a pre-determined reference value or range of reference values) as provided herein determines that the patient has AD with a sensitivity of at least 75%, 80%, 85%, 90%, 95%, 99% or 100%. In another embodiment, the brain injury is AD such that detection of an elevated level of sAPPβ in serum obtained from the patient as compared to a control (e.g., a pre-determined reference value or range of reference values) as provided herein determines that the patient has AD with a specificity of at least 75%, 80%, 85%, 90%, 95%, 99% or 100%. The pre-determined reference value for these embodiments can be the cut-off values shown in Table 22B. In yet another embodiment, the brain injury is AD such that detection of an elevated level of sAPPβ in serum obtained from the patient as compared to a control (e.g., a pre-determined reference value or range of reference values) as provided herein determines that the patient has AD with a sensitivity of at least 80%, and a specificity of at least 80%. The pre-determined reference value for this embodiment can be the cut-off values shown in Table 22B. In some cases, a reference values of about 0.29 ng/mL is used to attain a sensitivity of at least 80% and a specificity of at least 80%.

In one embodiment, the brain injury is AD such that detection of an elevated level of NFL in serum obtained from the patient as compared to a control (e.g., a pre-determined reference value or range of reference values) as provided herein determines that the patient has AD with a sensitivity of at least 55%, 60%, 65%, 70%, 75%, 80%, 85%, 90%, 95%, 99% or 100%. In another embodiment, the brain injury is AD such that detection of an elevated level of NFL in serum obtained from the patient as compared to a control (e.g., a pre-determined reference value or range of reference values) as provided herein determines that the patient has AD with a specificity of at least 50%, 55%, 60%, 65%, 70%, 75%, 80%, 85%, 90%, 95%, 99% or 100%. The pre-determined reference value for these embodiments can be the cut-off values shown in Table 22B. In yet another embodiment, the brain injury is AD such that detection of an elevated level of NFL in serum obtained from the patient as compared to a control (e.g., a pre-determined reference value or range of reference values) as provided herein determines that the patient has AD with a sensitivity of at least 60%, and a specificity of at least 55%. The pre-determined reference value for this embodiment can be the cut-off values shown in Table 22B. In some cases, a reference values of about 21.4 pg/mL is used to attain a sensitivity of at least 60% and a specificity of at least 55%.

In one embodiment, the brain injury's MCI and AD can be distinguished by comparing the level of ASC in serum obtained from the patient with MCI to a patient with AD (e.g., a pre-determined reference value or range of reference values). In some embodiments, this method determines a patient's brain injury (e.g. AD or MCI) with a sensitivity of at least 70%, 75%, 80%, 85%, 90%, 95%, 99% or 100%. In another embodiment, this method determines a patient's brain injury (e.g. AD or MCI) with a specificity of at least 60% 65%, 70%, 75%, 80%, 85%, 90%, 95%, 99% or 100%. The pre-determined reference value for these embodiments can be the cut-off values shown in Table 22C. In yet another embodiment, this method determines a patient's brain injury based on a level of ASC (e.g. AD or MCI) with a sensitivity of at least 70%, and a specificity of at least 60%. The pre-determined reference value(s) for this embodiment can be the cut-off values shown in Tables 22C. In some cases, a reference value of about 560 pg/mL can attain a sensitivity of at least 70% and a specificity of at least 60%.

In one embodiment, the brain injury's MCI and AD can be distinguished by comparing the level of Caspase-1 in serum obtained from the patient with MCI to a patient with AD (e.g., a pre-determined reference value or range of reference values). In some embodiments, this method determines a patient's brain injury (e.g. AD or MCI) with a sensitivity of at least 70%, 75%, 80%, 85%, 90%, 95%, 99% or 100%. In another embodiment, this method determines a patient's brain injury (e.g. AD or MCI) with a specificity of at least 60% 65%, 70%, 75%, 80%, 85%, 90%, 95%, 99% or 100%. The pre-determined reference value for these embodiments can be the cut-off values shown in Table 22C. In yet another embodiment, this method determines a patient's brain injury (e.g. AD or MCI) based on a level of Caspase-1 with a sensitivity of at least 70%, and a specificity of at least 60%. The pre-determined reference value(s) for this embodiment can be the cut-off values shown in Tables 22C. In some cases, a reference value of about 1.94 pg/mL can attain a sensitivity of at least 70% and a specificity of at least 60%.

In one embodiment, the brain injury's MCI and AD can be distinguished by comparing the level of IL-18 in serum obtained from the patient with MCI to a patient with AD (e.g., a pre-determined reference value or range of reference values). In some embodiments, this method determines a patient's brain injury (e.g. AD or MCI) with a sensitivity of at least 70%, 75%, 80%, 85%, 90%, 95%, 99% or 100%. In another embodiment, this method determines a patient's brain injury (e.g. AD or MCI) with a specificity of at least about 40%, 45%, 50%, 55%, 60% 65%, 70%, 75%, 80%, 85%, 90%, 95%, 99% or 100%. The pre-determined reference value for these embodiments can be the cut-off values shown in Table 22C. In yet another embodiment, this method determines a patient's brain injury (e.g. AD or MCI) based on a level of IL-18 with a sensitivity of at least 70%, and a specificity of at least 45%. The pre-determined reference value(s) for this embodiment can be the cut-off values shown in Tables 22C. In some cases, a reference value of about 290 pg/mL can attain a sensitivity of at least 70% and a specificity of at least 45%.

In one embodiment, the brain injury's MCI and AD can be distinguished by comparing the level of IL-1β in serum obtained from the patient with MCI to a patient with AD (e.g., a pre-determined reference value or range of reference values). In some embodiments, this method determines a patient's brain injury (e.g. AD or MCI) with a sensitivity of at least 70%, 75%, 80%, 85%, 90%, 95%, 99% or 100%. In another embodiment, this method determines a patient's brain injury (e.g. AD or MCI) with a specificity of at least 40%, 45%, 50%, 55%, 60%, 65%, 70%, 75%, 80%, 85%, 90%, 95%, 99% or 100%. The pre-determined reference value for these embodiments can be the cut-off values shown in Table 22C. In yet another embodiment, this method determines a patient's brain injury (e.g. AD or MCI) based on a level of IL-1β with a sensitivity of at least 75%, and a specificity of at least 40%. The pre-determined reference value(s) for this embodiment can be the cut-off values shown in Tables 22C. In some cases, a reference value of about 0.46 pg/mL can attain a sensitivity of at least 75% and a specificity of at least 40%.

In one embodiment, the brain injury's MCI and AD can be distinguished by comparing the level of sAPPα in serum obtained from the patient with MCI to a patient with AD (e.g., a pre-determined reference value or range of reference values). In some embodiments, this method determines a patient's brain injury (e.g. AD or MCI) with a sensitivity of at least 70%, 75%, 80%, 85%, 90%, 95%, 99% or 100%. In another embodiment, this method determines a patient's brain injury (e.g. AD or MCI) with a specificity of at least 40%, 45%, 50%, 55%, 60%, 65%, 70%, 75%, 80%, 85%, 90%, 95%, 99% or 100%. The pre-determined reference value for these embodiments can be the cut-off values shown in Table 22C. In yet another embodiment, this method determines a patient's brain injury (e.g. AD or MCI) based on a level of sAPPα with a sensitivity of at least 70%, and a specificity of at least 55%. The pre-determined reference value(s) for this embodiment can be the cut-off values shown in Tables 22C. In some cases, a reference value of about 8.84 ng/mL can attain a sensitivity of at least 70% and a specificity of at least 55%.

In one embodiment, the brain injury's MCI and AD can be distinguished by comparing the level of sAPPβ in serum obtained from the patient with MCI to a patient with AD (e.g., a pre-determined reference value or range of reference values). In some embodiments, this method determines a patient's brain injury (e.g. AD or MCI) with a sensitivity of at least 60%, 65%, 70%, 75%, 80%, 85%, 90%, 95%, 99% or 100%. In another embodiment, this method determines a patient's brain injury (e.g. AD or MCI) with a specificity of at least 40%, 45%, 50%, 55%, 60%, 65%, 70%, 75%, 80%, 85%, 90%, 95%, 99% or 100%. The pre-determined reference value for these embodiments can be the cut-off values shown in Table 22C. In yet another embodiment, this method determines a patient's brain injury (e.g. AD or MCI) based on a level of sAPPβ with a sensitivity of at least 60%, and a specificity of at least 45%. The pre-determined reference value(s) for this embodiment can be the cut-off values shown in Tables 22C. In some cases, a reference value of about 0.63 ng/mL can attain a sensitivity of at least 60% and a specificity of at least 45%.

In one embodiment, the brain injury's MCI and AD can be distinguished by comparing the level of NFL in serum obtained from the patient with MCI to a patient with AD (e.g., a pre-determined reference value or range of reference values). In some embodiments, this method determines a patient's brain injury (e.g. AD or MCI) with a sensitivity of at least 60%, 65%, 70%, 75%, 80%, 85%, 90%, 95%, 99% or 100%. In another embodiment, this method determines a patient's brain injury (e.g. AD or MCI) with a specificity of at least 40%, 45%, 50%, 55%, 60%, 65%, 70%, 75%, 80%, 85%, 90%, 95%, 99% or 100%. The pre-determined reference value for these embodiments can be the cut-off values shown in Table 22C. In yet another embodiment, this method determines a patient's brain injury (e.g. AD or MCI) based on a level of NFL with a sensitivity of at least 70%, and a specificity of at least 40%. The pre-determined reference value(s) for this embodiment can be the cut-off values shown in Tables 22C. In some cases, a reference value of about 33.9 pg/mL can attain a sensitivity of at least 70% and a specificity of at least 40%.

In another embodiment, the disease, disorder or condition associated with inflammation is an age-related disease. In one embodiment, the age-related disorder is AMD such that detection of an elevated level of ASC in serum obtained from the patient as compared to a control (e.g., a pre-determined reference value or range of reference values) as provided herein determines that the patient has AMD with a sensitivity of at least 75%, 80%, 90%, 95%, 99% or 100%. In another embodiment, the age-related disease is AMD such that detection of an elevated level of ASC in serum obtained from the patient as compared to a control (e.g., a pre-determined reference value or range of reference values) as provided herein determines that the patient has AMD with a specificity of at least 75%, 80%, 85%, 90%, 95%, 99% or 100%. The pre-determined reference value for these embodiments can be the cut-off values shown in Table 29. In yet another embodiment, the age-related disease is AMD such that detection of an elevated level of ASC in serum obtained from the patient as compared to a control (e.g., a pre-determined reference value or range of reference values) as provided herein determines that the patient has AMD with a sensitivity of at least 90%, and a specificity of at least 80%. The pre-determined reference value for this embodiment can be the cut-off values shown in Table 29. In some cases, a reference value of about 365.6 pg/mL can attain a sensitivity of at least 90% and a specificity of at least 85%.

In one embodiment, the age-related disease is AMD such that detection of an elevated level of caspase-1 in serum obtained from the patient as compared to a control (e.g., a pre-determined reference value or range of reference values) as provided herein determines that the patient has AMD with a sensitivity of at least 60%, 65%, 70%, 75%, 80%, 90%, 95%, 99% or 100%. In another embodiment, the age-related disease is AMD such that detection of an elevated level of caspase-1 in serum obtained from the patient as compared to a control (e.g., a pre-determined reference value or range of reference values) as provided herein determines that the patient has AMD with a specificity of at least 25%, 30%, 35%, 40%, 45%, 45% 50%, 55%, 60%, 65%, 70%, 75%, 80%, 85%, 90%, 95%, 99% or 100%. The pre-determined reference value for these embodiments can be the cut-off values shown in Table 29. In yet another embodiment, the age-related disease is AMD such that detection of an elevated level of caspase-1 in serum obtained from the patient as compared to a control (e.g., a pre-determined reference value or range of reference values) as provided herein determines that the patient has AMD with a sensitivity of at least 75%, and a specificity of at least 30%. The pre-determined reference value for this embodiment can be the cut-off values shown in Table 29. In some cases, a reference value of about 6.136 pg/mL can attain a sensitivity of at least 75% and a specificity of at least 30%.

In one embodiment, the age-related disease is AMD such that detection of an elevated level of IL-18 in serum obtained from the patient as compared to a control (e.g., a pre-determined reference value or range of reference values) as provided herein determines that the patient has AMD with a sensitivity of at least 70%, 75%, 80%, 90%, 95%, 99% or 100%. In another embodiment, the age-related disease is AMD such that detection of an elevated level of caspase-1 in serum obtained from the patient as compared to a control (e.g., a pre-determined reference value or range of reference values) as provided herein determines that the patient has AMD with a specificity of at least 45% 50%, 55%, 60%, 65%, 70%, 75%, 80%, 85%, 90%, 95%, 99% or 100%. The pre-determined reference value for these embodiments can be the cut-off values shown in Table 29. In yet another embodiment, the age-related disease is AMD such that detection of an elevated level of IL-18 in serum obtained from the patient as compared to a control (e.g., a pre-determined reference value or range of reference values) as provided herein determines that the patient has AMD with a sensitivity of at least 70%, and a specificity of at least 50%. The pre-determined reference value for this embodiment can be the cut-off values shown in Table 29. In some cases, a reference value of about 242.4 pg/mL can attain a sensitivity of at least 70% and a specificity of at least 50%.

In one embodiment, the age-related disease is AMD such that detection of an elevated level of IL-1β in serum obtained from the patient as compared to a control (e.g., a pre-determined reference value or range of reference values) as provided herein determines that the patient has AMD with a sensitivity of at least 45%, 50%, 55%, 60%, 65%, 70%, 75%, 80%, 90%, 95%, 99% or 100%. In another embodiment, the age-related disease is AMD such that detection of an elevated level of IL-1β in serum obtained from the patient as compared to a control (e.g., a pre-determined reference value or range of reference values) as provided herein determines that the patient has AMD with a specificity of at least 45% 50%, 55%, 60%, 65%, 70%, 75%, 80%, 85%, 90%, 95%, 99% or 100%. The pre-determined reference value for these embodiments can be the cut-off values shown in Table 29. In yet another embodiment, the age-related disease is AMD such that detection of an elevated level of IL-1β in serum obtained from the patient as compared to a control (e.g., a pre-determined reference value or range of reference values) as provided herein determines that the patient has AMD with a sensitivity of at least 55%, and a specificity of at least 50%. The pre-determined reference value for this embodiment can be the cut-off values shown in Table 29. In some cases, a reference value of about 0.842 pg/mL can attain a sensitivity of at least 55% and a specificity of at least 50%.

In another embodiment, the disease, disorder or condition associated with inflammation is a type of Nonalcoholic fatty liver disease (NAFLD). In one embodiment, the type of NAFLD is NASH such that detection of an elevated level of ASC in serum obtained from the patient as compared to a control (e.g., a pre-determined reference value or range of reference values) as provided herein determines that the patient has NASH with a sensitivity of at least 75%, 80%, 90%, 95%, 99% or 100%. In another embodiment, the disease associated with inflammation is NASH such that detection of an elevated level of ASC in serum obtained from the patient as compared to a control (e.g., a pre-determined reference value or range of reference values) as provided herein determines that the patient has NASH with a specificity of at least 65%, 70%, 75%, 80%, 85%, 90%, 95%, 99% or 100%. The pre-determined reference value for these embodiments can be the cut-off values shown in Table 34. In yet another embodiment, the disease associated with inflammation is NASH such that detection of an elevated level of ASC in serum obtained from the patient as compared to a control (e.g., a pre-determined reference value or range of reference values) as provided herein determines that the patient has AMD with a sensitivity of at least 80%, and a specificity of at least 60%. The pre-determined reference value for this embodiment can be the cut-off values shown in Table 34. In some cases, a reference value of about 394.9 pg/mL can attain a sensitivity of at least 80% and a specificity of at least 60%.

In one embodiment, the disease associated with inflammation is NASH such that detection of an elevated level of IL-18 in serum obtained from the patient as compared to a control (e.g., a pre-determined reference value or range of reference values) as provided herein determines that the patient has NASH with a sensitivity of at least 60%, 65%, 70%, 75%, 80%, 90%, 95%, 99% or 100%. In another embodiment, the disease associated with inflammation is NASH such that detection of an elevated level of IL-18 in serum obtained from the patient as compared to a control (e.g., a pre-determined reference value or range of reference values) as provided herein determines that the patient has NASH with a specificity of at least 25%, 30%, 35%, 40%, 45%, 45% 50%, 55%, 60%, 65%, 70%, 75%, 80%, 85%, 90%, 95%, 99% or 100%. The pre-determined reference value for these embodiments can be the cut-off values shown in Table 34. In yet another embodiment, the disease associated with inflammation is NASH such that detection of an elevated level of IL-18 in serum obtained from the patient as compared to a control (e.g., a pre-determined reference value or range of reference values) as provided herein determines that the patient has NASH with a sensitivity of at least 75%, and a specificity of at least 60%. The pre-determined reference value for this embodiment can be the cut-off values shown in Table 34. In some cases, a reference value of about 269.2 pg/mL can attain a sensitivity of at least 75% and a specificity of at least 60%. In any of the methods provided herein, the sensitivity and/or specificity of an inflammasome protein (e.g., ASC) for predicting or diagnosing a disease, disorder or condition associated with inflammation (e.g., NASH, MCI, AD, AMD, inflammaging, stroke, MS or TBI) is determined by calculation of area under curve (AUC) values with confidence intervals (e.g., 95%). The area under curve (AUC) can be determined from receiver operator characteristic (ROC) curves with confidence intervals of 95%.

In one embodiment, the disease, disorder or condition associated with inflammation is a brain injury. In one embodiment, the brain injury is MS such that detection of a level or concentration of at least one inflammasome protein in a biological sample obtained from the patient that is elevated by a pre-determined percentage over the level of the same at least one inflammasome protein in a biological sample obtained from a control subject is indicative of the patient as having MS. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. In one embodiment, the brain injury is MS such that detection of a level or concentration of ASC in serum obtained from the patient that is at least 50% higher than the level of ASC in a serum sample obtained from a control subject is indicative of the patient as having MS. In one embodiment, the brain injury is MS such that detection of a level or concentration of ASC in a sample obtained from the patient that is higher than the level of ASC in a sample obtained from a control subject is indicative of the patient as having MS, when said patient also has altered level or concentration of a known MS biomarker in a sample obtained from the patient as compared to the level of the known MS biomarker(s) in a sample obtained from a control subject known to not have AD.

In one embodiment, the brain injury is stroke such that detection of a level or concentration of at least one inflammasome protein in a biological sample obtained from the patient that is elevated by a pre-determined percentage over the level of the same at least one inflammasome protein in a biological sample obtained from a control subject is indicative of the patient as having stroke. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. In one embodiment, the brain injury is stroke such that detection of a level or concentration of ASC in serum obtained from the patient that is at least 70% higher than the level of ASC in a serum sample obtained from a control subject is indicative of the patient as having suffered a stroke. In one embodiment, the brain injury is stroke such that detection of a level or concentration of ASC in serum-derived EVs obtained from the patient that is at least 110% higher than the level of ASC in a serum-derived EVs sample obtained from a control subject is indicative of the patient as having suffered a stroke. In one embodiment, the brain injury is stroke such that detection of a level or concentration of ASC in a sample obtained from the patient that is higher than the level of ASC in a sample obtained from a control subject is indicative of the patient as having a stroke, when said patient also has altered level or concentration of a known stroke biomarker in a sample obtained from the patient as compared to the level of the known stroke biomarker(s) in a sample obtained from a control subject known to not have suffered a stroke.

In one embodiment, the brain injury is TBI such that detection of a level or concentration of at least one inflammasome protein in a biological sample obtained from the patient that is elevated by a pre-determined percentage over the level of the same at least one inflammasome protein in a biological sample obtained from a control subject is indicative of the patient as having TBI. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. In one embodiment, the brain injury is TBI such that detection of a level or concentration of ASC in serum obtained from the patient that is at least 50% higher than the level of ASC in a serum sample obtained from a control subject is indicative of the patient as having TBI. In one embodiment, the brain injury is TBI such that detection of a level or concentration of ASC in sample obtained from the patient that is higher than the level of ASC in a serum sample obtained from a control subject is indicative of the patient as having TBI, when said patient also has altered level or concentration of a known TBI biomarker in a sample obtained from the patient as compared to the level of the known TBI biomarker(s) in a sample obtained from a control subject known to not have TBI.

In one embodiment, the brain injury is MCI such that detection of a level or concentration of at least one inflammasome protein alone or in combination with at least one control biomarker protein in a biological sample obtained from the patient that is elevated by a pre-determined percentage over the level of the same at least one inflammasome protein in a biological sample obtained from a control subject is indicative of the patient as having MCI. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. The at least one control biomarker protein can be AB (1-42), AB (140), sAPPα, sAPPβ, T-Tau or NFL. In one embodiment, the brain injury is MCI such that detection of a level or concentration of ASC in serum obtained from the patient that is at least 50% higher than the level of ASC in a serum sample obtained from a control subject is indicative of the patient as having MCI. In one embodiment, the brain injury is MCI such that detection of a level or concentration of ASC in a sample obtained from the patient that is higher than the level of ASC in a sample obtained from a control subject is indicative of the patient as having MCI, when said patient also has altered level or concentration of a known MCI biomarker in a sample obtained from the patient as compared to the level of the known MCI biomarker(s) in a sample obtained from a control subject known to not have MCI.

In one embodiment, the brain injury is AD such that detection of a level or concentration of at least one inflammasome protein alone or in combination with at least one control biomarker protein in a biological sample obtained from the patient that is elevated by a pre-determined percentage over the level of the same at least one inflammasome protein and/or control biomarker protein in a biological sample obtained from a control subject is indicative of the patient as having AD. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. The at least one control biomarker protein can be AB₍₁₋₄₂₎, AB₍₁₋₄₀₎, sAPPα, sAPPβ, T-Tau or NFL. In one embodiment, the brain injury is AD such that detection of a level or concentration of ASC in serum obtained from the patient that is at least 50% higher than the level of ASC in a serum sample obtained from a control subject is indicative of the patient as having AD. In one embodiment, the brain injury is AD such that detection of a level or concentration of ASC in a sample obtained from the patient that is higher than the level of ASC in a sample obtained from a control subject is indicative of the patient as having AD, when said patient also has altered level or concentration of a known AD biomarker in a sample obtained from the patient as compared to the level of the known AD biomarker(s) in a sample obtained from a control subject known to not have AD.

In another embodiment, the disease, disorder or condition associated with inflammation is an age-related disease. In one embodiment, the age-related disease is AMD such that detection of a level or concentration of at least one inflammasome protein in a biological sample obtained from the patient that is elevated by a pre-determined percentage over the level of the same at least one inflammasome protein in a biological sample obtained from a control subject is indicative of the patient as having AMD. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. In one embodiment, the age-related disease is AMD such that detection of a level or concentration of ASC in serum obtained from the patient that is at least 50% higher than the level of ASC in a serum sample obtained from a control subject is indicative of the patient as having AMD. In one embodiment, the disease, disorder or condition associated with inflammation is AMD such that detection of a level or concentration of ASC in a sample obtained from the patient that is higher than the level of ASC in a sample obtained from a control subject is indicative of the patient as having AMD, when said patient also has an altered level or concentration of a known AMD biomarker in a sample obtained from the patient as compared to the level of the known AMD biomarker(s) in a sample obtained from a control subject known to not have AMD.

In one embodiment, the disease, disorder or condition associated with inflammation is NASH such that detection of a level or concentration of at least one inflammasome protein alone or in combination with at least one control biomarker protein in a biological sample obtained from the patient that is elevated by a pre-determined percentage over the level of the same at least one inflammasome protein and/or control biomarker protein in a biological sample obtained from a control subject is indicative of the patient as having NASH. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from IL-18 and ASC. The at least one control biomarker protein can be CRP (hs-CRP) or Gal-3. In one embodiment, the disease, disorder or condition associated with inflammation is NASH such that detection of a level or concentration of ASC in serum obtained from the patient that is at least 50% higher than the level of ASC in a serum sample obtained from a control subject is indicative of the patient as having NASH. In one embodiment, the disease, disorder or condition associated with inflammation is NASH such that detection of a level or concentration of ASC in a sample obtained from the patient that is higher than the level of ASC in a sample obtained from a control subject is indicative of the patient as having NASH, when said patient also has an increase in the level or concentration of a known NASH biomarker such as Gal-3 or CRP (hs-CRP) in a sample obtained from the patient as compared to the level of the known NASH biomarker(s) in a sample obtained from a control subject known to not have NASH.

The present invention also provides a method of determining a prognosis for a patient with inflammation or a disease, disorder or condition caused by or associated with inflammation (e.g., MCI, AD, AMD, inflammaging, stroke, MS or TBI). In one embodiment, the method comprises providing a biological sample obtained from the patient and measuring the level of at least one inflammasome protein alone or in combination with at least one control biomarker protein in the biological sample to prepare a protein profile as described above, wherein the inflammasome protein profile or the control biomarker protein profile is indicative of the prognosis of the patient. In some embodiments, an increase in the level of one or more inflammasome proteins (e.g., IL-18, NLRP1, ASC, caspase-1, or combinations thereof) relative to a pre-determined reference value or range of reference values is indicative of a poorer prognosis. For instance, an increase of about 20% to about 300% in the level of one or more inflammasome proteins relative to a pre-determined reference value or range of reference values is indicative of a poorer prognosis. In some cases, the inflammasome protein is ASC and the pre-determined reference values can be derived from Tables 7-9, 16, 22A-C or 23. In some embodiments, an increase in the level of one or more control biomarker proteins (e.g., AB₍₁₋₄₂₎, AB₍₁₋₄₀₎, sAPPα, sAPPβ, or NFL, or combinations thereof) relative to a pre-determined reference value or range of reference values is indicative of a poorer prognosis. For instance, an increase of about 20% to about 300% in the level of one or more control biomarker proteins relative to a pre-determined reference value or range of reference values is indicative of a poorer prognosis. In some embodiments, an increase in the level of one or more control biomarker proteins (e.g., AB₍₁₋₄₂₎, AB₍₁₋₄₀₎, sAPPα, sAPPβ, or NFL, or combinations thereof) and an increase in one or more inflammasome proteins relative to a pre-determined reference value or range of reference values is indicative of a poorer prognosis. For instance, an increase of about 20% to about 300% in the level of one or more control biomarker proteins and an increase of about 20% to about 300% in the level of one or more inflammasome proteins relative to a pre-determined reference value or range of reference values is indicative of a poorer prognosis.

In one embodiment, the expression of level of ASC in a biological sample obtained from a patient in any of the diagnostic methods provided herein is determined or detected through the use of any anti-ASC antibody known in the art and/or provided herein. In one embodiment, the anti-ASC is a monoclonal antibody or fragment thereof provided herein. In one embodiment, the anti-ASC antibody is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 19, or an amino acid sequence that is at least 95%, 96%, 97%, 98% or 99% identical to the amino acid sequence of SEQ ID NO: 19; and wherein the VL region amino acid sequence comprises SEQ ID NO: 30 or an amino acid sequence that is at least 95%, 96%, 97%, 98% or 99% identical to the amino acid sequence of SEQ ID NO: 30. In some cases, a monoclonal antibody or an antibody fragment derived therefrom comprising a VH region amino acid sequence comprising SEQ ID NO: 19 and a VL region amino acid sequence comprising SEQ ID NO: 30 can be referred to as IC-100.

Methods of Treatment

In one embodiment, provided herein are methods of treating patients suffering from or suspected of suffering from inflammation or a disease, disorder or condition caused by or associated with inflammation. Any method of treating provided herein can entail administering a treatment to the patients suffering from or suspected of suffering from the disease, disorder or condition caused by or associated with inflammation. In some cases, administration of the treatment in a method as provided herein can reduce inflammation in the patient. The reduction can be as compared to a control (e.g., untreated patient and/or patient prior to treatment). In some cases, the treatment is a standard of care treatment. In some cases, the treatment is a neuroprotective treatment. Such neuroprotective treatments can include drugs that reduce excitotoxicity, oxidative stress, and inflammation. Thus, suitable neuroprotective treatments include, but are not limited to, methylprednisolone, 17alpha-estradiol, 17beta-estradiol, ginsenoside, progesterone, simvastatin, deprenyl, minocycline, resveratrol, and other glutamate receptor antagonists (e.g. NMDA receptor antagonists) and antioxidants. In some embodiments, the treatments are antibodies against an inflammasome protein or binding fragments thereof, such as the antibodies directed against inflammasome proteins provided herein. In some cases, the treatment can be an extracellular vesicle (EV) uptake inhibitor. The EV uptake inhibitor can be any EV uptake inhibitor known in the art. In some cases, the EV uptake inhibitors can be selected from those found in Table 30. In some cases, the treatment is any combination of standard of care treatments, neuroprotective treatment, antibodies or fragments derived therefrom directed against an inflammasome protein and an EV uptake inhibitor.

In other embodiments, the methods of diagnosing or evaluating a patient as experiencing inflammation or having a disease, disorder or condition caused by or associated with inflammation further comprises administering a treatment for said inflammation or disease, disorder or condition caused by or associated with inflammation to the patient based on the measured level of said at least one inflammasome protein or at least one control biomarker protein or when a protein signature associated with inflammation or a disease, disorder or condition caused by or associated with inflammation is identified. The methods of diagnosing or evaluating a patient as having inflammation or a disease, disorder or condition caused by or associated with inflammation (e.g., NASH, MCI, stroke, inflammaging, AMD, MS, AD or TBI) can be ascertained using the methods described herein. In some embodiment, the methods of diagnosing or evaluating a patient having a disease, disorder or condition associated with inflammation further comprises administering a treatment to the patient based on the measured level of said at least one inflammasome protein or when a protein signature associated with a disease, disorder or condition associated with inflammation or a more severe disease, disorder or condition associated with inflammation is identified. In some cases, the treatment is a standard of care treatment. In some cases, the treatment is a neuroprotective treatment. In some cases, the treatments are antibodies against an inflammasome protein or binding fragments thereof, such as the antibodies directed against inflammasome proteins provided herein. In some cases, the treatment can be an extracellular vesicle (EV) uptake inhibitor. The EV uptake inhibitor can be any EV uptake inhibitor known in the art. In some cases, the EV uptake inhibitors can be selected from those found in Table 30. In some cases, the treatment is any combination of standard of care treatments, neuroprotective treatment, antibodies or fragments derived therefrom directed against an inflammasome protein and an EV uptake inhibitor. In some cases, administration of the treatment in a method as provided herein can reduce inflammation in the patient. The reduction can be as compared to a control (e.g., untreated patient and/or patient prior to treatment).

With respect to any of the method of treatment embodiments provided herein for treating inflammation or a disease, disorder or condition caused by or associated with inflammation. The inflammation can be an innate immune inflammation. The inflammation can be an inflammasome-related inflammation. The disease, disorder or condition can be selected from the group consisting of a brain injury, an age-related disease, inflammaging, an autoimmune, autoinflammatory, metabolic or neurodegenerative disease. In some cases, the disease, disorder or condition is inflammaging. In some cases, the disease, disorder or condition is NASH. In some cases, the age-related disease is age-related macular degeneration (AMD). In some cases, the disease, disorder or condition is a brain injury. The brain injury can be selected from the group consisting of traumatic brain injury (TBI), stroke and spinal cord injury (SCI). The autoimmune or neurodegenerative disease can be selected from amyotrophic lateral sclerosis (ALS), Alzheimer's disease, Parkinson's disease (PD), muscular dystrophy (MD), immune dysfunction muscular CNS breakdown, systemic lupus erythematosus, lupus nephritis, rheumatoid arthritis, inflammatory bowel disease (e.g., Crohn's Disease and ulcerative colitis) and multiple sclerosis (MS). The metabolic disease can be selected from metabolic syndrome, obesity, diabetes mellitus, diabetic nephropathy or diabetic kidney disease (DKD), insulin resistance, atherosclerosis, a lipid storage disorder, a glycogen storage disease, medium-chain acyl-coenzyme A dehydrogenase deficiency, non-alcoholic fatty liver disease (e.g., Nonalcoholic steatohepatitis (NASH)) and gout. The autoinflammatory disease can be cryopyrin-associated periodic syndrome (CAPS). CAPS can encompass familial cold autoinflammatory syndrome (FCAS), Muckle-Wells syndrome (MWS) and neonatal-onset multisystem inflammatory disease (NOMID).

In one embodiment, the brain injury (e.g., AD, MCI, TBI, stroke or MS) is MS and the standard of care treatment is selected from is selected from therapies directed towards modifying disease outcome, managing relapses, managing symptoms or any combination thereof. The therapies directed toward modifying disease outcome can be selected from beta-interferons, glatiramer acetate, fingolimod, teriflunomide, dimethyl fumarate, mitoxanthrone, ocrelizumab, alemtuzumab, daclizumab and natalizumab. The stroke can be ischemic stroke, transient ischemic stroke or hemorrhagic stroke.

In another embodiment, the brain injury (e.g., AD, MCI, TBI, stroke or MS) is ischemic stroke or transient ischemic stroke and the standard of care treatment is selected from tissue plasminogen activator (tPA), antiplatelet medicine, anticoagulants, a carotid artery angioplasty, carotid endarterectomy, intra-arterial thrombolysis and mechanical clot removal in cerebral ischemia (MERCI) or a combination thereof. In still another embodiment, the brain injury (e.g., TBI, stroke or MS) is hemorrhagic stroke and the standard of care treatment is an aneurysm clipping, coil embolization or arteriovenous malformation (AVM) repair.

In another embodiment, the brain injury (e.g., AD, MCI, TBI, stroke or MS) is TBI and the standard of care treatment is selected from diuretics, anti-seizure drugs, coma inducing drugs, surgery and/or rehabilitation. Diuretics can be used to reduce the amount of fluid in tissues and increase urine output. Diuretics, given intravenously to people with traumatic brain injury, can help reduce pressure inside the brain. An anti-seizure drug may be given during the first week to avoid any additional brain damage that might be caused by a seizure. Continued anti-seizure treatments are used only if seizures occur. Coma-inducing drugs can sometimes be used drugs to put people into temporary comas because a comatose brain needs less oxygen to function. This can be especially helpful if blood vessels, compressed by increased pressure in the brain, are unable to supply brain cells with normal amounts of nutrients and oxygen. The severity of the TBI can be assessed using the Glasgow Coma Scale. This 15-point test can help a doctor or other emergency medical personnel assess the initial severity of a brain injury by checking a person's ability to follow directions and move their eyes and limbs. The coherence of speech can also provide important clues. Abilities are scored from three to 15 in the Glasgow Coma Scale. Higher scores mean less severe injuries.

In yet another embodiment, the brain injury (e.g., AD, MCI, TBI, stroke or MS) is MCI and the standard of care treatment is selected from computerized cognitive training, group memory training, individual errorless learning sessions, family memory strategy interventions, DHA (docosahexaenoic acid), EPA (eicosapentanoic acid), ginko biloba, donepezil, rivastigimine, triflusal, Huannao Yicong capsules, piribedil, nicotine patch, vitamin E, vitamins B12 & B6, folic acid, rofecoxib, galantamine, cholinesterase inhibitors memantine, lithium, Wuzi Yanzong granules, ginseng, and exercise.

In yet another embodiment, the brain injury (e.g., AD, MCI, TBI, stroke or MS) is AD and the standard of care treatment is selected from computerized cognitive training, group memory training, individual errorless learning sessions, family memory strategy interventions, DHA (docosahexaenoic acid), EPA (eicosapentanoic acid), ginko biloba, donepezil, rivastigimine, triflusal, Huannao Yicong capsules, piribedil, nicotine patch, vitamin E, vitamins B12 & B6, folic acid, rofecoxib, galantamine, cholinesterase inhibitors memantine, lithium, Wuzi Yanzong granules, ginseng, and exercise. The standard of care treatment can be selected from cholinesterase inhibitors and memantine (Namenda). The cholinesterase inhibitors can be selected from donepezil (Aricept), galantamine (Razadyne) and rivastigmine (Exelon).

In one embodiment, the autoimmune disease is RA and the standard of care treatment can be selected from nonsteroidal anti-inflammatory drugs (NSAIDs), steroids (e.g., prednisone), disease-modifying antirheumatic drugs (DMARDs) and biologic agents. NSAIDs can include ibuprofen (Advil, Motrin IB) and naproxen sodium (Aleve). DMARDs can include methotrexate (Trexall, Otrexup, others), leflunomide (Arava), hydroxychloroquine (Plaquenil) and sulfasalazine (Azulfidine). Biologic agents can include abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz).

In one embodiment, the autoimmune disease is lupus nephritis and the standard of care treatment can include medicines to control blood pressure and/or a special diet low in protein and salt. Additionally, the standard of care treatment for lupus nephritis can be treatments for lupus such as, for example, nonsteroidal anti-inflammatory drugs (NSAIDs), antimalarial drugs, corticosteroids (e.g., prednisone; methylprednisolone), immunosuppressants, or biologic agents. Examples of NSAIDs can include naproxen sodium (Aleve) and ibuprofen (Advil, Motrin IB, others). An example of an antimalarial drug can be hydroxychloroquine (Plaquenil). Examples of immunosuppressants can include azathioprine (Imuran, Azasan), mycophenolate mofetil (CellCept) and methotrexate (Trexall). Examples of biologics can include belimumab (Benlysta) or rituximab (Rituxan).

In one embodiment, the metabolic disease is NASH and the standard of care treatment can include lifestyle changes such as losing weight, increasing exercise, avoiding liver damaging drugs, lowering cholesterol and/or managing diabetes. NASH is a type of Nonalcoholic fatty liver disease (NAFLD). NAFLD is an umbrella term for a range of liver conditions affecting people who drink little to no alcohol. The main characteristic of NAFLD is too much fat stored in liver cells and is marked by liver inflammation, which may progress to scarring and irreversible damage. This damage can be similar to the damage caused by heavy alcohol use. At its most severe, nonalcoholic steatohepatitis can progress to cirrhosis and liver failure.

In one embodiment, the metabolic disease is diabetic neuropathy and the standard of care treatment can include lifestyle changes such as losing weight, increasing exercise, lowering cholesterol, controlling protein in urine, fostering bone health, controlling high blood pressure, managing diabetes, kidney dialysis or transplant. Diabetic nephropathy is a serious kidney-related complication of type 1 diabetes and type 2 diabetes that can also be referred to as diabetic kidney disease (DKD).

In one embodiment, the autoimmune disease is IBD and the standard of care treatment can include anti-inflammatory drugs, immune system suppressors, antibiotics, anti-diarrheal medications, pain relievers, iron supplements and calcium and vitamin D supplements. Antibiotics can include ciprofloxacin (Cipro) and metronidazole (Flagyl). Examples of immunosuppressant drugs can include azathioprine (Azasan, Imuran), mercaptopurine (Purinethol, Purixan), cyclosporine (Gengraf, Neoral, Sandimmune) and methotrexate (Trexall). Other examples of immunosuppressants can include tumor necrosis factor (TNF)-alpha inhibitors, or biologics such as, for example, infliximab (Remicade), adalimumab (Humira), golimumab (Simponi), natalizumab (Tysabri), vedolizumab (Entyvio) and ustekinumab (Stelara). Anti-inflammatories can include corticosteroids and aminosalicylates, such as, for example, mesalamine (Asacol HD, Delzicol), balsalazide (Colazal) and olsalazine (Dipentum). IBD is an umbrella term used to describe disorders that involve chronic inflammation of an individual's digestive tract. IBD can include ulcerative colitis and Crohn's disease. Ulcerative colitis is along-lasting inflammation and sores (ulcers) in the innermost lining of your large intestine (colon) and rectum, while Crohn's disease is characterized by inflammation of the lining of the digestive tract, which often spreads deep into affected tissues.

In one embodiment, the autoinflammatory disease is CAPS and the standard of care treatment can include biologic agents that target interleukin-1 as well as physical therapy, splints to treat joint deformities, and nonsteroidal anti-inflammatory drugs, corticosteroids or methotrexate to reduce symptoms. Cryopyrin-associated periodic syndromes (CAPS), also called cryopyrin-associated autoinflammatory syndrome consists of three autoinflammatory diseases related to a defect in the same gene (i.e., NLRP3): neonatal onset multisystem inflammatory disease (NOMID), Muckle-Wells syndrome (MWS) and familial cold autoinflammatory syndrome (FCAS). NOMID is characterized by fever with inflammation in multiple organs. Early symptoms of NOMID can include a hive-like rash that does not itch; inflammation of the membrane surrounding the brain, which causes headache, blindness or hearing loss; bulging appearance to the eyes; and episodes of vomiting. After age 1, half of children with NOMID can develop joint pain and swelling. MWS is characterized by symptoms that come and go, including skin rash, red eyes, joint pain and severe headaches with vomiting. Episodes last between one and three days. Hearing loss, which may be complete, often occurs by the teenage years. FCAS is characterized by fever, chills, nausea, extreme thirst, headache and joint pain.

In one embodiment, the invention contemplates use of an antibody or an active fragment thereof in a method for treating inflammation or a disease, disorder or condition caused by or associated with inflammation in a subject, wherein the antibody or the active fragment thereof is directed against a component of a mammalian inflammasome or an antigen or epitope derived therefrom. In another embodiment, the agent to be administered is an antisense RNA or siRNA directed against a component of a mammalian inflammasome. The inflammasome component can be a component of any inflammasome known in the art, such as, for example, the NAPL1, NALP2, NALP3, NLRC4 or AIM2 inflammasome. In a typical embodiment, the antibody specifically binds to ASC or an antigen or epitope derived therefrom. However, an antibody against any other component of a mammalian inflammasome (e.g., the NALP1, NALP2, NALP3, NLRC4 or AIM2 inflammasome) may be used.

An antibody as described herein can be a monoclonal or polyclonal antibody or active fragments thereof. Said antibodies or active fragments can be chimeric, human or humanized as described herein.

In one embodiment, the antibody or the active fragment thereof is directed against a component of a mammalian inflammasome or an antigen or epitope derived therefrom specifically binds to at least one component (e.g., ASC, AIM2) of a mammalian inflammasome (e.g., the AIM2 inflammasome). Exemplary antibodies directed against components of a mammalian inflammasome for use in the methods herein can be those found in U.S. Pat. No. 8,685,400, the contents of which are herein incorporated by reference in its entirety. In one embodiment, the antibodies or antibody fragments thereof provided herein can be used in a method for reducing inflammation in a mammal as described in U.S. Pat. No. 8,685,400, the contents of which are herein incorporated by reference in their entirety. Use of the antibody or antibody fragment thereof in a method for treating inflammation can reduce inflammation. Use of the antibody or antibody fragment thereof (in a method for treating inflammation can reduce innate immune or inflammasome-related inflammation in the patient. The reduction can be as compared to a control (e.g., untreated patient and/or patient prior to treatment). In one embodiment, the antibody or antibody fragment derived therefrom is used to treat inflammation or a disease, disorder or condition caused by or associated with inflammation. The inflammation can be an innate immune inflammation. The inflammation can be an inflammasome-related inflammation. The disease, disorder or condition can be selected from the group consisting of a brain injury, an age-related disease, inflammaging, an autoimmune, autoinflammatory, metabolic or neurodegenerative disease. In some cases, the disease, disorder or condition is inflammaging. In some cases, the age-related disease is age-related macular degeneration (AMD). In some cases, the disease, disorder or condition is a brain injury. The brain injury can be selected from the group consisting of traumatic brain injury (TBI), stroke and spinal cord injury (SCI). The autoimmune or neurodegenerative disease can be selected from amyotrophic lateral sclerosis (ALS), Alzheimer's disease, Parkinson's disease (PD), muscular dystrophy (MD), immune dysfunction muscular CNS breakdown, systemic lupus erythematosus, lupus nephritis, rheumatoid arthritis, inflammatory bowel disease (e.g., Crohn's Disease and ulcerative colitis) and multiple sclerosis (MS). The metabolic disease can be selected from metabolic syndrome, obesity, diabetes mellitus, diabetic nephropathy or diabetic kidney disease (DKD), insulin resistance, atherosclerosis, a lipid storage disorder, a glycogen storage disease, medium-chain acyl-coenzyme A dehydrogenase deficiency, non-alcoholic fatty liver disease (e.g., Nonalcoholic steatohepatitis (NASH)) and gout. The autoinflammatory disease can be cryopyrin-associated periodic syndrome (CAPS). CAPS can encompass familial cold autoinflammatory syndrome (FCAS), Muckle-Wells syndrome (MWS) and neonatal-onset multisystem inflammatory disease (NOMID). The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In one embodiment, the antibody or antibody fragment derived therefrom is used to treat MS by administering the antibody or antibody fragment derived therefrom to a patient suffering from or suspected of suffering from MS. In some cases, the administering the antibody or the antibody fragment thereof reduces levels of at least inflammatory cytokine. The administration of the antibody or the antibody fragment thereof can result in inhibition of inflammasome activation in the subject. In some cases, the antibody or antibody fragment thereof can be directed against ASC. In some cases, the administration of the antibody or the antibody fragment thereof results in a reduction in the activity of ASC as compared to a control. The control can be an untreated subject. The administration can be intracerebroventricularly, intraperitoneally, intravenously or by inhalation. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In one embodiment, the antibody or antibody fragment derived therefrom is used to treat PD by administering the antibody or antibody fragment derived therefrom to a patient suffering from or suspected of suffering from PD. In some cases, the administering the antibody or the antibody fragment thereof reduces levels of at least inflammatory cytokine. The administration of the antibody or the antibody fragment thereof can result in inhibition of inflammasome activation in the subject. In some cases, the antibody or antibody fragment thereof can be directed against ASC. In some cases, the administration of the antibody or the antibody fragment thereof results in a reduction in the activity of ASC as compared to a control. The control can be an untreated subject. The administration can be intracerebroventricularly, intraperitoneally, intravenously or by inhalation. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In one embodiment, the antibody or antibody fragment derived therefrom is used to treat lupus nephritis by administering the antibody or antibody fragment derived therefrom to a patient suffering from or suspected of suffering from lupus nephritis. In some cases, the administering the antibody or the antibody fragment thereof reduces levels of at least inflammatory cytokine. The administration of the antibody or the antibody fragment thereof can result in inhibition of inflammasome activation in the subject. In some cases, the antibody or antibody fragment thereof can be directed against ASC. In some cases, the administration of the antibody or the antibody fragment thereof results in a reduction in the activity of ASC as compared to a control. The control can be an untreated subject. The administration can be intracerebroventricularly, intraperitoneally, intravenously or by inhalation. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In one embodiment, the antibody or antibody fragment derived therefrom is used to treat diabetic nephropathy by administering the antibody or antibody fragment derived therefrom to a patient suffering from or suspected of suffering from diabetic nephropathy. In some cases, the administering the antibody or the antibody fragment thereof reduces levels of at least inflammatory cytokine. The administration of the antibody or the antibody fragment thereof can result in inhibition of inflammasome activation in the subject. In some cases, the antibody or antibody fragment thereof can be directed against ASC. In some cases, the administration of the antibody or the antibody fragment thereof results in a reduction in the activity of ASC as compared to a control. The control can be an untreated subject. The administration can be intracerebroventricularly, intraperitoneally, intravenously or by inhalation. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)2, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In one embodiment, the antibody or antibody fragment derived therefrom is used to treat NASH by administering the antibody or antibody fragment derived therefrom to a patient suffering from or suspected of suffering from NASH. In some cases, the administering the antibody or the antibody fragment thereof reduces levels of at least inflammatory cytokine. The administration of the antibody or the antibody fragment thereof can result in inhibition of inflammasome activation in the subject. In some cases, the antibody or antibody fragment thereof can be directed against ASC. In some cases, the administration of the antibody or the antibody fragment thereof results in a reduction in the activity of ASC as compared to a control. The control can be an untreated subject. The administration can be intracerebroventricularly, intraperitoneally, intravenously or by inhalation. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In one embodiment, the antibody or antibody fragment derived therefrom is used to treat CAPS by administering the antibody or antibody fragment derived therefrom to a patient suffering from or suspected of suffering from CAPS. In some cases, the administering the antibody or the antibody fragment thereof reduces levels of at least inflammatory cytokine. The administration of the antibody or the antibody fragment thereof can result in inhibition of inflammasome activation in the subject. In some cases, the antibody or antibody fragment thereof can be directed against ASC. In some cases, the administration of the antibody or the antibody fragment thereof results in a reduction in the activity of ASC as compared to a control. The control can be an untreated subject. The administration can be intracerebroventricularly, intraperitoneally, intravenously or by inhalation. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In one embodiment, the antibody or antibody fragment derived therefrom is used to treat AMD by administering the antibody or antibody fragment derived therefrom to a patient suffering from or suspected of suffering from AMD. In some cases, the administering the antibody or the antibody fragment thereof reduces levels of at least inflammatory cytokine. The administration of the antibody or the antibody fragment thereof can result in inhibition of inflammasome activation in the subject. In some cases, the antibody or antibody fragment thereof can be directed against ASC. In some cases, the administration of the antibody or the antibody fragment thereof results in a reduction in the activity of ASC as compared to a control. The control can be an untreated subject. The administration can be intracerebroventricularly, intraperitoneally, intravenously or by inhalation. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In one embodiment, the antibody or antibody fragment derived therefrom is used to treat inflammaging or age-related inflammation by administering the antibody or antibody fragment derived therefrom to a patient suffering from or suspected of suffering from inflammaging or age-related inflammation. In some cases, the administering the antibody or the antibody fragment thereof reduces levels of at least inflammatory cytokine. The administration of the antibody or the antibody fragment thereof can result in inhibition of inflammasome activation in the subject. In some cases, the antibody or antibody fragment thereof can be directed against ASC. In some cases, the administration of the antibody or the antibody fragment thereof results in a reduction in the activity of ASC as compared to a control. The control can be an untreated subject. The administration can be intracerebroventricularly, intraperitoneally, intravenously or by inhalation. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

The antibody or antibody fragment thereof of this embodiment can be present in a composition such as, for example, a pharmaceutical composition as provided herein. The composition can further include at least one pharmaceutically acceptable carrier or diluent. In one embodiment, a method provided herein for treating inflammation or a disorder, disease or condition caused by or associated with inflammation comprises: providing a therapeutically effective amount of a composition comprising an antibody or an active fragment thereof as provided herein that specifically binds to at least one component (e.g., ASC) of a mammalian inflammasome (e.g., AIM2 inflammasome); and administering the composition to the mammal suffering from inflammation, wherein administering the composition to the mammal results in a reduction of caspase-1 activation in the mammal. In some cases, the antibody or fragment thereof is used in combination with one or more other agents in the methods of treatment provided herein. The other agents can be any agent provided herein (e.g., an extracellular vesicle (EV) uptake inhibitor) and/or antibodies or antibody fragments directed against other inflammasome components (e.g., IL-18, caspase-1, NALP1, AIM2, etc.). The EV uptake inhibitors can be selected from those found in Table 30.

In one embodiment, the agent to be administered in the method of treatments provided herein is an EV uptake inhibitor. The EV uptake inhibitor can be a compound, antisense RNA, siRNA, peptide, antibody or an active fragment thereof as provided herein or a combination thereof. The compound or peptide can be one or more compounds selected from heparin, α-difluoromethylornithine (DFMO), Enoxaparin, Asialofetuin, Human receptor-associated protein (RAP), RGD (Arg-Gly-Asp) peptide, Cytochalasin D, Cytochalasin B, Ethylenediaminetetra acetic acid (EDTA), Latrunculin A, Latrunculin B, NSC23766, Dynasore, Chlorpromazine, 5-(N-Ethyl-N-isopropyl)amiloride (EIPA), Amiloride, Bafilomycin A Monensin and Chloroquine, Annexin-V, Wortmannin, LY294002, Methyl-β-cyclodextrin (MβCD), Filipin, Simvastatin, Fumonisin B1 and N-butyldeoxynojirimycin hydrochloride, U0126 or a proton pump inhibitor. The EV uptake inhibitor antibody or an active fragment thereof as provided herein can be one or more antibodies or active fragments thereof directed against protein targets listed in Table 30. A composition for treating and/or reducing inflammation using an EV uptake inhibitor can further include at least one pharmaceutically acceptable carrier or diluent.

TABLE 30
Exemplary targets and corresponding antibodies for use in
blocking EV uptake.

| Gene Symbol | Gene Name | Exemplary Antibodies |
|---|---|---|
| ICAM-1 | Intercellular | Invitrogen ICAM-1 antibody (Life |
| | Adhesion | Technologies, 07-5403); CD54 |
| | Molecule 1 | (ICAM-1) Monoclonal Antibody |
| | | (R6.5), eBioscience ™ |
| LFA-1 | Lymphocyte | Abbiotec LFA-1 antibody (Abbiotec, |
| | function- | 250944); Developmental Studies |
| | associated | Hybridoma Bank LFA-1 antibody |
| | antigen 1 | (Developmental Studies Hybridoma |
| | | Bank, MHM24) |
| TIM-4 | T-cell | BioLegend TIMD4 antibody |
| | membrane | (BioLegend, 354004); LifeSpan |
| | protein 4 | Biosciences TIMD4 antibody |
| | | (Lifespan Biosciences, LS-B1413) |
| MFG-E8 | Milk Fat | MBL International MFGE8 antibody |
| | Globule-EGF | (MBL, D199-3); Santa Cruz |
| | Factor 8 | Biotechnology MFGE8 antibody |
| | Protein | (Santa Cruz, sc-8029); MBL |
| | | International MFGE8 antibody (MBL, |
| | | 18A2-G10) |
| DC-SIGN | Dendritic | Invitrogen DC SIGN antibody |
| | Cell-Specific | (eBioscience, eB-h209, 17-2099-41); |
| | Intercellular | BD Biosciences DC SIGN antibody |
| | adhesion | (BD, DCN46, 551186) |
| | molecule-3- | |
| | Grabbing Non- | |
| | integrin | |
| DEC205 | cluster of | EMD Millipore LY75 antibody |
| | differentiation | (Millipore, HD30); BioLegend |
| | 205 | LY75 antibody (BioLegend, 342203) |
| H-2Kb | MHC Class | BioLegend H2-K1 antibody |
| | I (H-2Kd) | (BioLegend, 28-8-6, 114603); |
| | | BioLegend H2-K1 antibody |
| | | (BioLegend, 28-14-8, 14-5999-85) |
| Tspan8 | Tetraspanin-8 | R and D Systems TSPAN8 antibody |
| | | (R&D Systems, MAB4734) |
| Tspan29 | Tetraspanin-29 | Santa Cruz Biotechnology CD9 |
| | | antibody (Santa Cruz, sc-59140); |
| | | Invitrogen CD9 antibody |
| | | (eBioscience, eBioSN4; BD |
| | | Biosciences CD9 antibody (BD |
| | | Pharmingen, 555370) |
| ITGAL | Integrin subunit | TS1/22.1.1.13.3; M17/4.4.11.9 |
| | alpha L | |
| ITGAM | Integrin subunit | CD11b Monoclonal Antibody |
| | alpha M | (VIM12)( CD11B00); BD Biosciences |
| | | CD11b antibody (BD Pharmingen, |
| | | ICRF44; 555385) |
| ITGAX | Integrin subunit | Anti-Integrin αX Antibody, clone |
| | alpha X | N418 (MAB1399Z); BD Biosciences |
| | | CD11c antibody (BD Bioscience, B- |
| | | ly6; 560369) |
| CD44 | Cluster of | Invitrogen CD44 antibody |
| | differentiation 44 | (eBioscience, VFF-7; MA1-82392); |
| | | Invitrogen CD44 antibody |
| | | (eBioscience, IM7; MA1-10225); |
| | | Invitrogen CD44 antibody |
| | | (eBioscience, 5F12; MA5-12394); BD |
| | | Biosciences CD44 antibody (BD |
| | | Biosciences, 515; 550990 OR 550988) |
| ITGA3 | Integrin | EMD Millipore integrin alpha3 |
| | subunit alpha 3 | antibody (Millipore, P1B5; |
| | | MAB 1952Z OR MAB 1952P) |
| ITGA4 | Integrin | Bio X Cell ITGA4 antibody |
| | subunit alpha 4 | (BioXcell, PS/2) (BE0071-5MG); BD |
| | | Biosciences ITGA4 antibody (BD |
| | | Biosciences, 561892); BD Biosciences |
| | | ITGA4 antibody (BD, 340976); EMD |
| | | Millipore ITGA4 antibody (Millipore, |
| | | P4C2; MAB1955) |
| ITGAV | Integrin subunit | Abcam integrin alpha v antibody |
| | alpha V | (Abcam, ab77906); Abcam integrin |
| | | alpha v antibody (Abcam, ab78289); |
| | | Abcam integrin alpha v antibody |
| | | (Abcam, ab16821); Invitrogen integrin |
| | | alpha v antibody (Thermo Fisher |
| | | Scientific, 272-17E6, MA1-91669); R |
| | | & D Systems integrin alpha v |
| | | antibody (R&D Systems, MAB2528) |
| ITGB3 | Integrin subunit | Abcam integrin beta3 antibody |
| | beta 3 | (Abcam, ab78289); Abnova integrin |
| | | beta3 antibody (Abnova, MHF4, |
| | | MAB7098) |
| SELL | Selectin L | BioLegend CD62L antibody |
| | | (Biolegend, 304804); BioLegend |
| | | CD62L antibody (Biolegend, 304810) |
| CD81 | CD81 molecule | BD Biosciences CD81 antibody (BD |
| | | Pharmingen, 555675); R and D |
| | | Systems CD81 antibody (R&D |
| | | Systems, MAB4615) |
| LRP1 | LDL receptor | Invitrogen LRP1 antibody (Life |
| | related protein 1 | Technologies, 37-7600); Invitrogen |
| | | LRP1 antibody (Thermo Fisher, MA1- |
| | | 27198) |
| VCAM1 | vascular cell | Invitrogen VCAM-1 antibody (Caltag, |
| | adhesion | IG11B1; MA5-16429); |
| | molecule 1 | Immunotech anti-VCAM-1 |
| | | antibody |
| CD151 | CD151 | BD Biosciences CD151 antibody |
| | molecule (Raph | (Becton Dickinson, 556056); |
| | blood group) | Epitomics CD151 antibody |
| | | (Epitomics, 5901-1) |

In one embodiment, the antibodies or active fragments thereof for use in the treatment methods provided herein are antibodies or active fragments thereof that bind specifically to Apoptosis-associated Spec-like protein containing a Caspase Activating Recruitment Domain (ASC) or domains or portions thereof. Any suitable anti-ASC antibody can be used, and several are commercially available. Examples of anti-ASC antibodies for use in the methods herein can be those found in U.S. Pat. No. 8,685,400, the contents of which are herein incorporated by reference in its entirety. Examples of commercially available anti-ASC antibodies for use in the methods provided herein include, but are not limited to 04-147 Anti-ASC, clone 2E1-7 mouse monoclonal antibody from Millipore Sigma, AB3607—Anti-ASC Antibody from Millipore Sigma, orb194021 Anti-ASC from Biorbyt, LS-C331318-50 Anti-ASC from LifeSpan Biosciences, AF3805 Anti-ASC from R & D Systems, NBP1-78977 Anti-ASC from Novus Biologicals, 600-401-Y67 Anti-ASC from Rockland Immunochemicals, D086-3 Anti-ASC from MBL International, AL177 anti-ASC from Adipogen, monoclonal anti-ASC (clone o93E9) antibody, anti-ASC antibody (F-9) from Santa Cruz Biotechnology, anti-ASC antibody (B-3) from Santa Cruz Biotechnology, ASC polyclonal antibody—ADI-905-173 from Enzo Life Sciences, or A161 Anti-Human ASC-Leinco Technologies. The human ASC protein can be accession number NP_037390.2 (Q9ULZ3-1), NP_660183 (Q9ULZ3-2) or Q9ULZ3-3. The rat ASC protein can be accession number NP_758825 (BAC43754). The mouse ASC protein can be accession number NP_075747.3. In one embodiment, the antibody binds to a PYRIN-PAAD-DAPIN domain (PYD) or a portion or fragment thereof of a mammalian ASC protein (e.g. human, mouse or rat ASC). In this embodiment, an antibody as described herein specifically binds to an amino acid sequence having at least 65% (e.g., 65, 70, 75, 80, 85%) sequence identity with a PYD domain or fragment thereof of human, mouse or rat ASC. In one embodiment, the antibody binds to a C-terminal caspase-recruitment domain (CARD) or a portion or fragment thereof of a mammalian ASC protein (e.g. human, mouse or rat ASC). In this embodiment, an antibody as described herein specifically binds to an amino acid sequence having at least 65% (e.g., 65, 70, 75, 80, 85%) sequence identity with a CARD domain or fragment thereof of human, mouse or rat ASC. In still another embodiment, the antibody binds to a portion or fragment thereof of a mammalian ASC protein sequence (e.g. human, mouse or rat ASC) located between the PYD and CARD domains. In another embodiment, a composition for treating and/or reducing inflammation in the CNS and/or lungs of a mammal includes an antibody that specifically binds to a region of rat ASC, e.g., amino acid sequence ALRQTQPYLVTDLEQS (SEQ ID NO:1) (i.e., residues 178-193 of rat ASC, accession number BAC43754). In this embodiment, an antibody as described herein specifically binds to an amino acid sequence having at least 65% (e.g., 65, 70, 75, 80, 85%) sequence identity with amino acid sequence ALRQTQPYLVTDLEQS (SEQ ID NO:1) of rat ASC. In another embodiment, a composition for treating and/or reducing inflammation in the CNS and/or lungs of a mammal includes an antibody that specifically binds to a region of human ASC, e.g., amino acid sequence RESQSYLVEDLERS (SEQ ID NO:2). In still another embodiment, a composition for treating and/or reducing inflammation in the CNS and/or lungs of a mammal includes an antibody that specifically binds to a region of human ASC, e.g., amino acid sequence KKFKLKLLSVPLREGYGRIPR (SEQ ID NO: 5; i.e., residues 21-41 of human ASC) or 5-10, 10-15 or 15-20 amino acids of SEQ ID NO: 5. In one embodiment, the antibody specifically binds to an amino acid sequence having at least 85% sequence identity with amino acid sequence SEQ ID NO:1 or SEQ ID NO:2. In another embodiment, the antibody or fragment thereof binds to an amino acid sequence having at least 85%, 86%, 87%, 88%, 89%, 90%, 91%, 92%, 93%, 94%, 95%, 96%, 97%, 98%, 99% or 100% sequence identity with amino acid sequence KKFKLKLLSVPLREGYGRIPR (SEQ ID NO: 5). In yet another embodiment, the antibody or fragment thereof binds to an amino acid sequence KKFKLKLLSVPLREGYGRIPR (SEQ ID NO: 5) or 2, 3, 4, 5, 6, 7, 8, 9, 10, 11, 12, 13, 14, 15, 16, 17, 18, 19 or 20 amino acids of SEQ ID NO: 5. In a still further embodiment, the antibody or fragment thereof binds to 2-5, 5-10, 10-15 or 15-20 amino acids of SEQ ID NO: 5. In some embodiments, an epitope of ASC (e.g., epitope with amino acid SEQ ID NO: 5) bound by an antibody or antibody fragment is continuous. In some embodiments, an epitope of ASC (e.g., epitope with amino acid SEQ ID NO: 5) bound by an antibody or antibody fragment is discontinuous. In some cases, the antibody or the antibody fragment thereof provided herein inhibits or reduces the activity of ASC. The antibody or antibody fragment derived therefrom can be a monoclonal antibody or derived from a monoclonal antibody. The antibody or antibody fragment derived therefrom can be a polyclonal antibody or derived from a polyclonal antibody. The antibody fragment can be a Fab, an F(ab′)₂, a Fab′, a scFv, a single domain antibody, a diabody or a single chain camelid antibody. The antibody or antibody fragment derived therefrom (e.g., monoclonal antibody or the antibody fragment thereof) can be human, humanized or chimeric.

In certain embodiments, the antibodies and antibody fragments that bind specifically to ASC are monoclonal antibodies or are derived from monoclonal antibodies that comprise one or more amino acid sequences shown Table 31. Also provided herein are isolated nucleic acid molecules encoding the monoclonal antibodies or the antibody fragments thereof that comprise nucleic acid sequences shown in Table 31. In some cases, provided herein are expression vectors comprising the nucleic acid molecules of Table 31. The expression vectors can comprise heavy chain or light chain constant regions. An example of a light chain and heavy chain expression vector system for use in the compositions and methods provided herein is the Antitope pANT expression vector system for IgG4 (S241P) heavy and kappa light chain. The nucleic acid molecule for the heavy or light chain can be operatively linked to regulatory sequences suitable for expression of the nucleic acid segments in a host cell.

TABLE 31
Variable Heavy and Variable Light (Kappa) Chain Sequences of anti-ASC
antibody or antibody fragments thereof of the invention.

Heavy Chain (H) CDR1 Amino Acid Sequence
TSGMGVS (SEQ ID NO: 6)
Heavy Chain(H) CDR1 Nucleic Acid Sequence
ACTAGTGGAATGGGTGTGAGC (SEQ ID NO: 9)
Heavy Chain (H) CDR2 Amino Acid Sequence
HIYWDDDKRYNPSLKS (SEQ ID NO: 7)
Heavy Chain (H) CDR2 Nucleic Acid Sequence
CACATTTATTGGGATGATGATAAGCGCTACAACCCATCTCTGAAGAGC (SEQ ID NO: 10)
Heavy Chain (H) CDR3 Amino Acid Sequence
STPIVANAMDY (SEQ ID NO: 8)
Heavy Chain (H) CDR3 Nucleic Acid Sequence
AGCACCCCCATCGTGGCCAACGCCATGGACTAC (SEQ ID NO: 11)
Light (Kappa) (L) Chain CDR1 Amino Acid Sequence
KASQSVDYDGDSYMN (SEQ ID NO: 12)
Light (Kappa) (L) Chain CDR1 Nucleic Acid Sequence
AAGGCCAGCCAGAGTGTTGACTACGACGGCGACAGTTACATGAAT (SEQ ID NO: 15)
Light (Kappa) (L) Chain CDR2 Amino Acid Sequence
AASNLES (SEQ ID NO: 13)
Light (Kappa) (L) Chain CDR2 Nucleic Acid Sequence
GCCGCATCTAACCTGGAATCC (SEQ ID NO: 16)
Light (Kappa) (L) Chain CDR3 Amino Acid Sequence
QQSNEDPYT (SEQ ID NO: 14)
Light (Kappa) (L) Chain CDR3 Nucleic Acid Sequence
CAGCAATCTAATGAGGACCCTTACACT (SEQ ID NO: 17)
Variable Heavy (VH) 1 Chain Amino Acid Sequence
QVT LKE SGP AIV KPT QTL TLT CSF SGF SLS TSG MGV SWI RQP SGK GLE WLA
HIY WDD DKR YNP SLK SRL TIS KDS SKN QVV LKI TSV DPV DTA TYS CAR STP
IVA NAM DYW GQG TSV TVSS (SEQ ID NO: 18)
Variable Heavy (VH) 1 Chain Nucleic Acid Sequence
CAGGTCACCTTGAAGGAGTCTGGTCCTGCCATCGTGAAACCCACACAGACCCTCA
CGCTGACCTGCAGCTTCTCTGGGTTCTCACTCAGCACTAGTGGAATGGGTGTGAGC
TGGATCCGTCAGCCCTCAGGAAAGGGCCTGGAGTGGCTTGCACACATTTATTGGG
ATGATGATAAGCGCTACAACCCATCTCTGAAGAGCAGGCTCACCATCTCCAAGGA
CAGCTCCAAAAACCAGGTGGTCCTTAAAATCACCAGCGTGGACCCTGTGGACACA
GCCACATATTCCTGTGCACGGAGCACCCCCATCGTGGCCAACGCCATGGACTACT
GGGGCCAAGGAACCAGCGTCACCGTCTCCTCA (SEQ ID NO: 23)
Variable Heavy (VH) 2 Chain Amino Acid Sequence
QVTLKESGPALVKPTQTLTLTCSFSGFSLSTSGMGVSWIRQPAGKGLEWLAHIYWDD
DKRYNPSLKSRLTISKDSSKNQVVLTMTNMDPVDTATYSCARSTPIVANAMDYWGQ
GTLVTVSS (SEQ ID NO: 19)
Variable Heavy (VH) 2 Chain Nucleic Acid Sequence
CAGGTCACCTTGAAGGAGTCTGGTCCTGCCCTGGTGAAACCCACACAGACCCTCA
CGCTGACCTGCAGCTTCTCTGGGTTCTCACTCAGCACTAGTGGAATGGGTGTGAGC
TGGATCCGTCAGCCCGCCGGAAAGGGCCTGGAGTGGCTTGCACACATTTATTGGG
ATGATGATAAGCGCTACAACCCATCTCTGAAGAGCAGGCTCACCATCTCCAAGGA
CAGCTCCAAAAACCAGGTGGTCCTTACAATGACCAACATGGACCCTGTGGACACA
GCCACATATTCCTGTGCACGGAGCACCCCCATCGTGGCCAACGCCATGGACTACT
GGGGCCAAGGAACCCTGGTCACCGTCTCCTCA (SEQ ID NO: 24)
Variable Heavy (VH) 3 Chain Amino Acid Sequence
QVTLKESGPALVKPTQTLTLTCSFSGFSLSTSGMGVSWIRQPAGKGLEWLAHIYWDD
DKRYNPSLKSRLTISKDSSKNQVVLTMTNMDPVDTATYYCARSTPIVANAMDYWGQ
GTLVTVSS (SEQ ID NO: 20)
Variable Heavy (VH) 3 Chain Nucleic Acid Sequence
CAGGTCACCTTGAAGGAGTCTGGTCCTGCCCTGGTGAAACCCACACAGACCCTCA
CGCTGACCTGCAGCTTCTCTGGGTTCTCACTCAGCACTAGTGGAATGGGTGTGAGC
TGGATCCGTCAGCCCGCCGGAAAGGGCCTGGAGTGGCTTGCACACATTTATTGGG
ATGATGATAAGCGCTACAACCCATCTCTGAAGAGCAGGCTCACCATCTCCAAGGA
CAGCTCCAAAAACCAGGTGGTCCTTACAATGACCAACATGGACCCTGTGGACACA
GCCACATATTACTGTGCACGGAGCACCCCCATCGTGGCCAACGCCATGGACTACT
GGGGCCAAGGAACCCTGGTCACCGTCTCCTCA (SEQ ID NO: 25)
Variable Heavy (VH) 4 Chain Amino Acid Sequence
QVTLKESGPALVKPTQTLTLTCTFSGFSLSTSGMGVSWIRQPAGKGLEWLAHIYWDD
DKRYNPSLKSRLTISKDTSKNQVVLTMTNMDPVDTATYYCARSTPIVANAMDYWGQ
GTLVTVSS (SEQ ID NO: 21)
Variable Heavy (VH) 4 Chain Nucleic Acid Sequence
CAGGTCACCTTGAAGGAGTCTGGTCCTGCCCTGGTGAAACCCACACAGACCCTCA
CGCTGACCTGCACCTTCTCTGGGTTCTCACTCAGCACTAGTGGAATGGGTGTGAGC
TGGATCCGTCAGCCCGCCGGAAAGGGCCTGGAGTGGCTTGCACACATTTATTGGG
ATGATGATAAGCGCTACAACCCATCTCTGAAGAGCAGGCTCACCATCTCCAAGGA
CACCTCCAAAAACCAGGTGGTCCTTACAATGACCAACATGGACCCTGTGGACACA
GCCACATATTACTGTGCACGGAGCACCCCCATCGTGGCCAACGCCATGGACTACT
GGGGCCAAGGAACCCTGGTCACCGTCTCCTCA (SEQ ID NO: 26)
Variable Heavy (VH) Chimeric (O) Chain Amino Acid Sequence
QVTLKESGPGILQPSQTLSLTCSFSGFSLSTSGMGVSWIRQPSGKGLEWLAHIYWDDD
KRYNPSLKSRLTISKDSSSNQVFLKITSVDTADTATYSCARSTPIVANAMDYWGQGTS
VTVSS (SEQ ID NO: 22)
Variable Heavy (VH) Chimeric (O) Chain Nucleic Acid Sequence
CAGGTTACTCTGAAAGAGTCTGGCCCTGGGATATTGCAGCCCTCCCAGACCCTCA
GTCTGACTTGTTCTTTCTCTGGGTTTTCACTGAGCACTTCTGGTATGGGTGTGAGCT
GGATTCGTCAGCCTTCAGGAAAGGGTCTGGAGTGGCTGGCACACATTTACTGGGA
TGATGACAAGCGCTATAACCCATCCCTGAAGAGCCGGCTCACAATCTCCAAGGAT
TCCTCCAGCAACCAGGTCTTCCTCAAGATCACCAGTGTGGACACTGCAGATACTGC
CACATACTCCTGTGCTCGAAGTACTCCGATTGTAGCTAATGCTATGGACTACTGGG
GTCAAGGAACCTCAGTCACCGTCTCCTCA (SEQ ID NO: 27)
Variable Kappa Light (VL) 1 Chain Amino Acid Sequence
DIVLTQSPDSLAVSLGERATINCKASQSVDYDGDSYMNWYQQKPGQPPKLLIYAASN
LESGIPARFSGSGSGTDFTLTISSLQEEDVATYYCQQSNEDPYTFGQGTKLEIK (SEQ ID
NO: 28)
Variable Kappa Light (VL) 1 Chain Nucleic Acid Sequence
GACATCGTGCTGACCCAGTCTCCAGACTCCCTGGCTGTGTCTCTGGGCGAGAGGG
CCACCATCAACTGCAAGGCCAGCCAGAGTGTTGACTACGACGGCGACAGTTACAT
GAATTGGTACCAGCAGAAACCAGGACAGCCTCCTAAGCTGCTCATTTACGCCGCA
TCTAACCTGGAATCCGGCATCCCTGCCCGATTCAGTGGCAGCGGGTCTGGGACAG
ATTTCACTCTCACCATCAGCAGCCTGCAGGAGGAAGATGTGGCAACTTATTACTGT
CAGCAATCTAATGAGGACCCTTACACTTTTGGCCAGGGGACCAAGCTGGAGATCA
AA (SEQ ID NO: 32)
Variable Kappa Light (VL) 2 Chain Amino Acid Sequence
DIVLTQSPDSLAVSLGERATINCKASQSVDYDGDSYMNWYQQKPGQPPKLLIYAASN
LESGIPARFSGSGSGTDFTLTISSLQPEDVATYYCQQSNEDPYTFGQGTKLEIK (SEQ ID
NO: 29)
Variable Kappa Light (VL) 2 Chain Nucleic Acid Sequence
GACATCGTGCTGACCCAGTCTCCAGACTCCCTGGCTGTGTCTCTGGGCGAGAGGG
CCACCATCAACTGCAAGGCCAGCCAGAGTGTTGACTACGACGGCGACAGTTACAT
GAATTGGTACCAGCAGAAACCAGGACAGCCTCCTAAGCTGCTCATTTACGCCGCA
TCTAACCTGGAATCCGGCATCCCTGCCCGATTCAGTGGCAGCGGGTCTGGGACAG
ATTTCACTCTCACCATCAGCAGCCTGCAGCCTGAAGATGTGGCAACTTATTACTGT
CAGCAATCTAATGAGGACCCTTACACTTTTGGCCAGGGGACCAAGCTGGAGATCA
AA (SEQ ID NO: 33)
Variable Kappa Light (VL) 3 Chain Amino Acid Sequence
DIVMTQSPDSLAVSLGERATINCKASQSVDYDGDSYMNWYQQKPGQPPKLLIYAASN
LESGIPARFSGSGSGTDFTLTISSLQPEDVATYYCQQSNEDPYTFGQGTKLEIK (SEQ ID
NO: 30)
Variable Kappa Light (VL) 3 Chain Nucleic Acid Sequence
GACATCGTGATGACCCAGTCTCCAGACTCCCTGGCTGTGTCTCTGGGCGAGAGGG
CCACCATCAACTGCAAGGCCAGCCAGAGTGTTGACTACGACGGCGACAGTTACAT
GAATTGGTACCAGCAGAAACCAGGACAGCCTCCTAAGCTGCTCATTTACGCCGCA
TCTAACCTGGAATCCGGCATCCCTGCCCGATTCAGTGGCAGCGGGTCTGGGACAG
ATTTCACTCTCACCATCAGCAGCCTGCAGCCTGAAGATGTGGCAACTTATTACTGT
CAGCAATCTAATGAGGACCCTTACACTTTTGGCCAGGGGACCAAGCTGGAGATCA
AA (SEQ ID NO: 34)
Variable Kappa Light (VL) Chimeric (O) Chain Amino Acid Sequence
DIVLTQSPASLAVSLGQRATISCKASQSVDYDGDSYMNWYQQKPGQPPKLLIYAASN
LESGIPARFSGSGSGTDFTLNIHPVEEEDAATYYCQQSNEDPYTFGGGTKLEIK (SEQ ID
NO: 31)
Variable Kappa Light (VL) Chimeric (O) Chain Nucleic Acid Sequence
GACATTGTGCTGACCCAATCTCCAGCTTCTTTGGCTGTGTCTCTAGGGCAGAGGGC
CACCATCTCCTGCAAGGCCAGCCAAAGTGTTGATTATGATGGTGATAGTTATATGA
ACTGGTACCAACAGAAACCAGGACAGCCACCCAAACTCCTCATCTATGCTGCATC
CAATCTAGAATCTGGCATCCCAGCCAGGTTTAGTGGCAGTGGGTCTGGGACAGAC
TTCACCCTCAACATCCATCCTGTGGAGGAGGAGGATGCTGCAACCTATTACTGTCA
GCAAAGTAATGAGGAcCCGTACACGTTCGGAGGGGGGACCAAGCTGGAAATAAA
A (SEQ ID NO: 35)

In one embodiment, the monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 18, 19, 20, 21, 22, or an amino acid sequence that is at least 95%, 96%, 97%, 98% or 99% identical to the amino acid sequence of SEQ ID NO: 18, 19, 20, 21, or 22.

In one embodiment, the monoclonal antibody or an antibody fragment thereof that binds specifically ASC, wherein the antibody or the antibody fragment thereof comprises a heavy chain variable (VH) region and a light or kappa chain variable (VL) region, wherein the VL region amino acid sequence comprises SEQ ID NO: 28, 29, 30, 31, or an amino acid sequence that is at least 95%, 96%, 97%, 98% or 99% identical to the amino acid sequence of SEQ ID NO: 28, 29, 30 or 31.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 18, 19, 20, 21, 22, or an amino acid sequence that is at least 95%, 96%, 97%, 98% or 99% identical to the amino acid sequence of SEQ ID NO: 18, 19, 20, 21 or 22; and wherein the VL region amino acid sequence comprises SEQ ID NO: 28, 29, 30, 31, or an amino acid sequence that is at least 95%, 96%, 97%, 98% or 99% identical to the amino acid sequence of SEQ ID NO: 28, 29, 30 or 31

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 18, or an amino acid sequence that is at least 95%, 96%, 97%, 98% or 99% identical to the amino acid sequence of SEQ ID NO: 18; and wherein the VL region amino acid sequence comprises SEQ ID NO: 28 or an amino acid sequence that is at least 95%, 96%, 97%, 98% or 99% identical to the amino acid sequence of SEQ ID NO: 28.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 18, or an amino acid sequence that is at least 95%, 96%, 97%, 98% or 99% identical to the amino acid sequence of SEQ ID NO: 18; and wherein the VL region amino acid sequence comprises SEQ ID NO: 29 or an amino acid sequence that is at least 95%, 96%, 97%, 98% or 99% identical to the amino acid sequence of SEQ ID NO: 29.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 18, or an amino acid sequence that is at least 95%, 96%, 97%, 98% or 99% identical to the amino acid sequence of SEQ ID NO: 18; and wherein the VL region amino acid sequence comprises SEQ ID NO: 30 or an amino acid sequence that is at least 95%, 96%, 97%, 98% or 99% identical to the amino acid sequence of SEQ ID NO: 30.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 18, or an amino acid sequence that is at least 95%, 96%, 97%, 98% or 99% identical to the amino acid sequence of SEQ ID NO: 18; and wherein the VL region amino acid sequence comprises SEQ ID NO: 31 or an amino acid sequence that is at least 95%, 96%, 97%, 98% or 99% identical to the amino acid sequence of SEQ ID NO: 31.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 19, or an amino acid sequence that is at least 95%, 96%, 97%, 98% or 99% identical to the amino acid sequence of SEQ ID NO: 19; and wherein the VL region amino acid sequence comprises SEQ ID NO: 28 or an amino acid sequence that is at least 95%, 96%, 97%, 98% or 99% identical to the amino acid sequence of SEQ ID NO: 28.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 19, or an amino acid sequence that is at least 95%, 96%, 97%, 98% or 99% identical to the amino acid sequence of SEQ ID NO: 19; and wherein the VL region amino acid sequence comprises SEQ ID NO: 29 or an amino acid sequence that is at least 95%, 96%, 97%, 98% or 99% identical to the amino acid sequence of SEQ ID NO: 29.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 19, or an amino acid sequence that is at least 95%, 96%, 97%, 98% or 99% identical to the amino acid sequence of SEQ ID NO: 19; and wherein the VL region amino acid sequence comprises SEQ ID NO: 30 or an amino acid sequence that is at least 95%, 96%, 97%, 98% or 99% identical to the amino acid sequence of SEQ ID NO: 30. In some cases, a monoclonal antibody or an antibody fragment derived therefrom comprising a VH region amino acid sequence comprising SEQ ID NO: 19 and a VL region amino acid sequence comprising SEQ ID NO: 30 can be referred to as IC-100.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 19, or an amino acid sequence that is at least 95%, 96%, 97%, 98% or 99% identical to the amino acid sequence of SEQ ID NO: 19; and wherein the VL region amino acid sequence comprises SEQ ID NO: 31 or an amino acid sequence that is at least 95%, 96%, 97%, 98% or 99% identical to the amino acid sequence of SEQ ID NO: 31.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 20, or an amino acid sequence that is at least 95%, 96%, 97%, 98% or 99% identical to the amino acid sequence of SEQ ID NO: 20; and wherein the VL region amino acid sequence comprises SEQ ID NO: 28 or an amino acid sequence that is at least 95%, 96%, 97%, 98% or 99% identical to the amino acid sequence of SEQ ID NO: 28.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 20, or an amino acid sequence that is at least 95%, 96%, 97%, 98% or 99% identical to the amino acid sequence of SEQ ID NO: 20; and wherein the VL region amino acid sequence comprises SEQ ID NO: 29 or an amino acid sequence that is at least 95%, 96%, 97%, 98% or 99% identical to the amino acid sequence of SEQ ID NO: 29.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 20, or an amino acid sequence that is at least 95%, 96%, 97%, 98% or 99% identical to the amino acid sequence of SEQ ID NO: 20; and wherein the VL region amino acid sequence comprises SEQ ID NO: 30 or an amino acid sequence that is at least 95%, 96%, 97%, 98% or 99% identical to the amino acid sequence of SEQ ID NO: 30.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 20, or an amino acid sequence that is at least 95%, 96%, 97%, 98% or 99% identical to the amino acid sequence of SEQ ID NO: 20; and wherein the VL region amino acid sequence comprises SEQ ID NO: 31 or an amino acid sequence that is at least 95%, 96%, 97%, 98% or 99% identical to the amino acid sequence of SEQ ID NO: 31.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 21, or an amino acid sequence that is at least 95%, 96%, 97%, 98% or 99% identical to the amino acid sequence of SEQ ID NO: 21; and wherein the VL region amino acid sequence comprises SEQ ID NO: 28 or an amino acid sequence that is at least 95%, 96%, 97%, 98% or 99% identical to the amino acid sequence of SEQ ID NO: 28.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 21, or an amino acid sequence that is at least 95%, 96%, 97%, 98% or 99% identical to the amino acid sequence of SEQ ID NO: 21; and wherein the VL region amino acid sequence comprises SEQ ID NO: 29 or an amino acid sequence that is at least 95%, 96%, 97%, 98% or 99% identical to the amino acid sequence of SEQ ID NO: 29.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 21, or an amino acid sequence that is at least 95%, 96%, 97%, 98% or 99% identical to the amino acid sequence of SEQ ID NO: 21; and wherein the VL region amino acid sequence comprises SEQ ID NO: 30 or an amino acid sequence that is at least 95%, 96%, 97%, 98% or 99% identical to the amino acid sequence of SEQ ID NO: 30.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 21, or an amino acid sequence that is at least 95%, 96%, 97%, 98% or 99% identical to the amino acid sequence of SEQ ID NO: 21; and wherein the VL region amino acid sequence comprises SEQ ID NO: 31 or an amino acid sequence that is at least 95%, 96%, 97%, 98% or 99% identical to the amino acid sequence of SEQ ID NO: 31.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 22, or an amino acid sequence that is at least 95%, 96%, 97%, 98% or 99% identical to the amino acid sequence of SEQ ID NO: 22; and wherein the VL region amino acid sequence comprises SEQ ID NO: 28 or an amino acid sequence that is at least 95%, 96%, 97%, 98% or 99% identical to the amino acid sequence of SEQ ID NO: 28.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 22, or an amino acid sequence that is at least 95%, 96%, 97%, 98% or 99% identical to the amino acid sequence of SEQ ID NO: 22; and wherein the VL region amino acid sequence comprises SEQ ID NO: 29 or an amino acid sequence that is at least 95%, 96%, 97%, 98% or 99% identical to the amino acid sequence of SEQ ID NO: 29.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 22, or an amino acid sequence that is at least 95%, 96%, 97%, 98% or 99% identical to the amino acid sequence of SEQ ID NO: 22; and wherein the VL region amino acid sequence comprises SEQ ID NO: 30 or an amino acid sequence that is at least 95%, 96%, 97%, 98% or 99% identical to the amino acid sequence of SEQ ID NO: 30.

In one embodiment, provided herein is a monoclonal antibody or an antibody fragment thereof that binds specifically ASC, wherein the antibody or the antibody fragment thereof comprises a heavy chain variable (VH) region and a light or kappa chain variable (VL) region, wherein the VH region amino acid sequence comprises SEQ ID NO: 22, or an amino acid sequence that is at least 95%, 96%, 97%, 98% or 99% identical to the amino acid sequence of SEQ ID NO: 22; and wherein the VL region amino acid sequence comprises SEQ ID NO: 31 or an amino acid sequence that is at least 95%, 96%, 97%, 98% or 99% identical to the amino acid sequence of SEQ ID NO: 31.

Further to the above embodiments, the invention contemplates use of the antibodies or antibody fragments thereof (e.g., monoclonal antibodies or antibody fragments thereof that bind ASC) in a method for treating inflammation or a disorder, disease or condition cause by or associated with inflammation in a subject as provided herein. The antibodies or antibody fragments thereof that bind specifically to ASC can be monoclonal antibodies or antibody fragments thereof that can comprise a heavy chain variable (VH) region and a light chain variable (VL) region, wherein the VH region amino acid sequence comprises HCDR1 of SEQ ID NO: 6, HCDR2 of SEQ ID NO: 7 and HCDR3 of SEQ ID NO: 8, or a variant thereof having at least one amino acid substitution in HCDR1, HCDR2, and/or HCDR3. In some embodiments, the monoclonal antibodies or antibody fragments thereof that bind specifically to ASC can comprise a light chain variable (VL) region and a heavy chain variable (VH) region, wherein the VL region amino acid sequence comprises LCDR1 of SEQ ID NO: 12, LCDR2 of SEQ ID NO: 13 and LCDR3 of SEQ ID NO: 14, or a variant thereof having at least one amino acid substitution in LCDR1, LCDR2, and/or LCDR3. In other embodiments, the monoclonal antibodies or an antibody fragments thereof that bind specifically to ASC can comprise a heavy chain variable (VH) region and a light chain variable (VL) region, wherein the VH region amino acid sequence comprises HCDR1 of SEQ ID NO: 6, HCDR2 of SEQ ID NO: 7 and HCDR3 of SEQ ID NO: 8, or a variant thereof having at least one amino acid substitution in HCDR1, HCDR2, and/or HCDR3; and wherein the VL region amino acid sequence comprises LCDR1 of SEQ ID NO: 12, LCDR2 of SEQ ID NO: 13 and LCDR3 of SEQ ID NO: 14, or a variant thereof having at least one amino acid substitution in LCDR1, LCDR2, and/or LCDR3. The antibodies or fragments thereof can be in a composition. The composition can be administered in a therapeutically effective amount. The therapeutically effective amount can be a dose as provided herein. The composition can be administered by any suitable route, e.g., by inhalation, intravenously, intraperitoneally, or intracerebroventricularly. The composition can further include at least one pharmaceutically acceptable carrier or diluent. The composition can further comprise an additional therapeutic agent. The additional therapeutic agent can be an extracellular vesicle (EV) uptake inhibitor and/or an antibody or an active fragment thereof as provided herein that binds to a component of an inflammasome or a combination thereof. The EV uptake inhibitor can be selected from Table 30. The inflammation can be an innate immune inflammation. The inflammation can be an inflammasome-related inflammation. The disease, disorder or condition can be selected from the group consisting of a brain injury, an age-related disease, inflammaging, an autoimmune, autoinflammatory, metabolic or neurodegenerative disease. In some cases, the disease, disorder or condition is inflammaging. In some cases, the age-related disease is age-related macular degeneration (AMD). In some cases, the disease, disorder or condition is a brain injury. The brain injury can be selected from the group consisting of traumatic brain injury (TBI), stroke and spinal cord injury (SCI). The autoimmune or neurodegenerative disease can be selected from amyotrophic lateral sclerosis (ALS), Alzheimer's disease, Parkinson's disease (PD), muscular dystrophy (MD), immune dysfunction muscular CNS breakdown, systemic lupus erythematosus, lupus nephritis, rheumatoid arthritis, inflammatory bowel disease (e.g., Crohn's Disease and ulcerative colitis) and multiple sclerosis (MS). The metabolic disease can be selected from metabolic syndrome, obesity, diabetes mellitus, diabetic nephropathy or diabetic kidney disease (DKD), insulin resistance, atherosclerosis, a lipid storage disorder, a glycogen storage disease, medium-chain acyl-coenzyme A dehydrogenase deficiency, non-alcoholic fatty liver disease (e.g., Nonalcoholic steatohepatitis (NASH)) and gout. The autoinflammatory disease can be cryopyrin-associated periodic syndrome (CAPS). CAPS can encompass familial cold autoinflammatory syndrome (FCAS), Muckle-Wells syndrome (MWS) and neonatal-onset multisystem inflammatory disease (NOMID).

The success of, or response to, a treatment in a method provided herein for treating inflammation or a disease, disorder or condition caused by or associated with inflammation (e.g., antibody treatment, standard of care and/or neuroprotective treatment) can also be monitored by measuring the levels of at least one inflammasome protein. Accordingly, in some embodiments, the methods of treating or evaluating or diagnosing a patient with inflammation or a disease, disorder or condition caused by or associated with inflammation further comprises measuring the level of at least one inflammasome protein alone or in combination with at least one control biomarker protein in a biological sample obtained from the patient following treatment, preparing a treatment protein signature associated with a positive response to the treatment, wherein the treatment protein signature comprises a reduced level of at least one inflammasome protein and/or a reduced level of at least one control biomarker protein, and identifying patients exhibiting the presence of the treatment protein signature as responding positively to the treatment. A reduction in the level, abundance, or concentration of one or more inflammasome proteins (e.g. ASC, IL-18, caspase-8, caspase-11 or caspase-1) can be indicative of the efficacy of the treatment in the patient. A reduction in the level, abundance, or concentration of one or more control biomarker proteins (e.g., Gal-3, CRP (hs-CRP), AB₍₁₋₄₂₎, AB₍₁₋₄₀₎, sAPPα, sAPPβ, or NFL, or combinations thereof) can be indicative of the efficacy of the treatment in the patient. The one or more inflammasome proteins measured in the sample obtained following treatment may be the same as or different than the inflammasome proteins measured in the sample obtained prior to treatment. The one or more control biomarker proteins measured in the sample obtained following treatment may be the same as or different than the control biomarker proteins measured in the sample obtained prior to treatment. The inflammasome protein levels may also be used to adjust dosage or frequency of a treatment. The control biomarker protein levels may also be used to adjust dosage or frequency of a treatment. The inflammasome protein levels can be ascertained using the methods and techniques provided herein. The control biomarker protein levels can be ascertained using the methods and techniques provided herein.

In another embodiment, a composition for treating or reducing inflammation includes an antibody or an active fragment thereof as provided herein that specifically binds to NLRP1 or a domain or portion thereof. Any suitable anti-NLRP1 antibody can be used, and several are commercially available. Examples of anti-NLRP1 antibodies for use in the methods herein can be those found in U.S. Pat. No. 8,685,400, the contents of which are herein incorporated by reference in its entirety. Examples of commercially available anti-NLRP1 antibodies for use in the methods provided herein include, but are not limited to human NLRP1 polyclonal antibody AF6788 from R&D Systems, EMD Millipore rabbit polyclonal anti-NLRP1 ABF22, Novus Biologicals rabbit polyclonal anti-NLRP1 NB100-56148, Sigma-Aldrich mouse polyclonal anti-NLRP1 SAB1407151, Abcam rabbit polyclonal anti-NLRP1 ab3683, Biorbyt rabbit polyclonal anti-NLRP1 orb325922 my BioSource rabbit polyclonal anti-NLRP1 MBS7001225, R&D systems sheep polyclonal AF6788, Aviva Systems mouse monoclonal anti-NLRP1 oaed00344, Aviva Systems rabbit polyclonal anti-NLRP1 ARO54478_P050, Origene rabbit polyclonal anti-NLRP1 APO7775PU-N, Antibodies online rabbit polyclonal anti-NLRP1 ABIN768983, Prosci rabbit polyclonal anti-NLRP1 3037, Proteintech rabbit polyclonal anti-NLRP1 12256-1-AP, Enzo mouse monoclonal anti-NLRP1 ALX-804-803-C100, Invitrogen mouse monoclonal anti-NLRP1 MA1-25842, GeneTex mouse monoclonal anti-NLRP1 GTX16091, Rockland rabbit polyclonal anti-NLRP1 200-401-CX5, or Cell Signaling Technology rabbit polyclonal anti-NLRP1 4990. The human NLRP1 protein can be accession number AAH51787, NP_001028225, NP_055737, NP_127497, NP_127499, or NP_127500. In one embodiment, the antibody binds to a Pyrin, NACHT, LRR1-6, FIIND or CARD domain or a portion or fragment thereof of a mammalian NLRP1 protein (e.g. human NLRP1). In this embodiment, an antibody as described herein specifically binds to an amino acid sequence having at least 65% (e.g., 65%, 70%, 75%, 80%, 85%) sequence identity with a specific domain (e.g., Pyrin, NACHT, LRR1-6, FIIND or CARD) or fragment thereof of human NLRP1. In one embodiment, a chicken anti-NLRP1 polyclonal that was custom-designed and produced by Ayes Laboratories is used for reducing inflammation. This antibody can be directed against the following amino acid sequence in human NLRP1: CEYYTEIREREREKSEKGR (SEQ ID NO: 3) or the following amino acid sequence in rat NALP1: MEE SQS KEE SNT EG-cys (SEQ ID NO: 4). In one embodiment, an antibody that binds to a NLRP1 domain or fragment thereof as described herein inhibits NLRP1 activity in cells, e.g., Type II alveolar cells of a mammal.

In yet another embodiment, a composition for reducing inflammation in a mammal includes an antibody or an active fragment thereof as provided herein that specifically binds to AIM2 or a domain thereof. Any suitable anti-AIM2 antibody can be used, and several are commercially available. Examples of commercially available anti-AIM2 antibodies for use in the methods provided herein include, but are not limited to a rabbit polyclonal anti-AIM2 cat. Number 20590-1-AP from Proteintech, Abcam anti-AIMS antibody (ab119791), rabbit polyclonal anti-AIM2 (N-terminal region) Cat. Number AP3851 from ECM biosciences, rabbit polyclonal anti-ASC Cat. Number E-AB-30449 from Elabsciences, Anti-AIM2 mouse monoclonal antibody called AIM2 Antibody (3C4G11) with catalog number sc-293174 from Santa Cruz Biotechnology, mouse monoclonal AIM2 antibody with catalog number TA324972 from Origene, AIM2 monoclonal antibody (10M2B3) from Thermofisher Scientific, AIM2 rabbit polyclonal antibody ABIN928372 or ABIN760766 from Antibodies-online, Biomatix coat anti-AIM2 polyclonal antibody with cat. Number CAE02153. Anti-AIM2 polyclonal antibody (OABF01632) from Aviva Systems Biology, rabbit polyclonal anti-AIM2 antibody LS-C354127 from LSBio-C354127, rabbit monoclonal anti-AIM2 antibody from Cell Signaling Technology, with cat number MA5-16259. Rabbit polyclonal anti-AIM2 monoclonal antibody from Fab Gennix International Incorporated, Cat. Number AIM2 201AP, My BioSource rabbit polyclonal anti-AIM2 cat number MBS855320, Signalway rabbit polyclonal anti AIM2 catalog number 36253, Novus Biological rabbit polyclonal anti-AIM2 catalog number 43900002, GeneTex rabbit polyclonal anti-AIM2 GTX54910, Prosci, rabbit polyclonal anti-AIM2 26-540, Biorbyt mouse monoclonal anti-AIM2 orb333902, Abcam rabbit polyclonal anti-AIM2 ab93015), Abcam rabbit polyclonal anti-AIM2 ab76423, Sigma Aldrich mouse polyclonal anti-AIM2 SAB1406827, or Biolegend anti-AIM2 3B10. The human AIM2 protein can be accession number NX_014862, NP004824, XP016858337, XP005245673, AAB81613, BAF84731 or AAH10940. In one embodiment, the antibody binds to a Pyrin or HIN-200 domain or a portion or fragment thereof of a mammalian AIM2 protein (e.g. human AIM2). In this embodiment, an antibody as described herein specifically binds to an amino acid sequence having at least 65% (e.g., 65%, 70%, 75%, 80%, 85%) sequence identity with a specific domain (e.g., Pyrin or HIN-200) or fragment thereof of human AIM2. In one embodiment, an antibody that binds to an AIM2 domain or fragment thereof as described herein inhibits AIM2 activity in cells, e.g., Type II alveolar cells of a mammal.

Anti-inflammasome (e.g., Anti-ASC, anti-NLRP1 or anti-AIM2) antibodies as described herein can include polyclonal and monoclonal rodent antibodies, polyclonal and monoclonal human antibodies, or any portions thereof, having at least one antigen binding region of an immunoglobulin variable region, which antibody specifically binds to a component of a mammalian inflammasome (e.g., AIM2 inflammasome) such as, for example, ASC, NLRP1 or AIM2. In some cases, the antibody is specific for ASC such that an antibody is specific for ASC if it is produced against an epitope of the polypeptide and binds to at least part of the natural or recombinant protein.

In certain embodiments, an antibody provided herein comprises a polypeptide having one or more amino acid substitutions, deletions or insertions. For example, an anti-ASC monoclonal antibody or an ASC binding antibody fragment comprises a polypeptide having one or more amino acid substitutions, deletions or insertions as compared to a polypeptide having an amino acid sequence of one or more of SEQ ID NOs: 6-8, 12-14, 18-22 or 28-31. An antibody provided herein may have 1, 2, 3, 4, 5, 6, 7, 8, 9, 10, or more amino acid substitutions, deletions or insertions. For example, an anti-ASC monoclonal antibody or an ASC binding antibody fragment may have 1, 2, 3, 4, 5, 6, 7, 8, 9, 10, or more amino acid substitutions, deletions or insertions. Substitutions, deletions or insertions may be introduced by standard techniques, such as site-directed mutagenesis or PCR-mediated mutagenesis of a nucleic acid molecule encoding a polypeptide of an anti-ASC antibody or an ASC-binding antibody fragment.

In certain embodiments, conservative amino acid substitutions are made at one or more positions in the amino acid sequences of antibodies or antibody fragments disclosed herein. A “conservative amino acid substitution” is one in which the amino acid residue is replaced with an amino acid residue having a similar side chain. In certain embodiments, conservative amino acid substitutions are made only in the FR sequences and not in the CDR sequences of an antibody or antibody fragment. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan; histidine). Thus, for example, an amino acid residue in a polypeptide of an anti-ASC monoclonal antibody or an ASC binding antibody fragment may be replaced with another amino acid residue from the same side chain family. In certain embodiments, a string of amino acids can be replaced with a structurally similar string that differs in order and/or composition of side chain family members. Those skilled in the art will be able to evaluate whether an anti-ASC monoclonal antibody or an ASC binding antibody fragment comprising a polypeptide having one or more amino acid substitutions, deletions or insertions as compared to a polypeptide having an amino acid sequence of one or more of SEQ ID NOs: 6-8, 12-14, 18-22 or 28-31 binds ASC protein by utilizing routine, art-recognized methods including, but not limited to, ELISAs, Western blots, phage display, etc.

Calculations of sequence homology or identity (the terms are used interchangeably herein) between sequences may be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In an exemplary embodiment, the length of a reference sequence aligned for comparison purposes is at least 30%, 40%, 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman et al. ((1970) J. Mol. Biol. 48:444-453) algorithm which has been incorporated into the GAP program in the GCG software package (available at www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at www.gcg.com), using a NWSgapdna CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation of the invention) is a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. ((1989) CABIOS 4:11-17) which has been incorporated into the ALIGN program (version 2.0), using a PAM120 weight residue table, a gap length penalty of 12 and a gap penalty of 4.

In certain aspects, an antibody is a monoclonal antibody. In other aspects, an antibody is a polyclonal antibody. The term “monoclonal antibody” refers to a population of antibody molecules that contain only one species of an antigen binding site capable of immunoreacting with a particular epitope of an antigen. A monoclonal antibody composition thus typically displays a single binding affinity for a particular protein with which it immunoreacts.

In some aspects, an antibody of the invention (an anti-ASC monoclonal antibody or an ASC binding antibody fragment) is humanized, chimeric or human.

In some embodiments, an antibody of the invention is a humanized antibody.

“Humanized antibody” as the term is used herein refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. In some instances, Fv framework region (FR) residues of the human immunoglobulin are replaced by corresponding non-human residues. Furthermore, the humanized antibody may comprise residues that are found neither in the human form of the antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The FR region can be modified in any manner known in the art and/or provided herein. The modifications can confer desirable properties such as increased half-life and/or improved expression in host cells. In one embodiment, the FR region(s) can be modified or mutated as described in US20150232557, which is herein incorporated by reference. Other forms of humanized antibodies can have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody. The humanized antibody optimally also will comprise at least a portion of an immunoglobulin constant region or domain (Fc), typically that of a human immunoglobulin.

Humanized antibodies are typically less immunogenic to humans, relative to non-humanized antibodies, and thus offer therapeutic benefits in certain situations. For example, the antibody constant region can be engineered such that it is immunologically inert (e.g., does not trigger complement lysis). See, e.g. PCT Publication No. PCT/GB99/01441; UK Patent Application No. 9809951.8, each of which is incorporated herein by reference in its entirety. Those skilled in the art will be aware of humanized antibodies, and will also be aware of suitable techniques for their generation. See for example, Hwang, W. Y. K., et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and Selick et al., WO 90/07861, each of which is incorporated herein by reference in its entirety. Other methods of humanizing antibodies that may also be utilized are disclosed by Daugherty et al., Nucl. Acids Res. 19:2471-2476, 1991, and in U.S. Pat. Nos. 6,180,377; 6,054,297; 5,997,867; 5,866,692; 6,210,671; and 6,350,861; and in PCT Publication No. WO 01/27160, each of which is incorporated herein by reference in its entirety. For example, an anti-ASC antibody or anti-ASC antigen-binding fragment of the invention may comprise a VH region amino acid sequence that comprises HCDR1 of SEQ ID NO: 6, HCDR2 of SEQ ID NO: 7 and HCDR3 of SEQ ID NO: 8; and a VL region amino acid sequence that comprises LCDR1 of SEQ ID NO: 12, LCDR2 of SEQ ID NO: 13 and LCDR3 of SEQ ID NO: 14; and one or more human framework region sequences.

In some embodiments, an antibody for use in the methods provided herein is a chimeric antibody and binds specifically ASC. In some cases, the anti-ASC chimeric antibody reduces the activity of ASC. “Chimeric antibody” as the term is used herein refers to an antibody that has been engineered to comprise at least one human constant region. For example, one or all the variable regions of the light chain(s) and/or one or all the variable regions of the heavy chain(s) of a mouse antibody (e.g., a mouse monoclonal antibody) may each be joined to a human constant region, such as, without limitation an IgG1 human constant region. Chimeric antibodies are typically less immunogenic to humans, relative to non-chimeric antibodies, and thus offer therapeutic benefits in certain situations. Those skilled in the art will be aware of chimeric antibodies, and will also be aware of suitable techniques for their generation. See, for example, Cabilly et al., U.S. Pat. No. 4,816,567; Shoemaker et al., U.S. Pat. No. 4,978,775; Beavers et al., U.S. Pat. No. 4,975,369; and Boss et al., U.S. Pat. No. 4,816,397, each of which is incorporated herein by reference in its entirety. For example, an antibody or antigen-binding fragment of the invention may comprise a VH region comprising SEQ ID NO: 22; a VL region comprising SEQ ID NO: 31, and a human constant region.

As used herein, the terms “immunological binding,” and “immunological binding properties” refer to the non-covalent interactions of the type which occur between an immunoglobulin molecule (e.g., antibody) and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody for use in the methods provided herein is said to specifically bind to an epitope (e.g., ASC fragment with amino acid SEQ ID NO: 5) when the equilibrium binding constant (K_{d}) is ≤10 μM, ≤10 nM, ≤10 nM, and ≤100 pM to about 1 pM, as measured by assays such as radioligand binding assays or similar assays known to those skilled in the art.

In certain aspects, an antibody for use in the methods provided herein is monovalent or bivalent and comprises a single or double chain. Functionally, the binding affinity of an antibody may be within the range of 10⁻⁵ M to 10⁻¹² M. For example, the binding affinity of an antibody is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M or from 10⁻⁵ M to 10⁻⁶ M.

Methods for determining monoclonal antibody specificity and affinity by competitive inhibition can be found in Harlow, et al., Antibodies: A Laboratory Manual, Cold Spring Harbor Laboratory Press, Cold Spring Harbor, N.Y., 1988, Colligan et al., eds., Current Protocols in Immunology, Greene Publishing Assoc. and Wiley Interscience, N.Y., (1992, 1993), and Muller, Meth. Enzymol. 92:589-601, 1983, which references are entirely incorporated herein by reference.

Anti-inflammasome (e.g., Anti-ASC and anti-AIM2) antibodies for use in the methods provided herein can be routinely made according to methods such as, but not limited to inoculation of an appropriate animal with the polypeptide or an antigenic fragment, in vitro stimulation of lymphocyte populations, synthetic methods, hybridomas, and/or recombinant cells expressing nucleic acid encoding such anti-ASC or anti-NLR1 antibodies. Immunization of an animal using purified recombinant ASC or peptide fragments thereof, e.g., residues 178-193 (SEQ ID NO:1) of rat ASC (e.g., accession number BAC43754), SEQ ID NO:2 of human ASC or residues 21-41 (SEQ ID NO: 5) of human ASC (e.g., accession number NP_037390.2), is an example of a method of preparing anti-ASC antibodies. Similarly, immunization of an animal using purified recombinant NLRP1 or peptide fragments thereof, e.g., residues MEE SQS KEE SNT EG-cys (SEQ ID NO:4) of rat NALP1 or SEQ ID NO:3 of human NALP1, is an example of a method of preparing anti-NLRP1 antibodies.

Monoclonal antibodies that specifically bind ASC or NLRP1 may be obtained by methods known to those skilled in the art. See, for example Kohler and Milstein, Nature 256:495-497, 1975; U.S. Pat. No. 4,376,110; Ausubel et al., eds., Current Protocols in Molecular Biology, Greene Publishing Assoc. and Wiley Interscience, N.Y., (1987, 1992); Harlow and Lane ANTIBODIES: A Laboratory Manual Cold Spring Harbor Laboratory Press, Cold Spring Harbor, N.Y., 1988; Colligan et al., eds., Current Protocols in Immunology, Greene Publishing Assoc. and Wiley Interscience, N.Y., (1992, 1993), the contents of which are incorporated entirely herein by reference. Such antibodies may be of any immunoglobulin class including IgG, IgM, IgE, IgA, GILD and any subclass thereof. A hybridoma producing a monoclonal antibody of the present invention may be cultivated in vitro, in situ or in vivo. In one embodiment, a hybridoma producing an anti-ASC monoclonal antibody of the present disclosure is the ICCN1.OH hybridoma. In another embodiment, a hybridoma producing an anti-ASC monoclonal antibody of the present disclosure produces monoclonal antibodies comprising a heavy chain variable (VH) region and a light chain variable (VL) region, wherein the VH region amino acid sequence comprises HCDR1 of SEQ ID NO: 6, HCDR2 of SEQ ID NO: 7 and HCDR3 of SEQ ID NO: 8, or a variant thereof having at least one amino acid substitution in HCDR1, HCDR2, and/or HCDR3. In another embodiment, a hybridoma producing an anti-ASC monoclonal antibody of the present disclosure produces monoclonal antibodies comprising a heavy chain variable (VH) region and a light chain variable (VL) region, wherein the VL region amino acid sequence comprises LCDR1 of SEQ ID NO: 12, LCDR2 of SEQ ID NO: 13 and LCDR3 of SEQ ID NO: 14, or a variant thereof having at least one amino acid substitution in LCDR1, LCDR2, and/or LCDR3. In yet another embodiment, a hybridoma producing an anti-ASC monoclonal antibody of the present disclosure produces monoclonal antibodies comprising a heavy chain variable (VH) region and a light chain variable (VL) region, wherein the VH region amino acid sequence comprises HCDR1 of SEQ ID NO: 6, HCDR2 of SEQ ID NO: 7 and HCDR3 of SEQ ID NO: 8, or a variant thereof having at least one amino acid substitution in HCDR1, HCDR2, and/or HCDR3 and wherein the VL region amino acid sequence comprises LCDR1 of SEQ ID NO: 12, LCDR2 of SEQ ID NO: 13 and LCDR3 of SEQ ID NO: 14, or a variant thereof having at least one amino acid substitution in LCDR1, LCDR2, and/or LCDR3.

### Administration of Compositions

The compositions for use in the methods provided herein may be administered to mammals (e.g., rodents, humans) in any suitable formulation. For example, anti-ASC antibodies may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions for use in the methods provided herein may be administered to mammals by any conventional technique. Typically, such administration will be by inhalation or parenteral (e.g., intravenous, subcutaneous, intratumoral, intramuscular, intraperitoneal, or intrathecal introduction). The compositions may also be administered directly to a target site by, for example, surgical delivery to an internal or external target site, or by catheter to a site accessible by a blood vessel. The compositions may be administered in a single bolus, multiple injections, or by continuous infusion (e.g., intravenously, by peritoneal dialysis, pump infusion). For parenteral administration, the compositions can be formulated in a sterilized pyrogen-free form.

### Effective Doses

The compositions described above can be administered to a mammal (e.g., a rat, human) in an effective amount, that is, an amount capable of producing a desirable result in a treated mammal (e.g., reducing inflammation in the CNS of a mammal subjected to a traumatic injury to the CNS or stroke or having an autoimmune, autoinflammatory, metabolic, neurodegenerative or CNS disease). Such a therapeutically effective amount can be determined as described below. The therapeutically effective amount of a composition comprising an agent as provided herein (e.g., a monoclonal antibody or antibody fragment derived therefrom as provided herein such as, for example, IC-100) can generally be about 0.001, 0.005, 0.01, 0.05, 0.1, 0.5, 1, 2, 4, 6, 8, 10, 15, 20, 25, 30, 35, 40, 45, 50, 55, 60, 65, 70, 75, 80, 85, 90, 95, 100, 125, 150, 175 or 200 mg/kg of patient body weight. The therapeutically effective amount of a composition comprising an agent as provided herein (e.g., a monoclonal antibody or antibody fragment derived therefrom as provided herein such as, for example, IC-100) can generally be about 0.001 to about 200 mg/kg of patient body weight. The therapeutically effective amount of a composition comprising an agent as provided herein (e.g., a monoclonal antibody or antibody fragment derived therefrom as provided herein such as, for example, IC-100) can generally be about 0.001 mg/kg to about 0.01 mg/kg, about 0.01 mg/kg to about 0.1 mg/kg, about 0.1 mg/kg to about 1 mg/kg, about 1 mg/kg to about 10 mg/kg, about 10 mg/kg to about 25 mg/kg, about 25 mg/kg to about 50 mg/kg, about 50 mg/kg to about 75 mg/kg, about 75 mg/kg to about 100 mg/kg, about 100 mg/kg to about 125 mg/kg, about 125 mg/kg to about 150 mg/kg, about 150 mg/kg to about 175 mg/kg or about 175 mg/kg to about 200 mg/kg of the subject's body weight. The composition comprising an agent as provided herein (e.g., a monoclonal antibody or antibody fragment derived therefrom as provided herein such as, for example, IC-100) can be administered in single or multiple doses.

Toxicity and therapeutic efficacy of the compositions for use in the methods provided herein can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. In some cases, the compositions provided herein exhibit large therapeutic indices. While those that exhibit toxic side effects may be used, care should be taken to design a delivery system that minimizes the potential damage of such side effects. In some cases, the dosage of compositions provided herein lies within a range that includes an ED₅₀ with little or no toxicity. The dosage may vary within this range depending upon the dosage form employed and the route of administration utilized.

As is well known in the medical and veterinary arts, dosage for any one subject depends on many factors, including the subject's size, body surface area, age, the particular composition to be administered, time and route of administration, general health, and other drugs being administered concurrently.

Sample Types

In any of the methods provided herein, the “biological sample” can refer to any bodily fluid or tissue obtained from a patient or subject. A biological sample can include, but is not limited to, whole blood, red blood cells, plasma, serum, peripheral blood mononuclear cells (PBMCs), urine, saliva, tears, buccal swabs, CSF, CNS microdialysate, and nerve tissue. In one embodiment, the biological sample is CSF, saliva, serum, plasma, or urine. In certain embodiments, the biological sample is CSF. In another embodiment, the biological sample is serum-derived extracellular vesicles (EVs). The EVs can be isolated from serum by any method known in the art. It should be noted that a biological sample obtained from a patient or test subject can be of the same type as a biological sample obtained from a control subject.

Kits

Also provided herein are kits for preparing a protein profile associated with a disease, disorder or condition associated with inflammation (e.g., NASH, AD, AMD, inflammaging, MCI, stroke, MS or TBI). The kits may include a reagent for measuring at least one inflammasome protein alone or in combination with at least one control biomarker proteins and instructions for measuring said at least one inflammasome protein alone and/or at least one control biomarker protein for assessing the severity of a disease, disorder or condition associated with inflammation (e.g., NASH, AD, AMD, MCI, inflammaging, stroke, MS or TBI) in a patient. As used herein, a “reagent” refers to the components necessary for detecting or quantitating one or more proteins by any one of the methods described herein. For instance, in some embodiments, kits for measuring one or more inflammasome proteins alone or in combination with at least one control biomarker proteins can include reagents for performing liquid or gas chromatography, mass spectrometry, immunoassays, immunoblots, or electrophoresis to detect one or more inflammasome proteins and/or control biomarker proteins as described herein. In some embodiments, the kit includes reagents for measuring one or more inflammasome proteins selected from IL-18, ASC, caspase-1, caspase-8, caspase-11, or combinations thereof. In some embodiments, the kit includes reagents for measuring one or more control biomarker proteins selected from Gal-3, CRP (hs-CRP), AB₍₁₋₄₂₎, AB₍₁₋₄₀₎, sAPPα, sAPPβ, or NFL, or combinations thereof.

In one embodiment, the kit comprises a labeled-binding partner that specifically binds to one or more inflammasome proteins and/or one or more control biomarker proteins, wherein said one or more inflammasome proteins are selected from the group consisting of IL-18, ASC, caspase-1, caspase-8, caspase-11 and combinations thereof, and wherein the one or more control biomarker proteins is selected from the group consisting of Gal-3, CRP (hs-CRP), AB₍₁₋₄₂₎, AB₍₁₋₄₀₎, sAPPα, sAPPβ, and NFL. Suitable binding partners for specifically binding to inflammasome proteins or control biomarker proteins include, but are not limited to, antibodies and fragments thereof, aptamers, peptides, and the like. In certain embodiments, the binding partners for detecting ASC are antibodies or fragments thereof. The antibodies directed to ASC can be any antibodies known in the art and/or commercially available. Examples of anti-ASC antibodies for use in the methods provided herein are described herein. In certain embodiments, the binding partners for detecting ASC are antibodies or fragments thereof, aptamers, or peptides that specifically bind to the amino acid sequence of SEQ ID NO: 1 or SEQ ID NO: 2 of rat ASC and human ASC, respectively. In certain embodiments, the binding partners for detecting IL-18 are antibodies or fragments thereof. The antibodies to IL-18 can be any antibodies known in the art and/or commercially available, such as those, for example, provided herein. In certain embodiments, the binding partners for detecting caspase-1 are antibodies or fragments thereof. The antibodies to caspase-1 can be any antibodies known in the art and/or commercially available, such as those, for example, provided herein. In certain embodiments, the binding partners for detecting IL-1beta are antibodies or fragments thereof. The antibodies to IL-1beta can be any antibodies known in the art and/or commercially available, such as those, for example, provided herein. The antibodies to NFL can be any antibodies known in the art and/or commercially available, such as those, for example, provided herein. In certain embodiments, the binding partners for detecting NFL are antibodies or fragments thereof. The antibodies to NFL can be any antibodies known in the art and/or commercially available, such as those, for example, provided herein. The antibodies to sAPPα can be any antibodies known in the art and/or commercially available, such as those, for example, provided herein. In certain embodiments, the binding partners for detecting sAPPα are antibodies or fragments thereof. The antibodies to sAPPα can be any antibodies known in the art and/or commercially available, such as those, for example, provided herein. The antibodies to sAPPβ can be any antibodies known in the art and/or commercially available, such as those, for example, provided herein. In certain embodiments, the binding partners for detecting sAPPβ are antibodies or fragments thereof. The antibodies to sAPPβ can be any antibodies known in the art and/or commercially available, such as those, for example, provided herein. Labels that can be conjugated to the binding partner include metal nanoparticles (e.g., gold, silver, copper, platinum, cadmium, and composite nanoparticles), fluorescent labels (e.g., fluorescein, Texas-Red, green fluorescent protein, yellow fluorescent protein, cyan fluorescent protein, Alexa dye molecules, etc.), and enzyme labels (e.g., alkaline phosphatase, horseradish peroxidase, beta-galactosidase, beta-lactamase, galactose oxidase, lactoperoxidase, luciferase, myeloperoxidase, and amylase).

### EXAMPLES

The present invention is further illustrated by the following specific examples. The examples are provided for illustration only and should not be construed as limiting the scope of the invention in any way.

### Example 1: Examination of Inflammasome Proteins as Biomarkers of Multiple Sclerosis (MS)

Multiple sclerosis (MS) is an autoimmune disease that affects the brain and spinal cord. Important to the care of patients with MS is the need for biomarkers that can predict disease onset, disease exacerbation as well as response to treatment¹.

The inflammasome is a key mediator of the innate immune response that in the CNS was first described to mediate inflammation after spinal cord injury². The inflammasome is a multiprotein complex involved in the activation of caspase-1 and the processing of the pro-inflammatory cytokines IL-1β and IL-18³.

In this example, the expression level of inflammasome proteins in serum samples from patients with MS are determined. Further, an examination of the sensitivity and specificity of inflammasome signaling proteins as biomarkers of MS was examined.

### Materials and Methods

### Participants:

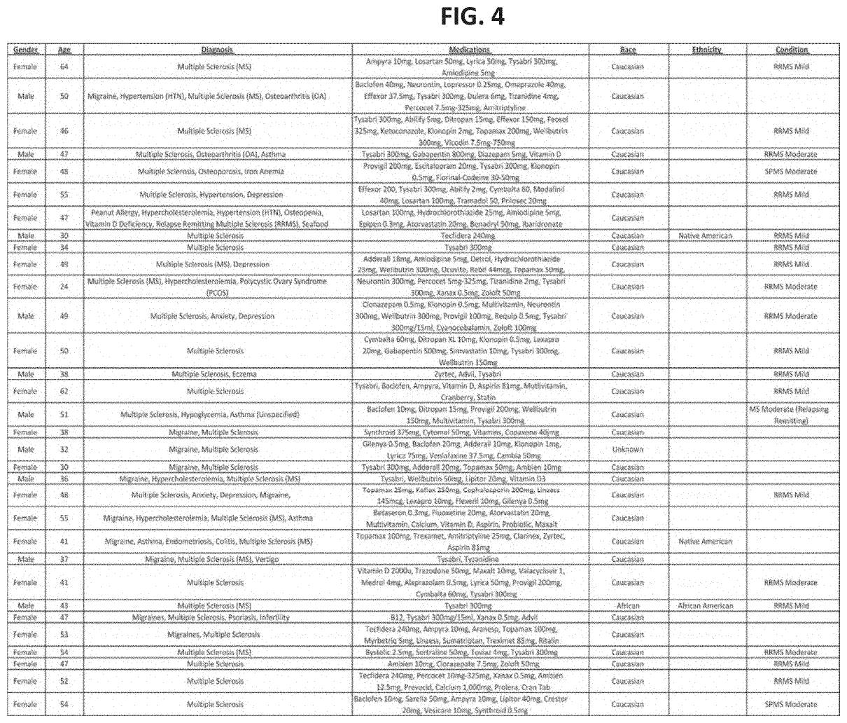

In this study, serum samples were analyzed from 120 normal donors and 32 patients that were diagnosed with MS. Samples were purchased from BioreclamationIVT. The normal donor group consisted of samples obtained from 60 male and 60 female donors in the age range of 20 to 70 years old. The age range in the MS group consisted of samples obtained from patients in the age range of 24 to 64 years old (FIG. 4).

### Protein Assay:

Concentration of inflammasome proteins ASC, IL-1β and IL-18 in serum was analyzed using a Simple Plex and a Simple Plex Explorer software. Results shown correspond to the mean of each sample run in triplicates. It should be noted that any system/instrument known in the art can be used to measure the levels of proteins (e.g., inflammasome proteins) in bodily fluids.

### Biomarker Analyses:

Prism 7 software (GraphPad) was used to analyze the data obtained from the Simple Plex Explorer Software. Comparisons between groups were carried after identifying outliers followed by determination of the area under the receiver operator characteristic (ROC) curve, as well as the 95% confidence interval (CI). The p-value of significance used was <0.05. Sensitivity and specificity of each biomarker was obtained for a range of different cut-off points. Samples that yielded a protein value below the level of detection of the assay were not included in the analyses for that analyte.

ROC curves are summarized as the area under the curve (AUC). A perfect AUC value is 1.0, where 100% of subjects in the population will be correctly classified as having MS or not. In contrast, an AUC of 0.5 signifies that subjects are randomly classified as either positive or negative for MS, which has no clinical utility. It has been suggested that an AUC between 0.9 to 1.0 applies to an excellent biomarker; from 0.8 to 0.9, good; 0.7 to 0.8 fair; 0.6 to 0.7, poor and 0.5 to 0.6, fail.

### Results

### Caspase-1, ASC and IL-18 are Elevated in the Serum of MS Patients

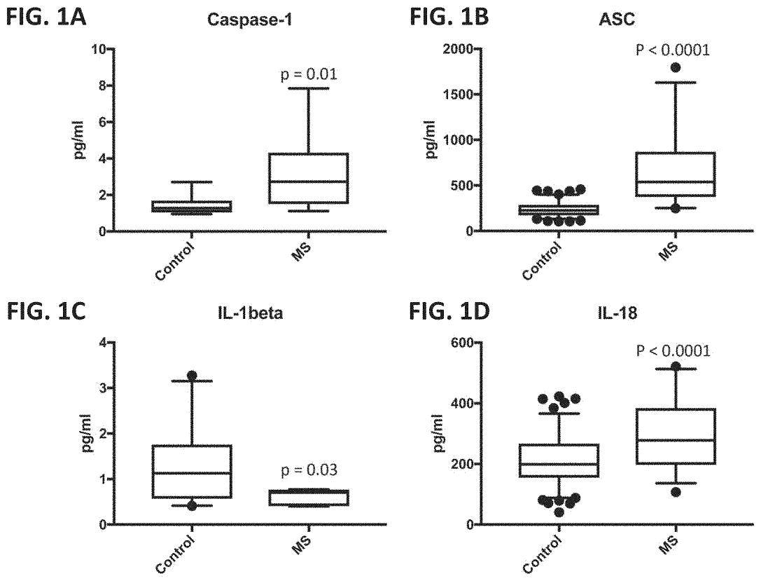

Serum samples from MS patients were analyzed and compared to serum from healthy/control individuals using a Simple Plex assay (Protein Simple) for the protein expression of the inflammasome signaling proteins caspase-1, ASC, IL-1β and IL-18 (FIG. 1A-1D). The protein levels of caspase-1, ASC and IL-18 in the serum of MS patients was higher than in the control group. However, the levels of IL-1β were lower in the MS than controls. These findings were consistent with previous reports indicating a role for the inflammasome in the pathology of MS^{6, 8, 11}.

### ASC and Caspase-1 are Good Serum Biomarkers of MS

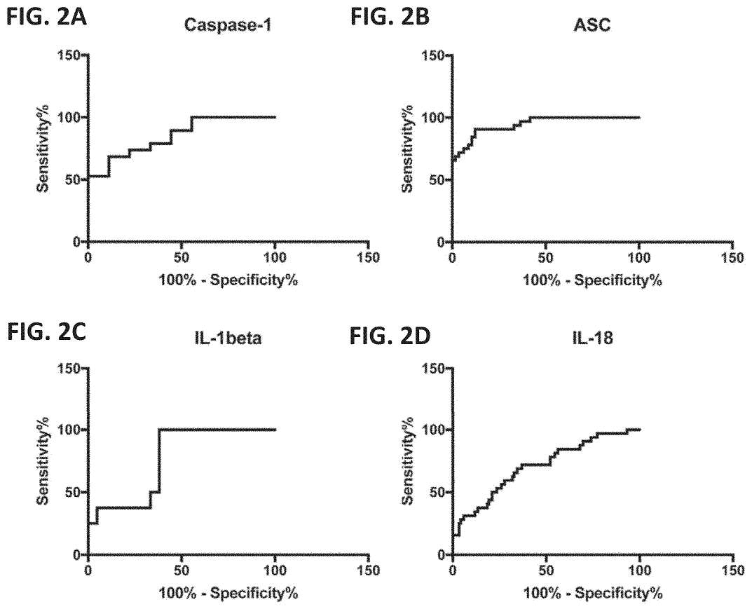

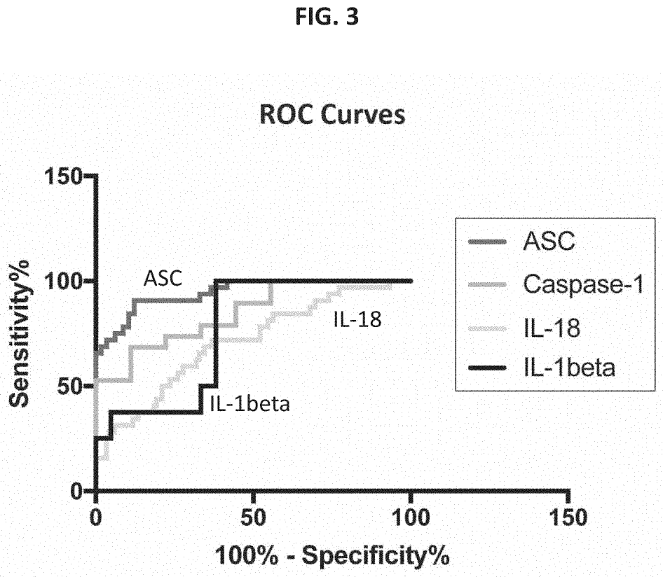

To then determine if these inflammasome signaling proteins have the potential to be reliable biomarkers for MS pathology, the area under the curve (AUC) for caspase-1 (FIG. 2A), ASC (FIG. 2B), IL-1beta (FIG. 2C) and IL-18 (FIG. 2D) were determined. Of the three proteins measured, ASC was shown to be the best biomarker (FIG. 3) with an AUC of 0.9448 and a CI between 0.9032 to 0.9864 (Table 1). In addition, caspase-1 with an AUC of 0.848 and a CI between 0.703 and 0.9929 is also promising biomarker of MS.

TABLE 1
ROC analysis results for inflammasome signaling proteins in serum.

| BIOMARKER | AREA | STD. ERROR | 95% C.I. | P VALUE |
|---|---|---|---|---|
| Caspase-1 | 0.848 | 0.07394 | 0.703 to 0.9929 | 0.0034 |
| ASC | 0.9448 | 0.02122 | 0.9032 to 0.9864 | <0.0001 |
| IL-1beta | 0.7619 | 0.0925 | 0.5806 to 0.9432 | 0.0318 |
| IL-18 | 0.7075 | 0.05216 | 0.6052 to 0.8097 | 0.0003 |

Furthermore, the cut-off point for ASC was 352.4 pg/ml with 84% sensitivity and 90% sensitivity (Table 2). For caspase-1, the cut-off point was 1.302 pg/ml with 89% sensitivity and 56% specificity (Table 2). Moreover, we found that in regards to ASC for a 100% sensitivity the cut-off point was 247.2 pg/ml with 58.26% specificity, and for 100% specificity, the cut-off point was 465.1 pg/ml and a 65.63% sensitivity. In the case of caspase-1, for 100% sensitivity, the cut-off point was 1.111 pg/ml with 44.44% specificity. For 100% specificity, the cut-off point was 2.718 pg/ml with 52.63% sensitivity. Thus, these findings indicate that caspase-1 and ASC can be biomarkers for MS.

TABLE 2
Cut-off point analyses for inflammasome signaling proteins in serum.

| | Cut-off point | Sensitivity | Specificity |
|---|---|---|---|
| Biomarker | (pg/ml) | (%) | (%) |
| Caspase-1 | >1.302 | 89 | 56 |
| ASC | >352.4 | 84 | 90 |
| IL-1beta | <0.825 | 100 | 62 |
| IL-18 | >190.1 | 84 | 44 |

### Conclusions:

In this study, a statistically significant higher level of IL-18 was detected in the serum of MS patients when compared to healthy subjects. In addition, the AUC for IL-18 in the cohort of patients was 0.7075 with a CI between 0.6052 to 0.8097 and a sensitivity of 84%, however, the specificity was only 44% when the cut-off point was 190.1 pg/ml. When the cut-off point was 104.2 pg/ml the sensitivity was 100% but the specificity was only 6.723%. Similarly, when the cut-off point was 427.2 pg/ml, the specificity was 100% but the sensitivity was only 15.63%.

Further, the levels of IL-1β were significantly lower in the MS group than the control group. The AUC was 0.7619 with a CI between 0.5806 to 0.9432. The sensitivity was 100% when the cut-off point was 0.825 with 62% specificity.

Higher protein levels of caspase-1 was also found in the serum of MS patients. Importantly, the AUC for caspase-1 was 0.848 with a CI between 0.703 to 0.9929. With a cut-off point of 1.302 pg/ml the sensitivity was 89% with 56% specificity. Moreover, with a 100% sensitivity the cut-off point was 1.111 pg/ml with 44.44% specificity; whereas with 100% specificity, the sensitivity was 52.63% with a cut-off point of 2.718 pg/ml.

Moreover, in this example, ASC was the most promising biomarker with an AUC of 0.9448 and a narrow CI between 0.9032 to 0.9864. A cut-off point of 352.4 pg/ml resulted in 84% sensitivity and 90% specificity. When the cut-off point was 247.2 pg/ml, the sensitivity was 100% and the specificity 58%.

Thus, based on these findings caspase-1 and ASC are promising biomarker with a high AUC value and a high sensitivity. Importantly, a combination of caspase-1 and ASC as biomarkers for MS with other diagnostic criteria may further increase the sensitivity of these biomarkers for MS beyond what is described in this example. Some clinically used biomarkers such as serum aquaporin 4 antibodies (AQP4-IgG), which is used to differentiate between patients with MS and patients with neuromyelitis optica, have a median sensitivity of 62.3% with a range between 12.5% to 100%, depending on the assay used for the measurements.²⁹

Since the 1960s immunoglobulin (Ig) G oligoclonal bands (OCB) have been used as a classic biomarker in the diagnosis of MS. 30 However, the specificity of IgG-OCB is only 61%, as a result, other diagnostic criteria is needed to clinically determine the diagnosis of MS, 31 yet CSF-restricted IgG-OCB is a good predictor for conversion from CIS to CDMS, independently of MRI 32. Similar results have been obtained when analyzing IgM-OCB. 33 Interestingly, IgG against measles, rubella and varicella zoster (MRZ) are present in the CSF of MS patients, thus MRZ-specific IgG have the potential to be used as biomarkers of MS diagnosis.³⁴

Importantly, in this study, caspase-1 and ASC have been identified as potential biomarkers of MS pathology with high AUC values; 0.9448 and 0.848, respectively with sensitivities above 80% and in the case of ASC a specificity of 90%.

### INCORPORATION BY REFERENCE

The following references are incorporated by reference in their entireties for all purposes.
1. Compston A. The pathogenesis and basis for treatment in multiple sclerosis. Clin Neurol Neurosurg. 2004; 106:246-8.
2. de Rivero Vaccari J P, Lotocki G, Marcillo A E, Dietrich W D and Keane R W. A molecular platform in neurons regulates inflammation after spinal cord injury. J Neurosci. 2008; 28:3404-14.
3. de Rivero Vaccari J P, Dietrich W D and Keane R W. Activation and regulation of cellular inflammasomes: gaps in our knowledge for central nervous system injury. J Cereb Blood Flow Metab. 2014; 34:369-75.
4. Ming X, Li W, Maeda Y, Blumberg B, Raval S, Cook S D and Dowling P C. Caspase-1 expression in multiple sclerosis plaques and cultured glial cells. J Neurol Sci. 2002; 197:9-18.
5. Cao Y, Goods B A, Raddassi K, Nepom G T, Kwok W W, Love J C and Hafler D A. Functional inflammatory profiles distinguish myelin-reactive T cells from patients with multiple sclerosis. Sci Transl Med. 2015; 7:287ra74.
6. Furlan R, Martino G, Galbiati F, Poliani P L, Smiroldo S, Bergami A, Desina G, Comi G, Flavell R, Su M S and Adorini L. Caspase-1 regulates the inflammatory process leading to autoimmune demyelination. J Immunol. 1999; 163:2403-9.
7. Inoue M, Williams K L, Gunn M D and Shinohara M L. NLRP3 inflammasome induces chemotactic immune cell migration to the CNS in experimental autoimmune encephalomyelitis. Proc Natl Acad Sci USA. 2012; 109:10480-5.
8. Gris D, Ye Z, Iocca H A, Wen H, Craven R R, Gris P, Huang M, Schneider M, Miller S D and Ting J P. NLRP3 plays a critical role in the development of experimental autoimmune encephalomyelitis by mediating Th1 and Th17 responses. J Immunol. 2010; 185:974-81.
9. Brand F J, 3rd, Forouzandeh M, Kaur H, Travascio F and de Rivero Vaccari J P. Acidification changes affect the inflammasome in human nucleus pulposus cells. J Inflamm (Lond). 2016; 13:29.
10. Xia J, Broadhurst D I, Wilson M and Wishart D S. Translational biomarker discovery in clinical metabolomics: an introductory tutorial. Metabolomics. 2013; 9:280-299.
11. Dumas A, Amiable N, de Rivero Vaccari J P, Chae J J, Keane R W, Lacroix S and Vallieres L. The inflammasome pyrin contributes to pertussis toxin-induced IL-1beta synthesis, neutrophil intravascular crawling and autoimmune encephalomyelitis. PLoS Pathog. 2014; 10:e1004150.
12. Katsavos S and Anagnostouli M. Biomarkers in Multiple Sclerosis: An Up-to-Date Overview. Mult Scler Int. 2013; 2013:340508.
13. Kuhle J, Disanto G, Dobson R, Adiutori R, Bianchi L, Topping J, Bestwick J P, Meier U C, Marta M, Dalla Costa G, Runia T, Evdoshenko E, Lazareva N, Thouvenot E, Iaffaldano P, Direnzo V, Khademi M, Piehl F, Comabella M, Sombekke M, Killestein J, Hegen H, Rauch S, D'Alfonso S, Alvarez-Cermeno J C, Kleinova P, Horakova D, Roesler R, Lauda F, Llufriu S, Avsar T, Uygunoglu U, Altintas A, Saip S, Menge T, Rajda C, Bergamaschi R, Moll N, Khalil M, Marignier R, Dujmovic I, Larsson H, Malmestrom C, Scarpini E, Fenoglio C, Wergeland S, Laroni A, Annibali V, Romano S, Martinez A D, Carra A, Salvetti M, Uccelli A, Torkildsen O, Myhr K M, Galimberti D, Rejdak K, Lycke J, Frederiksen J L, Drulovic J, Confavreux C, Brassat D, Enzinger C, Fuchs S, Bosca I, Pelletier J, Picard C, Colombo E, Franciotta D, Derfuss T, Lindberg R, Yaldizli O, Vecsei L, Kieseier B C, Hartung H P, Villoslada P, Siva A, Saiz A, Tumani H, Havrdova E, Villar L M, Leone M, Barizzone N, Deisenhammer F, Teunissen C, Montalban X, Tintore M, Olsson T, Trojano M, Lehmann S, Castelnovo G, Lapin S, Hintzen R, Kappos L, Furlan R, Martinelli V, Comi G, Ramagopalan S V and Giovannoni G. Conversion from clinically isolated syndrome to multiple sclerosis: A large multicentre study. Mult Scler. 2015; 21:1013-24.
14. Lublin F D. New multiple sclerosis phenotypic classification. Eur Neurol. 2014; 72 Suppl 1:1-5.
15. Milo R and Miller A. Revised diagnostic criteria of multiple sclerosis. Autoimmun Rev. 2014; 13:518-24.
16. Inoue M, Chen P H, Siecinski S, Li Q J, Liu C, Steinman L, Gregory S G, Benner E and Shinohara M L. An interferon-beta-resistant and NLRP3 inflammasome-independent subtype of EAE with neuronal damage. Nat Neurosci. 2016; 19:1599-1609.
17. Inoue M, Williams K L, Oliver T, Vandenabeele P, Rajan J V, Miao E A and Shinohara M L. Interferon-beta therapy against EAE is effective only when development of the disease depends on the NLRP3 inflammasome. Sci Signal. 2012; 5:ra38.
18. Chen Y C, Chen S D, Miao L, Liu Z G, Li W, Zhao Z X, Sun X J, Jiang G X and Cheng Q. Serum levels of interleukin (IL)-18, IL-23 and IL-17 in Chinese patients with multiple sclerosis. J Neuroimmunol. 2012; 243:56-60.
19. Losy J and Niezgoda A. IL-18 in patients with multiple sclerosis. Acta Neurol Scand. 2001; 104:171-3.
20. Levesque S A, Pare A, Mailhot B, Bellver-Landete V, Kebir H, Lecuyer M A, Alvarez J I, Prat A, de Rivero Vaccari J P, Keane R W and Lacroix S. Myeloid cell transmigration across the CNS vasculature triggers IL-1beta-driven neuroinflammation during autoimmune encephalomyelitis in mice. J Exp Med. 2016; 213:929-49.
21. Dujmovic I, Mangano K, Pekmezovic T, Quattrocchi C, Mesaros S, Stojsavljevic N, Nicoletti F and Drulovic J. The analysis of IL-1 beta and its naturally occurring inhibitors in multiple sclerosis: The elevation of IL-1 receptor antagonist and IL-1 receptor type II after steroid therapy. J Neuroimmunol. 2009; 207:101-6.
22. Hauser S L, Doolittle T H, Lincoln R, Brown R H and Dinarello C A. Cytokine accumulations in CSF of multiple sclerosis patients: frequent detection of interleukin-1 and tumor necrosis factor but not interleukin-6. Neurology. 1990; 40:1735-9.
23. Maimone D, Gregory S, Amason B G and Reder A T. Cytokine levels in the cerebrospinal fluid and serum of patients with multiple sclerosis. J Neuroimmunol. 1991; 32:67-74.
24. Tsukada N, Miyagi K, Matsuda M, Yanagisawa N and Yone K. Tumor necrosis factor and interleukin-1 in the CSF and sera of patients with multiple sclerosis. J Neurol Sci. 1991; 104:230-4.
25. Huang W X, Huang P and Hillert J. Increased expression of caspase-1 and interleukin-18 in peripheral blood mononuclear cells in patients with multiple sclerosis. Mult Scler. 2004; 10:482-7.
26. de Rivero Vaccari J P, Dietrich W D and Keane R W. Therapeutics targeting the inflammasome after central nervous system injury. Transl Res. 2016; 167:35-45.
27. de Rivero Vaccari J P, Lotocki G, Alonso O F, Bramlett H M, Dietrich W D and Keane R W. Therapeutic neutralization of the NLRP1 inflammasome reduces the innate immune response and improves histopathology after traumatic brain injury. J Cereb Blood Flow Metab. 2009; 29:1251-61.
28. Shaw P J, Lukens J R, Burns S, Chi H, McGargill M A and Kanneganti T D. Cutting edge: critical role for PYCARD/ASC in the development of experimental autoimmune encephalomyelitis. J Immunol. 2010; 184:4610-4.
29. Jarius S and Wildemann B. Aquaporin-4 antibodies (NMO-IgG) as a serological marker of neuromyelitis optica: a critical review of the literature. Brain Pathol. 2013; 23:661-83.
30. Stangel M, Fredrikson S, Meinl E, Petzold A, Stuve O and Tumani H. The utility of cerebrospinal fluid analysis in patients with multiple sclerosis. Nat Rev Neurol. 2013; 9:267-76.
31. Teunissen C E, Malekzadeh A, Leurs C, Bridel C and Killestein J. Body fluid biomarkers for multiple sclerosis—the long road to clinical application. Nat Rev Neurol. 2015; 11:585-96.
32. Tintore M, Rovira A, Rio J, Tur C, Pelayo R, Nos C, Tellez N, Perkal H, Comabella M, Sastre-Garriga J and Montalban X. Do oligoclonal bands add information to MRI in first attacks of multiple sclerosis? Neurology. 2008; 70:1079-83.
33. Villar L M, Masjuan J, Gonzalez-Porque P, Plaza J, Sadaba M C, Roldan E, Bootello A and Alvarez-Cermeno J C. Intrathecal IgM synthesis predicts the onset of new relapses and a worse disease course in MS. Neurology. 2002; 59:555-9.
34. Brettschneider J, Tumani H, Kiechle U, Muche R, Richards G, Lehmensiek V, Ludolph A C and Otto M. IgG antibodies against measles, rubella, and varicella zoster virus predict conversion to multiple sclerosis in clinically isolated syndrome. PLoS One. 2009; 4:e7638.

### Example 2: Examination of Inflammasome Proteins as Biomarkers of Stroke Introduction

A biomarker is a characteristic that can be measured objectively and evaluated as an indicator of normal or pathologic biological processes⁹. Thus, in the context of stroke, biomarkers in blood or other body fluids can be used as indicators of stroke onset. However, to date, there is no biomarker available that is regularly used in the diagnosis and management of stroke. To this end, cytokines such as IL-10 or tumor necrosis factor as well as other inflammatory proteins such as C-reactive protein, high-mobility group box-1 or heat shock proteins have been considered as potential candidates for further biomarker analyses in stroke patients¹⁰⁻¹².

In this example, a Simple Plex Assay (Protein Simple) was used to analyze serum and serum-derived EV samples from stroke patients and control donors for inflammasome protein levels of caspase-1, apoptosis-associated speck-like protein containing a caspase-recruitment domain (ASC), Interleukin (IL)-1beta. Receiver operator characteristic (ROC) curves and associated confidence intervals were calculated following analysis of the serum and serum-derived EV samples from patients after stroke and from healthy unaffected donors to measure sensitivity and specificity of inflammasome proteins to establish the potential of inflammasome signaling proteins as biomarkers of stroke.

### Methods

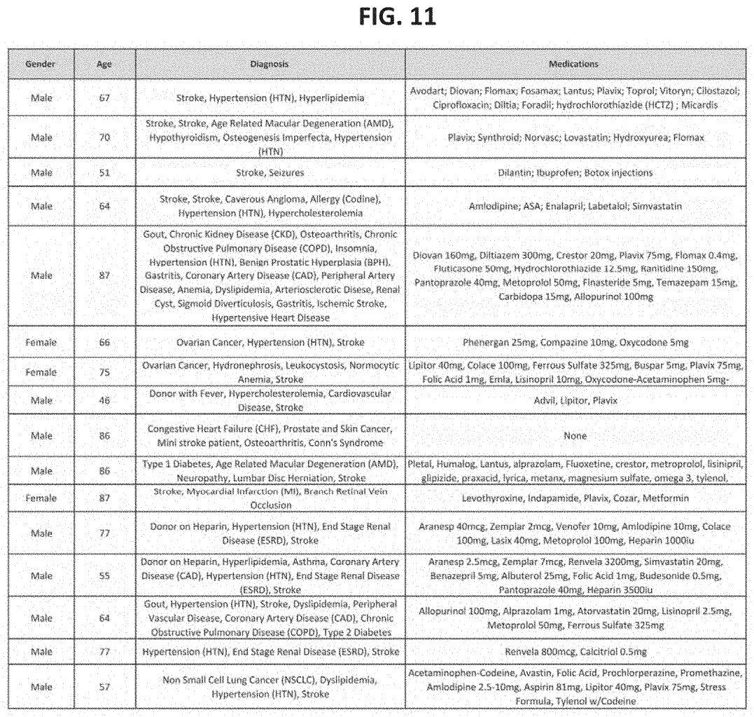

Participants: In this example, serum samples from 80 normal donors and 16 patients that were diagnosed with stroke were analyzed. Samples were purchased from BioreclamationIVT. The normal donor group consisted of samples obtained from 40 male and 40 female donors in the age range of 46 to 70 years old. The age range in the stroke group consisted of samples obtained from patients in the age range of 46 to 87 years old (FIG. 11).

### Isolation of EV:

By Total Exosome Isolation from Serum kit (Invitrogen): Total Exosome Isolation from serum was used according to the manufacturer's instructions (Invitrogen). Briefly, 100 ul of each sample was centrifuged at 2000×g for 30 minutes. The supernatant was then incubated with 20 ul of Total Exosome Isolation reagent for 30 minutes at 4° C. followed by centrifugation at 10,000×g for 10 minutes at room temperature. Supernatants were discarded and the pellet was resuspended in 50 ul of PBS.

By ExoQuick: EV were isolated from serum samples using ExoQuick (EQ, System Biosciences) as described in6. Briefly, 100 ul of each sample was centrifuged at 3,000×g for 15 minutes. The supernatant was then incubated with 24.23 ul of ExoQuick Exosome Precipitation Solution (for serum) for 30 min at 4° C. followed by centrifugation at 1,500×g for 30 minutes. Supernatants were discarded and residual EQ solution was centrifuged at 1,500×g for 5 minutes. The pellet was then resuspended in 50 ul of PBS.

### Protein Assay:

To determine the protein concentration of caspase-1, ASC, IL-1β and IL-18 in serum and serum-derived EV, a Simple Plex assay was run and analyzed with Simple Plex Explorer software. Results shown correspond to the mean of each sample run in triplicates. It should be noted that any system/instrument known in the art can be used to measure the levels of proteins (e.g., inflammasome proteins) in bodily fluids.

### Protein Quantification

To quantify the protein concentration in isolated EV, the Pierce Coomassie (Bradford) Protein Assay Kit (ThermoFisher Scientific, Inc.) was used according to the manufacturer's instructions. Serum-derived EV were lysed (1:1 dilution) in lysis buffer as described.⁶

### Nanoparticle Tracking Analysis (NTA)

EV were analyzed by NanoSight NS300 (Malvern Instruments Company, Nanosight, and Malvern, United Kingdom). Isolated exosomes were diluted in PBS (1:1000) for analysis, and three 90 second videos were then recorded. Data were analyzed using Nanosight NTA 2.3 Analytical Software (Malvern Instruments Company) with a detection threshold optimized for each sample and a screen gain set at 10 to track as many particles as possible while maintaining minimal background. At least three independent measurements were performed for each isolated sample.

### Immunoblotting

For detection of inflammasome signaling proteins in isolated EV, EV were resuspended in protein lysis buffer and resolved by immunoblotting as described in 15. Briefly, following lysis of the pellet proteins were resolved in 10-20% Criterion TGX Stain-Free precasted gels (Bio-Rad), using antibodies (1:1000 dilution) to NLRP3 (Novus Biologicals), caspase-1 (Novus Biologicals), ASC (Santa Cruz), IL-1beta (Cell Signaling), IL-18 (Abcam), CD81 (Thermo Scientific) and NCAM (Sigma). Quantification of band density was done using the UN-SCAN-IT gel 5.3 Software (Silk Scientific Corporation). Ten ul of sample was loaded. Chemilluminescence substrate (LumiGlo, Cell Signaling) in membranes was imaged using the ChemiDoc Touch Imaging System (BioRad).

### Gel Imaging

Total protein in the Criterion TGX Stain-Free precasted gels was imaged using the ChemiDoc Touch Imaging System (BioRad) by placing the gel in the tray of the ChemiDoc Touch following protein transfer. The image was then adjusted in the screen to show the entirety of the gel and running the Stain-Free Blot setting in the application window.

### Statistical Analyses

Statistical comparisons between the Invitrogen and ExoQuick isolation procedures were done using a two-tailed student t-test.

### Electron Microscopy Procedures

EV were loaded onto formvar-carbon coated grids. A 10 ul drop of the sample was then placed on clean parafilm and the grid was floated (face-down) for 30 min. Subsequent steps were also performed by floating the grid on a 10 ul bubble. The EV-loaded grid was then rinsed with 0.1 M Millonig's phosphate buffer (Electron Microscopy Sciences) for 5 min. Excess fluid was drained. Then the grid was placed into 2% glutaraldehyde for 5 min. Subsequent washes were done to remove excess glutaraldehyde by rinsing with 0.1 M Millonig's phosphate buffer for 5 min followed by distilled water for 2 min seven times on seven different bubbles. The grid was then transferred to a 0.4% Uranyl Acetate solution for 5 min. Grids were allowed to dry for imaging. Images were acquired with a Joel JEM-1400 transmission electron microscope, at a voltage of 80 kV, and a digital Gatan camera.

### Biomarker Analyses

Data were analyzed using Prism 7 software (GraphPad). Comparisons between groups for protein levels were carried by first identifying outliers followed by an unpaired t-test and then determining the area under the ROC curve, as well as the 95% confidence interval and the p-value (p-value of significance used was <0.05). Finally, sensitivity, specificity, positive predictive value (PPV), negative predictive value (NPV) and accuracy of each biomarker was obtained for a range of different cut-off points. Samples that yielded a protein value below the level of detection of the assay were not included in the analyses for that particular analyte.

### Results

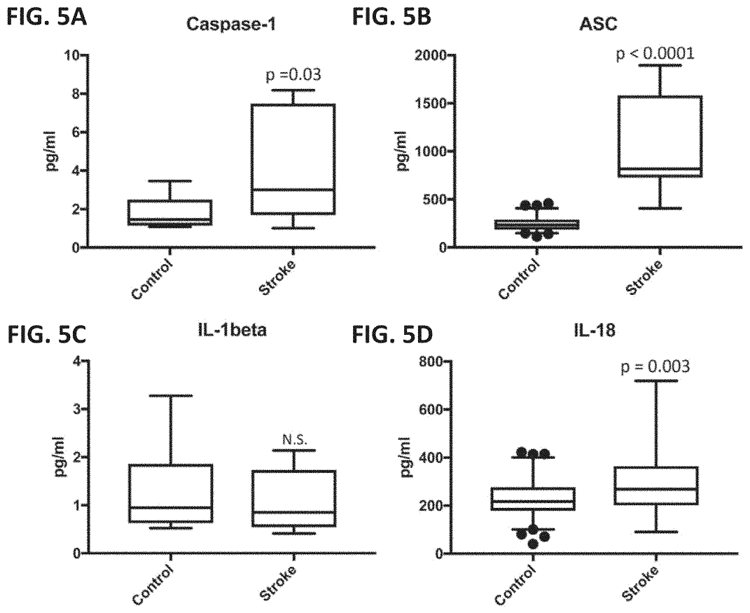

Caspase-1, ASC and IL-18 are elevated in the serum of stroke patients: To determine the protein levels of inflammasome proteins in serum from stroke patients and control donors, serum samples were analyzed with a Simple Plex system. Protein levels of caspase-1, ASC and IL-18 were higher in the serum of stroke patients when compared to the control samples, whereas levels of IL-1 were not significantly different (FIG. 5A-5D). These findings confirm previous data showing that the inflammasome is involved in the inflammatory response after stroke^{4, 16}.

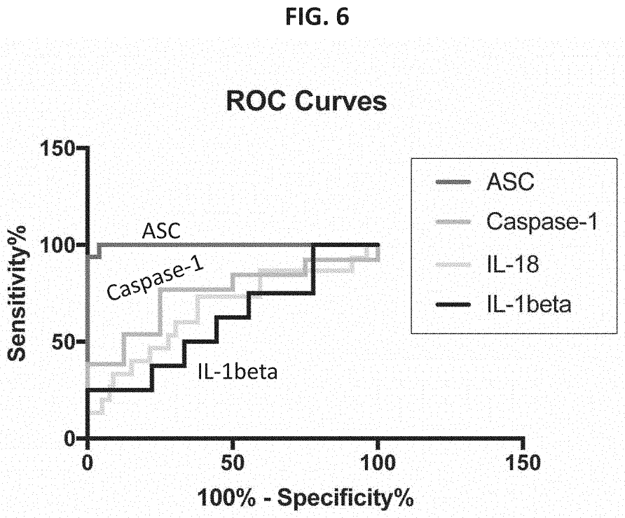

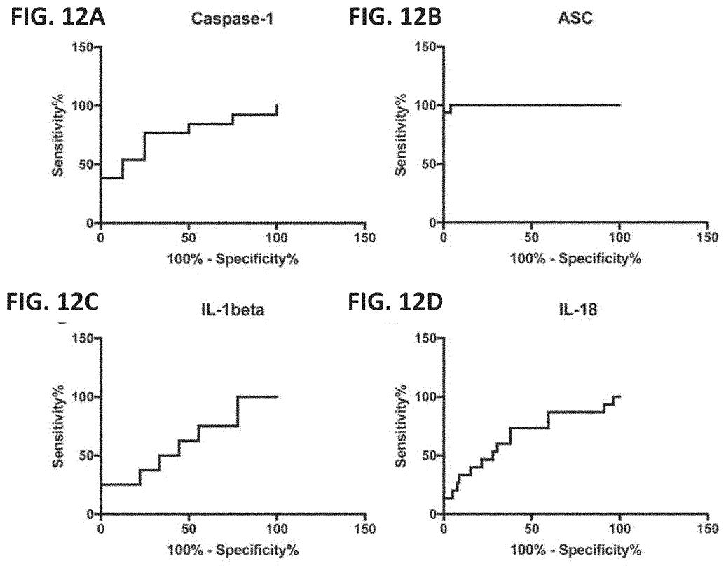

ASC as a serum biomarker of stroke: Higher levels of inflammasome proteins in serum from stroke patients may not be enough proof to show that inflammasome proteins are good biomarkers of stroke. Thus, an ROC analysis was performed (FIG. 6 and FIG. 12A-12D) to determine the AUC. The AUC for ASC was 0.9975 with a confidence interval between 0.9914 to 1.004 (Table 3). The cut-off point for ASC was 404.8 pg/ml with a sensitivity of 100% and a specificity of 96% (Table 4). Thus, ASC appears to be a reliable biomarker of stroke.

TABLE 3
ROC analysis results for inflammasome signaling proteins in serum.

| BIO- | | STD. | | |
|---|---|---|---|---|
| MARKER | AREA | ERROR | 95% C.I. | P VALUE |
| Caspase-1 | 0.75 | 0.1087 | 0.5369 to 0.9631 | 0.05 |
| ASC | 0.9975 | 0.003 | 0.9914 to 1.004 | <0.0001 |
| IL-1beta | 0.6111 | 0.1407 | 0.3353 to 0.8869 | 0.44 |
| IL-18 | 0.6675 | 0.082 | 0.5059 to 0.8291 | 0.04 |

TABLE 4
Cut-off point analyses for inflammasome signaling proteins in serum.

| | Cut-off point | Sensitivity | Specificity |
|---|---|---|---|
| Biomarker | (pg/ml) | (%) | (%) |
| Caspase-1 | >1.412 | 85 | 50 |
| ASC | >404.8 | 100 | 96 |
| IL-1beta | <0.984 | 63 | 56 |
| IL-18 | >244.6 | 73 | 62 |

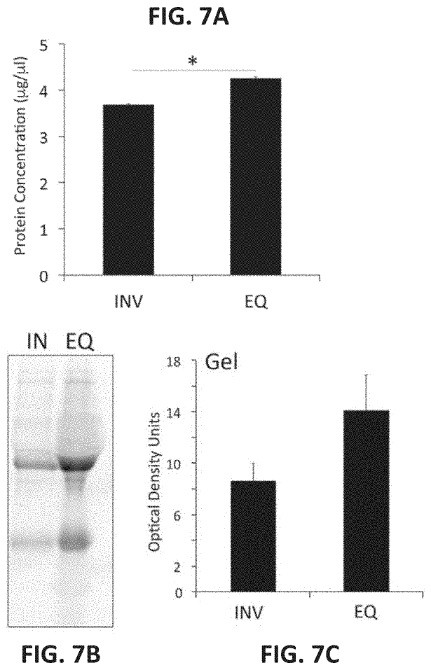

Amount of protein loaded in Isolated EV from stroke patients: To calculate the amount of protein present in the isolated exosomes from serum samples, a BCA assay was performed from isolates obtained by the Invitrogen method and the EQ method. The data indicated that the EQ method was able to isolate more protein than the Invitrogen method (FIG. 7A-7C).

To visualize how much protein was loaded in a gel during immunoblot analysis, the Stain-Free Blot setting of the ChemiDoc Touch Imaging System was used. The representative image in FIG. 7B showed that when 10 ul was loaded of the serum-derived EV re-suspended in lysis buffer containing a protease inhibitor cocktail (Sigma), the lanes corresponding to the Invitrogen kit had less protein than the lane corresponding to the EQ kit; however, there was no statistical significant difference between the groups.

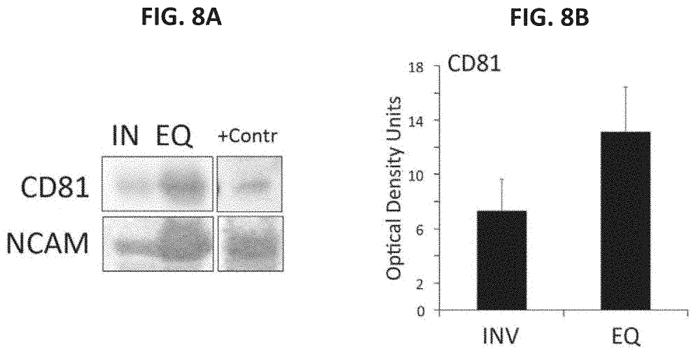

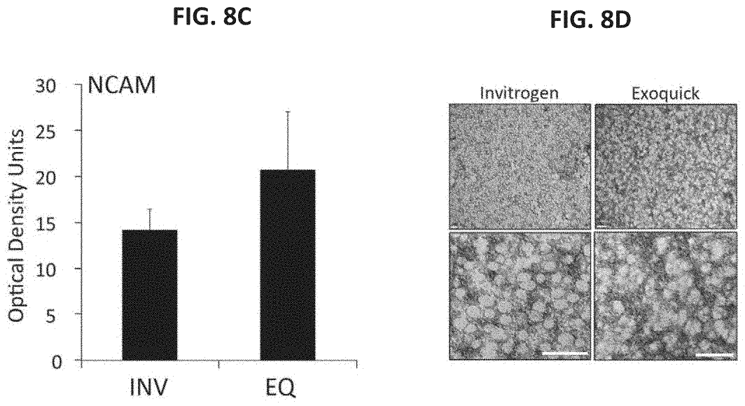

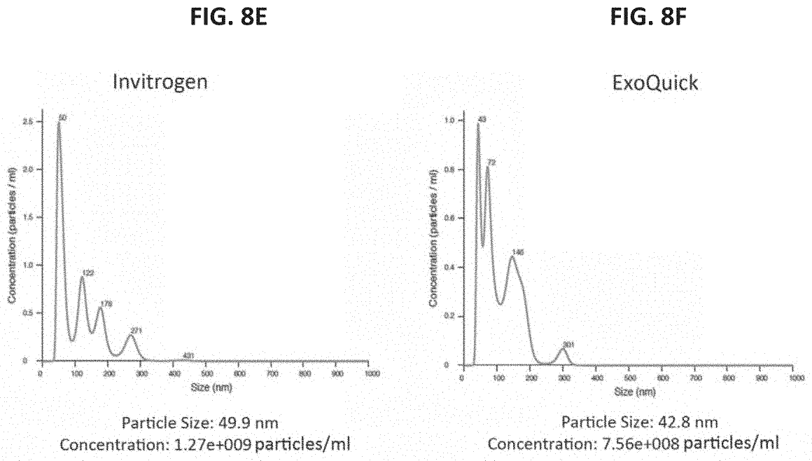

Invitrogen's kit and EQ isolate CD81- and NCAM-positive EV from the serum of patients with stroke: To determine if inflammasome proteins present in EV are promising biomarkers of stroke, EV from the serum of stroke patients was isolated. Two different techniques of EV isolation was used to identify the most suitable method to isolate, inflammasome-containing EV. In addition, the tetraspanin protein CD81, a marker of EV {Andreu, 2014 #33} as well as and neural cell adhesion molecule (NCAM) a marker of neuronal-derived EV was used to demonstrate that the isolated EV are brain derived {Vella, 2016 #36}. Accordingly, both methods, the one from Invitrogen and EQ, were able to isolate CD81- and (NCAM)-positive EV (FIG. 8A). However, although the EQ seem to isolate higher levels of these proteins, there was no statistical significant difference between the two groups (FIG. 8B and FIG. 8C). EV-positive control isolate (System Biosciences) was run in parallel.

Electron microscopy was performed on the EV isolated by the two techniques and found that the Invitrogen kit gave more uniformed and round vesicles (FIG. 8D). In addition, NTA analyses revealed that the particle size was in the 40 to 50 nm range for both techniques, and the particle concentration of EV with the Invitrogen method was 1.27e+009 particles/ml and with EQ, 7.56+008 particles/ml (FIG. 8E and FIG. 8F). Taken together, based on the particle size and uniformity of vesicles, as determined by electron microscopy, it seems that the Invitrogen method is more suitable to isolate EV.

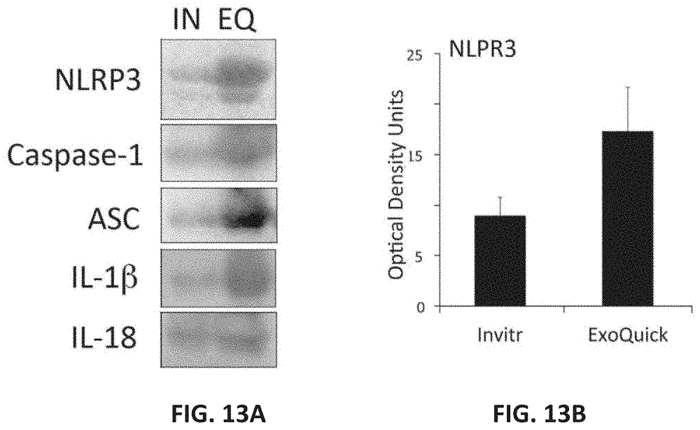

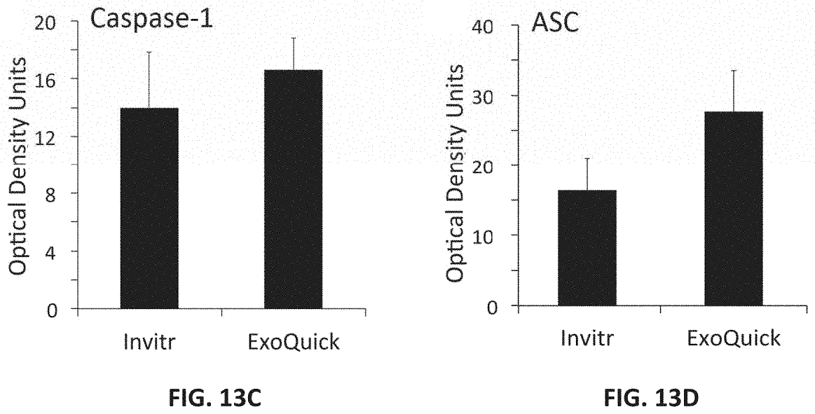

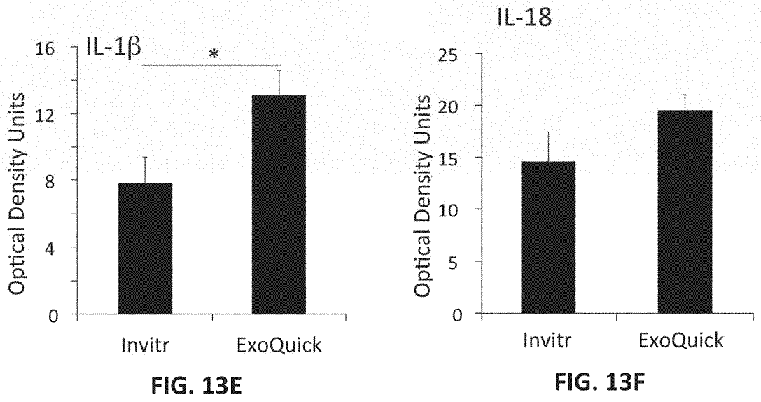

Invitrogen's kit and EQ isolate inflammasome-positive EV from the serum of patients with stroke: It has been previously shown that inflammasome proteins are present in EV6. The levels of inflammasome protein expression was compared by the two different methods and found no statistical significant difference in NLPR3, caspase-1, ASC and IL-18 levels between the two different methods. However, the EQ method was able to isolate EV with higher levels of IL-1beta than the Invitrogen method (see FIG. 13A-13F).

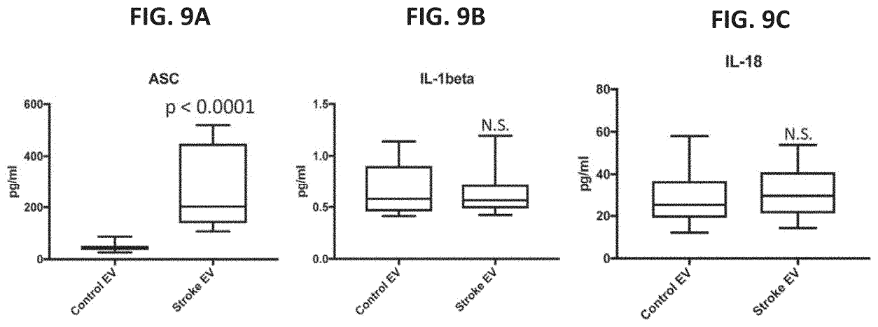

ASC is elevated in EV isolated from the serum of stroke patients: EV from the serum of 16 aged-matched donors and the 16 stroke samples (FIG. 11) was isolated and analyzed inflammasome protein levels in these isolated EV with the Simple Plex technology. The protein levels of ASC remained higher in serum-derived EV from stroke samples when compared to controls (FIG. 9A-9C). However, the levels of IL-1beta and IL-18 were not significantly different between the two groups, while the levels of caspase-1 in these isolated EV was below the limit of detection of these assay for this analyte.

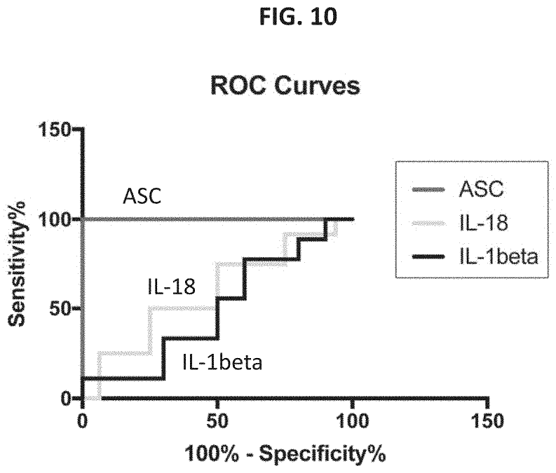

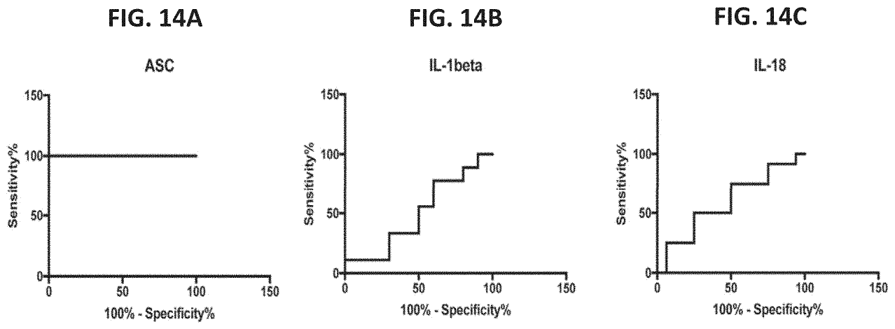

ASC in serum-derived EV is a good biomarker of stroke: To determine if inflammasome proteins in serum-derived EV can be viable biomarkers of stroke, an ROC analysis (see FIG. 14A-14C) was conducted and found that ASC is a reliable biomarker of stroke (FIG. 10) with an AUC of 1 (Table 5) and a cut-off point of 97.57 pg/ml (Table 6).

TABLE 5
ROC analysis results for inflammasome signaling proteins
in serum-derived EV.

| BIOMARKER | AREA | STD. ERROR | 95% C.I. | P VALUE |
|---|---|---|---|---|
| ASC | 1 | 0 | 1 | <0.0001 |
| IL-1beta | 0.5 | 0.1375 | 0.2303 to | >0.9999 |
| | | | 0.7697 | |
| IL-18 | 0.5938 | 0.1109 | 0.3763 to | 0.4034 |
| | | | 0.8112 | |

TABLE 6
Cut-off analyses for inflammasome signaling proteins in serum-derived EV.

| | Cut-off point | Sensitivity | Specificity |
|---|---|---|---|
| Biomarker | (pg/ml) | (%) | (%) |
| ASC | >97.57 | 100 | 100 |
| IL-1beta | >0.5585 | 56 | 50 |
| IL-18 | >23.66 | 75 | 50 |

### Conclusion

In this example, it was shown that ASC is a reliable biomarker of stroke onset. The area under the curve (AUC) for ASC in serum was 0.9975 with a confidence interval between 0.9914 to 1.004. This AUC value was higher than the other inflammasome signaling proteins analyzed in this study: caspase-1 (0.75), IL-1beta (0.6111) and IL-18 (0.6675), indicating that ASC is a superior biomarker to the other inflammasome proteins that were looked at in this study. The cut-off point for ASC was 404.8 pg/ml with 100% sensitivity and a 96% specificity with the cohort of samples used. Importantly, the AUC was increased to 1 when analyzing serum-derived EV samples from a small subset of patients. Accordingly, the cut-off point for ASC in serum-derived EV was found to be 97.57 pg/ml.

In this study, the Invitrogen kit was able to provide better quality EV as visualized by electron microscopy and by NTA analysis of isolated vesicles, despite obtained higher levels of protein isolation with the EQ kit. Importantly, both methods were efficient at isolating EV containing inflammasome proteins

In conclusion, these studies highlight the potential of inflammasome proteins, particularly ASC as a biomarker of stroke in serum and serum-derived EV.

### INCORPORATION BY REFERENCE

The following references are incorporated by reference in their entireties for all purposes.
1. Xu X and Jiang Y. The Yin and Yang of innate immunity in stroke. Biomed Res Int. 2014; 2014:807978.
2. Neumann S, Shields N J, Balle T, Chebib M and Clarkson A N. Innate Immunity and Inflammation Post-Stroke: An alpha7-Nicotinic Agonist Perspective. Int J Mol Sci. 2015; 16:29029-46.
3. Brand F J, 3rd, de Rivero Vaccari J C, Mejias N H, Alonso O F and de Rivero Vaccari J P. RIG-I contributes to the innate immune response after cerebral ischemia. J Inflamm (Lond). 2015; 12:52.
4. Abulafia D P, de Rivero Vaccari J P, Lozano J D, Lotocki G, Keane R W and Dietrich W D. Inhibition of the inflammasome complex reduces the inflammatory response after thromboembolic stroke in mice. J Cereb Blood Flow Metab. 2009; 29:534-44.
5. de Rivero Vaccari J P, Dietrich W D and Keane R W. Therapeutics targeting the inflammasome after central nervous system injury. Transl Res. 2016; 167:35-45.
6. de Rivero Vaccari J P, Brand F, 3rd, Adamczak S, Lee S W, Perez-Barcena J, Wang M Y, Bullock M R, Dietrich W D and Keane R W. Exosome-mediated inflammasome signaling after central nervous system injury. J Neurochem. 2016; 136 Suppl 1:39-48.
7. Zhang Z G and Chopp M. Exosomes in stroke pathogenesis and therapy. J Clin Invest. 2016; 126:1190-7.
8. Ji Q, Ji Y, Peng J, Zhou X, Chen X, Zhao H, Xu T, Chen L and Xu Y. Increased Brain-Specific MiR-9 and MiR-124 in the Serum Exosomes of Acute Ischemic Stroke Patients. PLoS One. 2016; 11:e0163645.
9. Biomarkers Definitions Working G. Biomarkers and surrogate endpoints: preferred definitions and conceptual framework. Clin Pharmacol Ther. 2001; 69:89-95.
10. Whiteley W, Chong W L, Sengupta A and Sandercock P. Blood markers for the prognosis of ischemic stroke: a systematic review. Stroke. 2009; 40:e380-9.
11. Bustamante A, Simats A, Vilar-Bergua A, Garcia-Berrocoso T and Montaner J. Blood/Brain Biomarkers of Inflammation After Stroke and Their Association With Outcome: From C-Reactive Protein to Damage-Associated Molecular Patterns. Neurotherapeutics. 2016; 13:671-684.
12. Katan M and Elkind M S. Inflammatory and neuroendocrine biomarkers of prognosis after ischemic stroke. Expert Rev Neurother. 2011; 11:225-39.
13. Adamczak S, Dale G, de Rivero Vaccari J P, Bullock M R, Dietrich W D and Keane R W. Inflammasome proteins in cerebrospinal fluid of brain-injured patients as biomarkers of functional outcome: clinical article. J Neurosurg. 2012; 117:1119-25.
14. Brand F J, 3rd, Forouzandeh M, Kaur H, Travascio F and de Rivero Vaccari J P. Acidification changes affect the inflammasome in human nucleus pulposus cells. J Inflamm (Lond). 2016; 13:29.
15. de Rivero Vaccari J C, Brand F J, 3rd, Berti A F, Alonso O F, Bullock M R and de Rivero Vaccari J P. Mincle signaling in the innate immune response after traumatic brain injury. Journal of neurotrauma. 2015; 32:228-36.
16. de Rivero Vaccari J P, Patel H H, Brand F J, 3rd, Perez-Pinzon M A, Bramlett H M and Raval A P. Estrogen receptor beta signaling alters cellular inflammasomes activity after global cerebral ischemia in reproductively senescence female rats. J Neurochem. 2016; 136:492-6.
17. de Rivero Vaccari J P, Dietrich W D and Keane R W. Activation and regulation of cellular inflammasomes: gaps in our knowledge for central nervous system injury. J Cereb Blood Flow Metab. 2014; 34:369-75.
18. de Rivero Vaccari J P, Dietrich W D and Keane R W. Therapeutics targeting the inflammasome after central nervous system injury. Translational research: the journal of laboratory and clinical medicine. 2015.
19. de Rivero Vaccari J P, Lotocki G, Alonso O F, Bramlett H M, Dietrich W D and Keane R W. Therapeutic neutralization of the NLRP1 inflammasome reduces the innate immune response and improves histopathology after traumatic brain injury. J Cereb Blood Flow Metab. 2009; 29:1251-61.
20. de Rivero Vaccari J P, Lotocki G, Marcillo A E, Dietrich W D and Keane R W. A molecular platform in neurons regulates inflammation after spinal cord injury. J Neurosci. 2008; 28:3404-14.
21. Fann D Y, Lee S Y, Manzanero S, Tang S C, Gelderblom M, Chunduri P, Bernreuther C, Glatzel M, Cheng Y L, Thundyil J, Widiapradja A, Lok K Z, Foo S L, Wang Y C, Li Y I, Drummond G R, Basta M, Magnus T, Jo D G, Mattson M P, Sobey C G and Arumugam T V. Intravenous immunoglobulin suppresses NLRP1 and NLRP3 inflammasome-mediated neuronal death in ischemic stroke. Cell Death Dis. 2013; 4:e790.
22. Minkiewicz J, de Rivero Vaccari J P and Keane R W. Human astrocytes express a novel NLRP2 inflammasome. Glia. 2013; 61:1113-21.
23. Sun X, Song X, Zhang L, Sun J, Wei X, Meng L and An J. NLRP2 is highly expressed in a mouse model of ischemic stroke. Biochem Biophys Res Commun. 2016; 479:656-662.
24. Ma Q, Chen S, Hu Q, Feng H, Zhang J H and Tang J. NLRP3 inflammasome contributes to inflammation after intracerebral hemorrhage. Ann Neurol. 2014; 75:209-19.
25. Fann D Y, Lim Y A, Cheng Y L, Lok K Z, Chunduri P, Baik S H, Drummond G R, Dheen S T, Sobey C G, Jo D G, Chen C L and Arumugam T V. Evidence that N F-kappaB and MAPK Signaling Promotes NLRP Inflammasome Activation in Neurons Following Ischemic Stroke. Mol Neurobiol. 2017.
26. Zhang N, Zhang X, Liu X, Wang H, Xue J, Yu J, Kang N and Wang X. Chrysophanol inhibits NALP3 inflammasome activation and ameliorates cerebral ischemia/reperfusion in mice. Mediators Inflamm. 2014; 2014:370530.
27. Mendis S, Davis S and Norrving B. Organizational update: the world health organization global status report on noncommunicable diseases 2014; one more landmark step in the combat against stroke and vascular disease. Stroke. 2015; 46:e121-2.
28. Esenwa C C and Elkind M S. Inflammatory risk factors, biomarkers and associated therapy in ischaemic stroke. Nat Rev Neurol. 2016; 12:594-604.
29. Ridker P M and Haughie P. Prospective studies of C-reactive protein as a risk factor for cardiovascular disease. J Investig Med. 1998; 46:391-5.
30. Rosenson R S and Stafforini D M. Modulation of oxidative stress, inflammation, and atherosclerosis by lipoprotein-associated phospholipase A2. J Lipid Res. 2012; 53:1767-82.
31. Oei H H, van der Meer I M, Hofman A, Koudstaal P J, Stijnen T, Breteler M M and Witteman J C. Lipoprotein-associated phospholipase A2 activity is associated with risk of coronary heart disease and ischemic stroke: the Rotterdam Study. Circulation. 2005; 111:570-5.
33. Barber M, Langhorne P, Rumley A, Lowe G D and Stott D J. Hemostatic function and progressing ischemic stroke: D-dimer predicts early clinical progression. Stroke. 2004; 35:1421-5.
34. Turaj W, Slowik A, Dziedzic T, Pulyk R, Adamski M, Strojny J and Szczudlik A. Increased plasma fibrinogen predicts one-year mortality in patients with acute ischemic stroke. J Neurol Sci. 2006; 246:13-9.
35. Mathivanan S, Ji H and Simpson R J. Exosomes: extracellular organelles important in intercellular communication. J Proteomics. 2010; 73:1907-20.
36. Le Pecq J B. Dexosomes as a therapeutic cancer vaccine: from bench to bedside. Blood Cells Mol Dis. 2005; 35:129-35.
37. Kourembanas S. Exosomes: vehicles of intercellular signaling, biomarkers, and vectors of cell therapy. Annu Rev Physiol. 2015; 77:13-27.
38. Thery C, Zitvogel L and Amigorena S. Exosomes: composition, biogenesis and function. Nat Rev Immunol. 2002; 2:569-79.
39. Campos J H, Soares R P, Ribeiro K, Andrade A C, Batista W L and Torrecilhas A C. Extracellular Vesicles: Role in Inflammatory Responses and Potential Uses in Vaccination in Cancer and Infectious Diseases. J Immunol Res. 2015; 2015:832057.
40. Hurley J H, Boura E, Carlson L A and Rozycki B. Membrane budding. Cell. 2010; 143:875-87.
41. Thery C, Ostrowski M and Segura E. Membrane vesicles as conveyors of immune responses. Nat Rev Immunol. 2009; 9:581-93.
42. Vella L J, Sharples R A, Nisbet R M, Cappai R and Hill A F. The role of exosomes in the processing of proteins associated with neurodegenerative diseases. Eur Biophys J. 2008; 37:323-32.
43. Izquierdo-Useros N, Naranjo-Gomez M, Erkizia I, Puertas M C, Borras F E, Blanco J and Martinez-Picado J. HIV and mature dendritic cells: Trojan exosomes riding the Trojan horse? PLoS Pathog. 2010; 6:e1000740.
44. Luga V, Zhang L, Viloria-Petit A M, Ogunjimi A A, Inanlou M R, Chiu E, Buchanan M, Hosein A N, Basik M and Wrana J L. Exosomes mediate stromal mobilization of autocrine Wnt-PCP signaling in breast cancer cell migration. Cell. 2012; 151:1542-56.
45. Robbins P D and Morelli A E. Regulation of immune responses by extracellular vesicles. Nat Rev Immunol. 2014; 14:195-208.
46. Rekker K, Saare M, Roost A M, Kubo A L, Zarovni N, Chiesi A, Salumets A and Peters M. Comparison of serum exosome isolation methods for microRNA profiling. Clin Biochem. 2014; 47:135-8.
47. Taylor D D, Zacharias W and Gercel-Taylor C. Exosome isolation for proteomic analyses and RNA profiling. Methods Mol Biol. 2011; 728:235-46.
48. Caradec J, Kharmate G, Hosseini-Beheshti E, Adomat H, Gleave M and Guns E. Reproducibility and efficiency of serum-derived exosome extraction methods. Clin Biochem. 2014; 47:1286-92.

TABLE 7
Cut-off values for ASC levels in serum for Multiple Sclerosis (MS).

| | Sensi- | | Speci- | | Like- |
|---|---|---|---|---|---|
| Cutoff | tivity | | ficity | | lihood |
| (pg/ml) | % | 95% CI | % | 95% CI | ratio |
| >105.8 | 100 | 89.11% to | 0.8696 | 0.02201% to 4.75% | 1.009 |
| | | 100% | | | |
| >107.9 | 100 | 89.11% to | 1.739 | 0.2113% to 6.141% | 1.018 |
| | | 100% | | | |
| >112.1 | 100 | 89.11% to | 2.609 | 0.5412% to 7.435% | 1.027 |
| | | 100% | | | |
| >123.3 | 100 | 89.11% to | 3.478 | 0.9557% to 8.667% | 1.036 |
| | | 100% | | | |
| >132.4 | 100 | 89.11% to | 4.348 | 1.427% to 9.855% | 1.045 |
| | | 100% | | | |
| >133 | 100 | 89.11% to | 5.217 | 1.939% to 11.01% | 1.055 |
| | | 100% | | | |
| >134.2 | 100 | 89.11% to | 6.087 | 2.482% to 12.14% | 1.065 |
| | | 100% | | | |
| >135.2 | 100 | 89.11% to | 6.957 | 3.051% to 13.25% | 1.075 |
| | | 100% | | | |
| >135.5 | 100 | 89.11% to | 7.826 | 3.641% to 14.34% | 1.085 |
| | | 100% | | | |
| >135.8 | 100 | 89.11% to | 8.696 | 4.249% to 15.41% | 1.095 |
| | | 100% | | | |
| >136.1 | 100 | 89.11% to | 9.565 | 4.872% to 16.47% | 1.106 |
| | | 100% | | | |
| >139.2 | 100 | 89.11% to | 10.43 | 5.509% to 17.52% | 1.117 |
| | | 100% | | | |
| >142.6 | 100 | 89.11% to | 11.3 | 6.158% to 18.55% | 1.127 |
| | | 100% | | | |
| >143.3 | 100 | 89.11% to | 12.17 | 6.818% to 19.58% | 1.139 |
| | | 100% | | | |
| >144.6 | 100 | 89.11% to | 13.04 | 7.488% to 20.6% | 1.15 |
| | | 100% | | | |
| >146.2 | 100 | 89.11% to | 13.91 | 8.167% to 21.61% | 1.162 |
| | | 100% | | | |
| >147.5 | 100 | 89.11% to | 14.78 | 8.854% to 22.61% | 1.173 |
| | | 100% | | | |
| >148.9 | 100 | 89.11% to | 15.65 | 9.548% to 23.6% | 1.186 |
| | | 100% | | | |
| >150.4 | 100 | 89.11% to | 16.52 | 10.25% to 24.59% | 1.198 |
| | | 100% | | | |
| >151.4 | 100 | 89.11% to | 17.39 | 10.96% to 25.57% | 1.211 |
| | | 100% | | | |
| >151.8 | 100 | 89.11% to | 18.26 | 11.67% to 26.55% | 1.223 |
| | | 100% | | | |
| >154.3 | 100 | 89.11% to | 19.13 | 12.39% to 27.52% | 1.237 |
| | | 100% | | | |
| >158.2 | 100 | 89.11% to | 20 | 13.12% to 28.48% | 1.25 |
| | | 100% | | | |
| >160.8 | 100 | 89.11% to | 20.87 | 13.85% to 29.44% | 1.264 |
| | | 100% | | | |
| >164 | 100 | 89.11% to | 21.74 | 14.59% to 30.4% | 1.278 |
| | | 100% | | | |
| >168 | 100 | 89.11% to | 22.61 | 15.33% to 31.35% | 1.292 |
| | | 100% | | | |
| >170.2 | 100 | 89.11% to | 23.48 | 16.08% to 32.29% | 1.307 |
| | | 100% | | | |
| >171.2 | 100 | 89.11% to | 24.35 | 16.83% to 33.23% | 1.322 |
| | | 100% | | | |
| >172.2 | 100 | 89.11% to | 25.22 | 17.58% to 34.17% | 1.337 |
| | | 100% | | | |
| >173.4 | 100 | 89.11% to | 26.09 | 18.34% to 35.1% | 1.353 |
| | | 100% | | | |
| >175.6 | 100 | 89.11% to | 26.96 | 19.11% to 36.03% | 1.369 |
| | | 100% | | | |
| >178.5 | 100 | 89.11% to | 27.83 | 19.87% to 36.95% | 1.386 |
| | | 100% | | | |
| >180.9 | 100 | 89.11% to | 28.7 | 20.65% to 37.88% | 1.402 |
| | | 100% | | | |
| >182.1 | 100 | 89.11% to | 29.57 | 21.42% to 38.79% | 1.42 |
| | | 100% | | | |
| >183.3 | 100 | 89.11% to | 30.43 | 22.2% to 39.71% | 1.438 |
| | | 100% | | | |
| >184.4 | 100 | 89.11% to | 31.3 | 22.98% to 40.62% | 1.456 |
| | | 100% | | | |
| >184.9 | 100 | 89.11% to | 32.17 | 23.77% to 41.53% | 1.474 |
| | | 100% | | | |
| >185.7 | 100 | 89.11% to | 33.04 | 24.56% to 42.43% | 1.494 |
| | | 100% | | | |
| >186.5 | 100 | 89.11% to | 33.91 | 25.35% to 43.33% | 1.513 |
| | | 100% | | | |
| >188.9 | 100 | 89.11% to | 34.78 | 26.14% to 44.23% | 1.533 |
| | | 100% | | | |
| >191.1 | 100 | 89.11% to | 35.65 | 26.94% to 45.12% | 1.554 |
| | | 100% | | | |
| >191.9 | 100 | 89.11% to | 36.52 | 27.74% to 46.01% | 1.575 |
| | | 100% | | | |
| >193.1 | 100 | 89.11% to | 37.39 | 28.55% to 46.9% | 1.597 |
| | | 100% | | | |
| >195.2 | 100 | 89.11% to | 38.26 | 29.35% to 47.79% | 1.62 |
| | | 100% | | | |
| >196.6 | 100 | 89.11% to | 39.13 | 30.16% to 48.67% | 1.643 |
| | | 100% | | | |
| >197.2 | 100 | 89.11% to | 40 | 30.98% to 49.55% | 1.667 |
| | | 100% | | | |
| >198.7 | 100 | 89.11% to | 40.87 | 31.79% to 50.43% | 1.691 |
| | | 100% | | | |
| >202.1 | 100 | 89.11% to | 41.74 | 32.61% to 51.3% | 1.716 |
| | | 100% | | | |
| >207.2 | 100 | 89.11% to | 42.61 | 33.44% to 52.17% | 1.742 |
| | | 100% | | | |
| >210 | 100 | 89.11% to | 43.48 | 34.26% to 53.04% | 1.769 |
| | | 100% | | | |
| >211.1 | 100 | 89.11% to | 44.35 | 35.09% to 53.91% | 1.797 |
| | | 100% | | | |
| >214.3 | 100 | 89.11% to | 45.22 | 35.92% to 54.77% | 1.825 |
| | | 100% | | | |
| >216.8 | 100 | 89.11% to | 46.09 | 36.75% to 55.63% | 1.855 |
| | | 100% | | | |
| >218.1 | 100 | 89.11% to | 46.96 | 37.59% to 56.49% | 1.885 |
| | | 100% | | | |
| >220.4 | 100 | 89.11% to | 47.83 | 38.43% to 57.34% | 1.917 |
| | | 100% | | | |
| >224.1 | 100 | 89.11% to | 48.7 | 39.27% to 58.19% | 1.949 |
| | | 100% | | | |
| >227.1 | 100 | 89.11% to | 49.57 | 40.11% to 59.04% | 1.983 |
| | | 100% | | | |
| >228.8 | 100 | 89.11% to | 50.43 | 40.96% to 59.89% | 2.018 |
| | | 100% | | | |
| >230.8 | 100 | 89.11% to | 51.3 | 41.81% to 60.73% | 2.054 |
| | | 100% | | | |
| >231.7 | 100 | 89.11% to | 52.17 | 42.66% to 61.57% | 2.091 |
| | | 100% | | | |
| >232.6 | 100 | 89.11% to | 53.04 | 43.51% to 62.41% | 2.13 |
| | | 100% | | | |
| >233.5 | 100 | 89.11% to | 53.91 | 44.37% to 63.25% | 2.17 |
| | | 100% | | | |
| >238.2 | 100 | 89.11% to | 54.78 | 45.23% to 64.08% | 2.212 |
| | | 100% | | | |
| >243.1 | 100 | 89.11% to | 55.65 | 46.09% to 64.91% | 2.255 |
| | | 100% | | | |
| >244 | 100 | 89.11% to | 56.52 | 46.96% to 65.74% | 2.3 |
| | | 100% | | | |
| >244.7 | 100 | 89.11% to | 57.39 | 47.83% to 66.56% | 2.347 |
| | | 100% | | | |
| >247.2 | 100 | 89.11% to | 58.26 | 48.7% to 67.39% | 2.396 |
| | | 100% | | | |
| >249.6 | 96.88 | 83.78% to | 58.26 | 48.7% to 67.39% | 2.321 |
| | | 99.92% | | | |
| >250.2 | 96.88 | 83.78% to | 59.13 | 49.57% to 68.21% | 2.37 |
| | | 99.92% | | | |
| >250.5 | 96.88 | 83.78% to | 60 | 50.45% to 69.02% | 2.422 |
| | | 99.92% | | | |
| >250.7 | 96.88 | 83.78% to | 60.87 | 51.33% to 69.84% | 2.476 |
| | | 99.92% | | | |
| >251.6 | 96.88 | 83.78% to | 61.74 | 52.21% to 70.65% | 2.532 |
| | | 99.92% | | | |
| >252.4 | 96.88 | 83.78% to | 62.61 | 53.1% to 71.45% | 2.591 |
| | | 99.92% | | | |
| >253.2 | 96.88 | 83.78% to | 63.48 | 53.99% to 72.26% | 2.653 |
| | | 99.92% | | | |
| >254.9 | 93.75 | 79.19% to | 63.48 | 53.99% to 72.26% | 2.567 |
| | | 99.23% | | | |
| >257.2 | 93.75 | 79.19% to | 64.35 | 54.88% to 73.06% | 2.63 |
| | | 99.23% | | | |
| >259 | 93.75 | 79.19% to | 65.22 | 55.77% to 73.86% | 2.695 |
| | | 99.23% | | | |
| >260.8 | 93.75 | 79.19% to | 66.09 | 56.67% to 74.65% | 2.764 |
| | | 99.23% | | | |
| >263 | 93.75 | 79.19% to | 66.96 | 57.57% to 75.44% | 2.837 |
| | | 99.23% | | | |
| >264.2 | 90.63 | 74.98% to | 66.96 | 57.57% to 75.44% | 2.743 |
| | | 98.02% | | | |
| >267.1 | 90.63 | 74.98% to | 67.83 | 58.47% to 76.23% | 2.817 |
| | | 98.02% | | | |
| >270.9 | 90.63 | 74.98% to | 68.7 | 59.38% to 77.02% | 2.895 |
| | | 98.02% | | | |
| >272.3 | 90.63 | 74.98% to | 69.57 | 60.29% to 77.8% | 2.978 |
| | | 98.02% | | | |
| >272.7 | 90.63 | 74.98% to | 70.43 | 61.21% to 78.58% | 3.065 |
| | | 98.02% | | | |
| >273.3 | 90.63 | 74.98% to | 71.3 | 62.12% to 79.35% | 3.158 |
| | | 98.02% | | | |
| >277.9 | 90.63 | 74.98% to | 72.17 | 63.05% to 80.13% | 3.257 |
| | | 98.02% | | | |
| >282.9 | 90.63 | 74.98% to | 73.04 | 63.97% to 80.89% | 3.362 |
| | | 98.02% | | | |
| >283.9 | 90.63 | 74.98% to | 73.91 | 64.9% to 81.66% | 3.474 |
| | | 98.02% | | | |
| >286.3 | 90.63 | 74.98% to | 74.78 | 65.83% to 82.42% | 3.594 |
| | | 98.02% | | | |
| >289.3 | 90.63 | 74.98% to | 75.65 | 66.77% to 83.17% | 3.722 |
| | | 98.02% | | | |
| >290.4 | 90.63 | 74.98% to | 76.52 | 67.71% to 83.92% | 3.86 |
| | | 98.02% | | | |
| >294.2 | 90.63 | 74.98% to | 77.39 | 68.65% to 84.67% | 4.008 |
| | | 98.02% | | | |
| >298 | 90.63 | 74.98% to | 78.26 | 69.6% to 85.41% | 4.169 |
| | | 98.02% | | | |
| >300.4 | 90.63 | 74.98% to | 79.13 | 70.56% to 86.15% | 4.342 |
| | | 98.02% | | | |
| >302.7 | 90.63 | 74.98% to | 80 | 71.52% to 86.88% | 4.531 |
| | | 98.02% | | | |
| >304 | 90.63 | 74.98% to | 80.87 | 72.48% to 87.61% | 4.737 |
| | | 98.02% | | | |
| >310.4 | 90.63 | 74.98% to | 81.74 | 73.45% to 88.33% | 4.963 |
| | | 98.02% | | | |
| >318.3 | 90.63 | 74.98% to | 82.61 | 74.43% to 89.04% | 5.211 |
| | | 98.02% | | | |
| >321.9 | 90.63 | 74.98% to | 83.48 | 75.41% to 89.75% | 5.485 |
| | | 98.02% | | | |
| >324.4 | 90.63 | 74.98% to | 84.35 | 76.4% to 90.45% | 5.79 |
| | | 98.02% | | | |
| >326.2 | 90.63 | 74.98% to | 85.22 | 77.39% to 91.15% | 6.131 |
| | | 98.02% | | | |
| >328.7 | 90.63 | 74.98% to | 86.09 | 78.39% to 91.83% | 6.514 |
| | | 98.02% | | | |
| >331 | 90.63 | 74.98% to | 86.96 | 79.4% to 92.51% | 6.948 |
| | | 98.02% | | | |
| >335.3 | 90.63 | 74.98% to | 87.83 | 80.42% to 93.18% | 7.444 |
| | | 98.02% | | | |
| >343.6 | 87.5 | 71.01% to | 87.83 | 80.42% to 93.18% | 7.188 |
| | | 96.49% | | | |
| >349 | 84.38 | 67.21% to | 87.83 | 80.42% to 93.18% | 6.931 |
| | | 94.72% | | | |
| >351.1 | 84.38 | 67.21% to | 88.7 | 81.45% to 93.84% | 7.464 |
| | | 94.72% | | | |
| >352.4 | 84.38 | 67.21% to | 89.57 | 82.48% to 94.49% | 8.086 |
| | | 94.72% | | | |
| >353.5 | 81.25 | 63.56% to | 89.57 | 82.48% to 94.49% | 7.786 |
| | | 92.79% | | | |
| >354.2 | 78.13 | 60.03% to | 89.57 | 82.48% to 94.49% | 7.487 |
| | | 90.72% | | | |
| >356.7 | 78.13 | 60.03% to | 90.43 | 83.53% to 95.13% | 8.168 |
| | | 90.72% | | | |
| >364.1 | 78.13 | 60.03% to | 91.3 | 84.59% to 95.75% | 8.984 |
| | | 90.72% | | | |
| >375.2 | 75 | 56.6% to | 91.3 | 84.59% to 95.75% | 8.625 |
| | | 88.54% | | | |
| >381.9 | 75 | 56.6% to | 92.17 | 85.66% to 96.36% | 9.583 |
| | | 88.54% | | | |
| >383.7 | 75 | 56.6% to | 93.04 | 86.75% to 96.95% | 10.78 |
| | | 88.54% | | | |
| >386.6 | 75 | 56.6% to | 93.91 | 87.86% to 97.52% | 12.32 |
| | | 88.54% | | | |
| >391.8 | 71.88 | 53.25% to | 93.91 | 87.86% to 97.52% | 11.81 |
| | | 86.25% | | | |
| >396.9 | 71.88 | 53.25% to | 94.78 | 88.99% to 98.06% | 13.78 |
| | | 86.25% | | | |
| >400.4 | 71.88 | 53.25% to | 95.65 | 90.15% to 98.57% | 16.53 |
| | | 86.25% | | | |
| >406.6 | 71.88 | 53.25% to | 96.52 | 91.33% to 99.04% | 20.66 |
| | | 86.25% | | | |
| >423.8 | 68.75 | 49.99% to | 96.52 | 91.33% to 99.04% | 19.77 |
| | | 83.88% | | | |
| >437.2 | 68.75 | 49.99% to | 97.39 | 92.57% to 99.46% | 26.35 |
| | | 83.88% | | | |
| >437.7 | 68.75 | 49.99% to | 98.26 | 93.86% to 99.79% | 39.53 |
| | | 83.88% | | | |
| >441 | 65.63 | 46.81% to | 98.26 | 93.86% to 99.79% | 37.73 |
| | | 81.43% | | | |
| >451.3 | 65.63 | 46.81% to | 99.13 | 95.25% to 99.98% | 75.47 |
| | | 81.43% | | | |
| >465.1 | 65.63 | 46.81% to | 100 | 96.84% to 100% | |
| | | 81.43% | | | |
| >475.7 | 62.5 | 43.69% to | 100 | 96.84% to 100% | |
| | | 78.9% | | | |
| >480.7 | 59.38 | 40.64% to | 100 | 96.84% to 100% | |
| | | 76.3% | | | |
| >501.8 | 56.25 | 37.66% to | 100 | 96.84% to 100% | |
| | | 73.64% | | | |
| >522.9 | 53.13 | 34.74% to | 100 | 96.84% to 100% | |
| | | 70.91% | | | |
| >537.5 | 50 | 31.89% to | 100 | 96.84% to 100% | |
| | | 68.11% | | | |
| >560.5 | 46.88 | 29.09% to | 100 | 96.84% to 100% | |
| | | 65.26% | | | |
| >575.6 | 43.75 | 26.36% to | 100 | 96.84% to 100% | |
| | | 62.34% | | | |
| >621.7 | 40.63 | 23.7% to | 100 | 96.84% to 100% | |
| | | 59.36% | | | |
| >698.9 | 37.5 | 21.1% to | 100 | 96.84% to 100% | |
| | | 56.31% | | | |
| >740.4 | 34.38 | 18.57% to | 100 | 96.84% to 100% | |
| | | 53.19% | | | |
| >758.3 | 31.25 | 16.12% to | 100 | 96.84% to 100% | |
| | | 50.01% | | | |
| >814.6 | 28.13 | 13.75% to | 100 | 96.84% to 100% | |
| | | 46.75% | | | |
| >866.6 | 25 | 11.46% to | 100 | 96.84% to 100% | |
| | | 43.4% | | | |
| >888.7 | 21.88 | 9.277% to | 100 | 96.84% to 100% | |
| | | 39.97% | | | |
| >910.2 | 18.75 | 7.208% to | 100 | 96.84% to 100% | |
| | | 36.44% | | | |
| >927.1 | 15.63 | 5.275% to | 100 | 96.84% to 100% | |
| | | 32.79% | | | |
| >947 | 12.5 | 3.513% to | 100 | 96.84% to 100% | |
| | | 28.99% | | | |
| >961.3 | 9.375 | 1.977% to | 100 | 96.84% to 100% | |
| | | 25.02% | | | |
| >1252 | 6.25 | 0.7661% to | 100 | 96.84% to 100% | |
| | | 20.81% | | | |
| >1668 | 3.125 | 0.07909% to | 100 | 96.84% to 100% | |
| | | 16.22% | | | |

TABLE 8
Cut-off values for ASC levels in serum for Stroke.

| | Sensi- | | Speci- | | Like- |
|---|---|---|---|---|---|
| Cutoff | tivity | | ficity | | lihood |
| (pg/ml) | % | 95% CI | % | 95% CI | ratio |
| >128.7 | 100 | 79.41% to 100% | 1.333 | 0.03375% to | 1.014 |
| | | | | 7.206% | |
| >145.8 | 100 | 79.41% to 100% | 2.667 | 0.3246% to | 1.027 |
| | | | | 9.303% | |
| >148.9 | 100 | 79.41% to 100% | 4 | 0.8326% to | 1.042 |
| | | | | 11.25% | |
| >150.4 | 100 | 79.41% to 100% | 5.333 | 1.472% to 13.1% | 1.056 |
| >153.9 | 100 | 79.41% to 100% | 6.667 | 2.2% to 14.88% | 1.071 |
| >158.2 | 100 | 79.41% to 100% | 8 | 2.993% to 16.6% | 1.087 |
| >164.8 | 100 | 79.41% to 100% | 9.333 | 3.835% to 18.29% | 1.103 |
| >170.2 | 100 | 79.41% to 100% | 10.67 | 4.719% to 19.94% | 1.119 |
| >171.2 | 100 | 79.41% to 100% | 12 | 5.637% to 21.56% | 1.136 |
| >172.2 | 100 | 79.41% to 100% | 13.33 | 6.583% to 23.16% | 1.154 |
| >173.4 | 100 | 79.41% to 100% | 14.67 | 7.556% to 24.73% | 1.172 |
| >175.6 | 100 | 79.41% to 100% | 16 | 8.55% to 26.28% | 1.19 |
| >178.5 | 100 | 79.41% to 100% | 17.33 | 9.565% to 27.81% | 1.21 |
| >180.9 | 100 | 79.41% to 100% | 18.67 | 10.6% to 29.33% | 1.23 |
| >182.1 | 100 | 79.41% to 100% | 20 | 11.65% to 30.83% | 1.25 |
| >183.3 | 100 | 79.41% to 100% | 21.33 | 12.71% to 32.32% | 1.271 |
| >184.4 | 100 | 79.41% to 100% | 22.67 | 13.79% to 33.79% | 1.293 |
| >184.9 | 100 | 79.41% to 100% | 24 | 14.89% to 35.25% | 1.316 |
| >186.1 | 100 | 79.41% to 100% | 25.33 | 15.99% to 36.7% | 1.339 |
| >188.9 | 100 | 79.41% to 100% | 26.67 | 17.11% to 38.14% | 1.364 |
| >191.1 | 100 | 79.41% to 100% | 28 | 18.24% to 39.56% | 1.389 |
| >191.9 | 100 | 79.41% to 100% | 29.33 | 19.38% to 40.98% | 1.415 |
| >193.1 | 100 | 79.41% to 100% | 30.67 | 20.53% to 42.38% | 1.442 |
| >195.2 | 100 | 79.41% to 100% | 32 | 21.69% to 43.78% | 1.471 |
| >196.6 | 100 | 79.41% to 100% | 33.33 | 22.86% to 45.17% | 1.5 |
| >197.2 | 100 | 79.41% to 100% | 34.67 | 24.04% to 46.54% | 1.531 |
| >198.7 | 100 | 79.41% to 100% | 36 | 25.23% to 47.91% | 1.563 |
| >204.8 | 100 | 79.41% to 100% | 37.33 | 26.43% to 49.27% | 1.596 |
| >210 | 100 | 79.41% to 100% | 38.67 | 27.64% to 50.62% | 1.63 |
| >211.1 | 100 | 79.41% to 100% | 40 | 28.85% to 51.96% | 1.667 |
| >214.5 | 100 | 79.41% to 100% | 41.33 | 30.08% to 53.3% | 1.705 |
| >219.2 | 100 | 79.41% to 100% | 42.67 | 31.31% to 54.62% | 1.744 |
| >224.5 | 100 | 79.41% to 100% | 44 | 32.55% to 55.94% | 1.786 |
| >228.8 | 100 | 79.41% to 100% | 45.33 | 33.79% to 57.25% | 1.829 |
| >230.8 | 100 | 79.41% to 100% | 46.67 | 35.05% to 58.55% | 1.875 |
| >231.7 | 100 | 79.41% to 100% | 48 | 36.31% to 59.85% | 1.923 |
| >232.9 | 100 | 79.41% to 100% | 49.33 | 37.58% to 61.14% | 1.974 |
| >238.2 | 100 | 79.41% to 100% | 50.67 | 38.86% to 62.42% | 2.027 |
| >243.5 | 100 | 79.41% to 100% | 52 | 40.15% to 63.69% | 2.083 |
| >244.7 | 100 | 79.41% to 100% | 53.33 | 41.45% to 64.95% | 2.143 |
| >247.5 | 100 | 79.41% to 100% | 54.67 | 42.75% to 66.21% | 2.206 |
| >250.4 | 100 | 79.41% to 100% | 56 | 44.06% to 67.45% | 2.273 |
| >251.6 | 100 | 79.41% to 100% | 57.33 | 45.38% to 68.69% | 2.344 |
| >252.4 | 100 | 79.41% to 100% | 58.67 | 46.7% to 69.92% | 2.419 |
| >254.2 | 100 | 79.41% to 100% | 60 | 48.04% to 71.15% | 2.5 |
| >257.2 | 100 | 79.41% to 100% | 61.33 | 49.38% to 72.36% | 2.586 |
| >259 | 100 | 79.41% to 100% | 62.67 | 50.73% to 73.57% | 2.679 |
| >260.8 | 100 | 79.41% to 100% | 64 | 52.09% to 74.77% | 2.778 |
| >263.3 | 100 | 79.41% to 100% | 65.33 | 53.46% to 75.96% | 2.885 |
| >268.8 | 100 | 79.41% to 100% | 66.67 | 54.83% to 77.14% | 3 |
| >277.6 | 100 | 79.41% to 100% | 68 | 56.22% to 78.31% | 3.125 |
| >282.9 | 100 | 79.41% to 100% | 69.33 | 57.62% to 79.47% | 3.261 |
| >283.9 | 100 | 79.41% to 100% | 70.67 | 59.02% to 80.62% | 3.409 |
| >286.3 | 100 | 79.41% to 100% | 72 | 60.44% to 81.76% | 3.571 |
| >289.3 | 100 | 79.41% to 100% | 73.33 | 61.86% to 82.89% | 3.75 |
| >290.4 | 100 | 79.41% to 100% | 74.67 | 63.3% to 84.01% | 3.947 |
| >294.7 | 100 | 79.41% to 100% | 76 | 64.75% to 85.11% | 4.167 |
| >300.8 | 100 | 79.41% to 100% | 77.33 | 66.21% to 86.21% | 4.412 |
| >304 | 100 | 79.41% to 100% | 78.67 | 67.68% to 87.29% | 4.688 |
| >310.4 | 100 | 79.41% to 100% | 80 | 69.17% to 88.35% | 5 |
| >319.3 | 100 | 79.41% to 100% | 81.33 | 70.67% to 89.4% | 5.357 |
| >324.4 | 100 | 79.41% to 100% | 82.67 | 72.19% to 90.43% | 5.769 |
| >326.2 | 100 | 79.41% to 100% | 84 | 73.72% to 91.45% | 6.25 |
| >328.7 | 100 | 79.41% to 100% | 85.33 | 75.27% to 92.44% | 6.818 |
| >341.4 | 100 | 79.41% to 100% | 86.67 | 76.84% to 93.42% | 7.5 |
| >353.1 | 100 | 79.41% to 100% | 88 | 78.44% to 94.36% | 8.333 |
| >367.7 | 100 | 79.41% to 100% | 89.33 | 80.06% to 95.28% | 9.375 |
| >381.9 | 100 | 79.41% to 100% | 90.67 | 81.71% to 96.16% | 10.71 |
| >383.7 | 100 | 79.41% to 100% | 92 | 83.4% to 97.01% | 12.5 |
| >391.7 | 100 | 79.41% to 100% | 93.33 | 85.12% to 97.8% | 15 |
| >400.4 | 100 | 79.41% to 100% | 94.67 | 86.9% to 98.53% | 18.75 |
| >404.8 | 100 | 79.41% to 100% | 96 | 88.75% to 99.17% | 25 |
| >421.9 | 93.75 | 69.77% to 99.84% | 96 | 88.75% to 99.17% | 23.44 |
| >437.2 | 93.75 | 69.77% to 99.84% | 97.33 | 90.7% to 99.68% | 35.16 |
| >448 | 93.75 | 69.77% to 99.84% | 98.67 | 92.79% to 99.97% | 70.31 |
| >547.1 | 93.75 | 69.77% to 99.84% | 100 | 95.2% to 100% | |
| >646.2 | 87.5 | 61.65% to 98.45% | 100 | 95.2% to 100% | |
| >689 | 81.25 | 54.35% to 95.95% | 100 | 95.2% to 100% | |
| >733.3 | 75 | 47.62% to 92.73% | 100 | 95.2% to 100% | |
| >755.6 | 68.75 | 41.34% to 88.98% | 100 | 95.2% to 100% | |
| >769 | 62.5 | 35.43% to 84.8% | 100 | 95.2% to 100% | |
| >791.5 | 56.25 | 29.88% to 80.25% | 100 | 95.2% to 100% | |
| >818.2 | 50 | 24.65% to 75.35% | 100 | 95.2% to 100% | |
| >901 | 43.75 | 19.75% to 70.12% | 100 | 95.2% to 100% | |
| >1069 | 37.5 | 15.2% to 64.57% | 100 | 95.2% to 100% | |
| >1356 | 31.25 | 11.02% to 58.66% | 100 | 95.2% to 100% | |
| >1572 | 25 | 7.266% to 52.38% | 100 | 95.2% to 100% | |
| >1621 | 18.75 | 4.047% to 45.65% | 100 | 95.2% to 100% | |
| >1692 | 12.5 | 1.551% to 38.35% | 100 | 95.2% to 100% | |
| >1814 | 6.25 | 0.1581% to | 100 | 95.2% to 100% | |
| | | 30.23% | | | |

TABLE 9
Cut-off values for ASC levels in serum-derived extracellular vesicles (EVs) for Stroke.

| Cutoff | | | | | Likelihood |
|---|---|---|---|---|---|
| (pg/ml) | Sensitivity % | 95% CI | Specificity % | 95% CI | ratio |
| >28.56 | 100 | 79.41% to 100% | 6.25 | 0.1581% to 30.23% | 1.067 |
| >31.31 | 100 | 79.41% to 100% | 12.5 | 1.551% to 38.35% | 1.143 |
| >33.88 | 100 | 79.41% to 100% | 18.75 | 4.047% to 45.65% | 1.231 |
| >37.46 | 100 | 79.41% to 100% | 25 | 7.266% to 52.38% | 1.333 |
| >41.38 | 100 | 79.41% to 100% | 31.25 | 11.02% to 58.66% | 1.455 |
| >44.01 | 100 | 79.41% to 100% | 37.5 | 15.2% to 64.57% | 1.6 |
| >44.38 | 100 | 79.41% to 100% | 43.75 | 19.75% to 70.12% | 1.778 |
| >45.13 | 100 | 79.41% to 100% | 50 | 24.65% to 75.35% | 2 |
| >46.71 | 100 | 79.41% to 100% | 56.25 | 29.88% to 80.25% | 2.286 |
| >48.51 | 100 | 79.41% to 100% | 62.5 | 35.43% to 84.8% | 2.667 |
| >49.35 | 100 | 79.41% to 100% | 68.75 | 41.34% to 88.98% | 3.2 |
| >51.09 | 100 | 79.41% to 100% | 75 | 47.62% to 92.73% | 4 |
| >58.1 | 100 | 79.41% to 100% | 81.25 | 54.35% to 95.95% | 5.333 |
| >69.76 | 100 | 79.41% to 100% | 87.5 | 61.65% to 98.45% | 8 |
| >81.6 | 100 | 79.41% to 100% | 93.75 | 69.77% to 99.84% | 16 |
| >97.57 | 100 | 79.41% to 100% | 100 | 79.41% to 100% | |
| >114.9 | 93.75 | 69.77% to 99.84% | 100 | 79.41% to 100% | |
| >130.2 | 87.5 | 61.65% to 98.45% | 100 | 79.41% to 100% | |
| >138.7 | 81.25 | 54.35% to 95.95% | 100 | 79.41% to 100% | |
| >139 | 75 | 47.62% to 92.73% | 100 | 79.41% to 100% | |
| >143.6 | 68.75 | 41.34% to 88.98% | 100 | 79.41% to 100% | |
| >153.2 | 62.5 | 35.43% to 84.8% | 100 | 79.41% to 100% | |
| >165.6 | 56.25 | 29.88% to 80.25% | 100 | 79.41% to 100% | |
| >202.6 | 50 | 24.65% to 75.35% | 100 | 79.41% to 100% | |
| >261.5 | 43.75 | 19.75% to 70.12% | 100 | 79.41% to 100% | |
| >292.9 | 37.5 | 15.2% to 64.57% | 100 | 79.41% to 100% | |
| >361.4 | 31.25 | 11.02% to 58.66% | 100 | 79.41% to 100% | |
| >441.3 | 25 | 7.266% to 52.38% | 100 | 79.41% to 100% | |
| >459.4 | 18.75 | 4.047% to 45.65% | 100 | 79.41% to 100% | |
| >465.8 | 12.5 | 1.551% to 38.35% | 100 | 79.41% to 100% | |
| >493.5 | 6.25 | 0.1581% to 30.23% | 100 | 79.41% to 100% | |

### Example 3: Examination of Inflammasome Proteins as Biomarkers of Traumatic Brain Injury (TBI)

As defined by the US Center for Disease Control (“CDC), a traumatic brain injury (“TBI”) is “a disruption in the normal function of the brain that can be caused by a bump, blow, or jolt to the head, or penetrating head injury.” Important to the care of patients with TBI is the need for biomarkers that can predict onset, exacerbation as well as response to treatment. Additionally, there is a need for a minimally invasive method of harvesting these biomarkers for analysis.

The inflammasome is a key mediator of the innate immune response that in the CNS was first described to mediate inflammation after spinal cord injury². The inflammasome is a multiprotein complex involved in the activation of caspase-1 and the processing of the pro-inflammatory cytokines IL-1β and IL-18³.

In this example, the expression level of inflammasome proteins in serum samples from patients with TBI are determined. Further, an examination of the sensitivity and specificity of inflammasome signaling proteins as biomarkers of TBI was examined.

### Materials and Methods

### Participants:

In this study, serum samples were analyzed from 120 normal donors and 21 patients that were diagnosed with TBI. Samples were purchased from BioreclamationIVT. The normal donor group consisted of samples obtained from 60 male and 60 female donors in the age range of 20 to 70 years old. The age range in the TBI group consisted of samples obtained from patients in the age range of 24 to 64 years old. Additionally, twenty-one control cerebral spinal fluid (“CSF”) samples were obtained from BioreclamationIVT, 9 CSF samples were obtained from the cohort of patients.

### Protein Assay:

Concentration of inflammasome proteins ASC, IL-1β and IL-18 in serum and CSF was analyzed using a Simple Plex and a Simple Plex Explorer software. Results shown correspond to the mean of each sample run in triplicates. It should be noted that any system/instrument known in the art can be used to measure the levels of proteins (e.g., inflammasome proteins) in bodily fluids. Samples were collected three times a day for the first 5 days since patients arrived to the hospital. Samples were analyzed for the 1st, 2nd collection (Day 1) as well as 4th and 6th collections (Day 2)

### Biomarker Analyses:

Prism 7 software (GraphPad) was used to analyze the data obtained from the Simple Plex Explorer Software. Comparisons between groups were carried after identifying outliers followed by determination of the area under the receiver operator characteristic (ROC) curve, as well as the 95% confidence interval (CI). The p-value of significance used was <0.05. Sensitivity and specificity of each biomarker was obtained for a range of different cut-off points. Samples that yielded a protein value below the level of detection of the assay were not included in the analyses for that analyte.

ROC curves are summarized as the area under the curve (AUC). A perfect AUC value is 1.0, where 100% of subjects in the population will be correctly classified as having TBI or not. In contrast, an AUC of 0.5 signifies that subjects are randomly classified as either positive or negative for TBI, which has no clinical utility. It has been suggested that an AUC between 0.9 to 1.0 applies to an excellent biomarker; from 0.8 to 0.9, good; 0.7 to 0.8 fair; 0.6 to 0.7, poor and 0.5 to 0.6, fail.⁵

### Results

Caspase-1 and ASC are Elevated in the Serum of Patients after TBI

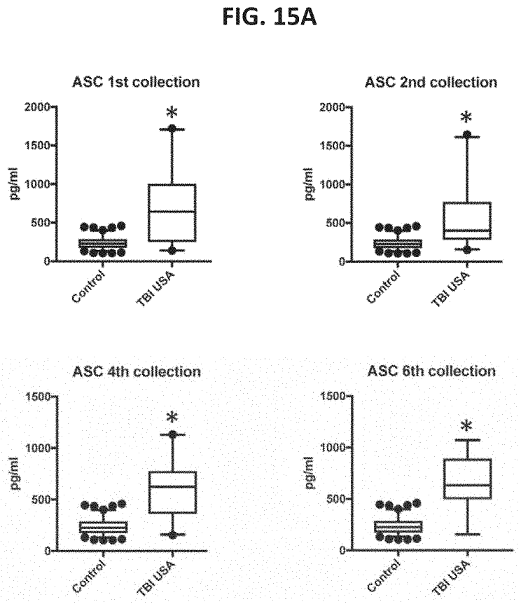

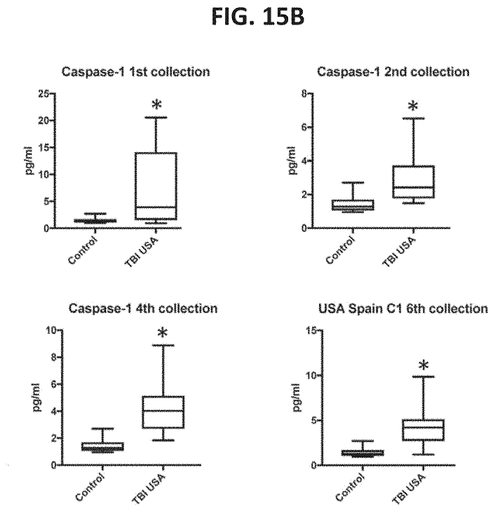

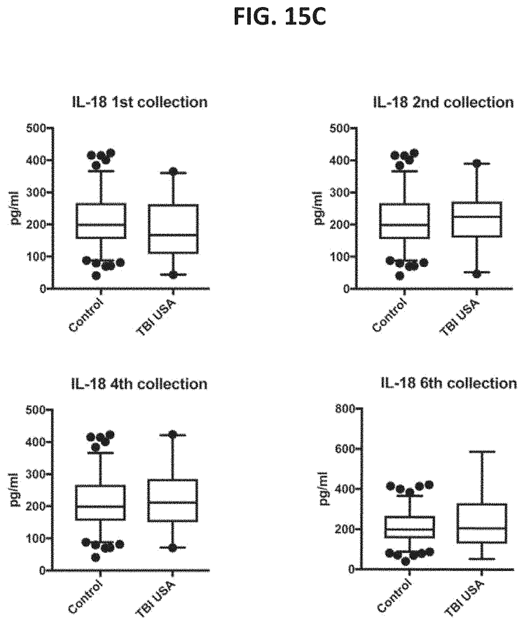

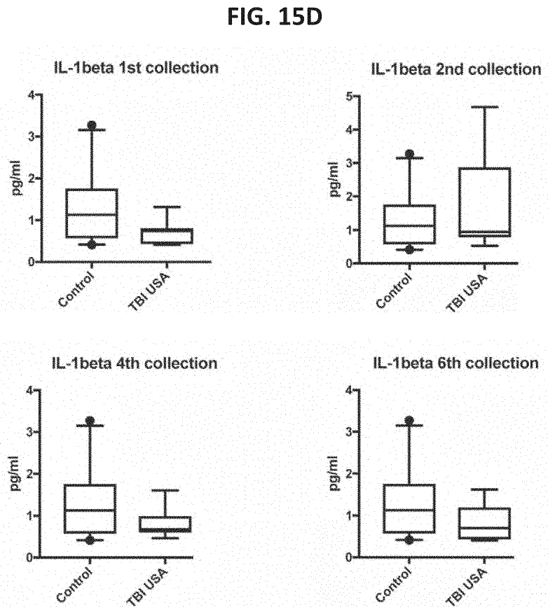

Serum samples from TBI patients were analyzed and compared to serum from healthy/control individuals using a Simple Plex assay (Protein Simple) for the protein expression of the inflammasome signaling proteins caspase-1, ASC, IL-1β and IL-18 (FIG. 15A-15D). The protein levels of caspase-1, ASC and IL-18 in the serum of TBI patients was higher than in the control group. However, the levels of IL-1β were lower in the TBI than controls.

### ASC and Caspase-1 are Good Serum Biomarkers of TBI

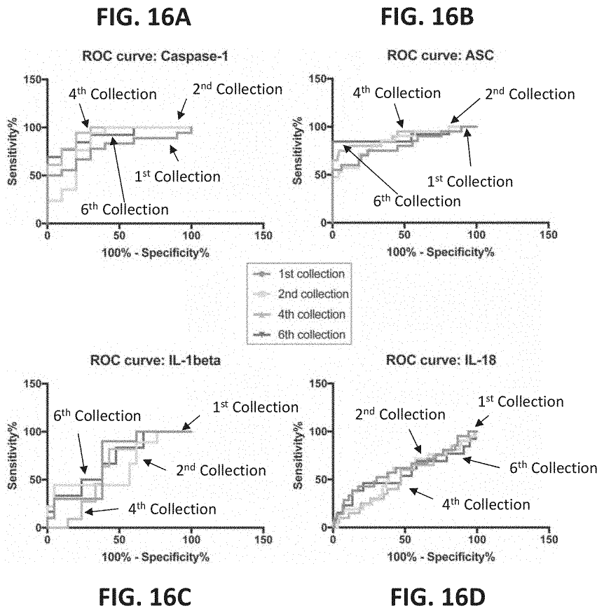

To then determine if these inflammasome signaling proteins have the potential to be reliable biomarkers for TBI pathology, the area under the curve (AUC) for caspase-1, ASC, IL-1β and IL-18 (FIG. 16A-D) were determined. Of the proteins measured, caspase-1 and ASC were shown to be the best biomarkers (FIGS. 16A and B) with an AUC of 0.93 (4th collection) and 0.90 (6th collection), respectively (Tables 10A-10D).

Table 10A-D: ROC analysis results for inflammasome signaling proteins Caspase-1 (Table 10A), ASC (Table 10B), IL-1β (Table 10C) and IL-18 (Table 10D) in serum including area, standard error (STD. ERROR), 95% confidence interval (CI) and p-value for collections 1^{st}, 2^{nd}, 4^{th} and 6^{th}.

TABLE 10A
ROC analysis for Caspase-1 in Serum.

| | | STD. | | |
|---|---|---|---|---|
| BIOMARKER | AUC | ERROR | 95% C.I. | P VALUE |
| 1^{st} Collection | 0.78 | 0.08772 | 0.6058 to 0.9497 | 0.01 |
| 2^{nd} Collection | 0.83 | 0.0479 | 0.8395 to 1.027 | 0.005 |
| 4^{th} Collection | 0.93 | 0.1407 | 0.8353 to 0.8869 | 0.0002 |
| 6^{th} Collection | 0.91 | 0.06065 | 0.7888 to 1.027 | 0.001 |

TABLE 10B
ROC analysis for ASC in Serum.

| | | STD. | | |
|---|---|---|---|---|
| BIOMARKER | AUC | ERROR | 95% C.I. | P VALUE |
| 1^{st} Collection | 0.80 | 0.06472 | 0.6762 to 0.9299 | <0.0001 |
| 2^{nd} Collection | 0.84 | 0.05026 | 0.7425 to 0.9395 | <0.0001 |
| 4^{th} Collection | 0.89 | 0.04898 | 0.7931 to 0.9851 | <0.0001 |
| 6^{th} Collection | 0.90 | 0.0697 | 0.759 to 1.032 | <0.0001 |

TABLE 10C
ROC analysis for IL-1β in Serum

| | | STD. | | |
|---|---|---|---|---|
| BIOMARKER | AUC | ERROR | 95% C.I. | P VALUE |
| 1^{st} Collection | 0.7 | 0.0965 | 0.5109 to 0.8891 | 0.0759 |
| 2^{nd} Collection | 0.64 | 0.1182 | 0.4085 to 0.8719 | 0.2304 |
| 4^{th} Collection | 0.6234 | 0.09765 | 0.432 to 0.8148 | 0.2582 |
| 6^{th} Collection | 0.6984 | 0.1162 | 0.4707 to 0.9261 | 0.1448 |

TABLE 10D
ROC analysis for IL-18 in Serum

| | | STD. | | |
|---|---|---|---|---|
| BIOMARKER | AUC | ERROR | 95% C.I. | P VALUE |
| 1^{st} Collection | 0.61 | 0.07475 | 0.4593 to 0.7524 | 0.1227 |
| 2^{nd} Collection | 0.55 | 0.07064 | 0.4082 to 0.6851 | 0.4966 |
| 4^{th} Collection | 0.51 | 0.0713 | 0.372 to 0.6515 | 0.8666 |
| 6^{th} Collection | 0.55 | 0.1015 | 0.3532 to 0.7509 | 0.5387 |

Furthermore, the cut-off point for caspase-1 was 1.943 pg/ml with 94% sensitivity and 89% specificity (Table 11A). For ASC, the cut-off point was 451.3 pg/ml with 85% sensitivity and 99% specificity (Table 11B). Moreover, we found that in regards to caspase-1 for 100% sensitivity, the cut-off point was 1.679 pg/ml with 78% specificity. For ASC, the cut-off point was 153.4 pg/ml and a 19% specificity (see Table 16 (4^{th} collection)). In the case of caspase-1, for 100% specificity, the cut-off point was 2.717 pg/ml with 78% sensitivity (see Table 15 (4^{th} collection)). For ASC with 100% specificity, the cut-off point was 462.4 pg/ml with 85% sensitivity (see Table 16 (4^{th} collection)). Thus, these findings indicate that caspase-1 and ASC are reliable serum biomarkers for TBI.

Table 11A-B: ROC analysis results for caspase-1 (Table 11A) and ASC (Table 11B) in serum including cut-off point in pg/ml, sensitivity and specificity, as well as positive and negative likelihood ratios (LR+/LR−).

TABLE 11A
ROC analysis for Caspase-1 in Serum.

| | Cut-off | | | | |
|---|---|---|---|---|---|
| | point | Sensitivity | Specificity | | |
| Biomarker | (pg/ml) | (%) | (%) | LR + | LR − |
| 1^{st} Collection | >1.439 | 83 | 67 | 2.50 | 0.25 |
| 2^{nd} Collection | >1.531 | 94 | 78 | 4.24 | 0.08 |
| 4^{th} Collection | >1.943 | 94 | 89 | 8.50 | 0.06 |
| 6^{th} Collection | >1.947 | 85 | 89 | 7.62 | 0.17 |

TABLE 11B
ROC analysis for ASC in Serum.

| | Cut-off | | | | |
|---|---|---|---|---|---|
| | point | Sensitivity | Specificity | | |
| Biomarker | (pg/ml) | (%) | (%) | LR + | LR − |
| 1^{st} Collection | >210 | 85 | 43 | 1.50 | 0.35 |
| 2^{nd} Collection | >275 | 81 | 72 | 2.91 | 0.26 |
| 4^{th} Collection | >339.4 | 80 | 88 | 6.57 | 0.23 |
| 6^{th} Collection | >451.3 | 85 | 99 | 97.26 | 0.16 |

ASC is Elevated in the Serum of Patients with Unfavorable Outcomes after TBI

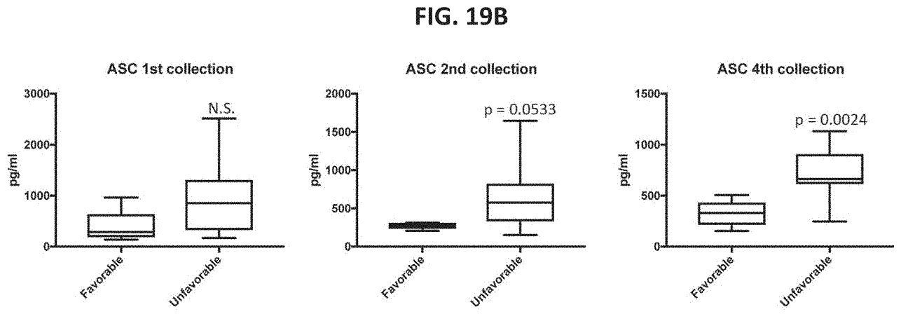

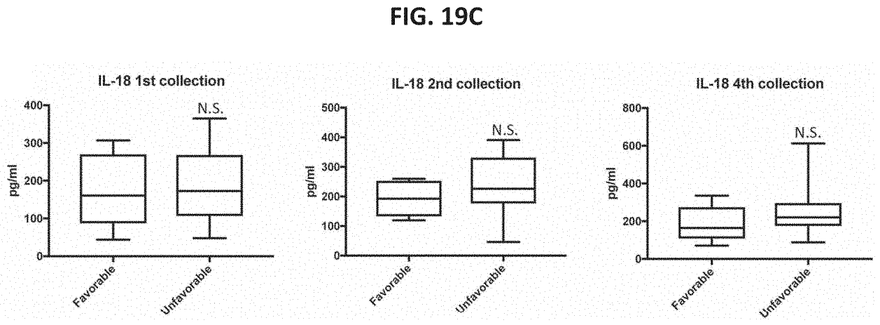

TBI patients were separated according to their clinical outcomes; either favorable or unfavorable outcomes based on the Glasgow Outcome Scale-Extended (GOSE) in which patients with a score of 6 to 8 were considered to have favorable outcomes and those with a score of 1 to 4 were considered to have unfavorable outcomes (Table s 12A and 12B). It was found that the protein level of ASC was higher in the serum of TBI patients with unfavorable outcomes when compared to the samples obtained from patients with favorable outcomes (FIG. 19B), whereas the caspase-1 (FIG. 19A) and IL-18 (FIG. 19C) levels were not statistically different between the two groups.

### ASC is a Good Prognostic Biomarker of TBI in Serum.

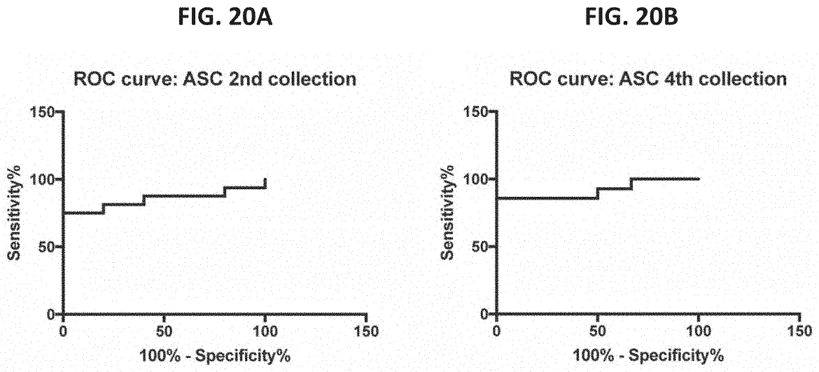

To determine if ASC can be used as prognostic biomarkers of TBI, we determined the AUC for ASC at the 2nd (FIG. 20A) and 4th collection (FIG. 20B). The AUC for ASC was 0.9167 in the 4th collection with a CI between 0.7914 and 1.042 (Table 12A). Furthermore, the cut-off point was 547.6 pg/ml with 86% sensitivity and 100% specificity (Table 12B and Table 19 (4^{th} collection). Thus, these findings indicated that ASC is a promising prognostic biomarker of TBI in serum.

Table 12A-B: ROC analysis results for ASC in serum for Favorable (Table 12A) vs Unfavorable (Table 12B) outcomes, including area, standard error (STD. ERROR), 95% confidence interval (CI), p-value (see Table 12A), cut-off point in pg/ml, sensitivity and specificity, as well as positive and negative likelihood ratios (LR+/LR−) (see Table 12B) for collections 1^{st}, 2^{nd} and 4^{th}.

TABLE 12A
ROC analysis for ASC in Serum (GOSE) for favorable outcome.

| | | STD. | | |
|---|---|---|---|---|
| BIOMARKER | AREA | ERROR | 95% C.I. | P VALUE |
| 1^{st} Collection | 0.7625 | 0.1133 | 0.544 to 0.9846 | 0.0829 |
| 2^{nd} Collection | 0.85 | 0.08355 | 0.6862 to 1.014 | 0.0208 |
| 4^{th} Collection | 0.9167 | 0.06391 | 0.7914 to 1.042 | 0.0039 |

TABLE 12B
ROC analysis for ASC in Serum (GOSE) for unfavorable outcome.

| | CUT-OFF | | | | |
|---|---|---|---|---|---|
| | POINT | SENSITIVITY | SPECIFICITY | | |
| BIOMARKER | (pg/ml) | (%) | (%) | LR + | LR − |
| 1^{st} Collection | >353.7 | 75 | 80 | 3.75 | 0.31 |
| 2^{nd} Collection | >311.2 | 81.25 | 80 | 4.06 | 0.23 |
| 4^{th} Collection | >547.6 | 85.71 | 100 | | 0.14 |

ASC and IL-18 are Elevated in the CSF of Patients after TBI.

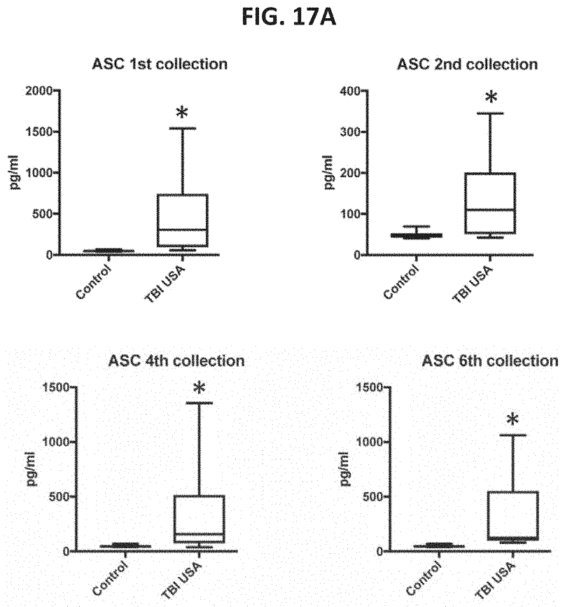

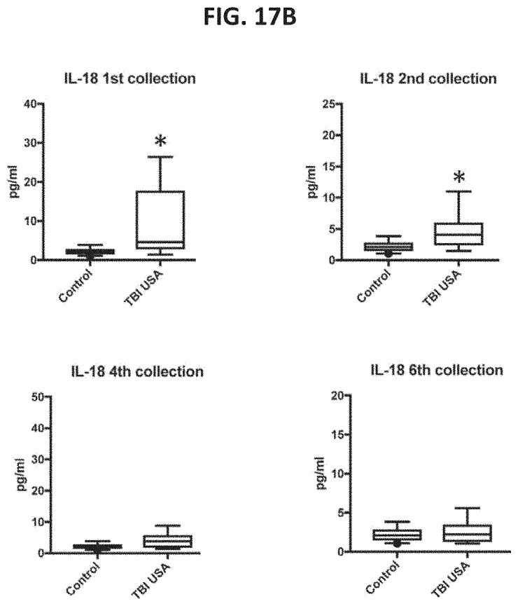

CSF samples from TBI patients were analyzed and compared to CSF from healthy/control individuals using a Simple Plex assay (Protein Simple) for the protein expression of the inflammasome signaling proteins ASC and IL-18 (FIGS. 17A and 17B). The protein levels of ASC and IL-18 in the serum of TBI patients were both higher than in the control group.

### ASC and IL-18 are Good CSF Biomarkers of TBI

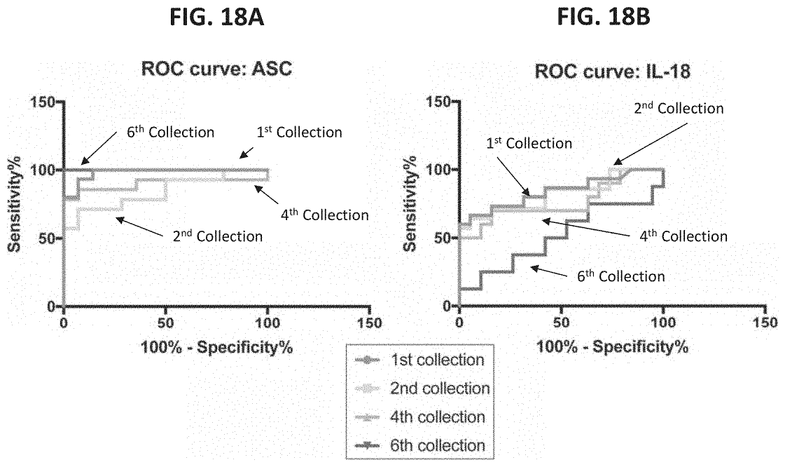

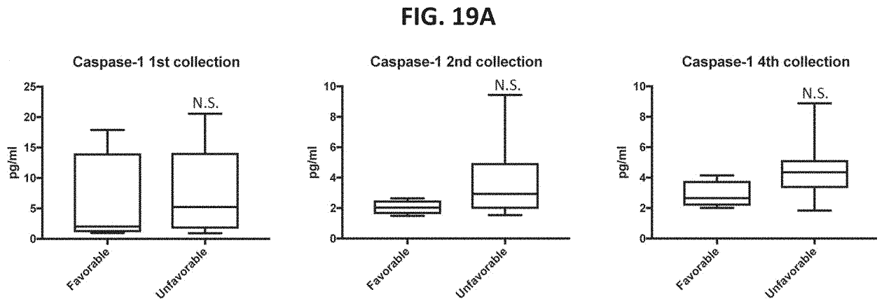

To then determine if these inflammasome signaling proteins have the potential to be reliable biomarkers for TBI pathology, the area under the curve (AUC) for ASC, and IL-18 (FIGS. 18A and 18B) in CSF were determined. ASC and IL-18 were shown to be the best biomarkers (FIGS. 18A and 18B) with an AUC of 1.0 (6th collection) and 0.84 (1^{st} collection), respectively (Tables 13A and 13B).

Tables 13A and 13B: ROC analysis results for ASC (Table 13A) and IL-18 (Table 13B) in CSF including cut-off point in pg/ml, sensitivity and specificity, as well as positive and negative likelihood ratios (LR+/LR−).

TABLE 13A
ROC analysis of ASC in CSF.

| | | STD. | | |
|---|---|---|---|---|
| BIOMARKER | AUC | ERROR | 95% C.I. | P VALUE |
| 1^{st} Collection | 0.981 | 0.0195 | 0.9427 to 1.019 | <0.0001 |
| 2^{nd} Collection | 0.8418 | 0.07661 | 0.6917 to 0.992 | 0.0021 |
| 4^{th} Collection | 0.898 | 0.07262 | 0.7556 to 1.04 | 0.0003 |
| 6^{th} Collection | 1 | 0 | 1 to 1 | 0.0001 |

TABLE 13B
ROC analysis of IL-18 in CSF.

| | | STD. | | |
|---|---|---|---|---|
| BIOMARKER | AUC | ERROR | 95% C.I. | P VALUE |
| 1^{st} Collection | 0.8404 | 0.0731 | 0.6971 to 0.9836 | 0.0008 |
| 2^{nd} Collection | 0.8195 | 0.07969 | 0.6634 to 0.9757 | 0.002 |
| 4^{th} Collection | 0.7632 | 0.1061 | 0.552 to 0.9711 | 0.9711 |
| 6^{th} Collection | 0.5132 | 0.1344 | 0.2498 to 0.7765 | 0.9154 |

Furthermore, the cut-off point for ASC, the cut-off point was 74.33 pg/ml with 100% sensitivity and 100% specificity (Table 14A and Table 17). For IL-18, the cut-off point was 2.722 pg/ml with 80% sensitivity and 68% specificity (Table 14B and Table 18). As shown in Table 18, in the case of IL-18, for 100% specificity, the cut-off point was 3.879 pg/ml with 60% sensitivity; for 100% sensitivity, the cut-off point was 1.358 pg/ml, with 16% specificity. Thus, these findings indicate that ASC and IL-18 are reliable serum biomarkers for TBI.

Table 14A-B: ROC analysis results for ASC (Table 14A) and IL-18 (Table 14B) in CSF including cut-off point in pg/ml, sensitivity and specificity, as well as positive and negative likelihood ratios (LR+/LR−).

TABLE 14A
ROC analysis for ASC in CSF

| | Cut-off | | | | |
|---|---|---|---|---|---|
| | point | Sensitivity | Specificity | | |
| Biomarker | (pg/ml) | (%) | (%) | LR + | LR − |
| 1^{st} Collection | >55.11 | 100 | 85.71 | 7 | 0 |
| 2^{nd} Collection | >50.25 | 78.57 | 64.29 | 2.20 | 0.33 |
| 4^{th} Collection | >64.58 | 85.71 | 92.86 | 12 | 0.15 |
| 6^{th} Collection | >74.33 | 100 | 100 | | 0 |

TABLE 14B
ROC analysis for IL-18 in CSF

| | Cut-off | | | | |
|---|---|---|---|---|---|
| | point | Sensitivity | Specificity | | |
| Biomarker | (pg/ml) | (%) | (%) | LR + | LR − |
| 1^{st} Collection | >2.722 | 80 | 68.42 | 2.53 | 0.29 |
| 2^{nd} Collection | >2.221 | 85.71 | 57.89 | 2.04 | 0.25 |
| 4^{th} Collection | >3.055 | 70 | 84.21 | 4.43 | 0.36 |
| 6^{th} Collection | >1.707 | 75 | 36.84 | 1.19 | 0.68 |

### Conclusions:

In this study, a statistically significant higher level of ASC and caspase-1 was detected in the serum of TBI patients when compared to healthy subjects. In this study, we show that ASC and IL-18 are reliable biomarkers for TBI in CSF with AUC values of 1.0 and 0.84, respectively. Most importantly, since obtaining CSF is a very invasive procedure, then our findings on serum are even more applicable to the typical clinical setting. Accordingly, we found that the AUC values for ASC was 0.90 and for caspase-1, 0.93. Thus caspase-1 and ASC should be considered as biomarkers in the care of patients with brain injury.

Moreover, the data showed that when comparing patients with unfavorable outcomes to patients with favorable outcomes chronically after TBI, the AUC for ASC was 0.92; thus, highlighting the usefulness of ASC as a TBI biomarker in serum, and, in this case, as a predictive biomarker of brain injury.

Thus, based on these findings ASC and caspase-1 are both promising biomarkers with a high AUC value, a high sensitivity and high specificity in serum. Additionally, based on these findings, ASC and IL-18 are both promising biomarkers with a high AUC value, a high sensitivity and high specificity in CSF. Importantly, ASC as a biomarker for TBI with other diagnostic criteria may further increase the sensitivity of ASC as a biomarker for TBI beyond what is described in this example.

Importantly, in this study, ASC has been identified as a potential biomarker of TBI pathology with a high AUC value of 0.9448 and with sensitivities above 80% and a specificity of over 90%.

### INCORPORATION BY REFERENCE

The following references are incorporated by reference in their entireties for all purposes.
1. Adamczak, S., Dale, G., De Rivero Vaccari, J. P., Bullock, M. R., Dietrich, W. D., and Keane, R. W. (2012). Inflammasome proteins in cerebrospinal fluid of brain-injured patients as biomarkers of functional outcome: clinical article. J Neurosurg 117, 1119-1125.
2. Brand, F. J., 3rd, Forouzandeh, M., Kaur, H., Travascio, F., and De Rivero Vaccari, J. P. (2016). Acidification changes affect the inflammasome in human nucleus pulposus cells. J Inflamm (Lond) 13, 29.
3. De Rivero Vaccari, J. P., Brand, F., 3rd, Adamczak, S., Lee, S. W., Perez-Barcena, J., Wang, M. Y., Bullock, M. R., Dietrich, W. D., and Keane, R. W. (2016). Exosome-mediated inflammasome signaling after central nervous system injury. J Neurochem 136 Suppl 1, 39-48.
4. Keane, R. W., Dietrich, W. D., and De Rivero Vaccari, J. P. (2018). Inflammasome Proteins As Biomarkers of Multiple Sclerosis. Front Neurol 9, 135.
5. Xia J, Broadhurst D I, Wilson M and Wishart D S. Translational biomarker discovery in clinical metabolomics: an introductory tutorial. Metabolomics. 2013; 9:280-299.

TABLE 15
Full ROC Data for caspase-1 4^{th} collection in serum

| Cutoff | Sensitivity | | Specificity | | Likelihood |
|---|---|---|---|---|---|
| (pg/ml) | % | 95% CI | % | 95% CI | ratio |
| >0.984 | 100 | 81.47% to 100% | 11.11 | 0.2809% to 48.25% | 1.125 |
| >1.048 | 100 | 81.47% to 100% | 22.22 | 2.814% to 60.01% | 1.286 |
| >1.091 | 100 | 81.47% to 100% | 33.33 | 7.485% to 70.07% | 1.5 |
| >1.19 | 100 | 81.47% to 100% | 44.44 | 13.7% to 78.8% | 1.8 |
| >1.338 | 100 | 81.47% to 100% | 55.56 | 21.2% to 86.3% | 2.25 |
| >1.461 | 100 | 81.47% to 100% | 66.67 | 29.93% to 92.51% | 3 |
| >1.679 | 100 | 81.47% to 100% | 77.78 | 39.99% to 97.19% | 4.5 |
| >1.853 | 94.44 | 72.71% to 99.86% | 77.78 | 39.99% to 97.19% | 4.25 |
| >1.943 | 94.44 | 72.71% to 99.86% | 88.89 | 51.75% to 99.72% | 8.5 |
| >2.293 | 88.89 | 65.29% to 98.62% | 88.89 | 51.75% to 99.72% | 8 |
| >2.577 | 83.33 | 58.58% to 96.42% | 88.89 | 51.75% to 99.72% | 7.5 |
| >2.643 | 77.78 | 52.36% to 93.59% | 88.89 | 51.75% to 99.72% | 7 |
| >2.717 | 77.78 | 52.36% to 93.59% | 100 | 66.37% to 100% | |
| >2.812 | 72.22 | 46.52% to 90.31% | 100 | 66.37% to 100% | |
| >3.174 | 66.67 | 40.99% to 86.66% | 100 | 66.37% to 100% | |
| >3.68 | 61.11 | 35.75% to 82.7% | 100 | 66.37% to 100% | |
| >3.947 | 55.56 | 30.76% to 78.47% | 100 | 66.37% to 100% | |
| >4.027 | 50 | 26.02% to 73.98% | 100 | 66.37% to 100% | |
| >4.105 | 44.44 | 21.53% to 69.24% | 100 | 66.37% to 100% | |
| >4.397 | 38.89 | 17.3% to 64.25% | 100 | 66.37% to 100% | |
| >4.71 | 33.33 | 13.34% to 59.01% | 100 | 66.37% to 100% | |
| >4.95 | 27.78 | 9.695% to 53.48% | 100 | 66.37% to 100% | |
| >5.139 | 22.22 | 6.409% to 47.64% | 100 | 66.37% to 100% | |
| >5.157 | 16.67 | 3.579% to 41.42% | 100 | 66.37% to 100% | |
| >5.59 | 11.11 | 1.375% to 34.71% | 100 | 66.37% to 100% | |
| >7.452 | 5.556 | 0.1406% to 27.29% | 100 | 66.37% to 100% | |

TABLE 16
Full ROC Data for ASC 6^{th} collection in serum

| Cutoff | Sensitivity | | Specificity | | Likelihood |
|---|---|---|---|---|---|
| (pg/ml) | % | 95% CI | % | 95% CI | ratio |
| >105.8 | 100 | 75.29% to 100% | 0.8696 | 0.02201% to 4.75% | 1.009 |
| >107.9 | 100 | 75.29% to 100% | 1.739 | 0.2113% to 6.141% | 1.018 |
| >112.1 | 100 | 75.29% to 100% | 2.609 | 0.5412% to 7.435% | 1.027 |
| >123.3 | 100 | 75.29% to 100% | 3.478 | 0.9557% to 8.667% | 1.036 |
| >132.4 | 100 | 75.29% to 100% | 4.348 | 1.427% to 9.855% | 1.045 |
| >133 | 100 | 75.29% to 100% | 5.217 | 1.939% to 11.01% | 1.055 |
| >134.2 | 100 | 75.29% to 100% | 6.087 | 2.482% to 12.14% | 1.065 |
| >135.2 | 100 | 75.29% to 100% | 6.957 | 3.051% to 13.25% | 1.075 |
| >135.5 | 100 | 75.29% to 100% | 7.826 | 3.641% to 14.34% | 1.085 |
| >135.8 | 100 | 75.29% to 100% | 8.696 | 4.249% to 15.41% | 1.095 |
| >136.1 | 100 | 75.29% to 100% | 9.565 | 4.872% to 16.47% | 1.106 |
| >139.2 | 100 | 75.29% to 100% | 10.43 | 5.509% to 17.52% | 1.117 |
| >142.6 | 100 | 75.29% to 100% | 11.3 | 6.158% to 18.55% | 1.127 |
| >143.3 | 100 | 75.29% to 100% | 12.17 | 6.818% to 19.58% | 1.139 |
| >144.6 | 100 | 75.29% to 100% | 13.04 | 7.488% to 20.6% | 1.15 |
| >146.2 | 100 | 75.29% to 100% | 13.91 | 8.167% to 21.61% | 1.162 |
| >147.5 | 100 | 75.29% to 100% | 14.78 | 8.854% to 22.61% | 1.173 |
| >148.9 | 100 | 75.29% to 100% | 15.65 | 9.548% to 23.6% | 1.186 |
| >150.4 | 100 | 75.29% to 100% | 16.52 | 10.25% to 24.59% | 1.198 |
| >151.4 | 100 | 75.29% to 100% | 17.39 | 10.96% to 25.57% | 1.211 |
| >151.8 | 100 | 75.29% to 100% | 18.26 | 11.67% to 26.55% | 1.223 |
| >153.4 | 100 | 75.29% to 100% | 19.13 | 12.39% to 27.52% | 1.237 |
| >155.5 | 92.31 | 63.97% to 99.81% | 19.13 | 12.39% to 27.52% | 1.141 |
| >158.2 | 92.31 | 63.97% to 99.81% | 20 | 13.12% to 28.48% | 1.154 |
| >160.8 | 92.31 | 63.97% to 99.81% | 20.87 | 13.85% to 29.44% | 1.167 |
| >164 | 92.31 | 63.97% to 99.81% | 21.74 | 14.59% to 30.4% | 1.179 |
| >168 | 92.31 | 63.97% to 99.81% | 22.61 | 15.33% to 31.35% | 1.193 |
| >170.2 | 92.31 | 63.97% to 99.81% | 23.48 | 16.08% to 32.29% | 1.206 |
| >171.2 | 92.31 | 63.97% to 99.81% | 24.35 | 16.83% to 33.23% | 1.22 |
| >172.2 | 92.31 | 63.97% to 99.81% | 25.22 | 17.58% to 34.17% | 1.234 |
| >173.4 | 92.31 | 63.97% to 99.81% | 26.09 | 18.34% to 35.1% | 1.249 |
| >175.6 | 92.31 | 63.97% to 99.81% | 26.96 | 19.11% to 36.03% | 1.264 |
| >178.5 | 92.31 | 63.97% to 99.81% | 27.83 | 19.87% to 36.95% | 1.279 |
| >180.9 | 92.31 | 63.97% to 99.81% | 28.7 | 20.65% to 37.88% | 1.295 |
| >182.1 | 92.31 | 63.97% to 99.81% | 29.57 | 21.42% to 38.79% | 1.311 |
| >183.3 | 92.31 | 63.97% to 99.81% | 30.43 | 22.2% to 39.71% | 1.327 |
| >184.4 | 92.31 | 63.97% to 99.81% | 31.3 | 22.98% to 40.62% | 1.344 |
| >184.9 | 92.31 | 63.97% to 99.81% | 32.17 | 23.77% to 41.53% | 1.361 |
| >185.7 | 92.31 | 63.97% to 99.81% | 33.04 | 24.56% to 42.43% | 1.379 |
| >186.5 | 92.31 | 63.97% to 99.81% | 33.91 | 25.35% to 43.33% | 1.397 |
| >188.9 | 92.31 | 63.97% to 99.81% | 34.78 | 26.14% to 44.23% | 1.415 |
| >191.1 | 92.31 | 63.97% to 99.81% | 35.65 | 26.94% to 45.12% | 1.435 |
| >191.9 | 92.31 | 63.97% to 99.81% | 36.52 | 27.74% to 46.01% | 1.454 |
| >193.1 | 92.31 | 63.97% to 99.81% | 37.39 | 28.55% to 46.9% | 1.474 |
| >195.2 | 92.31 | 63.97% to 99.81% | 38.26 | 29.35% to 47.79% | 1.495 |
| >196.6 | 92.31 | 63.97% to 99.81% | 39.13 | 30.16% to 48.67% | 1.516 |
| >197.2 | 92.31 | 63.97% to 99.81% | 40 | 30.98% to 49.55% | 1.538 |
| >198.7 | 92.31 | 63.97% to 99.81% | 40.87 | 31.79% to 50.43% | 1.561 |
| >202.1 | 92.31 | 63.97% to 99.81% | 41.74 | 32.61% to 51.3% | 1.584 |
| >207.2 | 92.31 | 63.97% to 99.81% | 42.61 | 33.44% to 52.17% | 1.608 |
| >210 | 92.31 | 63.97% to 99.81% | 43.48 | 34.26% to 53.04% | 1.633 |
| >211.1 | 92.31 | 63.97% to 99.81% | 44.35 | 35.09% to 53.91% | 1.659 |
| >213.9 | 92.31 | 63.97% to 99.81% | 45.22 | 35.92% to 54.77% | 1.685 |
| >216.3 | 84.62 | 54.55% to 98.08% | 45.22 | 35.92% to 54.77% | 1.545 |
| >216.8 | 84.62 | 54.55% to 98.08% | 46.09 | 36.75% to 55.63% | 1.569 |
| >218.1 | 84.62 | 54.55% to 98.08% | 46.96 | 37.59% to 56.49% | 1.595 |
| >220.4 | 84.62 | 54.55% to 98.08% | 47.83 | 38.43% to 57.34% | 1.622 |
| >224.1 | 84.62 | 54.55% to 98.08% | 48.7 | 39.27% to 58.19% | 1.649 |
| >227.1 | 84.62 | 54.55% to 98.08% | 49.57 | 40.11% to 59.04% | 1.678 |
| >228.8 | 84.62 | 54.55% to 98.08% | 50.43 | 40.96% to 59.89% | 1.707 |
| >230.8 | 84.62 | 54.55% to 98.08% | 51.3 | 41.81% to 60.73% | 1.738 |
| >231.7 | 84.62 | 54.55% to 98.08% | 52.17 | 42.66% to 61.57% | 1.769 |
| >232.6 | 84.62 | 54.55% to 98.08% | 53.04 | 43.51% to 62.41% | 1.802 |
| >233.5 | 84.62 | 54.55% to 98.08% | 53.91 | 44.37% to 63.25% | 1.836 |
| >238.2 | 84.62 | 54.55% to 98.08% | 54.78 | 45.23% to 64.08% | 1.871 |
| >243.1 | 84.62 | 54.55% to 98.08% | 55.65 | 46.09% to 64.91% | 1.908 |
| >244 | 84.62 | 54.55% to 98.08% | 56.52 | 46.96% to 65.74% | 1.946 |
| >244.7 | 84.62 | 54.55% to 98.08% | 57.39 | 47.83% to 66.56% | 1.986 |
| >247.5 | 84.62 | 54.55% to 98.08% | 58.26 | 48.7% to 67.39% | 2.027 |
| >250.2 | 84.62 | 54.55% to 98.08% | 59.13 | 49.57% to 68.21% | 2.07 |
| >250.5 | 84.62 | 54.55% to 98.08% | 60 | 50.45% to 69.02% | 2.115 |
| >250.7 | 84.62 | 54.55% to 98.08% | 60.87 | 51.33% to 69.84% | 2.162 |
| >251.6 | 84.62 | 54.55% to 98.08% | 61.74 | 52.21% to 70.65% | 2.212 |
| >252.4 | 84.62 | 54.55% to 98.08% | 62.61 | 53.1% to 71.45% | 2.263 |
| >254.2 | 84.62 | 54.55% to 98.08% | 63.48 | 53.99% to 72.26% | 2.317 |
| >257.2 | 84.62 | 54.55% to 98.08% | 64.35 | 54.88% to 73.06% | 2.373 |
| >259 | 84.62 | 54.55% to 98.08% | 65.22 | 55.77% to 73.86% | 2.433 |
| >260.8 | 84.62 | 54.55% to 98.08% | 66.09 | 56.67% to 74.65% | 2.495 |
| >263.3 | 84.62 | 54.55% to 98.08% | 66.96 | 57.57% to 75.44% | 2.561 |
| >267.1 | 84.62 | 54.55% to 98.08% | 67.83 | 58.47% to 76.23% | 2.63 |
| >270.9 | 84.62 | 54.55% to 98.08% | 68.7 | 59.38% to 77.02% | 2.703 |
| >272.3 | 84.62 | 54.55% to 98.08% | 69.57 | 60.29% to 77.8% | 2.78 |
| >272.7 | 84.62 | 54.55% to 98.08% | 70.43 | 61.21% to 78.58% | 2.862 |
| >273.3 | 84.62 | 54.55% to 98.08% | 71.3 | 62.12% to 79.35% | 2.949 |
| >277.9 | 84.62 | 54.55% to 98.08% | 72.17 | 63.05% to 80.13% | 3.041 |
| >282.9 | 84.62 | 54.55% to 98.08% | 73.04 | 63.97% to 80.89% | 3.139 |
| >283.9 | 84.62 | 54.55% to 98.08% | 73.91 | 64.9% to 81.66% | 3.244 |
| >286.3 | 84.62 | 54.55% to 98.08% | 74.78 | 65.83% to 82.42% | 3.355 |
| >289.3 | 84.62 | 54.55% to 98.08% | 75.65 | 66.77% to 83.17% | 3.475 |
| >290.4 | 84.62 | 54.55% to 98.08% | 76.52 | 67.71% to 83.92% | 3.604 |
| >294.2 | 84.62 | 54.55% to 98.08% | 77.39 | 68.65% to 84.67% | 3.743 |
| >298 | 84.62 | 54.55% to 98.08% | 78.26 | 69.6% to 85.41% | 3.892 |
| >300.4 | 84.62 | 54.55% to 98.08% | 79.13 | 70.56% to 86.15% | 4.054 |
| >302.7 | 84.62 | 54.55% to 98.08% | 80 | 71.52% to 86.88% | 4.231 |
| >304 | 84.62 | 54.55% to 98.08% | 80.87 | 72.48% to 87.61% | 4.423 |
| >310.4 | 84.62 | 54.55% to 98.08% | 81.74 | 73.45% to 88.33% | 4.634 |
| >318.3 | 84.62 | 54.55% to 98.08% | 82.61 | 74.43% to 89.04% | 4.865 |
| >321.9 | 84.62 | 54.55% to 98.08% | 83.48 | 75.41% to 89.75% | 5.121 |
| >324.4 | 84.62 | 54.55% to 98.08% | 84.35 | 76.4% to 90.45% | 5.406 |
| >326.2 | 84.62 | 54.55% to 98.08% | 85.22 | 77.39% to 91.15% | 5.724 |
| >328.7 | 84.62 | 54.55% to 98.08% | 86.09 | 78.39% to 91.83% | 6.082 |
| >331 | 84.62 | 54.55% to 98.08% | 86.96 | 79.4% to 92.51% | 6.487 |
| >340.6 | 84.62 | 54.55% to 98.08% | 87.83 | 80.42% to 93.18% | 6.951 |
| >351.1 | 84.62 | 54.55% to 98.08% | 88.7 | 81.45% to 93.84% | 7.485 |
| >353.1 | 84.62 | 54.55% to 98.08% | 89.57 | 82.48% to 94.49% | 8.109 |
| >356.7 | 84.62 | 54.55% to 98.08% | 90.43 | 83.53% to 95.13% | 8.846 |
| >370.3 | 84.62 | 54.55% to 98.08% | 91.3 | 84.59% to 95.75% | 9.731 |
| >381.9 | 84.62 | 54.55% to 98.08% | 92.17 | 85.66% to 96.36% | 10.81 |
| >383.7 | 84.62 | 54.55% to 98.08% | 93.04 | 86.75% to 96.95% | 12.16 |
| >390.2 | 84.62 | 54.55% to 98.08% | 93.91 | 87.86% to 97.52% | 13.9 |
| >396.9 | 84.62 | 54.55% to 98.08% | 94.78 | 88.99% to 98.06% | 16.22 |
| >400.4 | 84.62 | 54.55% to 98.08% | 95.65 | 90.15% to 98.57% | 19.46 |
| >419.6 | 84.62 | 54.55% to 98.08% | 96.52 | 91.33% to 99.04% | 24.33 |
| >437.2 | 84.62 | 54.55% to 98.08% | 97.39 | 92.57% to 99.46% | 32.44 |
| >441 | 84.62 | 54.55% to 98.08% | 98.26 | 93.86% to 99.79% | 48.65 |
| >451.3 | 84.62 | 54.55% to 98.08% | 99.13 | 95.25% to 99.98% | 97.31 |
| >462.4 | 84.62 | 54.55% to 98.08% | 100 | 96.84% to 100% | |
| >494.8 | 76.92 | 46.19% to 94.96% | 100 | 96.84% to 100% | |
| >545.1 | 69.23 | 38.57% to 90.91% | 100 | 96.84% to 100% | |
| >586.5 | 61.54 | 31.58% to 86.14% | 100 | 96.84% to 100% | |
| >619.6 | 53.85 | 25.13% to 80.78% | 100 | 96.84% to 100% | |
| >633.9 | 46.15 | 19.22% to 74.87% | 100 | 96.84% to 100% | |
| >736.9 | 38.46 | 13.86% to 68.42% | 100 | 96.84% to 100% | |
| >865.8 | 30.77 | 9.092% to 61.43% | 100 | 96.84% to 100% | |
| >892.6 | 23.08 | 5.038% to 53.81% | 100 | 96.84% to 100% | |
| >976.4 | 15.38 | 1.921% to 45.45% | 100 | 96.84% to 100% | |
| >1065 | 7.692 | 0.1946% to 36.03% | 100 | 96.84% to 100% | |

TABLE 17
Full ROC Data for ASC 6^{th} collection in CSF

| Cutoff | Sensitivity | | Specificity | | Likelihood |
|---|---|---|---|---|---|
| (pg/ml) | % | 95% CI | % | 95% CI | ratio |
| >40.63 | 100 | 63.06% to | 7.143 | 0.1807% to | 1.077 |
| | | 100% | | 33.87% | |
| >40.67 | 100 | 63.06% to | 14.29 | 1.779% to | 1.167 |
| | | 100% | | 42.81% | |
| >41.64 | 100 | 63.06% to | 21.43 | 4.658% to | 1.273 |
| | | 100% | | 50.8% | |
| >42.71 | 100 | 63.06% to | 28.57 | 8.389% to | 1.4 |
| | | 100% | | 58.1% | |
| >43.09 | 100 | 63.06% to | 35.71 | 12.76% to | 1.556 |
| | | 100% | | 64.86% | |
| >43.68 | 100 | 63.06% to | 42.86 | 17.66% to | 1.75 |
| | | 100% | | 71.14% | |
| >45.92 | 100 | 63.06% to | 50 | 23.04% to | 2 |
| | | 100% | | 76.96% | |
| >48.29 | 100 | 63.06% to | 57.14 | 28.86% to | 2.333 |
| | | 100% | | 82.34% | |
| >50.25 | 100 | 63.06% to | 64.29 | 35.14% to | 2.8 |
| | | 100% | | 87.24% | |
| >52.18 | 100 | 63.06% to | 71.43 | 41.9% to | 3.5 |
| | | 100% | | 91.61% | |
| >53.27 | 100 | 63.06% to | 78.57 | 49.2% to | 4.667 |
| | | 100% | | 95.34% | |
| >57.07 | 100 | 63.06% to | 85.71 | 57.19% to | 7 |
| | | 100% | | 98.22% | |
| >64.81 | 100 | 63.06% to | 92.86 | 66.13% to | 14 |
| | | 100% | | 99.82% | |
| >74.33 | 100 | 63.06% to | 100 | 76.84% to | |
| | | 100% | | 100% | |
| >84.74 | 87.5 | 47.35% to | 100 | 76.84% to | |
| | | 99.68% | | 100% | |
| >103.3 | 75 | 34.91% to | 100 | 76.84% to | |
| | | 96.81% | | 100% | |
| >117.3 | 62.5 | 24.49% to | 100 | 76.84% to | |
| | | 91.48% | | 100% | |
| >122.5 | 50 | 15.7% to | 100 | 76.84% to | |
| | | 84.3% | | 100% | |
| >268.5 | 37.5 | 8.523% to | 100 | 76.84% to | |
| | | 75.51% | | 100% | |
| >504.9 | 25 | 3.185% to | 100 | 76.84% to | |
| | | 65.09% | | 100% | |
| >830.8 | 12.5 | 0.316% to | 100 | 76.84% to | |
| | | 52.65% | | 100% | |

TABLE 18
Full ROC Data for IL-18 1^{st} collection in CSF

| Cutoff | Sensitivity | | Specificity | | Likelihood |
|---|---|---|---|---|---|
| (pg/ml) | % | 95% CI | % | 95% CI | ratio |
| >1.167 | 100 | 78.2% to | 5.263 | 0.1332% to | 1.056 |
| | | 100% | | 26.03% | |
| >1.298 | 100 | 78.2% to | 10.53 | 1.301% to | 1.118 |
| | | 100% | | 33.14% | |
| >1.358 | 100 | 78.2% to | 15.79 | 3.383% to | 1.188 |
| | | 100% | | 39.58% | |
| >1.406 | 93.33 | 68.05% to | 21.05 | 6.052% to | 1.182 |
| | | 99.83% | | 45.57% | |
| >1.499 | 93.33 | 68.05% to | 26.32 | 9.147% to | 1.267 |
| | | 99.83% | | 51.2% | |
| >1.608 | 93.33 | 68.05% to | 31.58 | 12.58% to | 1.364 |
| | | 99.83% | | 56.55% | |
| >1.737 | 93.33 | 68.05% to | 36.84 | 16.29% to | 1.478 |
| | | 99.83% | | 61.64% | |
| >1.844 | 86.67 | 59.54% to | 36.84 | 16.29% to | 1.372 |
| | | 98.34% | | 61.64% | |
| >1.91 | 86.67 | 59.54% to | 42.11 | 20.25% to | 1.497 |
| | | 98.34% | | 66.5% | |
| >2.024 | 86.67 | 59.54% to | 47.37 | 24.45% to | 1.647 |
| | | 98.34% | | 71.14% | |
| >2.11 | 86.67 | 59.54% to | 52.63 | 28.86% to | 1.83 |
| | | 98.34% | | 75.55% | |
| >2.188 | 86.67 | 59.54% to | 57.89 | 33.5% to | 2.058 |
| | | 98.34% | | 79.75% | |
| >2.474 | 80 | 51.91% to | 57.89 | 33.5% to | 1.9 |
| | | 95.67% | | 79.75% | |
| >2.698 | 80 | 51.91% to | 63.16 | 38.36% to | 2.171 |
| | | 95.67% | | 83.71% | |
| >2.722 | 80 | 51.91% to | 68.42 | 43.45% to | 2.533 |
| | | 95.67% | | 87.42% | |
| >2.758 | 73.33 | 44.9% to | 68.42 | 43.45% to | 2.322 |
| | | 92.21% | | 87.42% | |
| >2.817 | 73.33 | 44.9% to | 73.68 | 48.8% to | 2.787 |
| | | 92.21% | | 90.85% | |
| >2.865 | 73.33 | 44.9% to | 78.95 | 54.43% to | 3.483 |
| | | 92.21% | | 93.95% | |
| >2.945 | 73.33 | 44.9% to | 84.21 | 60.42% to | 4.644 |
| | | 92.21% | | 96.62% | |
| >3.23 | 66.67 | 38.38% to | 84.21 | 60.42% to | 4.222 |
| | | 88.18% | | 96.62% | |
| >3.586 | 66.67 | 38.38% to | 89.47 | 66.86% to | 6.333 |
| | | 88.18% | | 98.7% | |
| >3.747 | 66.67 | 38.38% to | 94.74 | 73.97% to | 12.67 |
| | | 88.18% | | 99.87% | |
| >3.806 | 60 | 32.29% to | 94.74 | 73.97% to | 11.4 |
| | | 83.66% | | 99.87% | |
| >3.879 | 60 | 32.29% to | 100 | 82.35% to | |
| | | 83.66% | | 100% | |
| >4.254 | 53.33 | 26.59% to | 100 | 82.35% to | |
| | | 78.73% | | 100% | |
| >5.826 | 46.67 | 21.27% to | 100 | 82.35% to | |
| | | 73.41% | | 100% | |
| >8.428 | 40 | 16.34% to | 100 | 82.35% to | |
| | | 67.71% | | 100% | |
| >10.31 | 33.33 | 11.82% to | 100 | 82.35% to | |
| | | 61.62% | | 100% | |
| >14.29 | 26.67 | 7.787% to | 100 | 82.35% to | |
| | | 55.1% | | 100% | |
| >18.52 | 20 | 4.331% to | 100 | 82.35% to | |
| | | 48.09% | | 100% | |
| >21.1 | 13.33 | 1.658% to | 100 | 82.35% to | |
| | | 40.46% | | 100% | |
| >24.64 | 6.667 | 0.1686% to | 100 | 82.35% to | |
| | | 31.95% | | 100% | |

TABLE 19
Full ROC Data for ASC 4^{th} collection in serum
(favorable vs. unfavorable)

| Cutoff | Sensitivity | | Specificity | | Likelihood |
|---|---|---|---|---|---|
| (pg/ml) | % | 95% CI | % | 95% CI | ratio |
| >194.1 | 100 | 76.84% to | 16.67 | 0.4211% to | 1.2 |
| | | 100% | | 64.12% | |
| >240.2 | 100 | 76.84% to | 33.33 | 4.327% to | 1.5 |
| | | 100% | | 77.72% | |
| >254.2 | 92.86 | 66.13% to | 33.33 | 4.327% to | 1.393 |
| | | 99.82% | | 77.72% | |
| >304.9 | 92.86 | 66.13% to | 50 | 11.81% to | 1.857 |
| | | 99.82% | | 88.19% | |
| >374.1 | 85.71 | 57.19% to | 50 | 11.81% to | 1.714 |
| | | 98.22% | | 88.19% | |
| >404.7 | 85.71 | 57.19% to | 66.67 | 22.28% to | 2.571 |
| | | 98.22% | | 95.67% | |
| >457.6 | 85.71 | 57.19% to | 83.33 | 35.88% to | 5.143 |
| | | 98.22% | | 99.58% | |
| >547.6 | 85.71 | 57.19% to | 100 | 54.07% to | |
| | | 98.22% | | 100% | |
| >605.1 | 78.57 | 49.2% to | 100 | 54.07% to | |
| | | 95.34% | | 100% | |
| >623.8 | 71.43 | 41.9% to | 100 | 54.07% to | |
| | | 91.61% | | 100% | |
| >636.5 | 64.29 | 35.14% to | 100 | 54.07% to | |
| | | 87.24% | | 100% | |
| >647 | 57.14 | 28.86% to | 100 | 54.07% to | |
| | | 82.34% | | 100% | |
| >663.7 | 50 | 23.04% to | 100 | 54.07% to | |
| | | 76.96% | | 100% | |
| >716.7 | 42.86 | 17.66% to | 100 | 54.07% to | |
| | | 71.14% | | 100% | |
| >769 | 35.71 | 12.76% to | 100 | 54.07% to | |
| | | 64.86% | | 100% | |
| >828.4 | 28.57 | 8.389% to | 100 | 54.07% to | |
| | | 58.1% | | 100% | |
| >944.7 | 21.43 | 4.658% to | 100 | 54.07% to | |
| | | 50.8% | | 100% | |
| >1061 | 14.29 | 1.779% to | 100 | 54.07% to | |
| | | 42.81% | | 100% | |
| >1118 | 7.143 | 0.1807% to | 100 | 54.07% to | |
| | | 33.87% | | 100% | |

### Example 4: Examination of Inflammasome Proteins as Biomarkers of Mild Cognitive Impairment (MCI) and Alzheimer's Disease (AD)

### Introduction

A biomarker is a characteristic that can be measured objectively and evaluated as an indicator of normal or pathologic biological processes¹. Important to the care of patients with MCI and AD are the need for biomarkers that can predict onset, exacerbation as well as response to treatment. Additionally, there is a need for a minimally invasive method of harvesting these biomarkers for analysis.

### Methods

### Participants:

In this example, samples were purchased from BioIVT. Sample donors were enrolled in the study “Prospective Collection of Samples for Research” sponsored by SeraTrials, LLC with IRB number 20170439. Here, serum samples from 72 normal male and female donors in the age range of 50 and 68 as well as from 32 male and female patients diagnosed with MCI (Table 20A) in the age range of 56 to 91 as well as 32 male and female patients diagnosed with Alzheimer's Disease in the age range of 47 to 87 (Table 20B). Donors were classified according to their ARIC MRI cognitive function scores. The scale was developed as part of the Atherosclerosis Risk in Communities (ARIC) study that recruited middle-aged individuals who underwent magnetic resonance imaging (MRI) to evaluate the risk factors of vascular problems in these individuals [40]. Cognitive testing was evaluated using the Delayed Word Recall Test, the Digit Symbol Subtest of the Wechsler Adult Intelligence Scale-Revised (WAIS-R) test, and the Controlled Oral Word Association (or Word Fluency) Test of the Multilingual Aphasia Examination.

TABLE 20A
Demographics of participants in MCI study

| | | | | | Historical |
|---|---|---|---|---|---|
| Age | Gender | Race | Diagnosis | Medications | Test |
| 83 | Male | Caucasian | Mild Cognitive Impairment | Omega 3 | ARIC |
| | | | (MCI), Prostate Cancer, | 1000 mg, Plavix | MRI |
| | | | Methicillin Resistant | 75 mg, Toprol | Cognitive |
| | | | Staphylococcus Aureus | 50 mg, Vitamin | Function |
| | | | Infection, Hyperlipidemia | B12-Folic Acid | Score = 18 |
| | | | (HLD), Hypertension | 0.5 mg-1 mg, | (Feb. 20, 2018) |
| | | | (HTN), Diverticulitis, | Vitamin D 400 iu, | |
| | | | Amnesia | Zetia 10 mg | |
| 81 | Female | Caucasian | Mild Cognitive Impairment | Aspirin 81 mg, | ARIC |
| | | | (MCI), Type 2 Diabetes, | Gabapentin | MRI |
| | | | Hypercholesterolemia | 100 m, Eliquis | Cognitive |
| | | | | 2.5 mg, Ranitidine | Function |
| | | | | 150 mg, Aricept | Score = 18 |
| | | | | 10 mg | (May 22, 2018) |
| 62 | Male | Caucasian | Mild Cognitive Impairment | Omeprazole | ARIC |
| | | | (MCI), Type 2 Diabetes, | 20 mg, Benicar | MRI |
| | | | Hypertension (HTN), | 40 mg-12.5 mg, | Cognitive |
| | | | Hyperlipidemia (HLD), | Metformin HCL | Function |
| | | | Asthma | 500 mg, Glucotrol | Score = 30 |
| | | | | XL 5 mg, | (May 15, 2018) |
| | | | | Singulair 10 mg, | |
| | | | | Clobetasol | |
| | | | | Propionate | |
| | | | | 0.05%, Glipizide | |
| | | | | 5 mg, Advair | |
| | | | | Diskus 250 mcg- | |
| | | | | 50 mcg, Crestor | |
| | | | | 10 mg, | |
| | | | | Ipratropium- | |
| | | | | Albuterol 0.5 mg- | |
| | | | | 2.5 mg/3 mL, | |
| | | | | Ventolin HFA | |
| | | | | 108 mcg | |
| 69 | Female | Caucasian | Mild Cognitive Impairment | Alendronate | ARIC |
| | | | (MCI), Asthma, Chronic | 70 mg, Meclizine | MRI |
| | | | Obstructive Pulmonary | 12.5 mg, Prozac | Cognitive |
| | | | Disease (COPD), | 40 mg, Seroquel | Function |
| | | | Hypertension (HTN) | 50 mg, Trilipix | Score = 21 |
| | | | | 54 mg | (May 30, 2018) |
| 75 | Male | Caucasian | Mild Cognitive Impairment | Vitamin B12 | ARIC |
| | | | (MCI), Colon Cancer | 2500 iu, Avastin, | MRI |
| | | | | Adrucil, | Cognitive |
| | | | | Amoxicillin | Function |
| | | | | 500 mg, Lisinopril | Score = 12 |
| | | | | 20 mg, Metformin | (Mar. 27, 2018) |
| | | | | HCL 500 mg | |
| 72 | Male | Caucasian | Mild Cognitive Impairment | Tamsulosin HCL | ARIC |
| | | | (MCI), Benign Prostatic | 0.4 mg, | MRI |
| | | | Hyperplasia (BPH), | Finasteride 5 mg, | Cognitive |
| | | | Lumbar Spondylosis, | Multivitamin, | Function |
| | | | Barrett's Esophagous, | Fish Oil 1000 mg, | Score = 15 |
| | | | Atrial Ectopy, | Viagra 100 mg, | (May 10, 2018) |
| | | | Hypertension (HTN) | Tramadol HCL | |
| | | | | 50 mg | |
| 64 | Male | Caucasian | Mild Cognitive Impairment | Zolpidem 10 mg, | ARIC |
| | | | (MCI), Type 2 Diabetes, | Cialis 5 mg, | MRI |
| | | | Hypertension (HTN), | Aspirin 81 mg, | Cognitive |
| | | | Hypercholesterolemia, | Tamsulosin | Function |
| | | | Benign Prostatic | 0.4 mg, | Score = 34 |
| | | | Hyperplasia (BPH) | Rosuvastatin | (Apr. 4, 2018) |
| | | | | 20 mg, Metformin | |
| | | | | 500 mg | |
| 84 | Female | Caucasian | Mild Cognitive Impairment | Simvastatin | ARIC |
| | | | (MCI), Hypertension | 20 mg, Potassium | MRI |
| | | | (HTN), Psychoses, | Chloride 10 mEq, | Cognitive |
| | | | Cellulitis, Mitral Valve | Amlodipine | Function |
| | | | Prolapse (MVP), | Besylate 2.5 mg, | Score = 8 |
| | | | Hyperlipidemia (HLD) | Dutasteride | (May 10, 2018) |
| | | | | 0.5 mg, Losartan | |
| | | | | Potassium | |
| | | | | 100 mg, Aspirin | |
| | | | | 81 mg, | |
| | | | | Furosemide | |
| | | | | 20 mg, Potassium | |
| | | | | Chloride 10 mEq, | |
| | | | | Avodart 0.4 mg, | |
| | | | | Amlodipine | |
| | | | | Besylate 2.5 mg, | |
| | | | | Ramipril 10 mg | |
| 68 | Female | Caucasian | Mild Cognitive Impairment | Tysabri, Lexapro, | ARIC |
| | | | (MCI), Multiple Sclerosis | Gabapentin | MRI |
| | | | | | Cognitive |
| | | | | | Function |
| | | | | | Score = 15 |
| | | | | | (Apr. 6, 2018) |
| 69 | Female | Caucasian | Mild Cognitive Impairment | Crestor 5 mg, | ARIC |
| | | | (MCI), | Omega 3, | MRI |
| | | | Hypercholesterolemia, | Zolpidem | Cognitive |
| | | | Hypertension (HTN), Type | Tartrate 5 mg, | Function |
| | | | 2 Diabetes, Premature | Glucosamine | Score = 33 |
| | | | Ventricular Contraction | 1500 mg, Fiber, | (May 1, 2018) |
| | | | | Calcium, | |
| | | | | Multivitamin, | |
| | | | | Zyrtec, | |
| | | | | Chlordiazepoxide- | |
| | | | | Clidinium 5 mg- | |
| | | | | 2.5 mg, | |
| | | | | Valacyclovir | |
| | | | | 500 mg, Lisinopril | |
| | | | | 10 mg, Janumet | |
| | | | | 50 mg-500 mg, | |
| | | | | Metoprolol | |
| | | | | Succinate 25 mg, | |
| | | | | Levothyroxine | |
| | | | | Sodium 100 mcg, | |
| | | | | Rosuvastatin | |
| | | | | Calcium 5 mg, | |
| | | | | Omega 3-Acid | |
| | | | | Ethyl Esters 1 g, | |
| | | | | Trazodone 50 mg | |
| 50 | Female | Caucasian | Mild Cognitive Impairment | None | ARIC |
| | | | (MCI), | | MRI |
| | | | Hypercholesterolemia | | Cognitive |
| | | | | | Function |
| | | | | | Score = 30 |
| | | | | | (Apr. 24, 2018) |
| 78 | Male | Caucasian | Mild Cognitive Impairment | Zaleplon 10 mg, | ARIC |
| | | | (MCI) | Lorazepam 1 mg, | MRI |
| | | | | Plavix 75 mg, | Cognitive |
| | | | | Aspirin, | Function |
| | | | | Allopurinol | Score = 24 |
| | | | | 300 mg, | (Apr. 27, 2018) |
| | | | | Levothyroxine | |
| | | | | Sodium 125 mcg, | |
| | | | | Atorvastatin | |
| | | | | Calcium 20 mg, | |
| | | | | Metformin HCL | |
| | | | | 1000 mg, | |
| | | | | Pantoprazole | |
| | | | | Sodium 40 mg | |
| 77 | Male | Caucasian | Mild Cognitive Impairment | Aciphex 20 mg, | ARIC |
| | | | (MCI), Hypertension | Citric Acid-D | MRI |
| | | | (HTN), Hyperlipidemia | Gluconic Acid, | Cognitive |
| | | | (HLD), Vitamin D | Avodart 0.5 mg, | Function |
| | | | Deficiency | Cozaar 100 mg, | Score = 24 |
| | | | | Ranitidine Acid | (May 9, 2018) |
| | | | | Reducer 75 mg, | |
| | | | | Polyethylene | |
| | | | | Glycol, Miralax, | |
| | | | | Symbicort | |
| | | | | 80 mcg-4.5 mcg, | |
| | | | | Proair 108 mcg, | |
| | | | | Ipratropium | |
| | | | | Bromide 0.03%, | |
| | | | | Prevacid 15 mg, | |
| | | | | Losartan | |
| | | | | Potassium | |
| | | | | 100 mg, | |
| | | | | Levocetirizine | |
| | | | | Dihydrochloride | |
| | | | | 5 mg, Cialis 5 mg, | |
| | | | | Albuterol, | |
| | | | | Rabeprazole | |
| | | | | Sodium 20 mg, | |
| | | | | Atorvastatin | |
| | | | | Calcium 20 mg | |
| 73 | Female | Caucasian | Mild Cognitive Impairment | Rabeprazole | ARIC |
| | | | (MCI), | Sodium 20 mg, | MRI |
| | | | Hypercholesterolemia, | Synthroid 75 mcg, | Cognitive |
| | | | Hypothyroidism, | Crestor 5 mg, | Function |
| | | | Hypothyroidism, | Zyrtec Allergy | Score = 37 |
| | | | Gastroesophageal Reflux | 10 mg, Aspirin, | (May 9, 2018) |
| | | | Disease (GERD), Vitamin | Calcium 150 mg, | |
| | | | D Deficiency, | CoQ10 400 mg, | |
| | | | Hypertension (HTN) | Aciphex 20 mg, | |
| | | | | Zenpep 3000 iu- | |
| | | | | 10,000 iu, | |
| | | | | Ipratropium | |
| | | | | Bromide 0.03%, | |
| | | | | Rosuvastatin | |
| | | | | Calcium 5 mg | |
| 71 | Male | Caucasian | Mild Cognitive Impairment | Epipen, | ARIC |
| | | | (MCI), Dyslipidemia, | Metoprolol | MRI |
| | | | Valvular Heart Disease, | Succinate ER | Cognitive |
| | | | Hypertension (HTN), | 50 mg, Zyrtec, | Function |
| | | | Hyperlipidemia (HLD), | Montelukast, | Score = 24 |
| | | | Aortic Aneurysm, | Pepcid, Tramadol | (May 10, 2018) |
| | | | Ulcerative Colitis (UC) | 50 mg, Diazepam | |
| | | | | 5 mg, Metamucil | |
| | | | | 48.57%, Aspirin | |
| | | | | 81 mg, Plavix | |
| | | | | 75 mg, Nexium | |
| | | | | 40 mg, Lipitor | |
| | | | | 10 mg, Asacol | |
| | | | | 800 mg | |
| 74 | Female | Caucasian | Mild Cognitive Impairment | Levothyroxine | ARIC |
| | | | (MCI), Asthma, Chronic | 75 mg, Metformin | MRI |
| | | | Obstructive Pulmonary | 500 mg, Losartan | Cognitive |
| | | | Disease (COPD), Type 2 | 10 mg, Symbicort, | Function |
| | | | Diabetes, | Proventil, | Score = 30 |
| | | | Hypercholesterolemia, | Calcium, Vitamin | (May 11, 2018) |
| | | | Congestive Heart Failure | D3, Zyrtec 10 mg | |
| | | | (CHF), Hypothyroidism | | |
| 75 | Male | Caucasian | Mild Cognitive Impairment | Patanase 0.6%, | ARIC |
| | | | (MCI), Neuropathy, | Timolol | MRI |
| | | | Benign Prostatic | Hemihydrate, | Cognitive |
| | | | Hyperplasia (BPH), | Latanoprost | Function |
| | | | Hypertension (HTN), | 0.005%, | Score = |
| | | | Rheumatoid Arthritis (RA), | Methotrexate, | Refused |
| | | | Sjogren's Syndrome, | Prednisone, Folic | (May 18, 2018) |
| | | | Glaucoma, Allergic | Acid, Vitamin D, | |
| | | | Rhinitis, Nasal | Finasteride 5 mg, | |
| | | | Obstruction, Type 2 | Tamsulosin HCL | |
| | | | Diabetes | 0.4 mg, | |
| | | | | Gabapentin | |
| | | | | 100 mg, Vicodin | |
| | | | | 5 mg-300 mg, | |
| | | | | Losartan | |
| | | | | Potassium 50 mg, | |
| | | | | Pilocarpine HCL | |
| | | | | 5 mg, Calcium | |
| | | | | 600 mg, Vitamin | |
| | | | | B12 100 mcg, | |
| | | | | Docusate Sodium | |
| | | | | 100 mg, Miralax, | |
| | | | | Polyethylene | |
| | | | | Glycol, Ventolin | |
| | | | | HFA 90 mcg, | |
| | | | | Azithromycin | |
| | | | | 250 mg, Lasix | |
| | | | | 20 mg, Levaquin | |
| | | | | 500 mg, Evoxac | |
| | | | | 30 mg | |
| 75 | Male | Caucasian | Mild Cognitive Impairment | Levothyroxine | ARIC |
| | | | (MCI), | Sodium 25 mcg, | MRI |
| | | | Hypercholesterolemia, | Crestor 40 mg | Cognitive |
| | | | Thyroid Disease | | Function |
| | | | | | Score = 35 |
| | | | | | (May 24, 2018) |
| 75 | Male | Caucasian | Mild Cognitive Impairment | Pravachol 40 mg, | ARIC |
| | | | (MCI), | Ocuvite, Viagra | MRI |
| | | | Hypercholesterolemia, Age | 50 mg | Cognitive |
| | | | Related Macular | | Function |
| | | | Degeneration (AMD), | | Score = 31 |
| | | | Erectile Dysfunction (ED) | | (Feb 19, 2018) |
| 75 | Female | Caucasian | Mild Cognitive Impairment | Metformin | ARIC |
| | | | (MCI), Type 2 Diabetes, | 500 mg, | MRI |
| | | | Hypertension (HTN), | Atorvastatin | Cognitive |
| | | | Dyslipidemia, Chronic | Calcium 20 mg, | Function |
| | | | Kidney Disease (CKD), | Cozaar 100 mg, | Score = 42 |
| | | | Pulmonary Nodule, | Aspirin 81 mg, | (May 1, 2018) |
| | | | Hyperlipidemia (HLD) | Hydrochlorothiazide | |
| | | | | 25 mg, Lipitor | |
| | | | | 20 mg | |
| 76 | Female | Caucasian | Mild Cognitive Impairment | Donepezil HCL | ARIC |
| | | | (MCI), Hyperlipidemia | 10 mg, | MRI |
| | | | (HLD), Hypertension | Levothyroxine | Cognitive |
| | | | (HTN), Gastroesophageal | Sodium 50 mcg, | Function |
| | | | Reflux Disease (GERD), | Tramadol HCL | Score = 7 |
| | | | Anxiety, Hypothyroidism | 50 mg, | (May 4, 2018) |
| | | | | Atorvastatin | |
| | | | | Calcium 20 mg, | |
| | | | | Omeprazole | |
| | | | | 20 mg, Losartan | |
| | | | | Potassium 50 mg, | |
| | | | | Aricept 10 mg, | |
| | | | | Paxil 20 mg, | |
| | | | | Namenda 10 mg | |
| 76 | Male | Caucasian | Mild Cognitive Impairment | Novolog, Lantus | ARIC |
| | | | (MCI), Hypertension | 100 iu/mL, | MRI |
| | | | (HTN), Type 2 Diabetes, | Metoprolol | Cognitive |
| | | | Peripheral Polyneuropathy, | Succinate 25 mg, | Function |
| | | | Benign Prostatic | Tacrolimus, | Score = 28 |
| | | | Hyperplasia (BPH) | Terazosin HCL | (May 15, 2018) |
| | | | | 10 mg, CellCept | |
| | | | | 250 mg, Aspirin | |
| | | | | 81 mg, | |
| | | | | Allopurinol | |
| | | | | 150 mg, | |
| | | | | Atorvastatin | |
| | | | | Calcium 10 mg, | |
| | | | | Losartan | |
| | | | | Potassium 100 mg | |
| 67 | Female | Caucasian | Mild Cognitive Impairment | Crestor 40 mg, | ARIC |
| | | | (MCI), Asthma, | Omeprazole | MRI |
| | | | Hypercholesterolemia | 20 mg | Cognitive |
| | | | | | Function |
| | | | | | Score = 40 |
| | | | | | (May 7, 2018) |
| 56 | Female | Caucasian/ | Mild Cognitive Impairment | Daily Vitamins, | ARIC |
| | | Japanese | (MCI) | Aspirin 81 mg | MRI |
| | | | | | Cognitive |
| | | | | | Function |
| | | | | | Score = 41 |
| | | | | | (May 8, 2018) |
| 58 | Female | Caucasian | Mild Cognitive Impairment | Simvastatin | ARIC |
| | | | (MCI), Hyperlipidemia | 20 mg, Caltrate | MRI |
| | | | (HLD) | 600 mg-Vitamin | Cognitive |
| | | | | D 800 iu, Vitamin | Function |
| | | | | D 2000 iu, | Score = 42 |
| | | | | Ibuprofen 800 mg, | (May 8, 2018) |
| | | | | Prolia 60 mg/mL | |
| 75 | Female | Caucasian | Mild Cognitive Impairment | Crestor 10 mg, | ARIC |
| | | | (MCI), AF, Dyslipidemia, | Armour Thyroid | MRI |
| | | | Hypertension (HTN), | 60 mg, Ramipril | Cognitive |
| | | | Hypothyroidism | 5 mg, | Function |
| | | | | Hydrochlorothiazide | Score = 31 |
| | | | | 25 mg, | (May 11, 2018) |
| | | | | Promethium | |
| | | | | 200 mg, | |
| | | | | Augmentin | |
| | | | | 875 mg-125 mg, | |
| | | | | Rosuvastatin | |
| | | | | Calcium 10 mg | |
| 84 | Female | Caucasian | Mild Cognitive Impairment | Cipro 500 mg, | ARIC |
| | | | (MCI), Venous | Ibuprofen 800 mg, | MRI |
| | | | Insufficiency, | Xanax 0.5 mg, | Cognitive |
| | | | Hyperlipidemia (HLD), | Fluconazole | Function |
| | | | Hypothyroidism, | 150 mg, | Score = 19 |
| | | | Parkinson's Disease (PD), | Carbidopa- | (May 11, 2018) |
| | | | Mitral Valve Prolapse | Levodopa 25 mg- | |
| | | | (MVP), Anxiety | 100 mg, | |
| | | | | Potassium | |
| | | | | Chloride 20 mEq, | |
| | | | | Simvastatin | |
| | | | | 20 mg, | |
| | | | | Furosemide | |
| | | | | 40 mg, | |
| | | | | Levothyroxine | |
| | | | | Sodium 75 mcg, | |
| | | | | Atenolol 25 mg, | |
| | | | | Lasix, Aspirin | |
| | | | | 81 mg, | |
| | | | | Acetaminophen | |
| | | | | 500 mg | |
| 88 | N/A | Caucasian | Mild Cognitive Impairment | Cozaar 100 mg, | ARIC |
| | | | (MCI), Hyperlipidemia | Crestor 10 mg, | MRI |
| | | | (HLD), Peripheral | Aspirin, Prilosec | Cognitive |
| | | | Vascular Disease, | 20 mg, | Function |
| | | | Hypertension (HTN), | Amlodipine | Score = 8 |
| | | | Hyperlipidemia, Mild | Besylate 5 mg, D3 | (May 22, 2018) |
| | | | Intermittent Asthma, | 1000 iu, Vitamin | |
| | | | Hypercholesterolemia, | C 100 mg, Multi | |
| | | | Type 2 Diabetes | for Him, | |
| | | | | Omeprazole | |
| | | | | 20 mg | |
| 71 | Male | Caucasian | Mild Cognitive Impairment | Aspirin 81 mg, | ARIC |
| | | | (MCI), Hypertension | Brimonidine | MRI |
| | | | (HTN), | 0.15%, Cialis | Cognitive |
| | | | Hypercholesterolemia, | 20 mg, | Function |
| | | | Chronic Kidney Disease | Dexamethasone | Score = 44 |
| | | | (CKD), Palsy of Conjugate | 4 mg/ml, | (May 24, 2018) |
| | | | Gaze, Short Term Memory, | Donepezil 5 mg, | |
| | | | Hyperlipidemia, Cervical | Fexofenadine | |
| | | | Spondylosis, Basal Cell | 180 mg, | |
| | | | Cancer (BCC), Complex | Lamotrigine | |
| | | | Partial Epileptic Seizure, | 200 mg, Lisinopril | |
| | | | Chronic Tremor, | 5 mg, Meloxicam | |
| | | | Lumbosacral Radiculitis, | 15 mg, | |
| | | | Allergic Rhinitis, Lumbar | Pramipexole | |
| | | | Arthritis, Arthritis, | 0.25 mg, | |
| | | | Bilateral Hearing Loss | Simvastatin | |
| | | | | 40 mg, Virtussin | |
| | | | | 10 mg-100 mg/5 ml | |
| 86 | Male | Caucasian | Mild Cognitive Impairment | Amlodipine 5 mg, | ARIC |
| | | | (MCI), Hypertensive Heart | Glimepiride 1 mg, | MRI |
| | | | and Renal Disease with | Nitroglycerin | Cognitive |
| | | | Congestive Heart Failure, | 0.2 mg, Potassium | Function |
| | | | Cyst and Pseudocyst of | Chloride 20 meq, | Score = 48 |
| | | | Pancreas, Benign Prostatic | Warfarin 2 mg | (May 17, 2018) |
| | | | Hyperplasia (BPH), Type 2 | | |
| | | | Diabetes, Chronic Kidney | | |
| | | | Disease (CKD), | | |
| | | | Hypokalemia, Chronic | | |
| | | | Systolic Heart Disease, | | |
| | | | Mitral Valve Prolapse | | |
| | | | (MVP), Atrial Fibrillation | | |
| | | | (AF), Hyperlipidemia, | | |
| | | | Sensorineural Hearing | | |
| | | | Loss, Left Bundle Branch | | |
| | | | Block, Pulmonary | | |
| | | | Hypertension (HTN), | | |
| | | | Hyperparathyroidism | | |
| 91 | Female | Caucasian | Mild Cognitive Impairment | Amlodipine | ARIC |
| | | | (MCI), Type 2 Diabetes, | Besylate 5 mg, | MRI |
| | | | Hypertension (HTN), | Atorvastatin | Cognitive |
| | | | Hypercholesterolemia, | Calcium 40 mg, | Function |
| | | | Benign Prostate | Coumadin, Plavix | Score = 31 |
| | | | Hyperplasia (BPH), | 75 mg, Toprol | (Mar. 13, 2018) |
| | | | Abdominal Aortic | 50 mg | |
| | | | Aneursym, Atrial | | |
| | | | Fibrillation (AF) | | |
| 88 | Male | Caucasian | Mild Cognitive Impairment | Trintellix 10 mg, | ARIC |
| | | | (MCI), | Aripiprazole | MRI |
| | | | Hypercholesterolemia, | 2.5 mg, | Cognitive |
| | | | Melanoma, Depression, | Rosuvastatin | Function |
| | | | Squamous Cell Carcinoma, | 20 mg, Modafinil | Score = 16 |
| | | | GERD, Hemorrhoids, TIA | 200 mg, | (Feb. 21, 2018) |
| | | | | Amphetamine | |
| | | | | 20 mg, Namenda | |
| | | | | 28 mg, | |
| | | | | Esomeprazole | |
| | | | | 20 mg, Lutein | |
| | | | | 5 mg, Vitamin D3 | |
| | | | | 1000 iu, Aspirin | |
| | | | | 81 mg, Vitamin | |
| | | | | B12 | |

TABLE 20B
Demographics of participants with AD

| Age | Gender | Race | Diagnosis | Medications |
|---|---|---|---|---|
| Male | 82 | Caucasian | Alzheimer's Disease | Aricept 10 mg, B Complex |
| | | | (AD), Gastroesophageal | 100 0.4 mg, Doxazosin 8 mg, |
| | | | Reflux Disease (GERD), | Finasteride 5 mg, Melatonin |
| | | | Benign Prostatic | 10 mg, Multivitamin 9 mg, |
| | | | Hyperplasia (BPH), Sleep | Omeprazole 20 mg, |
| | | | Apnea, Malignant Basal | Sertraline, Simvastatin 80 mg, |
| | | | Cell Neoplasm of Skin, | Vitamin D3 2000 iu, Voltaren |
| | | | Depression, Dermatitis, | 1% |
| | | | Osteoarthritis (OA), | |
| | | | Thrombocytopenia | |
| Male | 87 | Caucasian | Alzheimer's Disease | Cartia XT 120 mg, Prilosec |
| | | | (AD), Hypertension | 20 mg, Namenda 28XL, |
| | | | (HTN), Hyperlipidemia, | Exelon Patch 9.5 mg, Paxil |
| | | | Dementia | 20 mg |
| Female | 84 | Caucasian | Hypertension (HTN), | Cerefolin NAC 6 mg-200 mg, |
| | | | Vitamin D Deficiency, | Clopidogrel Bisulfate 75 mg, |
| | | | Hyperlipidemia (HLD), | Multivitamin, Galantamine |
| | | | Skin Cancer, Anemia, | Hydrobromide ER 16 mg, |
| | | | Alzheimer's Disease (AD) | Memantine HCL 10 mg, |
| | | | | Vitamin D3, Zolpidem |
| | | | | Tartrate 5 mg, Iron 325 mg, |
| | | | | Remeron 15 mg, Plavix 75 mg |
| Female | 76 | Caucasian | Hyperlipidemia (HLD), | Donepezil HCL 10 mg, |
| | | | Hypertension (HTN), | Levothyroxine Sodium |
| | | | Gastroesophageal Reflux | 50 mcg, Tramadol HCL |
| | | | Disease (GERD), | 50 mg, Atorvastatin Calcium |
| | | | Anxiety, Alzheimer's | 20 mg, Omeprazole 20 mg, |
| | | | Disease (AD), | Losartan Potassium 50 mg, |
| | | | Hypothyroidism | Aricept 10 mg, Paxil 20 mg, |
| | | | | Namenda 10 mg |
| Male | 47 | Caucasian | Alzheimer's Disease (AD) | Donepezil 10 mg |
| Male | 67 | African | Alzheimer's Disease (AD) | Rivastigmine 3 mg, |
| | | | | Multivitamin |
| Male | 61 | Caucasian | Alzheimer's Disease | Atorvastatin 40 mg, |
| | | | (AD), Type 2 Diabetes, | Gabapentin 300 mg, Aspirin |
| | | | Hypertension (HTN), | 81 mg, Razadyne 16 mg, |
| | | | Hypercholesterolemia | Metformin 500 mg |
| Female | 60 | African | Alzheimer's Disease | Clonidine 0.3 mg, Ambien, |
| | | | (AD), Hypertension | Quetiapine 300 mg |
| | | | (HTN) | |
| Male | 47 | N/A | Alzheimer's Disease | Gabapentin 300 mg |
| | | | (AD), Asthma, Anxiety | |
| Male | 60 | African | Alzheimer's Disease | Donepezil, Metformin, |
| | | | (AD), Type 2 Diabetes | Humalog |
| Male | 74 | Caucasian | Alzheimer's Disease | Aspirin 80 mg, Plavix 75 mg, |
| | | | (AD), Hypertension | Lisinopril 25 mg, Simvastatin |
| | | | (HTN), | 10 mg, Digoxin 30 mg, |
| | | | Hypercholesterolemia | Metoprolol 50 mg, Razadyne |
| | | | | 24 mg |
| Male | 50 | African | Alzheimer's Disease | Keppra 700 mg/50 mg, Exelon |
| | | | (AD), Seizures | Patch |
| Male | 67 | African | Alzheimer's Disease | Aspirin 81 mg, Lisinopril |
| | | | (AD), Hypertension | 5 mg, Metoprolol Succinate |
| | | | (HTN) | 500 mg |
| Male | 59 | Mixed | Alzheimer's Disease | Metoprolol 50 mg, |
| | | Race | (AD), Type 2 Diabetes, | Amlodipine/Benazepril |
| | | | Hypertension (HTN), | 10 mg/40 mg, Seroquel 50 mg, |
| | | | Anxiety | Aricept 23 mg, Creon |
| | | | | 36000 iu, Gabapentin 600 mg, |
| | | | | Prandin 2 mg, Metformin |
| | | | | 1000 mg |
| Male | 54 | African | Alzheimer's Disease | Donepezil 10 mg, |
| | | | (AD), HTN | Multivitamin, Atenolol 50 mg |
| Female | 58 | N/A | Alzheimer's Disease | Combivent 103 mcg, |
| | | | (AD), Asthma, | Symbicort 160 mcg, |
| | | | Hypertension (HTN), | Budesonide 0.5 mg, Singulair |
| | | | Hypercholesterolemia, | 10 mg, Prandin 2 mg, |
| | | | Rheumatoid Arthritis | Metoprolol 50 mg, Lotrel |
| | | | (RA), Type 2 Diabetes | 20 mg, Janumet 1000 mg, |
| | | | | Donepezil 10 mg, Maxzide |
| | | | | 37.5 mg |
| Male | 75 | Caucasian | Osteomyelitis, Type 2 | Hydrochlorothiazide 25 mg, |
| | | | Diabetes, Chronic Kidney | H7umalog 100 iu/mL, Lantus |
| | | | Disease (CKD), | 100 iu/mL, Metformin HCL |
| | | | Dyslipidemia, | 1000 mg, Testosterone |
| | | | Hypertension (HTN), | Cypionate 200 mg/mL, |
| | | | Erectile Dysfunction | Amlodipine Besylate 10 mg, |
| | | | (ED), Atherosclerosis, | Ventolin HFA 108 mcg, |
| | | | Alzheimer's Disease (AD) | Carvedilol 25 mg, Lipitor |
| | | | | 20 mg, Benazepril HCL |
| | | | | 40 mg, Azithromycin 250 mg, |
| | | | | Proair 108 mcg |
| Female | 75 | Caucasian | Alzheimer's Disease | Aricept 10 mg, Namenda |
| | | | (AD), Allergy (Seasonal) | 10 mg, Calcitrate 200 mg, |
| | | | | Centrum Silver, Cetirizine |
| | | | | 10 mg, Folic Acid 400 mcg, |
| | | | | Magnesium 250 mg |
| Female | 73 | Caucasian | Alzheimer's Disease | Vitamin D6, Folic Acid, |
| | | | (AD), Type 2 Diabetes, | Warfarin 5 mg, Losartan |
| | | | Hypercholesterolemia, | 50 mh-12.5 mg, Metformin |
| | | | Coronary Artery Disease | 500 mg, Aricept 10 mg |
| | | | (CAD) | |
| Male | 55 | N/A | Alzheimer's Disease | Losartan 12.5 mg, Meloxicam |
| | | | (AD), Hypertension | 15 mg, Norvasc 10 mg |
| | | | (HTN), Bilateral Carpal | |
| | | | Tunnel | |
| Male | 84 | Caucasian | Hypertension (HTN), | Metoprolol 25 mg, |
| | | | Hypercholesterolemia, | Atorvastatin 40 mg, Aspirin |
| | | | Alzheimer's Disease (AD) | 81 mg, Theragran |
| Male | 51 | African | Alzheimer's Disease | Hydrochlorothiazide 25 mg, |
| | | | (AD), Hypertension | Razadyne 16 mg |
| | | | (HTN), | |
| | | | Hypercholesterolemia | |
| Male | 64 | N/A | Alzheimer's Disease | Exelon 6 mg, Metformin |
| | | | (AD), Hypertension | 500 mg, Atorvastatin 40 mcg, |
| | | | (HTN), | Ramipril 10 mg, Lantus |
| | | | Hypercholesterolemia, | Solostar 100 iu |
| | | | Type 2 Diabetes | |
| Female | 84 | Caucasian | Hypertension (HTN), | Simvastatin 20 mg, Potassium |
| | | | Hallucinations, | Chloride 10 mEq, Amlodipine |
| | | | Psychoses, Cellulitis, | Besylate 2.5 mg, Dutasteride |
| | | | Dementia, Mitral Valve | 0.5 mg, Losartan Potassium |
| | | | Prolapse (MVP), | 100 mg, Aspirin 81 mg, |
| | | | Hyperlipidemia (HLD), | Furosemide 20 mg, Potassium |
| | | | Alzheimer's Disease (AD) | Chloride 10 mEq, Avodart |
| | | | | 0.4 mg, Amlodipine Besylate |
| | | | | 2.5 mg, Ramipril 10 mg |
| Female | 62 | Caucasian | Sporadic Alzheimer's | Topamax 150 mg, Vesicare |
| | | | Disease (AD), Asthma | 5 mg, Prozac 60 mg, Levoxyl |
| | | | | 75 mg, Xarelto 20 mg, |
| | | | | Hydrocodone- |
| | | | | Acetaminophen 5 mg-325 mg, |
| | | | | Butran Patch 15 mg, |
| | | | | Gabapentin 600 mg, Celebrex |
| | | | | 200 mg, Breo 100 mg, ProAir, |
| | | | | Bentyl 20 mg, Pantoprazole |
| | | | | 40 mg |
| Male | 68 | Caucasian | Alzheimer's Disease | NamEnda 5 mg, Tamsulosin |
| | | | (AD), Type 2 Diabetes, | HCL 0.4 mg, Atorvastatin |
| | | | Hypertension (HTN), | 40 mg, Valsartan 320 mg, |
| | | | Hypercholesterolemia, | Zetia 10 mg, Carvedilol |
| | | | Cerebrovascular Accident | 25 mg, Aspirin 325 mg, |
| | | | (CVA), Parkinsonism, | Bupropion HCl ER 200 mg, |
| | | | Peripheral Neuropathy, | Venlafaxine ER 150 mg, |
| | | | Hypothyroidism, Benign | Finasteride 5 mg, Synthroid |
| | | | Prostatic Hyperplasia | 50 mcg, Zolpidem 10 mg, |
| | | | (BPH), Depression, | Novolog 100 iu/mL, Lantus |
| | | | Anxiety, Glaucoma, | 100 iu/mL, Latanoprost |
| | | | Hernia | 0.005%, Azelastine 0.15%, |
| | | | | Glucagon 1 mg |
| Male | 72 | Caucasian | Hypertension (HTN), | Omega 3 350 mg-235 mg- |
| | | | Hypercholesterolemia, | 90 mg-597 mg, CoQ10 |
| | | | Alzheimer's Disease (AD) | 100 mg, Vitamin B Complex, |
| | | | | Aspirin 81 mg, Pravastatin |
| | | | | 20 mg, Losartan 50 mg, |
| | | | | Namenda XR 28 mg, |
| | | | | Donepezil 10 mg, |
| | | | | Crenizumab |
| Male | 79 | Caucasian | Asthma, Hypertension | Aspirin 81 mg, Amlodipine |
| | | | (HTN), | Besylate 10 mg/20 mg, |
| | | | Hypercholesterolemia, | Terazosin 2 mg, |
| | | | Basal Cell Cancer (BCC), | Hydrochlorothiazide 25 mg, |
| | | | Alzheimer's Disease (AD) | Atenolol 50 mg, |
| | | | | Multivitamin, Calcium, |
| | | | | Vitamin D, Atorvastatin |
| | | | | 40 mg |
| Female | 77 | Caucasian | Hypertension (HTN), | Vitamin D 2,000 iu, |
| | | | Allergic Rhinitis, | Omeprazole 20 mg, Tylenol |
| | | | Hematuria, Chronic | |
| | | | Kidney Disease (CKD), | |
| | | | Hypertensive | |
| | | | Nephropathy, | |
| | | | Hypercholesterolemia, | |
| | | | Menopausal, Osteopenia, | |
| | | | Gastroesophageal Reflux | |
| | | | Disease (GERD), Large | |
| | | | Hiatal Hernia, Gastritis, | |
| | | | Esophagitis, Basal Cell | |
| | | | Cancer (BCC), | |
| | | | Degenerative Joint | |
| | | | Disease, Rosacea, | |
| | | | Alzheimer's Disease | |
| | | | (AD), Obesity, Dyspepsia | |
| Male | 71 | Caucasian | Atrial Fibrillation, End | Lanthanum Carbonate |
| | | | Stage Renal Disease | 1000 mg, Midodrine 10 mg, |
| | | | (ESRD), Congestive | Sensipar 30 mg, Pantoprazole |
| | | | Heart Failure (CHF), | 40 mg, Pravastatin 40 mg, |
| | | | Coronary Artery Disease | Ventolin 90 mcg, Warfarin |
| | | | (CAD), Hyperlipidemia, | 3 mg |
| | | | Chronic Obstructive | |
| | | | Pulmonary Disease | |
| | | | (COPD), | |
| | | | Gastroesophageal Reflux | |
| | | | Disease (GERD), | |
| | | | Hyperparathyroidism, | |
| | | | Alzheimer's Disease (AD) | |
| Female | 82 | Caucasian | Type 2 Diabetes, | Digoxin 125 mcg, Potassium |
| | | | Hypothyroidism, | Chloride 20 meq, Metoprolol |
| | | | Coronary Artery Disease | Succinate 20 emq, |
| | | | (CAD), Atrial Fibrillation | Furosemide 20 mg, |
| | | | (AF), Hypertension | Levothyroxine Sodium |
| | | | (HTN), Alzheimer's | 88 mcg, Lipitor 20 mg, |
| | | | Disease (AD), | Memantine HCl 5 mg, |
| | | | Hyperlipidemia (HLD), | Lisinopril 10 mg, Xarelto |
| | | | Depression, Irritable | 15 mg, Amlodipine Besylate |
| | | | Bowel Syndrome (IBS), | 2.5 mg, Zoloft 50 mg, Aricept |
| | | | Cerebrovascular Accident | 10 mg, Metformin HCl |
| | | | (CVA), Coronary Artery | 500 mg |
| | | | Disease (CAD), Vertigo, | |
| Male | 78 | Caucasian | Chronic Kidney Disease | Humalog Mix 100 iu, Aspirin |
| | | | (CKD), Diabetic | 81 mg, Centrum Silver, L |
| | | | Nephropathy, Diabetic | Glutamine, Metoprolol |
| | | | Neuropathy, Coronary | Succinate 50 mg, Lipitor |
| | | | Artery Disease (CAD), | 20 mg, Novolog, Humulin N, |
| | | | History Of Myocardial | Gabapentin 100 mg, |
| | | | Infarction, | Alprazolam 0.5 mg, |
| | | | Hyperlipidemia (HLD), | Fluticasone Propionate |
| | | | Type 1 Diabetes, | Cream, Citalopram |
| | | | Depression, Age Related | Hydrobromide 20 mg, Cartia |
| | | | Macular Degeneration | XT 120 mg, Aricept 5 mg, |
| | | | (AMD), Alzheimer's | Citalopram Hydrobromide |
| | | | Disease (AD), Dementia, | 20 mg |
| | | | Acute Renal Failure | |
| | | | (ARF) | |

### Simple Plex Assay

Analysis of inflammasome protein (caspase-1, ASC, IL-1β and IL-18) and NfL protein concentration in serum samples from MCI, AD, and age-matched controls were performed using the Ella System (Protein System) as described in^{2, 3}.

MSD Multi-Spot sAPPα/sAPPβ Assay

Protein levels of soluble APPα and β (sAPPα/sAPPβ) were measured using the MSD 96-Well Multi-Spot sAPPα/sAPPβ Assay according to manufacturer instructions and read on the MESO Quickplex SQ 120 instrument. Briefly, the plate was coated with Blocker A solution prior to adding the samples and calibrators followed by addition of the detection antibody and ultimately reading of the plate in the MESO Quickplex SQ 120 instrument.

### Biomarker Analyses

Data obtained by the Simple Plex assay were analyzed with Prism 7 software (GraphPad). First, outliers were removed and receiver operating characteristics (ROC) were calculated, thus obtaining a 95% confidence interval, a standard deviation and a p-value. P-value of significance was considered at less than 0.05. A cut-off point was then obtained for a range of different specificities and sensitivities and their respective likelihood ratio as well as positive (PPV) and negative predictive values (NPV) and accuracy^{2, 3}.

### Statistical Analyses

Normality was tested by the Shapiro-Wilk normality test, and statistical difference between groups was tested by the Mann-Whitney test for non-normally distributed data and by a student t-test for normally distributed data when comparing two groups. When comparison was done between three groups, an ANOVA followed by a Kruskal-Wallis test was carried. P-value of significance was considered at less than 0.05. In addition, clustering was carried using hierarchical clustering and Gaussian Mixture Modelling using RStudio software with the following libraries: cluster, caret, factorextra, magrittr, ggplot2 and mclust.

### Linear Regression Analyses

Regression analysis between analytes was run using RStudio/RMarkdown with the following libraries: MASS, dplyr, ggplot, car and broom. Data were first plotted and then a linear model was fit between ASC and IL-18 and between sAPPα and sAPPβ. Following fitting of the different models, a Box-Cox transformation was carried on each data set and then the data were transformed accordingly. P-value of significance was considered at less than 0.05. Suitability of the models were then evaluated by residual analysis.

### Results

ASC and IL-18 are Elevated in the Serum of Patients with MCI and AD

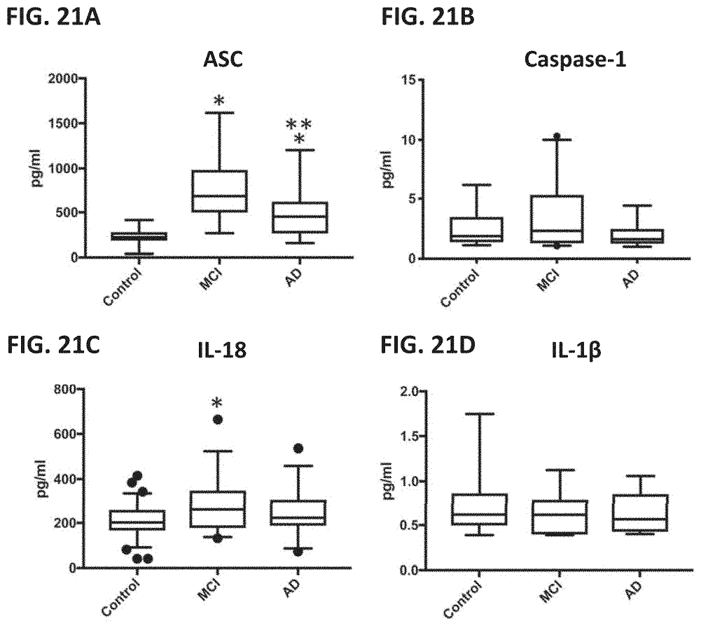

Serum samples from patients with MCI, patients with AD, and aged-matched healthy donors were analyzed for the protein expression levels of ASC (FIG. 21A), caspase-1 (FIG. 21B), IL-18 (FIG. 21C) and IL-1β (FIG. 21D). Here, the protein levels of ASC and IL-18 were found to be significantly higher in the MCI group when compared to the control group; thus suggesting an involvement of ASC and IL-18 in the pathology of MCI. Surprisingly, the protein levels of ASC were higher in MCI patients than in AD patients.

### ASC is a Promising Serum Biomarker of MCI and AD

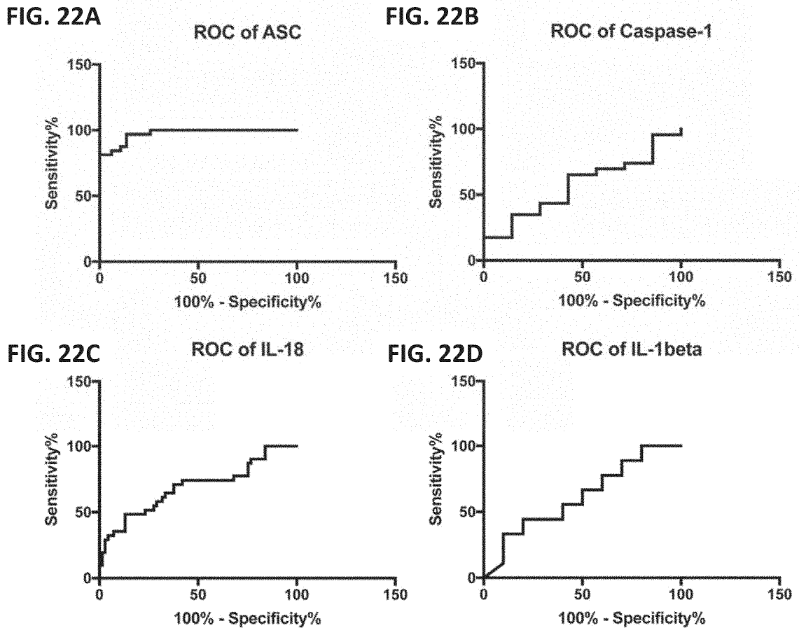

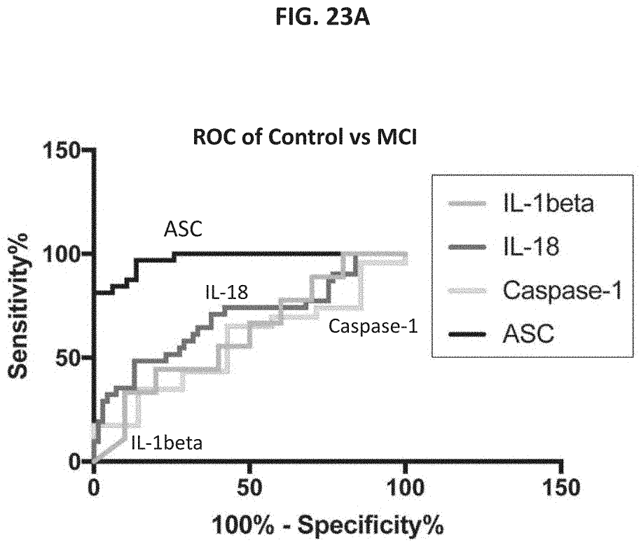

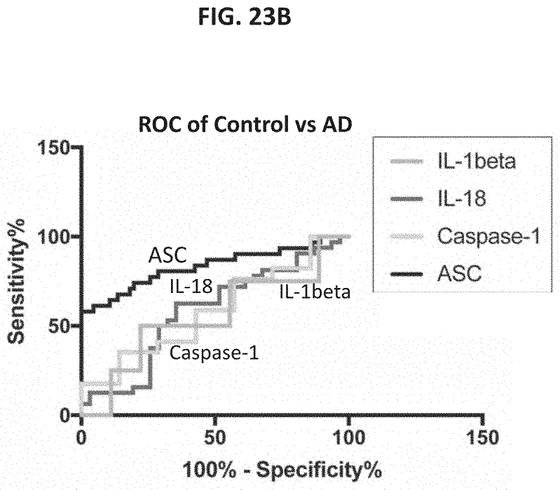

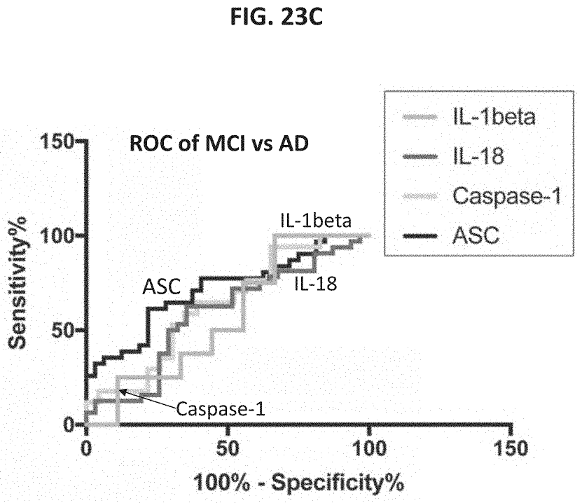

To determine if inflammasome signaling proteins can be used as biomarkers of MCI and AD, the area under the curve (AUC) was determined for caspase-1, ASC, IL-1β, and IL-18 for MCI, AD, and control groups. The AUC for caspase-1, ASC, IL-13, and IL-18 from the Control group versus the MCI group is shown in FIGS. 22A-D, respectively. FIG. 23A shows all of the ROC curves from FIG. 22A-22D superimposed onto each other. FIG. 23B shows the ROC curves for caspase-1, ASC, IL-1β, and IL-18 from the control group versus the AD group superimposed onto each other. FIG. 23C shows the ROC curves for caspase-1, ASC, IL-1β, and IL-18 from the MCI group versus the AD group superimposed onto each other. When comparing MCI patients to control, ASC presented the highest AUC of 0.974 (p<0.0001), compared to 0.9687 for sAPPα, 0.09068 for sAPPβ, 0.7734 for NFL, followed by IL-18 with an AUC of 0.6896 (p=0.0025) (Table 21A). The ROC results for inflammasome signaling proteins in serum in patients with AD versus control patients and in MCI versus AD patients is shown in Table 21B and Table 21C, respectively. When comparing MCI patients to AD patients, ASC had an AUC of 0.7157 compared to 0.6531 for sAPPα, 0.5247 for sAPPβ, and 0.5569 for NFL. Thus, ASC is a reliable serum biomarker for differentiating MCI vs AD.

The cut-off point for ASC in serum for Control samples versus MCI samples was 264.9 pg/ml with 100% sensitivity and 74% specificity (see Tables 22A and 23); whereas IL-18 had a cut-off point of 213.9 pg/ml with 74% sensitivity and 58% specificity (Tables 22A and 25). The cut-off point analyses for inflammasome signaling proteins in serum in control patients vs patients with AD is found in Table 22B, and the cut-off point analyses for inflammasome signaling proteins in serum in MCI patients vs AD patients is found in Table 22C.

In addition to Table 22A, the cut-off points and sensitivity/specificity data for caspase-1 and IL-1beta can be found in Tables 24 and 26, respectively.

TABLE 21A
ROC analysis results for inflammasome signaling proteins in
serum in patients with MCI vs Control.

| | | STD. | | |
|---|---|---|---|---|
| BIOMARKER | AREA | ERROR | 95% C.I. | P VALUE |
| ASC | 0.974 | 0.01301 | 0.9485 to | <0.0001 |
| | | | 0.9995 | |
| Caspase-1 | 0.5714 | 0.1174 | 0.3413 to | 0.5728 |
| | | | 0.8016 | |
| IL-18 | 0.6896 | 0.06086 | 0.5703 to | 0.0025 |
| | | | 0.8089 | |
| IL-1beta | 0.6167 | 0.1317 | 0.3585 to | 0.3913 |
| | | | 0.8749 | |
| sAPPalpha | 0.9687 | 0.0216 | 0.9263 to | <0.0001 |
| | | | 1.011 | |
| sAPPbeta | 0.9068 | 0.03784 | 0.8327 to | <0.0001 |
| | | | 0.981 | |
| NfL | 0.7734 | 0.05821 | 0.6594 to | 0.0002 |
| | | | 0.8875 | |

TABLE 21B
ROC analysis results for inflammasome signaling proteins in serum in
patients with AD vs Control.

| | | STD. | | |
|---|---|---|---|---|
| BIOMARKER | AREA | ERROR | 95% C.I. | P VALUE |
| ASC | 0.8328 | 0.05053 | 0.7338 to 0.9319 | <0.0001 |
| Caspase-1 | 0.6476 | 0.1290 | 0.3948 to 0.9005 | 0.2746 |
| IL-18 | 0.6105 | 0.06124 | 0.4905 to 0.7305 | 0.0749 |
| IL-1beta | 0.5556 | 0.1467 | 0.268 to 0.8431 | 0.7003 |
| sAPPalpha | 0.9563 | 0.02490 | 0.9074 to 1.005 | <0.0001 |
| sAPPbeta | 0.9185 | 0.03592 | 0.8481 to 0.9889 | <0.0001 |
| NfL | 0.7165 | 0.06817 | 0.5829 to 0.8501 | 0.0040 |

TABLE 21C
ROC analysis results for inflammasome signaling proteins
in serum in patients with MCI vs AD.

| | | STD. | | |
|---|---|---|---|---|
| BIOMARKER | AREA | ERROR | 95% C.I. | P VALUE |
| ASC | 0.7157 | 0.06472 | 0.5889 to 0.8426 | 0.0033 |
| Caspase-1 | 0.6812 | 0.08630 | 0.5120 to 0.8503 | 0.0620 |
| IL-18 | 0.5847 | 0.07332 | 0.441 to 0.7284 | 0.2482 |
| IL-1beta | 0.5694 | 0.1445 | 0.2862 to 0.8527 | 0.6304 |
| SAPPalpha | 0.6351 | 0.07146 | 0.4950 to 0.7752 | 0.0654 |
| sAPPbeta | 0.5247 | 0.07514 | 0.3774 to 0.6720 | 0.7401 |
| NfL | 0.5569 | 0.07502 | 0.4099 to 0.7040 | 0.4498 |

TABLE 22A
Cut-off point analyses for inflammasome signaling
proteins in serum in Control vs patients with MCI.

| | Cut-off | | | | | | |
|---|---|---|---|---|---|---|---|
| | point | Sensitivity | Specificity | PPV | NPV | Likelihood | Accuracy |
| Biomarker | (pg/ml) | (%) | (%) | (%) | (%) | Ratio | (%) |
| ASC | >264.9 | 100 | 74 | 65 | 100 | 3.882 | 83 |
| Caspase-1 | >1.753 | 65 | 43 | 79 | 27 | 1.141 | 60 |
| IL-18 | >213.9 | 74 | 58 | 44 | 83 | 1.765 | 63 |
| IL-1beta | <0.684 | 67 | 50 | 55 | 63 | 1.333 | 58 |
| sAPPalpha | >1.39 | 97 | 74 | 81 | 95 | 3.763 | 86 |
| | (ng/ml) | | | | | | |
| sAPPbeta | >0.2639 | 90 | 78 | 78 | 90 | 4.065 | 84 |
| | (ng/ml) | | | | | | |
| NfL | >24.15 | 72 | 75 | 71 | 75 | 2.875 | 74 |

TABLE 22B
Cut-off point analyses for inflammasome signaling
proteins in serum in Control vs patients with AD.

| | Cut-off | | | | | | |
|---|---|---|---|---|---|---|---|
| | point | Sensitivity | Specificity | PPV | NPV | Likelihood | Accuracy |
| Biomarker | (pg/ml) | (%) | (%) | (%) | (%) | Ratio | (%) |
| ASC | >258.7 | 81 | 71 | 57 | 89 | 2.801 | 74 |
| Caspase-1 | <1.781 | 67 | 57 | 79 | 41 | 1.556 | 64 |
| IL-18 | >196.5 | 72 | 42 | 37 | 76 | 1.24 | 51 |
| IL-1beta | <0.6935 | 75 | 44 | 55 | 67 | 1.35 | 59 |
| sAPPalpha | >2.573 | 91 | 91 | 92 | 90 | 10.57 | 91 |
| | (ng/ml) | | | | | | |
| sAPPbeta | >0.2906 | 83 | 81 | 80 | 85 | 4.5 | 82 |
| | (ng/ml) | | | | | | |
| NfL | >21.48 | 64 | 56 | 56 | 64 | 1.469 | 60 |

TABLE 22C
Cut-off point analyses for inflammasome signaling proteins
in serum in patients with MCI vs patients with AD.

| | Cut-off | | | | | | |
|---|---|---|---|---|---|---|---|
| | point | Sensitivity | Specificity | PPV | NPV | Likelihood | Accuracy |
| Biomarker | (pg/ml) | (%) | (%) | (%) | (%) | Ratio | (%) |
| ASC | <560.0 | 71 | 63 | 65 | 69 | 1.892 | 67 |
| Caspase-1 | <1.945 | 73 | 61 | 58 | 76 | 1.874 | 66 |
| IL-18 | >290.3 | 72 | 48 | 59 | 63 | 1.393 | 60 |
| IL-1beta | >0.462 | 75 | 44 | 55 | 67 | 1.35 | 59 |
| sAPPalpha | <8.846 | 72 | 55 | 64 | 63 | 1.592 | 64 |
| | (ng/ml) | | | | | | |
| sAPPbeta | >0.6364 | 60 | 45 | 49 | 56 | 1.094 | 52 |
| | ng/ml) | | | | | | |
| NfL | <33.92 | 71 | 44 | 53 | 64 | 1.27 | 57 |

TABLE 23
Cut-off point analyses for ASC in serum.

| | Sensi- | | Speci- | | Like- |
|---|---|---|---|---|---|
| Cutoff | tivity | | ficity | 95% | lihood |
| (pg/ml) | % | 95% CI | % | CI | ratio |
| >76.58 | 100 | 89.11% to 100% | 1.515 | 0.03835% to | 1.015 |
| | | | | 8.155% | |
| >127.1 | 100 | 89.11% to 100% | 3.03 | 0.3691% to | 1.031 |
| | | | | 10.52% | |
| >141.6 | 100 | 89.11% to 100% | 4.545 | 0.9474% to | 1.048 |
| | | | | 12.71% | |
| >145.8 | 100 | 89.11% to 100% | 6.061 | 1.676% to | 1.065 |
| | | | | 14.8% | |
| >148.9 | 100 | 89.11% to 100% | 7.576 | 2.506% to | 1.082 |
| | | | | 16.8% | |
| >152.9 | 100 | 89.11% to 100% | 9.091 | 3.41% to | 1.1 |
| | | | | 18.74% | |
| >158.1 | 100 | 89.11% to 100% | 10.61 | 4.372% to | 1.119 |
| | | | | 20.64% | |
| >159.9 | 100 | 89.11% to 100% | 12.12 | 5.381% to | 1.138 |
| | | | | 22.49% | |
| >164.5 | 100 | 89.11% to 100% | 13.64 | 6.43% to | 1.158 |
| | | | | 24.31% | |
| >169.3 | 100 | 89.11% to 100% | 15.15 | 7.512% to | 1.179 |
| | | | | 26.1% | |
| >171.1 | 100 | 89.11% to 100% | 16.67 | 8.625% to | 1.2 |
| | | | | 27.87% | |
| >173.4 | 100 | 89.11% to 100% | 18.18 | 9.764% to | 1.222 |
| | | | | 29.61% | |
| >177.1 | 100 | 89.11% to 100% | 19.7 | 10.93% to | 1.245 |
| | | | | 31.32% | |
| >180.6 | 100 | 89.11% to 100% | 21.21 | 12.11% to | 1.269 |
| | | | | 33.02% | |
| >182 | 100 | 89.11% to 100% | 22.73 | 13.31% to | 1.294 |
| | | | | 34.7% | |
| >183.5 | 100 | 89.11% to 100% | 24.24 | 14.54% to | 1.32 |
| | | | | 36.36% | |
| >185.5 | 100 | 89.11% to 100% | 25.76 | 15.78% to | 1.347 |
| | | | | 38.01% | |
| >188.8 | 100 | 89.11% to 100% | 27.27 | 17.03% to | 1.375 |
| | | | | 39.64% | |
| >191.5 | 100 | 89.11% to 100% | 28.79 | 18.3% to | 1.404 |
| | | | | 41.25% | |
| >193.1 | 100 | 89.11% to 100% | 30.3 | 19.59% to | 1.435 |
| | | | | 42.85% | |
| >194.6 | 100 | 89.11% to 100% | 31.82 | 20.89% to | 1.467 |
| | | | | 44.44% | |
| >196.4 | 100 | 89.11% to 100% | 33.33 | 22.2% to | 1.5 |
| | | | | 46.01% | |
| >197.6 | 100 | 89.11% to 100% | 34.85 | 23.53% to | 1.535 |
| | | | | 47.58% | |
| >198.1 | 100 | 89.11% to 100% | 36.36 | 24.87% to | 1.571 |
| | | | | 49.13% | |
| >199.7 | 100 | 89.11% to 100% | 37.88 | 26.22% to | 1.61 |
| | | | | 50.66% | |
| >201 | 100 | 89.11% to 100% | 39.39 | 27.58% to | 1.65 |
| | | | | 52.19% | |
| >203.1 | 100 | 89.11% to 100% | 40.91 | 28.95% to | 1.692 |
| | | | | 53.71% | |
| >210.3 | 100 | 89.11% to 100% | 42.42 | 30.34% to | 1.737 |
| | | | | 55.21% | |
| >216.1 | 100 | 89.11% to 100% | 43.94 | 31.74% to | 1.784 |
| | | | | 56.7% | |
| >217.9 | 100 | 89.11% to 100% | 45.45 | 33.14% to | 1.833 |
| | | | | 58.19% | |
| >219.1 | 100 | 89.11% to 100% | 46.97 | 34.56% to | 1.886 |
| | | | | 59.66% | |
| >220.4 | 100 | 89.11% to 100% | 48.48 | 35.99% to | 1.941 |
| | | | | 61.12% | |
| >223.3 | 100 | 89.11% to 100% | 50 | 37.43% to | 2 |
| | | | | 62.57% | |
| >226.3 | 100 | 89.11% to 100% | 51.52 | 38.88% to | 2.063 |
| | | | | 64.01% | |
| >229.5 | 100 | 89.11% to 100% | 53.03 | 40.34% to | 2.129 |
| | | | | 65.44% | |
| >232.3 | 100 | 89.11% to 100% | 54.55 | 41.81% to | 2.2 |
| | | | | 66.86% | |
| >233.4 | 100 | 89.11% to 100% | 56.06 | 43.3% to | 2.276 |
| | | | | 68.26% | |
| >237.3 | 100 | 89.11% to 100% | 57.58 | 44.79% to | 2.357 |
| | | | | 69.66% | |
| >241.8 | 100 | 89.11% to 100% | 59.09 | 46.29% to | 2.444 |
| | | | | 71.05% | |
| >243.9 | 100 | 89.11% to 100% | 60.61 | 47.81% to | 2.538 |
| | | | | 72.42% | |
| >247.1 | 100 | 89.11% to 100% | 62.12 | 49.34% to | 2.64 |
| | | | | 73.78% | |
| >250 | 100 | 89.11% to 100% | 63.64 | 50.87% to | 2.75 |
| | | | | 75.13% | |
| >251.6 | 100 | 89.11% to 100% | 65.15 | 52.42% to | 2.87 |
| | | | | 76.47% | |
| >252.7 | 100 | 89.11% to 100% | 66.67 | 53.99% to | 3 |
| | | | | 77.8% | |
| >254.5 | 100 | 89.11% to 100% | 68.18 | 55.56% to | 3.143 |
| | | | | 79.11% | |
| >257.2 | 100 | 89.11% to 100% | 69.7 | 57.15% to | 3.3 |
| | | | | 80.41% | |
| >259 | 100 | 89.11% to 100% | 71.21 | 58.75% to | 3.474 |
| | | | | 81.7% | |
| >260.8 | 100 | 89.11% to 100% | 72.73 | 60.36% to | 3.667 |
| | | | | 82.97% | |
| >264.9 | 100 | 89.11% to 100% | 74.24 | 61.99% to | 3.882 |
| | | | | 84.22% | |
| >272.4 | 96.88 | 83.78% to 99.92% | 74.24 | 61.99% to | 3.761 |
| | | | | 84.22% | |
| >280.5 | 96.88 | 83.78% to 99.92% | 75.76 | 63.64% to | 3.996 |
| | | | | 85.46% | |
| >287.5 | 96.88 | 83.78% to 99.92% | 77.27 | 65.3% to | 4.263 |
| | | | | 86.69% | |
| >293.1 | 96.88 | 83.78% to 99.92% | 78.79 | 66.98% to | 4.567 |
| | | | | 87.89% | |
| >298.4 | 96.88 | 83.78% to 99.92% | 80.3 | 68.68% to | 4.918 |
| | | | | 89.07% | |
| >308.7 | 96.88 | 83.78% to 99.92% | 81.82 | 70.39% to | 5.328 |
| | | | | 90.24% | |
| >320.8 | 96.88 | 83.78% to 99.92% | 83.33 | 72.13% to | 5.813 |
| | | | | 91.38% | |
| >326.2 | 96.88 | 83.78% to 99.92% | 84.85 | 73.9% to | 6.394 |
| | | | | 92.49% | |
| >330.5 | 96.88 | 83.78% to 99.92% | 86.36 | 75.69% to | 7.104 |
| | | | | 93.57% | |
| >337.7 | 93.75 | 79.19% to 99.23% | 86.36 | 75.69% to | 6.875 |
| | | | | 93.57% | |
| >341.9 | 90.63 | 74.98% to 98.02% | 86.36 | 75.69% to | 6.646 |
| | | | | 93.57% | |
| >348.5 | 87.5 | 71.01% to 96.49% | 86.36 | 75.69% to | 6.417 |
| | | | | 93.57% | |
| >356.6 | 87.5 | 71.01% to 96.49% | 87.88 | 77.51% to | 7.219 |
| | | | | 94.62% | |
| >367.5 | 87.5 | 71.01% to 96.49% | 89.39 | 79.36% to | 8.25 |
| | | | | 95.63% | |
| >378.6 | 84.38 | 67.21% to 94.72% | 89.39 | 79.36% to | 7.955 |
| | | | | 95.63% | |
| >381.9 | 84.38 | 67.21% to 94.72% | 90.91 | 81.26% to | 9.281 |
| | | | | 96.59% | |
| >383.6 | 84.38 | 67.21% to 94.72% | 92.42 | 83.2% to | 11.14 |
| | | | | 97.49% | |
| >386.8 | 84.38 | 67.21% to 94.72% | 93.94 | 85.2% to | 13.92 |
| | | | | 98.32% | |
| >390.2 | 81.25 | 63.56% to 92.79% | 93.94 | 85.2% to | 13.41 |
| | | | | 98.32% | |
| >397.1 | 81.25 | 63.56% to 92.79% | 95.45 | 87.29% to | 17.88 |
| | | | | 99.05% | |
| >403.4 | 81.25 | 63.56% to 92.79% | 96.97 | 89.48% to | 26.81 |
| | | | | 99.63% | |
| >409.2 | 81.25 | 63.56% to 92.79% | 98.48 | 91.84% to | 53.63 |
| | | | | 99.96% | |
| >414.2 | 81.25 | 63.56% to 92.79% | 100 | 94.56% to 100% | |
| >455.9 | 78.13 | 60.03% to 90.72% | 100 | 94.56% to 100% | |
| >498.6 | 75 | 56.6% to 88.54% | 100 | 94.56% to 100% | |
| >507.3 | 71.88 | 53.25% to 86.25% | 100 | 94.56% to 100% | |
| >520.5 | 68.75 | 49.99% to 83.88% | 100 | 94.56% to 100% | |
| >530.4 | 65.63 | 46.81% to 81.43% | 100 | 94.56% to 100% | |
| >551.7 | 62.5 5 | 43.69% to 78.9% | 100 | 94.56% to 100% | |
| >603.5 | 59.38 | 40.64% to 76.3% | 100 | 94.56% to 100% | |
| >646 | 56.25 | 37.66% to 73.64% | 100 | 94.56% to 100% | |
| >664.7 | 53.13 | 34.74% to 70.91% | 100 | 94.56% to 100% | |
| >681.2 | 50 | 31.89% to 68.11% | 100 | 94.56% to 100% | |
| >691 | 46.88 | 29.09% to 65.26% | 100 | 94.56% to 100% | |
| >698.5 | 43.75 | 26.36% to 62.34% | 100 | 94.56% to 100% | |
| >708.9 | 40.63 | 23.7% to 59.36% | 100 | 94.56% to 100% | |
| >723.1 | 37.5 | 21.1% to 56.31% | 100 | 94.56% to 100% | |
| >763.6 | 34.38 | 18.57% to 53.19% | 100 | 94.56% to 100% | |
| >809 | 31.25 | 16.12% to 50.01% | 100 | 94.56% to 100% | |
| >860.9 | 28.13 | 13.75% to 46.75% | 100 | 94.56% to 100% | |
| 956.1 | 25 | 11.46% to 43.4% | 100 | 94.56% to 100% | |
| >1012 | 21.88 | 9.277% to 39.97% | 100 | 94.56% to 100% | |
| >1109 | 18.75 | 7.208% to 36.44% | 100 | 94.56% to 100% | |
| >1253 | 15.63 | 5.275% to 32.79% | 100 | 94.56% to 100% | |
| >1307 | 12.5 | 3.513% to 28.99% | 100 | 94.56% to 100% | |
| >1333 | 9.375 | 1.977% to 25.02% | 100 | 94.56% to 100% | |
| >1410 | 6.25 | 0.7661% to | 100 | 94.56% to 100% | |
| | | 20.81% | | | |
| >1541 | 3.125 | 0.07909% to | 100 | 94.56% to 100% | |
| | | 16.22% | | | |

TABLE 24
Cut-off point analyses for caspase-1 in serum.

| | Sensi- | | Speci- | | Like- |
|---|---|---|---|---|---|
| Cutoff | tivity | | ficity | | lihood |
| (pg/ml) | % | 95% CI | % | 95% CI | ratio |
| >1.076 | 95.65 | 78.05% to | 0 | 0% to 40.96% | 0.9565 |
| | | 99.89% | | | |
| >1.136 | 95.65 | 78.05% to | 14.29 | 0.361% to 57.87% | 1.116 |
| | | 99.89% | | | |
| >1.171 | 91.3 | 71.96% to | 14.29 | 0.361% to 57.87% | 1.065 |
| | | 98.93% | | | |
| >1.177 | 86.96 | 66.41% to | 14.29 | 0.361% to 57.87% | 1.014 |
| | | 97.22% | | | |
| >1.197 | 82.61 | 61.22% to | 14.29 | 0.361% to 57.87% | 0.9638 |
| | | 95.05% | | | |
| >1.243 | 78.26 | 56.3% to | 14.29 | 0.361% to 57.87% | 0.913 |
| | | 92.54% | | | |
| >1.317 | 73.91 | 51.59% to | 14.29 | 0.361% to 57.87% | 0.8623 |
| | | 89.77% | | | |
| >1.387 | 73.91 | 51.59% to | 28.57 | 3.669% to 70.96% | 1.035 |
| | | 89.77% | | | |
| >1.468 | 69.57 | 47.08% to | 28.57 | 3.669% to 70.96% | 0.9739 |
| | | 86.79% | | | |
| >1.58 | 69.57 | 47.08% to | 42.86 | 9.899% to 81.59% | 1.217 |
| | | 86.79% | | | |
| >1.753 | 65.22 | 42.73% to | 42.86 | 9.899% to 81.59% | 1.141 |
| | | 83.62% | | | |
| >1.882 | 65.22 | 42.73% to | 57.14 | 18.41% to 90.1% | 1.522 |
| | | 83.62% | | | |
| >1.941 | 60.87 | 38.54% to | 57.14 | 18.41% to 90.1% | 1.42 |
| | | 80.29% | | | |
| >2.093 | 56.52 | 34.49% to | 57.14 | 18.41% to 90.1% | 1.319 |
| | | 76.81% | | | |
| >2.251 | 52.17 | 30.59% to | 57.14 | 18.41% to 90.1% | 1.217 |
| | | 73.18% | | | |
| >2.391 | 47.83 | 26.82% to | 57.14 | 18.41% to 90.1% | 1.116 |
| | | 69.41% | | | |
| >2.592 | 43.48 | 23.19% to | 57.14 | 18.41% to 90.1% | 1.014 |
| | | 65.51% | | | |
| >2.736 | 43.48 | 23.19% to | 71.43 | 29.04% to 96.33% | 1.522 |
| | | 65.51% | | | |
| >2.915 | 39.13 | 19.71% to | 71.43 | 29.04% to 96.33% | 1.37 |
| | | 61.46% | | | |
| >3.263 | 34.78 | 16.38% to | 71.43 | 29.04% to 96.33% | 1.217 |
| | | 57.27% | | | |
| >4.06 | 34.78 | 16.38% to | 85.71 | 42.13% to 99.64% | 2.435 |
| | | 57.27% | | | |
| >4.774 | 30.43 | 13.21% to | 85.71 | 42.13% to 99.64% | 2.13 |
| | | 52.92% | | | |
| >5.103 | 26.09 | 10.23% to | 85.71 | 42.13% to 99.64% | 1.826 |
| | | 48.41% | | | |
| >5.44 | 21.74 | 7.46% to | 85.71 | 42.13% to 99.64% | 1.522 |
| | | 43.7% | | | |
| >5.896 | 17.39 | 4.951% to | 85.71 | 42.13% to 99.64% | 1.217 |
| | | 38.78% | | | |
| >6.366 | 17.39 | 4.951% to | 100 | 59.04% to 100% | |
| | | 38.78% | | | |
| >6.624 | 13.04 | 2.775% to | 100 | 59.04% to 100% | |
| | | 33.59% | | | |
| >7.76 | 8.696 | 1.071% to | 100 | 59.04% to 100% | |
| | | 28.04% | | | |
| >9.548 | 4.348 | 0.11% to | 100 | 59.04% to 100% | |
| | | 21.95% | | | |

TABLE 25
Cut-off point analyses for IL-18 in serum.

| | Sensi- | | Speci- | | Like- |
|---|---|---|---|---|---|
| Cutoff | tivity | | ficity | | lihood |
| (pg/ml) | % | 95% CI | % | 95% CI | ratio |
| >40.42 | 100 | 88.78% to | 1.449 | 0.03669% to | 1.015 |
| | | 100% | | 7.812% | |
| >60.89 | 100 | 88.78% to | 2.899 | 0.353% to | 1.03 |
| | | 100% | | 10.08% | |
| >91.28 | 100 | 88.78% to | 4.348 | 0.9058% to | 1.045 |
| | | 100% | | 12.18% | |
| >104.2 | 100 | 88.78% to | 5.797 | 1.602% to | 1.062 |
| | | 100% | | 14.18% | |
| >109.7 | 100 | 88.78% to | 7.246 | 2.395% to | 1.078 |
| | | 100% | | 16.11% | |
| >114.8 | 100 | 88.78% to | 8.696 | 3.258% to | 1.095 |
| | | 100% | | 17.97% | |
| >118.1 | 100 | 88.78% to | 10.14 | 4.177% to | 1.113 |
| | | 100% | | 19.79% | |
| >121.1 | 100 | 88.78% to | 11.59 | 5.141% to | 1.131 |
| | | 100% | | 21.57% | |
| >124.2 | 100 | 88.78% to | 13.04 | 6.142% to | 1.15 |
| | | 100% | | 23.32% | |
| >126.5 | 100 | 88.78% to | 14.49 | 7.175% to | 1.169 |
| | | 100% | | 25.04% | |
| >129.7 | 100 | 88.78% to | 15.94 | 8.236% to | 1.19 |
| | | 100% | | 26.74% | |
| >136.2 | 96.77 | 83.3% to | 15.94 | 8.236% to | 1.151 |
| | | 99.92% | | 26.74% | |
| >141.2 | 93.55 | 78.58% to | 15.94 | 8.236% to | 1.113 |
| | | 99.21% | | 26.74% | |
| >147.3 | 90.32 | 74.25% to | 15.94 | 8.236% to | 1.075 |
| | | 97.96% | | 26.74% | |
| >152.8 | 90.32 | 74.25% to | 17.39 | 9.322% to | 1.093 |
| | | 97.96% | | 28.41% | |
| >154 | 90.32 | 74.25% to | 18.84 | 10.43% to | 1.113 |
| | | 97.96% | | 30.06% | |
| >155.4 | 90.32 | 74.25% to | 20.29 | 11.56% to | 1.133 |
| | | 97.96% | | 31.69% | |
| >156 | 90.32 | 74.25% to | 21.74 | 12.71% to | 1.154 |
| | | 97.96% | | 33.31% | |
| >157.8 | 90.32 | 74.25% to | 23.19 | 13.87% to | 1.176 |
| | | 97.96% | | 34.91% | |
| >161.1 | 87.1 | 70.17% to | 23.19 | 13.87% to | 1.134 |
| | | 96.37% | | 34.91% | |
| >163.5 | 87.1 | 70.17% to | 24.64 | 15.05% to | 1.156 |
| | | 96.37% | | 36.49% | |
| >164.8 | 83.87 | 66.27% to | 24.64 | 15.05% to | 1.113 |
| | | 94.55% | | 36.49% | |
| >166.8 | 80.65 | 62.53% to | 24.64 | 15.05% to | 1.07 |
| | | 92.55% | | 36.49% | |
| >169.1 | 77.42 | 58.9% to | 24.64 | 15.05% to | 1.027 |
| | | 90.41% | | 36.49% | |
| >170.8 | 77.42 | 58.9% to | 26.09 | 16.25% to | 1.047 |
| | | 90.41% | | 38.06% | |
| >171.8 | 77.42 | 58.9% to | 27.54 | 17.46% to | 1.068 |
| | | 90.41% | | 39.62% | |
| >172.8 | 77.42 | 58.9% to | 28.99 | 18.69% to | 1.09 |
| | | 90.41% | | 41.16% | |
| >175.2 | 77.42 | 58.9% to | 30.43 | 19.92% to | 1.113 |
| | | 90.41% | | 42.69% | |
| >177.3 | 77.42 | 58.9% to | 31.88 | 21.17% to | 1.137 |
| | | 90.41% | | 44.21% | |
| >178.3 | 74.19 | 55.39% to | 31.88 | 21.17% to | 1.089 |
| | | 88.14% | | 44.21% | |
| >178.9 | 74.19 | 55.39% to | 33.33 | 22.44% to | 1.113 |
| | | 88.14% | | 45.71% | |
| >179.8 | 74.19 | 55.39% to | 34.78 | 23.71% to | 1.138 |
| | | 88.14% | | 47.21% | |
| >182 | 74.19 | 55.39% to | 36.23 | 24.99% to | 1.163 |
| | | 88.14% | | 48.69% | |
| >188.3 | 74.19 | 55.39% to | 37.68 | 26.29% to | 1.191 |
| | | 88.14% | | 50.17% | |
| >194.4 | 74.19 | 55.39% to | 39.13 | 27.6% to | 1.219 |
| | | 88.14% | | 51.63% | |
| >196 | 74.19 | 55.39% to | 40.58 | 28.91% to | 1.249 |
| | | 88.14% | | 53.08% | |
| >197.4 | 74.19 | 55.39% to | 42.03 | 30.24% to | 1.28 |
| | | 88.14% | | 54.52% | |
| >198.4 | 74.19 | 55.39% to | 43.48 | 31.58% to | 1.313 |
| | | 88.14% | | 55.96% | |
| >199.3 | 74.19 | 55.39% to | 44.93 | 32.92% to | 1.347 |
| | | 88.14% | | 57.38% | |
| >200.6 | 74.19 | 55.39% to | 46.38 | 34.28% to | 1.384 |
| | | 88.14% | | 58.8% | |
| >201.3 | 74.19 | 55.39% to | 47.83 | 35.65% to | 1.422 |
| | | 88.14% | | 60.2% | |
| >201.9 | 74.19 | 55.39% to | 49.28 | 37.02% to | 1.463 |
| | | 88.14% | | 61.59% | |
| >202.6 | 74.19 | 55.39% to | 50.72 | 38.41% to | 1.506 |
| | | 88.14% | | 62.98% | |
| >206 | 74.19 | 55.39% to | 52.17 | 39.8% to | 1.551 |
| | | 88.14% | | 64.35% | |
| >210.9 | 74.19 | 55.39% to | 53.62 | 41.2% to | 1.6 |
| | | 88.14% | | 65.72% | |
| >212.9 | 74.19 | 55.39% to | 55.07 | 42.62% to | 1.651 |
| | | 88.14% | | 67.08% | |
| >213.4 | 74.19 | 55.39% to | 56.52 | 44.04% to | 1.706 |
| | | 88.14% | | 68.42% | |
| >213.9 | 74.19 | 55.39% to | 57.97 | 45.48% to | 1.765 |
| | | 88.14% | | 69.76% | |
| >215.4 | 70.97 | 51.96% to | 57.97 | 45.48% to | 1.689 |
| | | 85.78% | | 69.76% | |
| >217.2 | 70.97 | 51.96% to | 59.42 | 46.92% to | 1.749 |
| | | 85.78% | | 71.09% | |
| >219 | 70.97 | 51.96% to | 60.87 | 48.37% to | 1.814 |
| | | 85.78% | | 72.4% | |
| >222.8 | 70.97 | 51.96% to | 62.32 | 49.83% to | 1.883 |
| | | 85.78% | | 73.71% | |
| >226.4 | 67.74 | 48.63% to | 62.32 | 49.83% to | 1.798 |
| | | 83.32% | | 73.71% | |
| >227.6 | 64.52 | 45.37% to | 62.32 | 49.83% to | 1.712 |
| | | 80.77% | | 73.71% | |
| >228 | 64.52 | 45.37% to | 63.77 | 51.31% to | 1.781 |
| | | 80.77% | | 75.01% | |
| >231.4 | 64.52 | 45.37% to | 65.22 | 52.79% to | 1.855 |
| | | 80.77% | | 76.29% | |
| >236 | 64.52 | 45.37% to | 66.67 | 54.29% to | 1.935 |
| | | 80.77% | | 77.56% | |
| >239.1 | 61.29 | 42.19% to | 66.67 | 54.29% to | 1.839 |
| | | 78.15% | | 77.56% | |
| >241.3 | 61.29 | 42.19% to | 68.12 | 55.79% to | 1.922 |
| | | 78.15% | | 78.83% | |
| >241.9 | 58.06 | 39.08% to | 68.12 | 55.79% to | 1.821 |
| | | 75.45% | | 78.83% | |
| >242.1 | 58.06 | 39.08% to | 69.57 | 57.31% to | 1.908 |
| | | 75.45% | | 80.08% | |
| >243.9 | 58.06 | 39.08% to | 71.01 | 58.84% to | 2.003 |
| | | 75.45% | | 81.31% | |
| >246.8 | 54.84 | 36.03% to | 71.01 | 58.84% to | 1.892 |
| | | 72.68% | | 81.31% | |
| >248.8 | 54.84 | 36.03% to | 72.46 | 60.38% to | 1.992 |
| | | 72.68% | | 82.54% | |
| >251.7 | 51.61 | 33.06% to | 72.46 | 60.38% to | 1.874 |
| | | 69.85% | | 82.54% | |
| >255.4 | 51.61 | 33.06% to | 73.91 | 61.94% to | 1.978 |
| | | 69.85% | | 83.75% | |
| >258.5 | 51.61 | 33.06% to | 75.36 | 63.51% to | 2.095 |
| | | 69.85% | | 84.95% | |
| >260.2 | 51.61 | 33.06% to | 76.81 | 65.09% to | 2.226 |
| | | 69.85% | | 86.13% | |
| >267.9 | 48.39 | 30.15% to | 76.81 | 65.09% to | 2.087 |
| | | 66.94% | | 86.13% | |
| >276.4 | 48.39 | 30.15% to | 78.26 | 66.69% to | 2.226 |
| | | 66.94% | | 87.29% | |
| >278.7 | 48.39 | 30.15% to | 79.71 | 68.31% to | 2.385 |
| | | 66.94% | | 88.44% | |
| >281.6 | 48.39 | 30.15% to | 81.16 | 69.94% to | 2.568 |
| | | 66.94% | | 89.57% | |
| >283.7 | 48.39 | 30.15% to | 82.61 | 71.59% to | 2.782 |
| | | 66.94% | | 90.68% | |
| >285.8 | 48.39 | 30.15% to | 84.06 | 73.26% to | 3.035 |
| | | 66.94% | | 91.76% | |
| >288.5 | 48.39 | 30.15% to | 85.51 | 74.96% to | 3.339 |
| | | 66.94% | | 92.83% | |
| >290.1 | 48.39 | 30.15% to | 86.96 | 76.68% to | 3.71 |
| | | 66.94% | | 93.86% | |
| >292.5 | 45.16 | 27.32% to | 86.96 | 76.68% to | 3.462 |
| | | 63.97% | | 93.86% | |
| >295.3 | 41.94 | 24.55% to | 86.96 | 76.68% to | 3.215 |
| | | 60.92% | | 93.86% | |
| >296.8 | 38.71 | 21.85% to | 86.96 | 76.68% to | 2.968 |
| | | 57.81% | | 93.86% | |
| >299.5 | 35.48 | 19.23% to | 86.96 | 76.68% to | 2.72 |
| | | 54.63% | | 93.86% | |
| >302.9 | 35.48 | 19.23% to | 88.41 | 78.43% to | 3.06 |
| | | 54.63% | | 94.86% | |
| >305.4 | 35.48 | 19.23% to | 89.86 | 80.21% to | 3.498 |
| | | 54.63% | | 95.82% | |
| >309.4 | 35.48 | 19.23% to | 91.3 | 82.03% to | 4.081 |
| | | 54.63% | | 96.74% | |
| >313.4 | 35.48 | 19.23% to | 92.75 | 83.89% to | 4.897 |
| | | 54.63% | | 97.61% | |
| >320.4 | 32.26 | 16.68% to | 92.75 | 83.89% to | 4.452 |
| | | 51.37% | | 97.61% | |
| >327.9 | 32.26 | 16.68% to | 94.2 | 85.82% to | 5.565 |
| | | 51.37% | | 98.4% | |
| >333.1 | 32.26 | 16.68% to | 95.65 | 87.82% to | 7.419 |
| | | 51.37% | | 99.09% | |
| >340.1 | 29.03 | 14.22% to | 95.65 | 87.82% to | 6.677 |
| | | 48.04% | | 99.09% | |
| >343.7 | 29.03 | 14.22% to | 97.1 | 89.92% to | 10.02 |
| | | 48.04% | | 99.65% | |
| >346.4 | 25.81 | 11.86% to | 97.1 | 89.92% to | 8.903 |
| | | 44.61% | | 99.65% | |
| >349.3 | 22.58 | 9.594% to | 97.1 | 89.92% to | 7.79 |
| | | 41.1% | | 99.65% | |
| >367 | 19.35 | 7.452% to | 97.1 | 89.92% to | 6.677 |
| | | 37.47% | | 99.65% | |
| >390.1 | 19.35 | 7.452% to | 98.55 | 92.19% to | 13.35 |
| | | 37.47% | | 99.96% | |
| >397.5 | 16.13 | 5.452% to | 98.55 | 92.19% to | 11.13 |
| | | 33.73% | | 99.96% | |
| >402.6 | 12.9 | 3.63% to | 98.55 | 92.19% to | 8.903 |
| | | 29.83% | | 99.96% | |
| >410.8 | 9.677 | 2.042% to | 98.55 | 92.19% to | 6.677 |
| | | 25.75% | | 99.96% | |
| >415.7 | 9.677 | 2.042% to | 100 | 94.79% to | |
| | | 25.75% | | 100% | |
| >423.8 | 6.452 | 0.7911% to | 100 | 94.79% to | |
| | | 21.42% | | 100% | |
| >547.9 | 3.226 | 0.08164% to | 100 | 94.79% to | |
| | | 16.7% | | 100% | |

TABLE 26
Cut-off point analyses for IL-1beta in serum.

| | Sensi- | | Speci- | | Like- |
|---|---|---|---|---|---|
| Cutoff | tivity | | ficity | | lihood |
| (pg/ml) | % | 95% CI | % | 95% CI | ratio |
| <0.391 | 11.11 | 0.2809% to | 90 | 55.5% to | 1.111 |
| | | 48.25% | | 99.75% | |
| <0.3965 | 22.22 | 2.814% to | 90 | 55.5% to | 2.222 |
| | | 60.01% | | 99.75% | |
| <0.4105 | 33.33 | 7.485% to | 90 | 55.5% to | 3.333 |
| | | 70.07% | | 99.75% | |
| <0.434 | 33.33 | 7.485% to | 8 | 44.39% to | 1.667 |
| | | 70.07% | | 97.48% | |
| <0.5085 | 44.44 | 13.7% to | 80 | 44.39% to | 2.222 |
| | | 78.8% | | 97.48% | |
| <0.573 | 44.44 | 13.7% to | 70 | 34.75% to | 1.481 |
| | | 78.8% | | 93.33% | |
| <0.596 | 44.44 | 13.7% to | 60 | 26.24% to | 1.111 |
| | | 78.8% | | 87.84% | |
| <0.6165 | 55.56 | 21.2% to | 60 | 26.24% to | 1.389 |
| | | 86.3% | | 87.84% | |
| <0.644 | 55.56 | 21.2% to | 50 | 18.71% to | 1.111 |
| | | 86.3% | | 81.29% | |
| <0.684 | 66.67 | 29.93% to | 50 | 18.71% to | 1.333 |
| | | 92.51% | | 81.29% | |
| <0.712 | 66.67 | 29.93% to | 40 | 12.16% to | 1.111 |
| | | 92.51% | | 73.76% | |
| <0.791 | 77.78 | 39.99% to | 40 | 12.16% to | 1.296 |
| | | 97.19% | | 73.76% | |
| <0.8585 | 77.78 | 39.99% to | 30 | 6.674% to | 1.111 |
| | | 97.19% | | 65.25% | |
| <0.8685 | 88.89 | 51.75% to | 30 | 6.674% to | 1.27 |
| | | 99.72% | | 65.25% | |
| <1 | 88.89 | 51.75% to | 20 | 2.521% to | 1.111 |
| | | 99.72% | | 55.61% | |
| <1.436 | 100 | 66.37% to | 20 | 2.521% to | 1.25 |
| | | 100% | | 55.61% | |
| <1.822 | 100 | 66.37% to | 10 | 0.2529% to | 1.111 |
| | | 100% | | 44.5% | |

### Amyloid Precursor Proteins (APP) are Promising Serum Biomarkers of MCI and AD

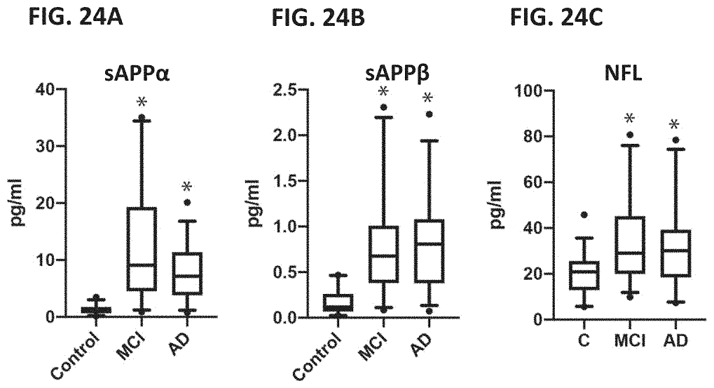

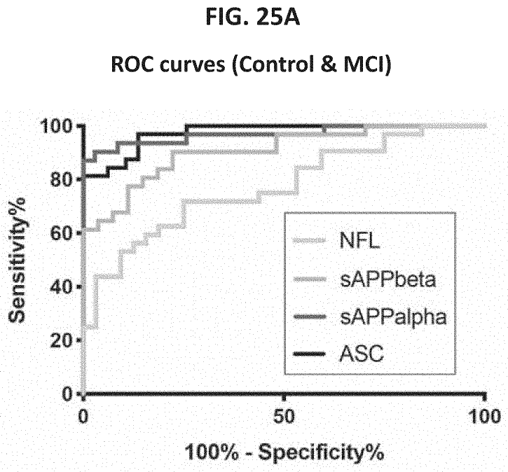

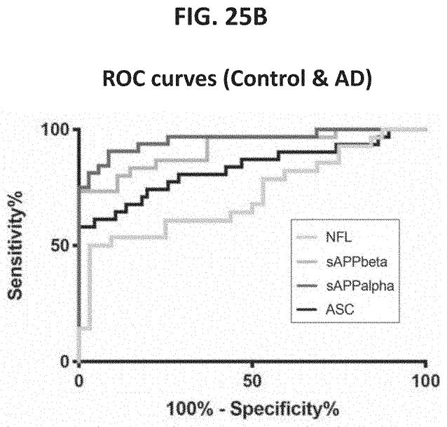

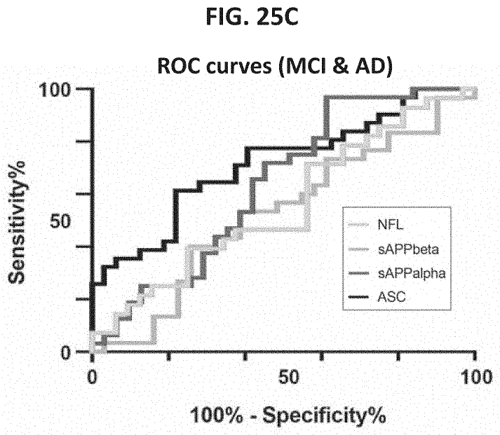

To determine if amyloid precursor proteins (APP) are biomarkers of MCI and AD, serum protein levels of ASC were compared to soluble amyloid precursor proteins α/β (sAPPα/β) for their ability to distinguish between MCI, AD, and controls. The protein levels of sAPPα (FIG. 24A) and sAPPβ (FIG. 24B) were higher in MCI and AD patients than in control subjects. In addition, for control vs MCI, the AUC for these two proteins was 0.9687 and 0.9068, respectively (FIG. 25A and Table 21A); whereas for control vs AD, the AUC were 0.9563 and 0.9185, respectively (FIG. 25B and Table 21B). In addition, for MCI vs AD, the AUC were 0.6351 and 0.5247 (FIG. 25C and Table 21C). For control vs MCI, the cut-off point for sAPPα was 1.39 ng/ml and 0.2639 ng/ml for sAPPβ (Table 22A). For control vs AD, the cut-off point for sAPPα was 2.573 ng/ml and 0.2906 ng/ml for sAPPβ (Table 22B). For MCI vs AD, for sAPPα was 8.846 ng/ml and 0.6364 ng/ml for sAPPβ (Table 22C).

In comparison, for control vs MCI, the cut-off point for ASC was 264.9 pg/ml with 100% sensitivity and 74% specificity; while, sAPPα had a cut-off point of 1.39 ng/ml with 97% sensitivity and 74% specificity, sAPPβ had a cut-off point of 0.2639 ng/ml with 90% sensitivity and 78% specificity (Table 22A).

For control vs AD, the cut-off point for ASC was 258.7 pg/ml with 81% sensitivity and 71% specificity; while, sAPPα had a cut-off point of 2.573 ng/ml with 91% sensitivity and 91% specificity, sAPPβ had a cut-off point of 0.2906 ng/ml with 83% sensitivity and 81% specificity (Table 22B).

For MCI vs AD, the cut-off point for ASC was 560.0 pg/ml with 71% sensitivity and 63% specificity; while, sAPPα had a cut-off point of 8.846 ng/ml with 72% sensitivity and 55% specificity, sAPPβ had a cut-off point of 0.6364 ng/ml with 60% sensitivity and 45% specificity (Table 22C).

### Neurofilament Light Chain (NFL) is a Serum Biomarker of MCI and AD

Serum protein levels of ASC to NFL were compared in control, MCI, and AD patients. Protein levels of NFL were higher in MCI patients than in control subjects (FIG. 24C). The AUC for Flaws 0.7734, whereas for ASC, it was 0.974, as above stated (FIG. 25A and Table 21A). The cut-off point for NFL was 24.15 pg/ml with a sensitivity of 72% and a specificity of 75% (Table 22A). In comparison, for control vs AD the AUC for NFL was 0.7165 and the cut-off point was 21.48 pg/ml with 64% sensitivity and 56% specificity (Table 21B and Table 22B). No significant difference between serum levels of NfL in MCI and AD was observed.

### Linear Regression Between ASC and IL-18

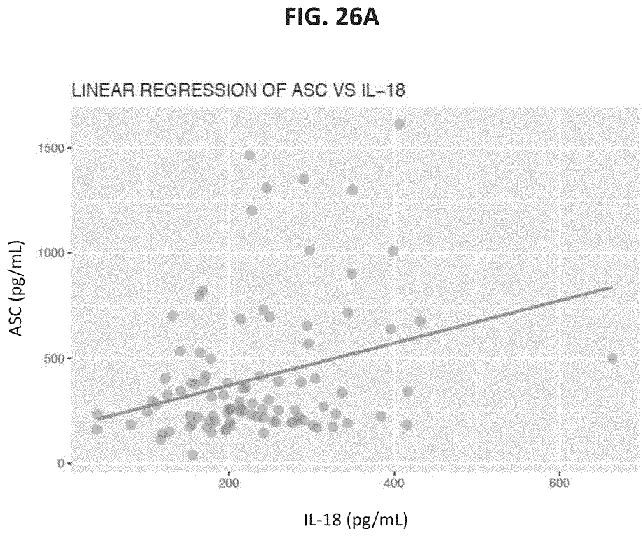

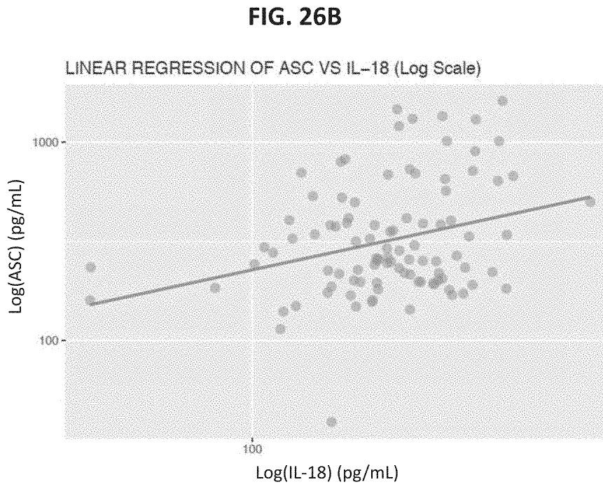

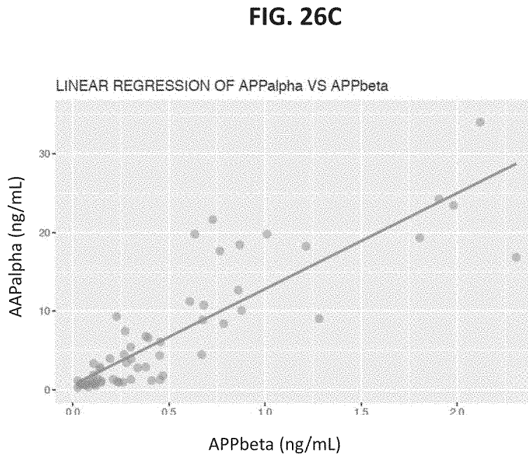

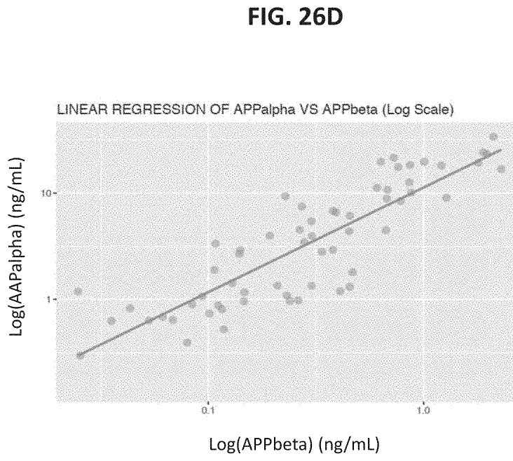

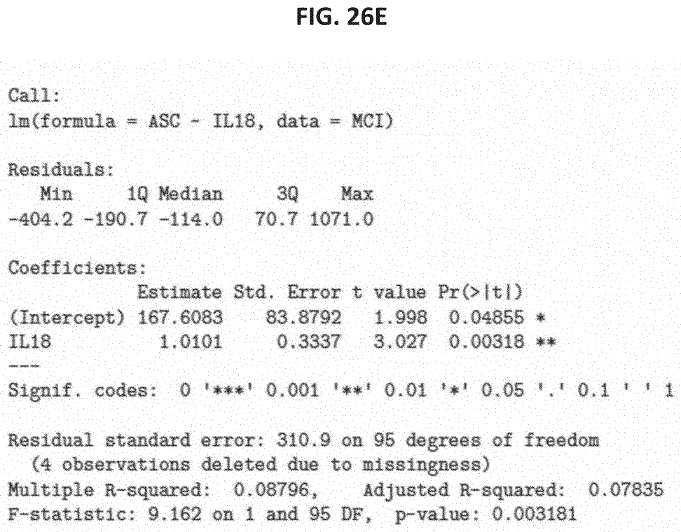

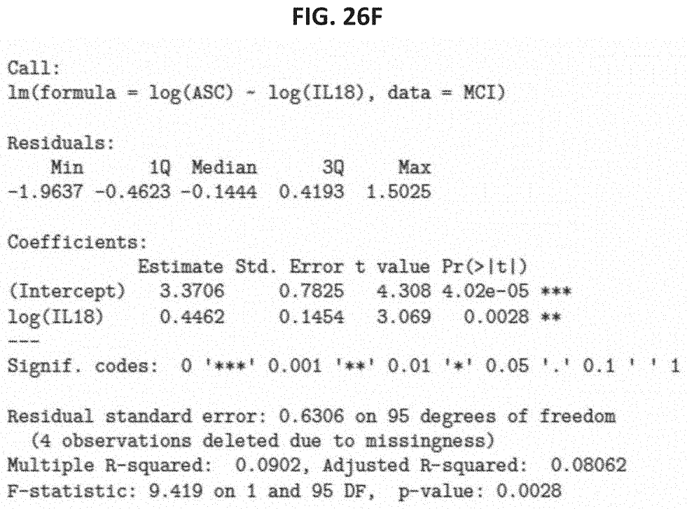

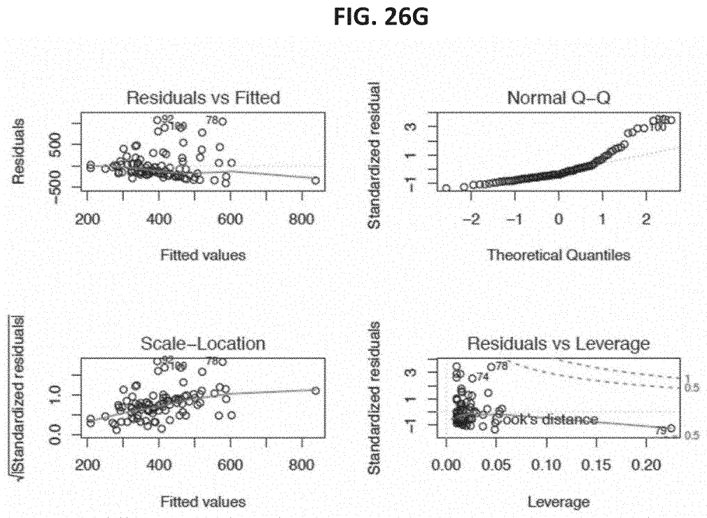

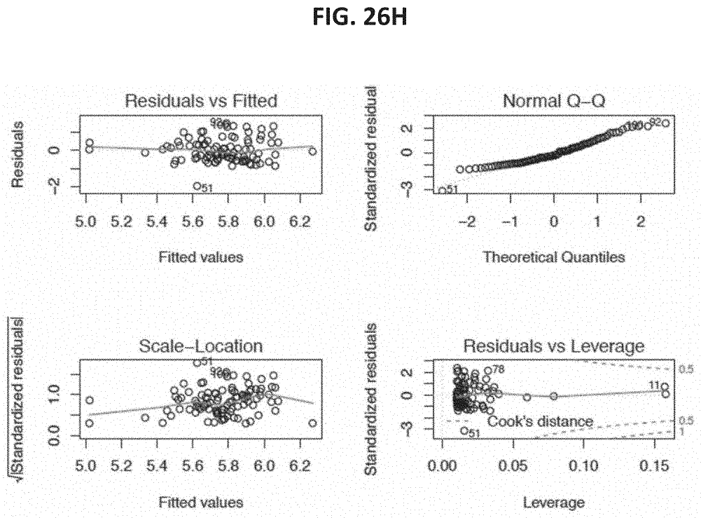

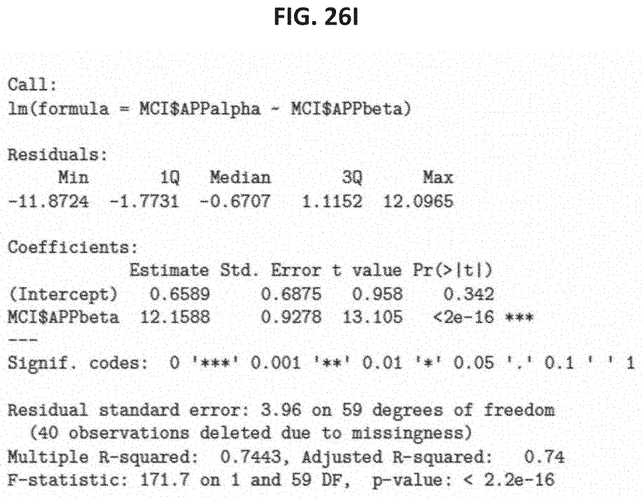

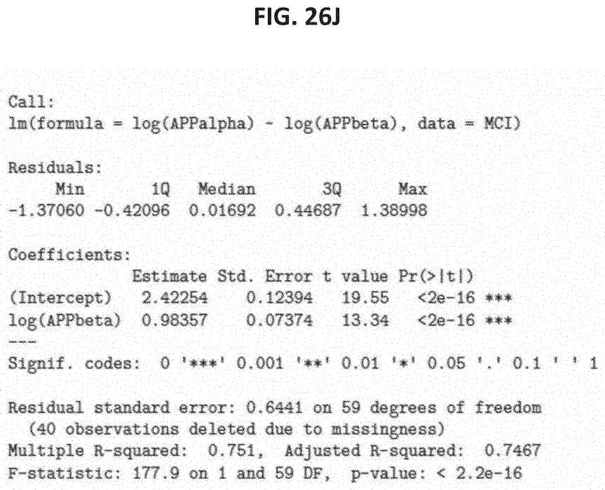

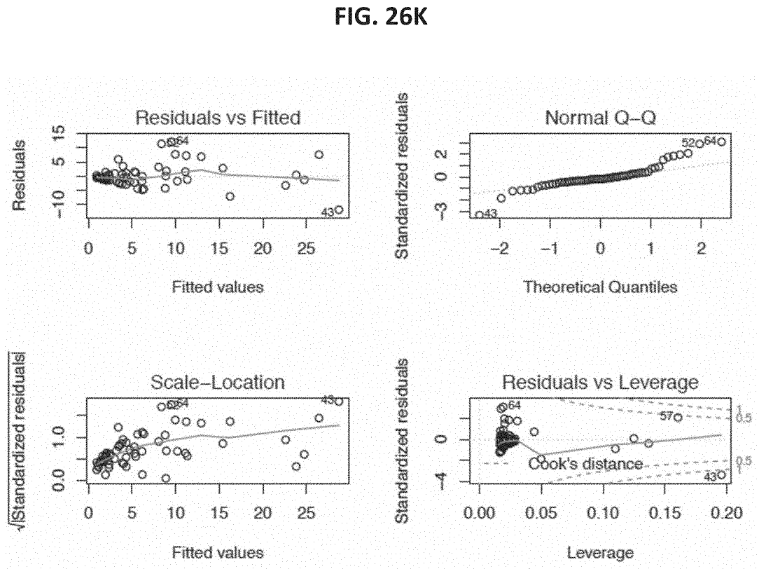

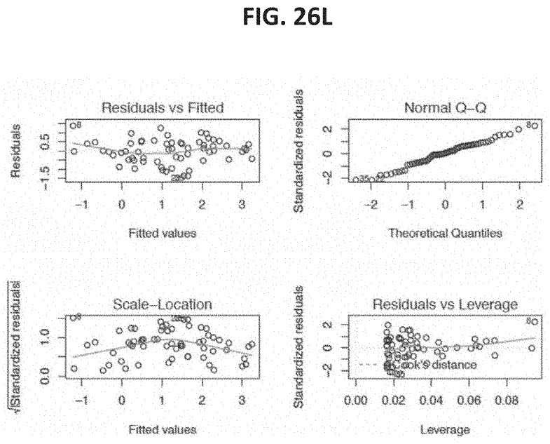

To determine the relation between ASC and IL-18 a linear regression analysis was carried out. Accordingly, data were plotted (FIG. 26A) and a linear model was fitted (FIG. 26E). We found that IL-18 protein levels have a statistically significant linear correlation with the protein levels of ASC (p-value=0.00318); however, this model relying on IL-18 expression only explained 8% of the protein levels in ASC. Moreover, a box-cox transformation suggested a logarithmic transformation (FIG. 26B). However, the adjusted R2 for this model was also around 8% (FIG. 26F), yet the logarithmically transformed model resulted in more normally distributed residuals (FIG. 26G and FIG. 26H). Thus, these findings suggest that the protein levels of ASC depend mostly on other proteins besides IL-18, and vice-versa.

Linear Regression Between sAPPα and sAPPβ

To determine the relation between sAPPα and sAPPβ a linear regression analysis was carried out. Accordingly, data were plotted (FIG. 26C) and a linear model was fitted (FIG. 26I), and we found that sAPPβ protein levels have a statistically significant linear correlation with the protein levels of sAPPα. Importantly, this model was able to explain 74% of the protein levels in sAPPα. A box-cox transformation suggested a logarithmic transformation (FIG. 26D). However, the adjusted R2 for this model was also around 74% (FIG. J), yet the logarithmically transformed model resulted in more normally distributed residuals (FIG. K and FIG. L). Thus, the protein levels of sAPPα and sAPPβ are strongly correlated with each other.

### Cluster Analysis Using ASC Protein Levels in Control, MCI, and AD Patients

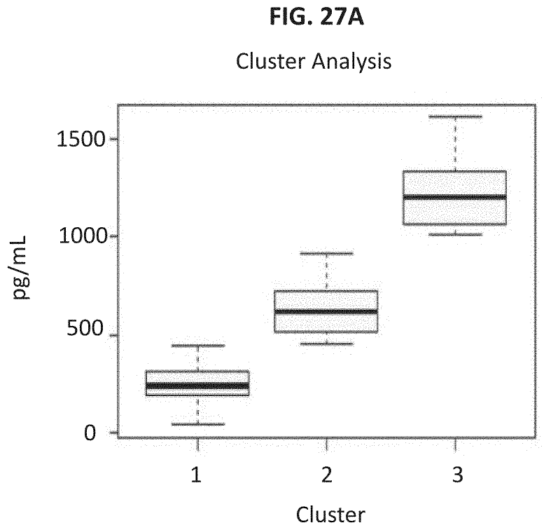

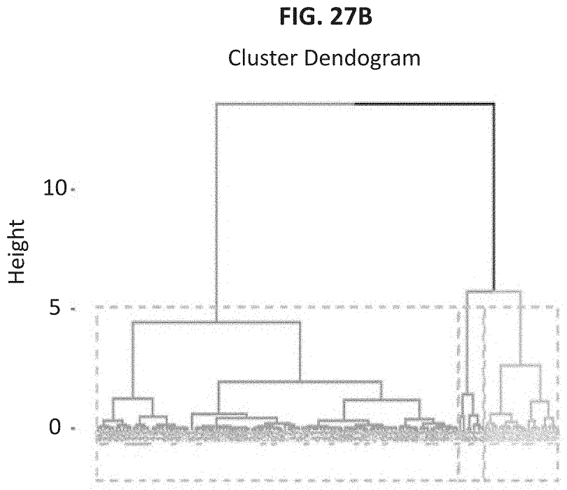

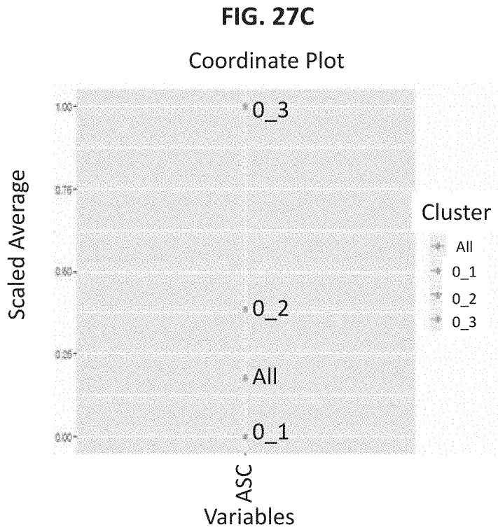

The serum of control, MCI, and AD patients, each of which contains ASC, was pooled into one group to perform a cluster analysis. Three different clusters were found using a Gaussian Mixture Modelling method (FIG. 27A) consistent with the three different cohorts of patients present (control, MCI and AD). In addition, a cluster dendrogram was obtained using hierarchical clustering in which three groups were identified (FIG. 27B), which was further corroborated in a coordinate plot (FIG. 27C). Thus, these findings indicate that ASC protein levels in serum can be used to stratify patients among control, MCI and AD cohorts.

### Conclusions

In this study, a statistically significant higher level of ASC and IL-18 was detected in the serum of MCI and AD patients when compared to healthy subjects. In this study, we show that ASC is a reliable biomarker for MCI and AD with AUC values of 0.974 for control vs MCI, 0.8328 for control vs AD, and 0.7157 for MCI vs AD.

Our findings in the control vs MCI groups that ASC has an AUC of 0.974 compared to 0.9687 for sAPPα, 0.9068 for sAPPβ and 0.7734 for NFL, suggest that ASC is a good biomarker comparable to sAPPα, sAPPβ, and NfL. Similar results were observed for control vs AD. However, when comparing MCI vs AD, ASC had an AUC of 0.7157, sAPPα, 0.6351, sAPPβ, 0.5247 and NFL had an AUC of 0.5569. Therefore, when differentiating between MCI and AD, ASC has the potential to be a more reliable serum biomarker.

Thus, based on these findings ASC is a promising biomarker with a high AUC value, a high sensitivity and high specificity in serum. Importantly, ASC as a biomarker for MCI and AD with other diagnostic criteria may further increase the sensitivity of ASC as a biomarker for MCI and AD beyond what is described in this example. Furthermore, in contrast to methods that use biomarkers in cerebrospinal fluid for diagnosis of AD and MCI, this method has identified serum biomarkers that may reliably be used to diagnose these brain injuries.

### INCORPORATION BY REFERENCE

The following references are incorporated by reference in their entireties for all purposes.
1.) Biomarkers Definitions Working G. Biomarkers and surrogate endpoints: preferred definitions and conceptual framework. Clin Pharmacol Ther. 2001; 69:89-95.
2.) Brand F J, 3rd, Forouzandeh M, Kaur H, Travascio F, & de Rivero Vaccari J P (2016) Acidification changes affect the inflammasome in human nucleus pulposus cells. J Inflamm (Lond) 13(1):29.
3.) Keane R W, Dietrich W D, & de Rivero Vaccari J P (2018) Inflammasome Proteins As Biomarkers of Multiple Sclerosis. Front Neurol 9:135
4.) Padovani, A.; Borroni, B.; Colciaghi, F.; Pettenati, C.; Cottini, E.; Agosti, C.; Lenzi, G. L.; Caltagirone, C.; Trabucchi, M.; Cattabeni, F.; Di Luca, M., Abnormalities in the pattern of platelet amyloid precursor protein forms in patients with mild cognitive impairment and Alzheimer disease. Arch Neurol 2002, 59, (1), 71-5.
5.) Petersen, R. C., Aging, mild cognitive impairment, and Alzheimer's disease. Neurol Clin 2000, 18, (4), 789-806.
6.) Petersen, R. C.; Smith, G. E.; Waring, S. C.; Ivnik, R. J.; Tangalos, E. G.; Kokmen, E., Mild cognitive impairment: clinical characterization and outcome. Arch Neurol 1999, 56, (3), 303-8.
7.) Bozoki, A.; Giordani, B.; Heidebrink, J. L.; Berent, S.; Foster, N. L., Mild cognitive impairments predict dementia in nondemented elderly patients with memory loss. Arch Neurol 2001, 58, (3), 411-6.
8.) Morris, J. C.; Storandt, M.; Miller, J. P.; McKeel, D. W.; Price, J. L.; Rubin, E. H.; Berg, L., Mild cognitive impairment represents early-stage Alzheimer disease. Arch Neurol 2001, 58, (3), 397-405.
9.) Blennow, K.; Hampel, H., CSF markers for incipient Alzheimer's disease. Lancet Neurol 2003, 2, (10), 605-13.
10.) Kanemaru, K.; Kameda, N.; Yamanouchi, H., Decreased CSF amyloid beta42 and normal tau levels in dementia with Lewy bodies. Neurology 2000, 54, (9), 1875-6.
11.) Sjogren, M.; Minthon, L.; Davidsson, P.; Granerus, A. K.; Clarberg, A.; Vanderstichele, H.; Vanmechelen, E.; Wallin, A.; Blennow, K., CSF levels of tau, beta-amyloid(1-42) and GAP-43 in frontotemporal dementia, other types of dementia and normal aging. J Neural Transm (Vienna) 2000, 107, (5), 563-79.
12.) Andreasen, N.; Sjogren, M.; Blennow, K., CSF markers for Alzheimer's disease: total tau, phospho-tau and Abeta42. World J Biol Psychiatry 2003, 4, (4), 147-55.
13.) Terajima, M.; Arai, H.; Itabashi, S.; Higuchi, M.; Zhu, C.; Kosaka, Y.; Nakagawa, T.; Sasaki, H., Elevated cerebrospinal fluid tau levels: implications for the early diagnosis of Alzheimer's disease. J Am Geriatr Soc 1996, 44, (8), 1012-3.
14.) Araki, W.; Hattori, K.; Kanemaru, K.; Yokoi, Y.; Omachi, Y.; Takano, H.; Sakata, M.; Yoshida, S.; Tsukamoto, T.; Murata, M.; Saito, Y.; Kunugi, H.; Goto, Y. I.; Nagaoka, U.; Nagao, M.; Komori, T.; Arima, K.; Ishii, K.; Murayama, S.; Matsuda, H.; Tachimori, H.; Araki, Y. M.; Mizusawa, H., Re-evaluation of soluble APP-alpha and APP-beta in cerebrospinal fluid as potential biomarkers for early diagnosis of dementia disorders. Biomark Res 2017, 5, 28.
15.) Zetterberg, H., Neurofilament Light: A Dynamic Cross-Disease Fluid Biomarker for Neurodegeneration. Neuron 2016, 91, (1), 1-3.
16.) Parbo, P.; Madsen, L. S.; Ismail, R.; Zetterberg, H.; Blennow, K.; Eskildsen, S. F.; Vorup-Jensen, T.; Brooks, D. J., Low plasma neurofilament light levels associated with raised cortical microglial activation suggest inflammation acts to protect prodromal Alzheimer's disease. Alzheimers Res Ther 2020, 12, (1), 3.
17.) Mayeli, M.; Mirshahvalad, S. M.; Aghamollaii, V.; Tafakhori, A.; Abdolalizadeh, A.; Rahmani, F., Plasma Neurofilament Light Chain Levels Are Associated With Cortical Hypometabolism in Alzheimer Disease Signature Regions. J Neuropathol Exp Neurol 2019.
18.) Pawelec, G.; Goldeck, D.; Derhovanessian, E, Inflammation, ageing and chronic disease. Current opinion in immunology 2014, 29, 23-8.
19.) Aden, K.; Rosenstiel, P., The Dark Age(ing) of the Inflammasome. Immunity 2017, 46, (2), 173-175.
20.) Latz, E.; Duewell, P., NLRP3 inflammasome activation in inflammaging. Semin Immunol 2018, 40, 61-73.
21.) Mawhinney, L. J.; de Rivero Vaccari, J. P.; Dale, G. A.; Keane, R. W.; Bramlett, H. M., Heightened inflammasome activation is linked to age-related cognitive impairment in Fischer 344 rats. BMC Neurosci 2011, 12, 123.
22.) Mejias, N. H.; Martinez, C. C.; Stephens, M. E.; de Rivero Vaccari, J. P., Contribution of the inflammasome to inflammaging. J Inflamm (Lond) 2018, 15, 23.
23.) Platnich, J. M.; Muruve, D. A., NOD-like receptors and inflammasomes: A review of their canonical and non-canonical signaling pathways. Arch Biochem Biophys 2019.
24.) Franklin, B. S.; Bossaller, L.; De Nardo, D.; Ratter, J. M.; Stutz, A.; Engels, G.; Brenker, C.; Nordhoff, M.; Mirandola, S. R.; Al-Amoudi, A.; Mangan, M. S.; Zimmer, S.; Monks, B. G.; Fricke, M.; Schmidt, R. E.; Espevik, T.; Jones, B.; Jarnicki, A. G.; Hansbro, P. M.; Busto, P.; Marshak-Rothstein, A.; Hornemann, S.; Aguzzi, A.; Kastenmuller, W.; Latz, E., The adaptor ASC has extracellular and ‘prionoid’ activities that propagate inflammation. Nat Immunol 2014, 15, (8), 727-37.
25.) Kerr, N.; Lee, S. W.; Perez-Barcena, J.; Crespi, C.; Ibanez, J.; Bullock, M. R.; Dietrich, W. D.; Keane, R. W.; de Rivero Vaccari, J. P., Inflammasome proteins as biomarkers of traumatic brain injury. PLoS One 2018, 13, (12), e0210128.
26.) Kerr, N.; Garcia-Contreras, M.; Abbassi, S.; Mejias, N. H.; Desousa, B. R.; Ricordi, C.; Dietrich, W. D.; Keane, R. W.; de Rivero Vaccari, J. P., Inflammasome Proteins in Serum and Serum-Derived Extracellular Vesicles as Biomarkers of Stroke. Front Mol Neurosci 2018, 11, 309.
27.) Syed, S. A.; Beurel, E.; Loewenstein, D. A.; Lowell, J. A.; Craighead, W. E.; Dunlop, B. W.; Mayberg, H. S.; Dhabhar, F.; Dietrich, W. D.; Keane, R. W.; de Rivero Vaccari, J. P.; Nemeroff, C. B., Defective Inflammatory Pathways in Never-Treated Depressed Patients Are Associated with Poor Treatment Response. Neuron 2018, 99, (5), 914-924 e3.
28.) de Rivero Vaccari, J. P.; Brand, F. J., 3rd; Sedaghat, C.; Mash, D. C.; Dietrich, W. D.; Keane, R. W., RIG-1 receptor expression in the pathology of Alzheimer's disease. J Neuroinflammation 2014, 11, 67.
29.) Brubaker, A. L.; Palmer, J. L.; Kovacs, E. J., Age-related Dysregulation of Inflammation and Innate Immunity: Lessons Learned from Rodent Models. Aging Dis 2011, 2, (5), 346-60.
30.) Liu, D.; Cao, B.; Zhao, Y.; Huang, H.; McIntyre, R. S.; Rosenblat, J. D.; Zhou, H., Soluble TREM2 changes during the clinical course of Alzheimer's disease: A meta-analysis. Neuroscience Letters 2018, 686, 10-16.
31.) Lynch, M. A., Age-related neuroinflammatory changes negatively impact on neuronal function. Front Aging Neurosci 2010, 1, 6.
32.) Calabrese, V.; Giordano, J.; Signorile, A.; Laura Ontario, M.; Castorina, S.; De Pasquale, C.; Eckert, G.; Calabrese, E. J., Major pathogenic mechanisms in vascular dementia: Roles of cellular stress response and hormesis in neuroprotection. J Neurosci Res 2016, 94, (12), 1588-1603.
33.) Singhal, G.; Jaehne, E. J.; Corrigan, F.; Toben, C.; Baune, B. T., Inflammasomes in neuroinflammation and changes in brain function: a focused review. Front Neurosci 2014, 8, 315.
34.) Weinstein, G.; Lutski, M.; Goldbourt, U.; Tanne, D., C-reactive protein is related to future cognitive impairment and decline in elderly individuals with cardiovascular disease. Archives of Gerontology and Geriatrics 2017, 69, 31-37.
35.) Sloane, J. A.; Hollander, W.; Moss, M. B.; Rosene, D. L.; Abraham, C. R., Increased microglial activation and protein nitration in white matter of the aging monkey⋆. Neurobiology of Aging 1999, 20, (4), 395-405.
36.) Prolla, T. A., DNA microarray analysis of the aging brain. Chem Senses 2002, 27, (3), 299-306.
37.) Adamczak, S.; Dale, G.; de Rivero Vaccari, J. P.; Bullock, M. R.; Dietrich, W. D.; Keane, R. W., Inflammasome proteins in cerebrospinal fluid of brain-injured patients as biomarkers of functional outcome: clinical article. Journal of neurosurgery 2012, 117, (6), 1119-25.
38.) Yap, J. K. Y.; Pickard, B. S.; Chan, E. W. L.; Gan, S. Y., The Role of Neuronal NLRP1 Inflammasome in Alzheimer's Disease: Bringing Neurons into the Neuroinflammation Game. Molecular neurobiology 2019.
39.) Yin, J.; Zhao, F.; Chojnacki, J. E.; Fulp, J.; Klein, W. L.; Zhang, S.; Zhu, X., NLRP3 Inflammasome Inhibitor Ameliorates Amyloid Pathology in a Mouse Model of Alzheimer's Disease. Molecular neurobiology 2018, 55, (3), 1977-1987.
40.) Saco, T.; Parthasarathy, P. T.; Cho, Y.; Lockey, R. F.; Kolliputi, N., Inflammasome: a new trigger of Alzheimer's disease. Frontiers in aging neuroscience 2014, 6, 80.
41.) Tan, M. S.; Yu, J. T.; Jiang, T.; Zhu, X. C.; Tan, L., The NLRP3 inflammasome in Alzheimer's disease. Molecular neurobiology 2013, 48, (3), 875-82.
42.) Knopman, D. S.; Penman, A. D.; Catellier, D. J.; Coker, L. H.; Shibata, D. K.; Sharrett, A. R.; Mosley, T. H., Jr., Vascular risk factors and longitudinal changes on brain MRI: the ARIC study. Neurology 2011, 76, (22), 1879-85.
43.) Cerhan, J. R.; Folsom, A. R.; Mortimer, J. A.; Shahar, E.; Knopman, D. S.; McGovern, P. G.; Hays, M. A.; Crum, L. D.; Heiss, G., Correlates of cognitive function in middle-aged adults. Atherosclerosis Risk in Communities (ARIC) Study Investigators. Gerontology 1998, 44, (2), 95-105.

### Example 5: Examination of Inflammasome Proteins as Biomarkers of Age-Related Macular Degeneration (AMD)

### Introduction

A biomarker is a characteristic that can be measured objectively and evaluated as an indicator of normal or pathologic biological processes. Important to the care of patients with AMD are the need for biomarkers that can screen for and diagnose AMD, detect exacerbation of AMD, and evaluate a patient's response to treatment.

### Methods

### Participants:

In this example, samples were purchased from BioIVT. Sample donors were enrolled in the study “Prospective Collection of Samples for Research” sponsored by SeraTrials, LLC with IRB number 20170439. The age range of donors was from 55 to 93 years old with 61 samples in the control no-AMD group and 32 in the AMD group (Table 27).

TABLE 27
Demographics of participants with AMD

| Gen- | | | | |
|---|---|---|---|---|
| der | Age | Race | Diagnosis | Medications |
| Fe- | 82 | Cau- | Dry Age Related Macular | Enalapril 10 mg, |
| male | | casian | Degeneration, | Vitamin B12 1000 |
| | | | Hypertension | mcg, Aspirin 81 mg |
| Fe- | 93 | Cau- | Wet Age Related Macular | Potassium Chloride |
| male | | casian | Degeneration (AMD), | 10 meq, Trazodone, |
| | | | Acute Macular | Caltrate, Centrum, |
| | | | Degeneration, | Ramipril 10 mg, |
| | | | Hypertension (HTN), | Simvastatin 20 mg, |
| | | | Hyperlipidemia | Amlodipine Besylate |
| | | | (HLD), Dementia, | 2.5 mg, Metropolol |
| | | | Anxiety , Hypercalcemia, | Tartrate, klor |
| | | | Depression, Hypokalemia | Con M10 1 meq, |
| | | | | Paxil 20 mg |
| Male | 71 | Cau- | Acute Macular | Vitamin C 500 mg, |
| | | casian | Degeneration, | Mucinex, |
| | | | Chronic Obstructive | Advair Diskus |
| | | | Pulmonary Disease | 500-50 mcg, Eylea, |
| | | | (COPD), Obstructive | Calcium, B Complex, |
| | | | Sleep Apnea (OSA), Type | Vitamin D3 50000 iu, |
| | | | 2 Diabetes, Obesity, | Stool Softener, Vitamin |
| | | | Venous Insufficiency, | B12, Nasacort |
| | | | Vitamin D | AQ 55 mcg/act, |
| | | | Deficiency, Wet Age | Singulair 10 mg, |
| | | | Related Macular | Prednisone |
| | | | Degeneration (AMD), | |
| | | | Slow Transit | |
| | | | Constipation | |
| Male | 79 | Cau- | Acute Macular | Gabapentin 600 |
| | | casian | Degeneration, Chronic | mg, Potassium |
| | | | Sciatic, Neuropathy, | Chloride, Triamcinolone |
| | | | Benign Prostatic | Acetonide 0.1%, |
| | | | Hyperplasia (BPH), | Fenofibrate |
| | | | Hypertension (HTN), | 160 mg, Ropinerole |
| | | | Hyperlipidemia (HLD), | HCI 2 mg, |
| | | | Osteoarthritis (OA), | Pantoprazole |
| | | | Wet Age Related Macular | Sodium 40 mg, |
| | | | Degeneration (AMD), | Enalapril Maleate |
| | | | Venous Insufficiency | 20 mg, Miralax, |
| | | | | Nexium, Oxycodone |
| | | | | HCI 10 mg, |
| | | | | Metamucil, |
| | | | | Ibuprofen 200 mg, |
| | | | | Antara, Vitamin |
| | | | | B Complex, |
| | | | | Tagamet 200 mg, |
| | | | | Cymbalta 60 mg |
| Male | 75 | African | Dry Age Related Macular | Metformin 500 |
| | | | Degeneration | mg, Simvastatin |
| | | | (Right Eye), Type 2 | 40 mg, Finasteride |
| | | | Diabetes, Hypertension | 5 mg, Aspirin 81 mg, |
| | | | (HTN), | Amlodipine 5 mg, |
| | | | Hypercholesterolemia, | Anagrelide 1 mg, |
| | | | Thrombocythemia, | Atenolol/Chlorthalidone |
| | | | Tonsillitis | 50 mg/25 mg, |
| | | | | Artificial Tears |
| Fe- | 65 | Cau- | Dry Age Related Macular | Metformin 1000 |
| male | | casian | Degeneration, | mg, Humalog |
| | | | Type 2 Diabetes, | 65 iu, Lantus 15 |
| | | | Hypertension (HTN), | iu, Metoprolol 50 mg, |
| | | | Hypercholesterolemia | Lisinopril 40 mg, |
| | | | | Gabapentin 300 mg, |
| | | | | Plavix 75 mg, Aspirin |
| | | | | 81 mg, Fenofibrate |
| | | | | 150 mg, Xarelto 20 mg |
| Fe- | 81 | Cau- | Dry Age Related Macular | Nutrof Total |
| male | | casian | Degeneration (AMD), | |
| | | | Geographic Atrophy | |
| Fe- | 85 | Cau- | Dry Age Related Macular | None |
| male | | casian | Degeneration | |
| | | | (AMD), Geographic | |
| | | | Atrophy (GA) | |
| Fe- | 77 | Cau- | Dry Age Related Macular | Slezavit |
| male | | casian | Degeneration (AMD), | |
| | | | Geographic Atrophy | |
| Male | 75 | Cau- | Dry Age Related Macular | None |
| | | casian | Degeneration (AMD), | |
| | | | Geographic Atrophy | |
| Male | 83 | Cau- | Wet Age Related Macular | Simvastatin |
| | | casian | Degeneration | 40 mg, Avastin |
| | | | (AMD), Enlarged | 1.25 mg |
| | | | Prostate, | |
| | | | Hypercholesterolemia | |
| Fe- | 91 | Cau- | Acute Wet Macular | Synthroid 100 mcg, |
| male | | casian | Degeneration, | Limbrel 500 mg, |
| | | | Polymyalgia Rhuematica, | Flexeril, |
| | | | Hyperlipidemia | Restasis 0.05%, |
| | | | (HLD), Hypertension | Clindamycin |
| | | | (HTN), Gastroesophageal | HCI 150 mg, Advil |
| | | | Reflux Disease | |
| | | | (GERD), Osteoporosis, | |
| | | | Hepatitis B (HBV), | |
| | | | Mitral Valve | |
| | | | Prolapse (MVP), | |
| | | | Leukopenia | |
| Male | 76 | Cau- | Dry Age Related Macular | Amlodipine 10 |
| | | casian | Degeneration, | mg, Furosemide |
| | | | Type 2 Diabetes, | 40 mg, Atorvastatin |
| | | | Hypertension (HTN), | 20 mg, Metformin |
| | | | Hypercholesterolemia, | 850 mg, Benicar |
| | | | Thyroid | 20 mg/12.5 mg, |
| | | | Disease | Hydrochlorothiazide |
| | | | | 25 mg, Artificial Tears, |
| | | | | Ofloxacin 0.3% |
| Fe- | 76 | Cau- | Wet Age Related Macular | Aspirin 81 mg, |
| male | | casian | Degeneration (AMD), | Lisinopril 20 mg, |
| | | | Hypertension | Amlodipine 5 mg, |
| | | | (HTN), Allergies, | Metoprolol |
| | | | Menorrhagia | 25 mg, Claritin 10 |
| | | | | mg, AREDS-2, |
| | | | | Artificial Tears, |
| | | | | Avastin 1.25 mg |
| Fe- | 71 | Cau- | Dry Age Related | AREDS 2 |
| male | | casian | Macular | Vitamin, Diltiazem |
| | | | Degeneration | 120 mg, Irbesartan |
| | | | (AMD), Hypertension | 150 mg, Zyrtec |
| | | | (HTN) | 10 mg |
| Male | 68 | Cau- | Age Related Macular | Advair, Aspirin |
| | | casian | Degeneration, | 81 mg, Eylea |
| | | | Asthma | |
| Fe- | 68 | Cau- | Dry Age-Related Macular | Aspirin 81 mg, |
| male | | casian | Degeneration, | Omega 3 1000 mg, |
| | | | Hypercholesterolemia, | AREDS-2, Artificial |
| | | | Post-Menopausal | Tears |
| Fe- | 77 | Cau- | Dry Age Related | Aspirin 81 mg, |
| male | | casian | Degeneration, | Plavix 75 mg, |
| | | | Type 2 Diabetes, | Synthroid 50 mcg, |
| | | | Hypertension (HTN), | Toprol ER |
| | | | Hypercholesterolemia, | 50 mg, Multivitamin, |
| | | | Hypothyroidism | Onglyza 2.5 mg, |
| | | | | Vitamin D3 1000 iu, |
| | | | | Repatha 140 mg, |
| | | | | Losartan 25 mg, |
| | | | | Rosuvastatin 10 mg |
| Male | 85 | Cau- | Wet Age Related | Sotalol 160 mg, |
| | | casian | Macular Degeneration | Warfarin 1 mg, |
| | | | (AMD), Hypertension | Lisinopril 40 mg, |
| | | | (HTN), Cardiovascular | Famotidine |
| | | | Disease, Aneurysm, | 20 mg, Eylea 2 mg |
| | | | Geographic Atrophy (GA) | |
| Fe- | 70 | Cau- | Wet Age Related Macular | Losartan 100 mg, |
| male | | casian | Degeneration (AMD), | Amlodipine 10 mg, |
| | | | | Citalopram 10 mg, |
| | | | Asthma, Hypertension | Albuterol 2.5 mg, |
| | | | (HTN), Depression | Artificial Tears, |
| | | | | Avastin 1.25 mg |
| Male | 79 | Cau- | Wet Age Related Macular | None |
| | | casian | Degeneration (AMD) | |
| Male | 72 | Cau- | Dry Age Related Macular | None |
| | | casian | Degeneration (AMD) | |
| Fe- | 75 | Cau- | Wet Age Related Macular | Tobramycin 3%, |
| male | | casian | Degeneration (AMD), | Aspirin 81 mg, |
| | | | Asthma, Type | Vitamin D 400 |
| | | | 2 Diabetes, | mg, Vitamin E |
| | | | Hypertension (HTN), | 100 iu, Neurontin |
| | | | Hypercholesterolemia, | 800 mg, Fish Oil |
| | | | Coronary | 1000 iu, Losartan |
| | | | Artery Disease | Hydrochlorothiazide, |
| | | | (CAD), Neuropathy | Metformin |
| | | | | 500 mg, Prilosec |
| | | | | 40 mg, Tylenol |
| | | | | 500 mg, Multivitamin, |
| | | | | Vitamin D3 2000 |
| | | | | iu, Turmeric, Eylea |
| Fe- | 79 | Cau- | Wet Age Related | Tylenol 325 mg, |
| male | | casian | Macular | Atorvastatin |
| | | | Degeneration | 80 mg, Colace |
| | | | (AMD), Type 1 | 100 mg, Cymbalta |
| | | | Diabetes, Hypertension | 60 mg, Diovan 40 mg, |
| | | | (HTN), | Neuron tin 300 mg, |
| | | | Hypercholesterolemia, | Humalog 100 mg, |
| | | | Atrial fibrillationÂ (AF), | Isosorbide 10 mg, |
| | | | Osteoarthritis | Lantus 100 mg, |
| | | | (OA), Major | Lasix 40 mg, |
| | | | Depressive Disorder | Melatonin 3 mg, |
| | | | (MDD), Systemic | Metoprolol 50 mg, |
| | | | Inflammatory | Muscle Cream, |
| | | | Response Syndrome, | Nitroglycerin, |
| | | | Polyneuropathy, | Oxycodone 10 mg, |
| | | | Cellulitis, Coronary | Pantoprazole 40 |
| | | | Artery Disease (CAD), | mg, Plavix 75 mg, |
| | | | Gastroesophageal Reflux | Synthroid 50 mg, |
| | | | Disease (GERD), | Vultaren Gel |
| | | | Peripheral Vascular | 1%, Avastin |
| | | | Disease | |
| Fe- | 83 | Cau- | Retinal Vein Occlusion, | Pravastatin Sodium 20 |
| male | | casian | Hyperlipidemia | mg, Amlodipine |
| | | | (HLD), Ischemic | Besylate 5 mg, |
| | | | Cerebrovascular Disease, | Aspirin 81 mg, Eylea |
| | | | Osteoarthritis (OA), | |
| | | | Hypertension (HTN), | |
| | | | Hypothyroidism, Macular | |
| | | | Edema, Dry | |
| | | | Senile Macular | |
| | | | Degeneration | |
| Male | 85 | Cau- | Wet Age Related | Aspirin, |
| | | casian | Macular | Nexium, Avastin |
| | | | Degeneration | 1.25 mg, Finasteride |
| | | | (AMD), Neovascular | 1 mg, Vitamin |
| | | | Age Related Macular | B Complex, |
| | | | Degeneration | Atorvastatin 10 mg, |
| | | | | Clopidogrel 75 |
| | | | | mg, Lisinopril 10 mg, |
| | | | | Resveratrol 250 mg, |
| | | | | Vitamin D 400 iu-77 mg |
| Male | 82 | Cau- | Wet Age Related Macular | Aspirin 81 mg, |
| | | casian | Degeneration (AMD), | Atorvastatin 20 mg, |
| | | | Hypertension (HTN), | Coumadin, Finasteride, |
| | | | Hypercholesterolemia, | Furosemide, Iron, |
| | | | Anemia, Coronary | Hydralazine, |
| | | | Artery Disease | Losartan, Metoprolol, |
| | | | (CAD) | Xanax, Preservision |
| | | | | AREDS, Eylea |
| Fe- | 90 | Cau- | Wet Age Related | Calcium 1,000 |
| male | | casian | Macular | mg, Centrum |
| | | | Degeneration (AMD), | Silver, Eliquis, |
| | | | Hypertension (HTN), | Fish Oil 1,000 mg, |
| | | | Hypercholesterolemia, | Taztia 120 mg, |
| | | | Osteoarthritis (OA) | Zocor 20 mg, Eylea |
| | | | | 2.0 mg |
| Fe- | 89 | Cau- | Wet Age Related Macular | Ketorlac .5%, Aspirin |
| male | | casian | Degeneration (AMD), | 81 mg, Atorvastatin 80 |
| | | | Hypertension (HTN), | mg, Metoprolol 50 mg, |
| | | | Hypercholesterolemia, | Spironolactone 25 mg, |
| | | | Cataract | Vitamin B12 |
| | | | | 2,500 mg, Vitamin |
| | | | | D3 1,000 iu, Eylea |
| Fe- | 91 | Cau- | Wet Age Related Macular | Aspirin 81 mg, |
| male | | casian | Degeneration | AREDS Eye |
| | | | (AMD), Hypertension | Vitamin, Calcium |
| | | | (HTN), Raynaud's | 500 mg, Multivitamin, |
| | | | Phenomenon, | Vitamin B12 |
| | | | Cataract | 2,500 mg, Vitamin D3 |
| | | | | 50,000 iu, Avastin |
| Fe- | 75 | Cau- | Wet Age Related | Ecotrin 81 mg, |
| male | | casian | Macular | Methimazole 5 mg, |
| | | | Degeneration (AMD), | Synthroid, Metoprolol |
| | | | Hypothyroidism, | ER 25 mg, |
| | | | Peripheral Vascular | Avastin |
| | | | Disease | |
| Male | 75 | Cau- | Wet Age Related Macular | Metoprolol 100 |
| | | casian | Degeneration | mg, Ramipril |
| | | | (AMD), Hypertension | 10 mg, Xarelto 15 |
| | | | (HTN), | mg, Oxybutynin |
| | | | Hypercholesterolemia, | 10 mg, Atorvastatin |
| | | | Atrial | 40 mg, Ropinirole |
| | | | Fibrillation (AF), | 12 mg, Clopidogrel |
| | | | Benign Prostate | 75 mg, Zolpidem 5 mg, |
| | | | Hyperplasia (BPH), | Tamsulosin 0.8 mg, |
| | | | Gastroesophageal | Omeprazole |
| | | | Reflux Disease (GERD), | 10 mg, Eylea 2 mg |
| | | | Hyperlipidemia | |
| | | | (HLD), Parkinson's | |
| | | | Disease (PD), Insomnia, | |
| | | | Geographic | |
| | | | Atrophy (GA) | |

### Simple Plex Assay

Concentrations of inflammasome proteins (caspase-1, ASC, IL-1β and IL-18) in serum samples from AMD and age-matched controls were analyzed using the Ella System (Protein System)^{13, 16} In short, 50 μl of diluted serum sample were loaded to each well of the cartridge, and 1 mL of washing buffer was loaded into specified wells. The assay was analyzed by Simple Plex Runner Software. Results shown are the mean of each sample run in triplicate.

### Biomarker Analyses

Data obtained from the Simple Plex assay was analyzed using Prism 7 software (GraphPad). Initially, outliers were removed, followed by the calculation of column statistics and the area under curve, which provided the specificity, sensitivity and likelihood ratio, as well as the 95% confidence interval, standard deviation, and p-value. A cut-off point was identified for the different ranges of specificities and sensitivities. Positive and negative predictive values were also calculated as well as the accuracy of the assay.

### Statistical Analyses

Normality was tested using the D'Agostino & Pearson omnibus and Shapiro-Wilk normality tests. Differences between groups were determined using the Mann-Whitney test for non-normally distributed data and a two-tailed t-test for data that were normally distributed. The p-value of significance was set at <0.05.

### Linear Regression Analyses

Linear regression analysis between ASC and IL-18 were run using RStudio/RMarkdown with the following libraries: MASS, dplyr, ggplot, car and broom. Data sets were transformed using a logarithmic transformation. An adjusted r-squared value was obtained to determine the approximate contribution of ASC to IL-18 protein levels. P-value of significance was set at <0.05. Suitability of the models were then evaluated by residual analysis.

### Logistic Regression

A binomial logistic regression analyses of the probability of a patient having AMD as determined by the protein levels of ASC as well as IL-18 were run using RStudio/RMarkdown. P-value of significance was set at <0.05. Suitability of the models were then evaluated by comparing the Akaike information criterion (AIC) value to other tested models.

### Results

ASC and IL-18 are Elevated in the Serum of Patients with AMD

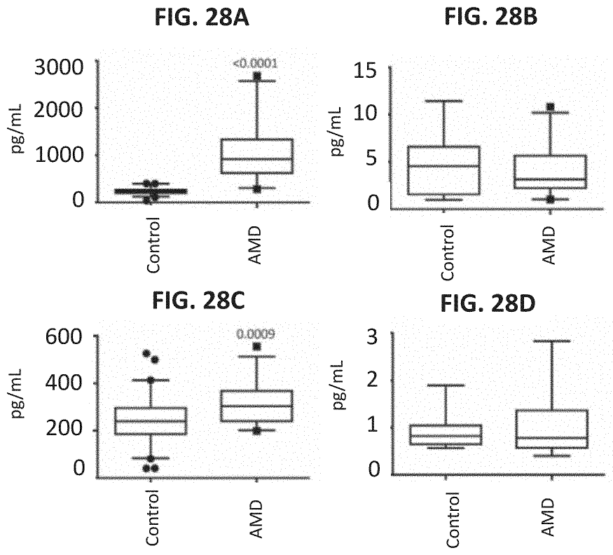

Serum samples from patients with AMD and aged-matched healthy donors were analyzed for the protein expression levels of ASC (FIG. 28A), caspase-1 (FIG. 28B), IL-18 (FIG. 28C) and IL-1β (FIG. 28D). ASC and IL-18 proteins were significantly higher in the AMD group when compared to the control group. This suggests that ASC and IL-18 play a role in the pathology of AMD.

### ASC as a Prominent Biomarker of AMD

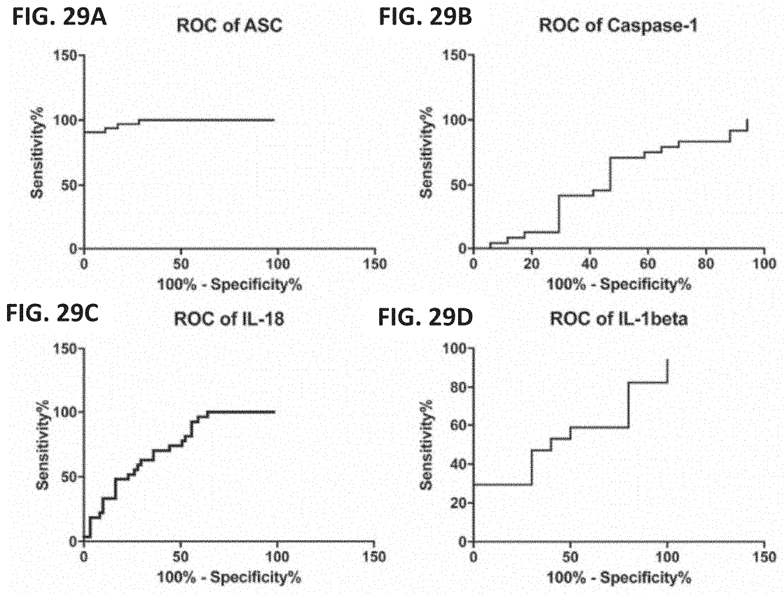

To determine if inflammasome signaling proteins may be used as biomarkers of AMD, the area under the curve (AUC) was calculated for ASC (FIG. 29A), caspase-1 (FIG. 29B), IL-18 (FIG. 29C) and IL-1β (FIG. 29D). Of the proteins that were analyzed, ASC had the highest AUC of 0.9823 (p<0.0001). IL-18 had an AUC of 0.7286 (p=0.0007) (Table 28). Moreover, ASC had a cut-off point of 365.6 pg/ml with 94% sensitivity and 89% specificity (Table 29). Comparatively, the cut-off point for IL-18 was 242.4 with a sensitivity of 74% and a specificity of 56% (Table 29).

TABLE 28
ROC analysis results for inflammasome signaling proteins
in serum in patients with AMD vs Control.

| Biomarker | Area | Std. Error | 95% C.I. | p-value |
|---|---|---|---|---|
| ASC | 0.9823 | 0.01183 | 0.9592 to 1.006 | <0.0001 |
| Caspase-1 | 0.5319 | 0.0967 | 0.3423 to 0.7214 | 0.7308 |
| IL-18 | 0.7286 | 0.0542 | 0.6224 to 0.8348 | 0.0007 |
| IL-1beta | 0.5294 | 0.1118 | 0.3103 to 0.7485 | 0.8018 |

TABLE 29
Cut-off point analyses for inflammasome signaling
proteins in serum of AMD patients

| | Cut-off | | | | | | |
|---|---|---|---|---|---|---|---|
| | point | Sensitivity | Specificity | | PPV | NPV | Accuracy |
| Biomarker | (pg/ml) | (%) | (%) | LR | (%) | (%) | (%) |
| ASC | >365.6 | 94 | 89 | 8.625 | 93 | 91 | 92 |
| Caspase-1 | <6.136 | 79 | 35 | 1.223 | 46 | 71 | 53 |
| IL-18 | >242.4 | 74 | 56 | 1.674 | 79 | 49 | 68 |
| IL-1beta | <0.842 | 59 | 50 | 1.176 | 41 | 67 | 53 |

### Differences Between Wet and Dry AMD

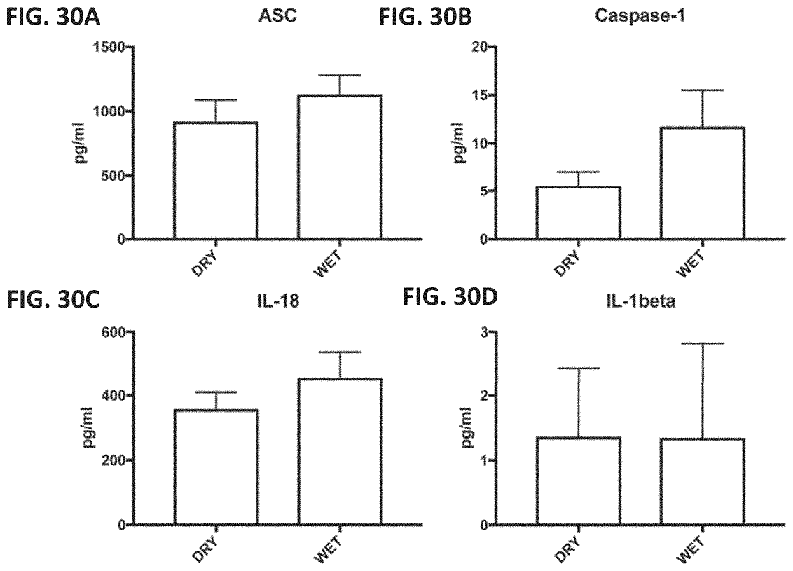

Patients were divided between dry and wet forms of the disease, and although there was not enough power to detect a difference between the small cohort of patients in these two groups, there was a trend for increased expression of ASC (FIG. 30A), caspase-1 (FIG. 30B) and IL-18 (FIG. 30C) in the serum of patients with the wet form of the disease when compared to the dry form, while the expression of IL1beta (FIG. 30D) shows no such trend.

### Linear Regression Between ASC and IL-18

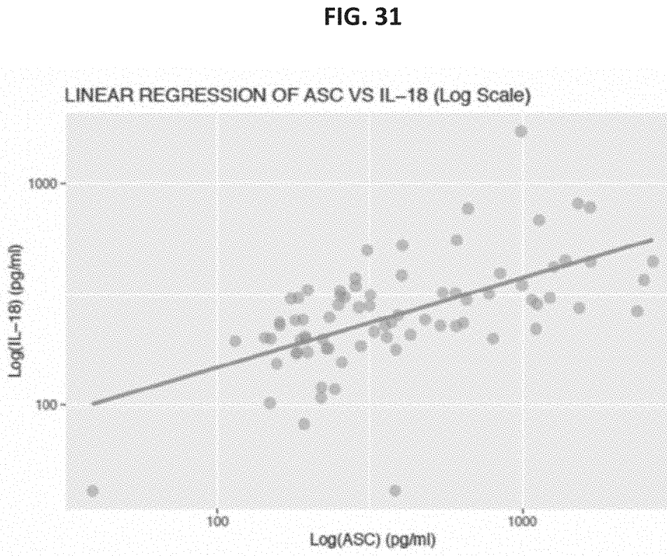

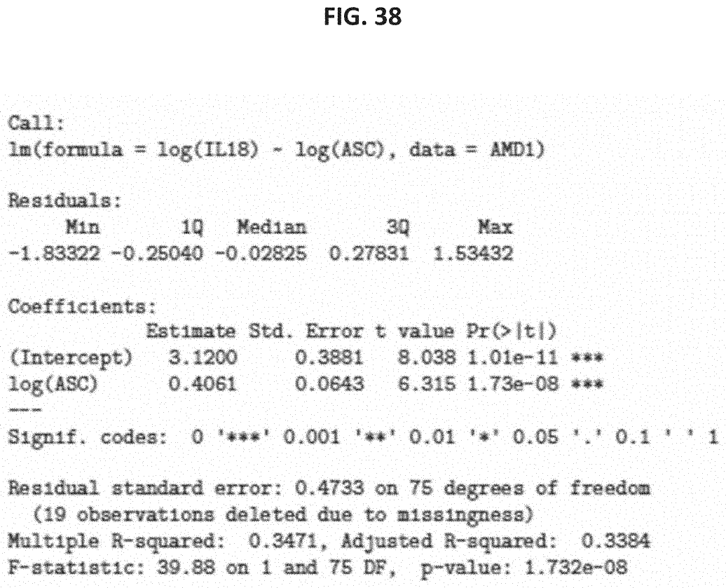

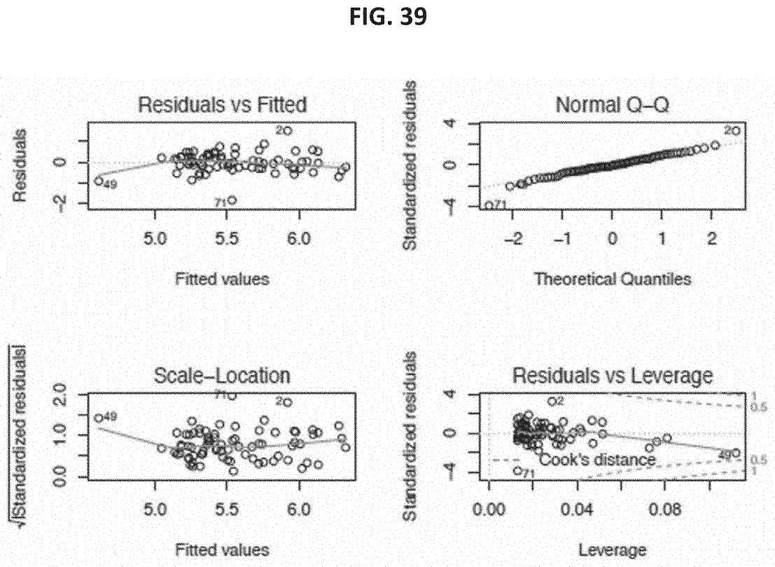

A linear regression analysis was run to determine the relationship between ASC and IL-18. A linear model was fit to the plotted data (FIG. 31). Levels of IL-18 had a statistically significant linear correlation with that of ASC (1.73e-08) with an adjusted R squared of 0.3384 (FIG. 38). A logarithmic transformation was used to normalized the distribution of the data. Further fitting of the model was evaluated by analyzing the residuals (FIG. 39). Thus, the results indicate that 34% of the levels of IL-18 could be explained by ASC. Thus, the data show that approximately a third of IL-18 present in serum can be explained by the levels of ASC, with the other two-thirds being due to other proteins that were not included in this statistical model.

### Logistic Regression Between AMD and ASC

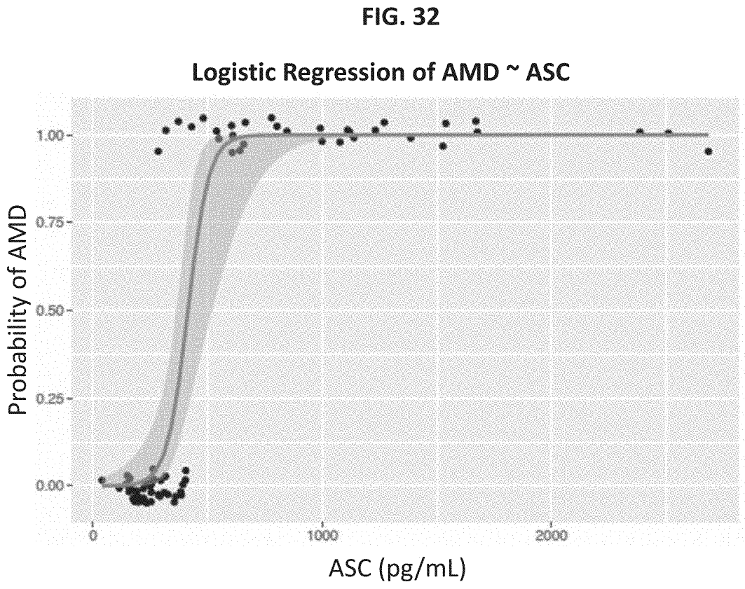

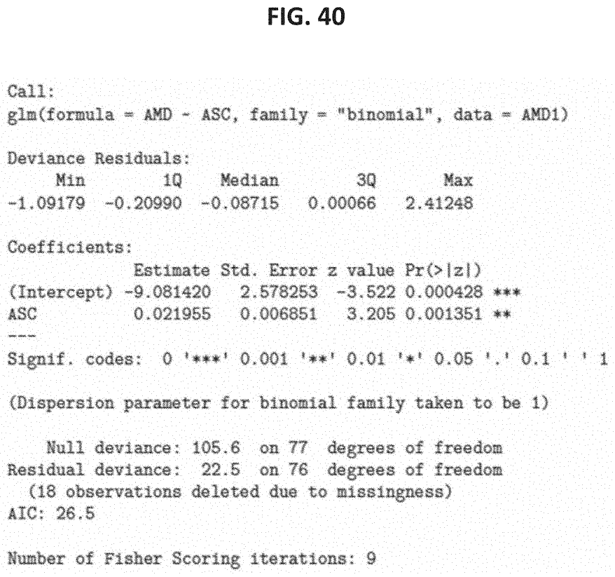

To predict the probability that protein levels of ASC contribute or not to the pathology of AMD, we run a binomial logistic regression for the proteins levels of ASC in serum of patients with and without an AMD diagnosis (FIG. 32). Accordingly, the odds of developing AMD increased with increased protein levels of ASC in serum as determined by an estimate coefficient of 0.022 (p=0.001351) (FIG. 40) and an exponentiated coefficient of 1.022.

### Logistic Regression Between AMD and IL-18

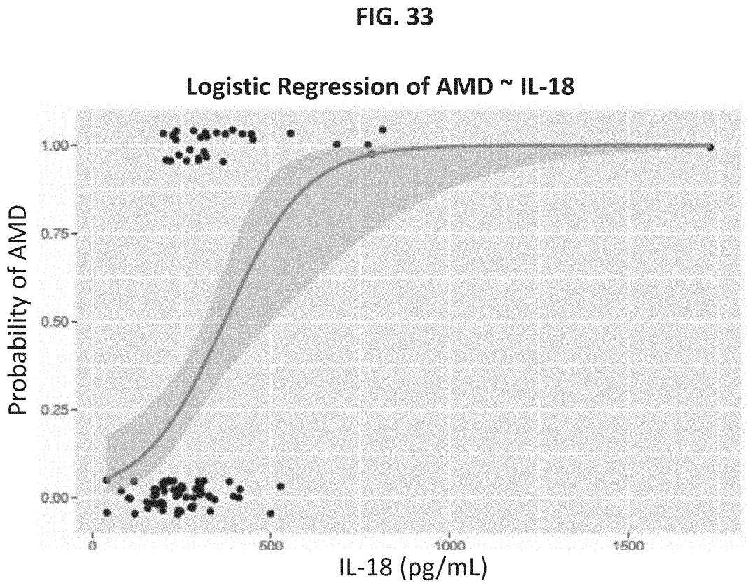

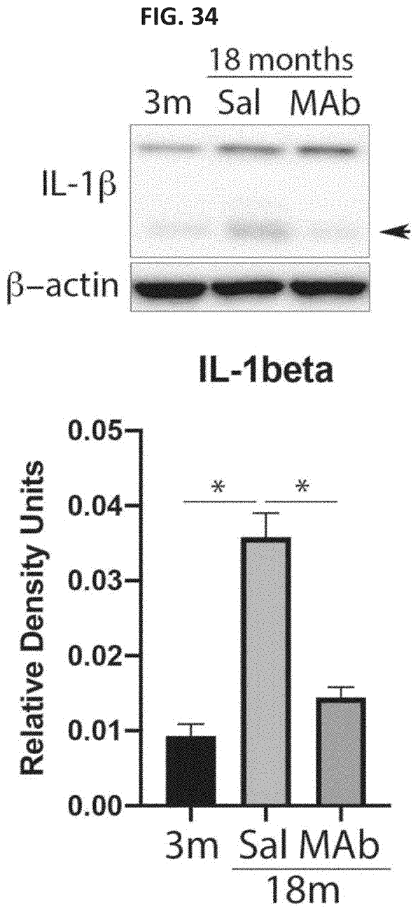

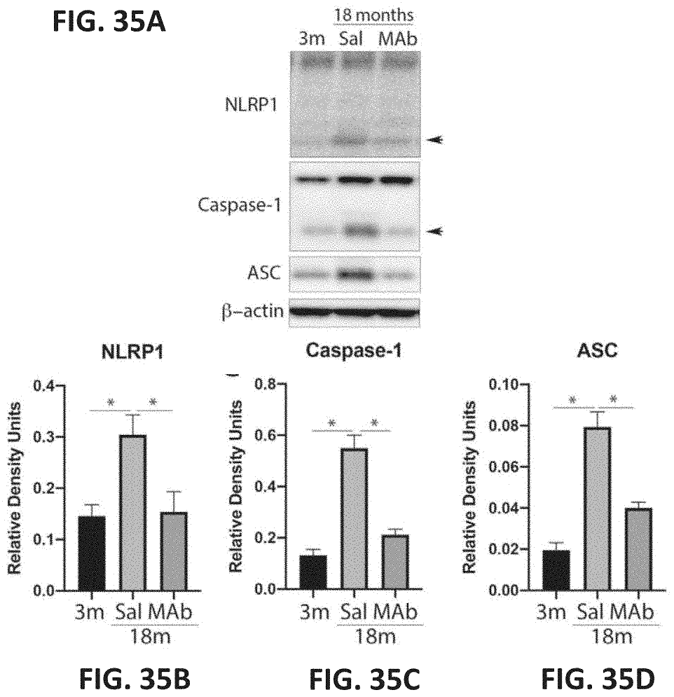

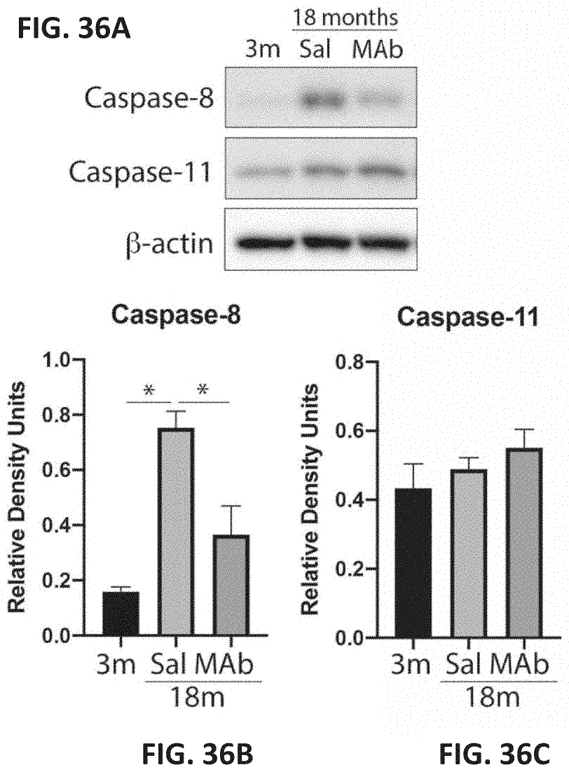

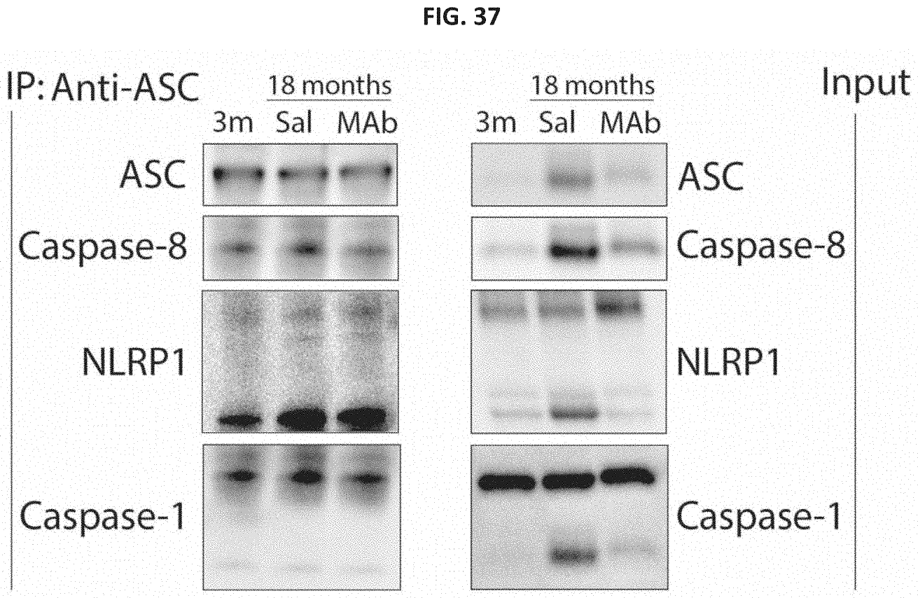

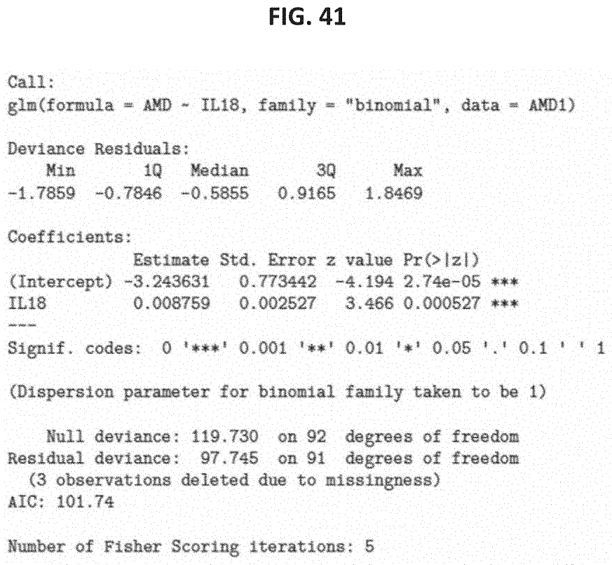

To predict the probability that protein levels of IL-18 contribute or not to the pathology of AMD, we run a binomial logistic regression for the proteins levels of IL-18 in serum of patients with and without an AMD diagnosis (FIG. 33). Accordingly, the odds of developing AMD increased with increased protein levels of IL-18 in serum as determined by an estimate coefficient of 0.009 (p=0.000527) (FIG. 41) and an exponentiated coefficient of 1.009.

### Conclusions

In this study, evidence that the inflammasome proteins ASC and IL-18 could be used as inflammatory biomarkers of AMD has been presented. Accordingly, in comparison to age-matched healthy donors, ASC and IL-18 were significantly higher in the serum of AMD patients. In addition, the AUC value for ASC (AUC: 0.982) provides argument for ASC being a strong biomarker in AMD.

ASC and IL-18 are useful individually, in combination, or with a platform of other proteins, for the diagnosis and prognosis of AMD.

In addition, to detecting higher levels of ASC and IL-18 in the serum of AMD patients compared to age-matched controls, and high AUC values for these proteins, we also divided out cohort of patients into those with wet and dry AMD and showed that there was a trend for higher levels of ASC, caspase-1 and IL-18 in the serum of patients with wet AMD.

Linear regression analysis between ASC and the pro-inflammatory cytokine IL-18 shows that 34% of IL-18 present in the serum of AMD patients is due to levels of ASC (FIG. 38). This suggests that a third of IL-18 can be accounted for due to ASC-dependent inflammasome activation, with other signaling pathways not included in this study responsible for the remainder of present IL-18 levels. Moreover, logistic regression analyses suggest that ASC and IL-18, individually, significantly contribute to the pathology of AMD.

### INCORPORATION BY REFERENCE

The following references are incorporated by reference in their entireties for all purposes.
1. Franklin B S, Bossaller L, De Nardo D, et al. The adaptor ASC has extracellular and ‘prionoid’ activities that propagate inflammation. Nat Immunol 2014; 15:727-737.
2. Wong W L, Su X, Li X, et al. Global prevalence of age-related macular degeneration and disease burden projection for 2020 and 2040: a systematic review and meta-analysis. Lancet Glob Health 2014; 2:e106-116.
3. Bird A C, Bressler N M, Bressler S B, et al. An international classification and grading system for age-related maculopathy and age-related macular degeneration. The International ARM Epidemiological Study Group. Surv Ophthalmol 1995; 39:367-374.
4. Zarbin M A. Current concepts in the pathogenesis of age-related macular degeneration. Arch Ophthalmol 2004; 122:598-614.
5. Ozaki E, Campbell M, Kiang A S, Humphries M, Doyle S L, Humphries P. Inflammation in age-related macular degeneration. Adv Exp Med Biol 2014; 801:229-235.
6. Pawelec G, Goldeck D, Derhovanessian E Inflammation, ageing and chronic disease. Curr Opin Immunol 2014; 29:23-28.
7. Mejias N H, Martinez C C, Stephens M E, de Rivero Vaccari J P. Contribution of the inflammasome to inflammaging. J Inflamm (Lond) 2018; 15:23.
8. Mawhinney L J, de Rivero Vaccari J P, Dale G A, Keane R W, Bramlett H M. Heightened inflammasome activation is linked to age-related cognitive impairment in Fischer 344 rats. BMC Neurosci 2011; 12:123.
9. Latz E, Duewell P. NLRP3 inflammasome activation in inflammaging. Semin Immunol 2018; 40:61-73.
10. Aden K, Rosenstiel P. The Dark Age(ing) of the Inflammasome. Immunity 2017; 46:173-175.
11. Platnich J M, Muruve D A. NOD-like receptors and inflammasomes: A review of their canonical and non-canonical signaling pathways. Arch Biochem Biophys 2019; 670:4-14.
12. Yonekawa Y, Miller J W, Kim I K. Age-Related Macular Degeneration: Advances in Management and Diagnosis. J Clin Med 2015; 4:343-359.
13. Keane R W, Dietrich W D, de Rivero Vaccari J P. Inflammasome Proteins As Biomarkers of Multiple Sclerosis. Front Neurol 2018; 9:135.
14. Kerr N, Garcia-Contreras M, Abbassi S, et al. Inflammasome Proteins in Serum and Serum-Derived Extracellular Vesicles as Biomarkers of Stroke. Front Mol Neurosci 2018; 11:309.
15. Kerr N, Lee S W, Perez-Barcena J, et al. Inflammasome proteins as biomarkers of traumatic brain injury. PLoS One 2018; 13: e0210128.
16. Brand F J, 3rd, Forouzandeh M, Kaur H, Travascio F, de Rivero Vaccari J P. Acidification changes affect the inflammasome in human nucleus pulposus cells. J Inflamm (Lond) 2016; 13:29.
17. Nassar K, Grisanti S, Elfar E, Luke J, Luke M, Grisanti S. Serum cytokines as biomarkers for age-related macular degeneration. Graefes Arch Clin Exp Ophthalmol 2015; 253:699-704.
18. Shen J, Choy D F, Yoshida T, et al. Interleukin-18 has antipermeablity and antiangiogenic activities in the eye: reciprocal suppression with VEGF. J Cell Physiol 2014; 229:974-983.
19. Ambati J, Fowler B J. Mechanisms of age-related macular degeneration. Neuron 2012; 75:26-39.
20. Gao J, Liu R T, Cao S, et al. NLRP3 inflammasome: activation and regulation in age-related macular degeneration. Mediators Inflamm 2015; 2015:690243.
21. Ildefonso C J, Biswal M R, Ahmed C M, Lewin A S. The NLRP3 Inflammasome and its Role in Age-Related Macular Degeneration. Adv Exp Med Biol 2016; 854:59-65.
22. Kerur N, Fukuda S, Banerjee D, et al. cGAS drives noncanonical-inflammasome activation in age-related macular degeneration. Nat Med 2018; 24:50-61.
23. Marneros A G. NLRP3 inflammasome blockade inhibits VEGF-A-induced age-related macular degeneration. Cell Rep 2013; 4:945-958.
24. Marneros A G. VEGF-A and the NLRP3 Inflammasome in Age-Related Macular Degeneration. Adv Exp Med Biol 2016; 854:79-85.
25. Puren A J, Fantuzzi G, Dinarello C A. Gene expression, synthesis, and secretion of interleukin 18 and interleukin 1beta are differentially regulated in human blood mononuclear cells and mouse spleen cells. Proc Natl Acad Sci USA 1999; 96:2256-2261.
26. Congdon N, O'Colmain B, Klaver C C, et al. Causes and prevalence of visual impairment among adults in the United States. Arch Ophthalmol 2004; 122:477-485.
27. Friedman D S, O'Colmain B J, Munoz B, et al. Prevalence of age-related macular degeneration in the United States. Arch Ophthalmol 2004; 122:564-572.
28. Rauch R, Weingessel B, Maca S M, Vecsei-Marlovits P V. Time to first treatment: The significance of early treatment of exudative age-related macular degeneration. Retina 2012; 32:1260-1264.
29. Schwartz R, Loewenstein A. Early detection of age related macular degeneration: current status. Int J Retina Vitreous 2015; 1:20.

### Example 6-Testing Monoclonal Antibody (mAb) Directed Against ASC as Treatment for Age-Related Inflammation and Alzheimer's Disease

### Background/Objective

Aging of the brain is a common-denominator in several neurodegenerative diseases¹. A factor associated with aging is cognitive decline. Cognitive decline is highly conserved among mammals, including humans, rodents, monkeys and dogs^{2, 3, 4}. Associated with the process of aging is chronic inflammation. Inflammaging, or aging-related inflammation, is a risk factor for morbidity and mortality in the elderly population, and it is regulated, in part, by the innate immune response. Targeting the inflammatory response in the aging brain has the potential to improve cognitive performance.

The objective of the experiments in this experiment were designed to demonstrate the therapeutic effect of inhibiting inflammation associated with aging in regards to improved cognitive performance and overall wellbeing in the context of aging.

In order to determine the utility of a humanized, anti-ASC monoclonal antibody (i.e., IC-100) in treating age-related inflammation (i.e., inflammaging), said antibody was administered to young (i.e., 3-months old) and aged (i.e., 18-months old) C57 BL/6 mice and the subsequent effect of said antibody treatment on inflammasome markers proteins in young vs. aged mice was assessed.

### Materials and Method

Animals

All animal procedures were approved by the Animal Care and Use Committee of the University of Miami (protocol 19-029). Animal procedures were carried according to Guide for the Care and Use of Laboratory Animals (U.S. Public Health). C57BL/6 male mice at 3 and 18 months old were treated with IC-100 (5 mg/kg) and saline intraperitoneally (i.p.) and sacrificed 3 days later. The brain cortex was then removed and protein lysates were obtained and at stored at −80° C. for biochemical analyses.

Immunoblotting

Analyses of inflammasome protein expression were measured by immunoblot analysis as previously described. Briefly, cortical lysates were resolved in 4-20% Criterion TGX Stain-Free precasted gels (Bio-Rad), using antibodies (1:1000 dilution) to NLRP1 (Novus Biologicals), caspase-1 (Novus Biologicals), ASC (Santa Cruz), IL-1β (Cell Signaling) and beta-actin (Sigma Aldric). Quantification of band densities was done using the UNSCAN-IT gel 6.3 Software (Silk Scientific Corporation) and membranes were imaged using the ChemiDoc Touch Imaging System (BioRad) following chemiluminescence.

Co-Immunoprecipitation

To assess the protein composition and association of proteins in the non-canonical inflammasome, a Protein G Kit (Miltenyi Biotec) was used according to manufacturer instructions using samples from young and aged mice. Briefly, 2 μg of IC-100 were added to 20 μg of brain cortical protein lysate and then mixed with 50 μl of Protein G MicroBeads in order to magnetically label the immune complex. Then the lysate was applied onto a μColumn in the magnetic field of the μMACS™ Separator (Miltenyi Biotec) followed by rinsing with lysis buffer (4λ) and RIPA buffer (1×) followed by elution with 20 μl of preheated (95° C.) 1× laemmli buffer and then with 50 μl of 1× laemmli buffer. Eluted protein in laemmli buffer was then resolved by immunoblotting as described. The input was run in parallel as a positive control.

Statistical Analyses

Following identification and removal of outliers, comparison between groups was done by a one-way ANOVA followed by Tukey's multiple comparison test. Data are presented as mean+/−SEM. P-value of significance was set to less than 0.05 in all tests.

### Results/Conclusions

IC-100 inhibits IL-1b-mediated inflammation in the cortex of aged mice. In this regard, the experiments in this example provided the first evidence of inflammasome activation in the hippocampus of aged rats, in which rats treated with a non-specific inflammasome inhibitor, showed decreased activation of caspase-1². Importantly, this effect was associated with improved spatial learning performance. Given the known role for the inflammasome and the inflammasome-mediated cell death mechanism of pyroptosis in inflammaging³, modulation of inflammation in the brain is a promising approach to improving cognitive performance in the elderly population.

### INCORPORATION BY REFERENCE

The following references are incorporated by reference in their entireties for all purposes.
1. Chen, M. et al. Internalized Cryptococcus neoformans Activates the Canonical Caspase-1 and the Noncanonical Caspase-8 Inflammasomes. J Immunol 195, 4962-4972 (2015).
2. Chi, W. et al. Caspase-8 promotes NLRP1/NLRP3 inflammasome activation and IL-1beta production in acute glaucoma. Proc Natl Acad Sci USA 111, 11181-11186 (2014).
3. Yankner, B. A., Lu, T. & Loerch, P. The aging brain. Annual review of pathology 3, 41-66 (2008).
4. Head, E. et al. Spatial learning and memory as a function of age in the dog. Behavioral neuroscience 109, 851-858 (1995).
5. Lai, Z. C., Moss, M. B., Killiany, R. J., Rosene, D. L. & Herndon, J. G. Executive system dysfunction in the aged monkey: spatial and object reversal learning. Neurobiology of aging 16, 947-954 (1995).
6. Mawhinney, L. J., de Rivero Vaccari, J. P., Dale, G. A., Keane, R. W. & Bramlett, H. M. Heightened inflammasome activation is linked to age-related cognitive impairment in Fischer 344 rats. BMC neuroscience 12, 123 (2011).
7. Mejias, N. H., Martinez, C. C., Stephens, M. E. & de Rivero Vaccari, J. P. Contribution of the inflammasome to inflammaging. J Inflamm (Lond) 15, 23 (2018).

### Example 7: Examination of Inflammasome Proteins as Biomarkers of Nonalcoholic Steatohepatitis (NASH)

### Introduction

A biomarker is a characteristic that can be measured objectively and evaluated as an indicator of normal or pathologic biological processes. Important to the care of patients with NASH are the need for biomarkers that can screen for and diagnose NASH, detect exacerbation of NASH, and evaluate a patient's response to treatment.

### Methods

### Simple Plex Assay

Concentrations of inflammasome proteins (C-Reactive Protein, ASC, Gal-3 and IL-18) in serum samples from NASH subjects and age-matched controls were analyzed using the Ella System (Protein System) as described previously herein. In short, 50 μl of diluted serum sample were loaded to each well of the cartridge, and 1 mL of washing buffer was loaded into specified wells. The assay was analyzed by Simple Plex Runner Software. Results shown are the mean of each sample run in triplicate.

### Biomarker Analyses

Data obtained from the Simple Plex assay was analyzed using Prism 7 software (GraphPad). Initially, outliers were removed, followed by the calculation of column statistics and the area under curve, which provided the specificity, sensitivity and likelihood ratio, as well as the 95% confidence interval, standard deviation, and p-value. A cut-off point was identified for the different ranges of specificities and sensitivities. Positive and negative predictive values were also calculated as well as the accuracy of the assay.

### Statistical Analyses

Normality was tested using the D'Agostino & Pearson omnibus and Shapiro-Wilk normality tests. Differences between groups were determined using the Mann-Whitney test for non-normally distributed data and a two-tailed t-test for data that were normally distributed. The p-value of significance was set at <0.05.

### Logistic Regression

A binomial logistic regression analyses of the probability of a patient having NASH as determined by the protein levels of ASC, IL-18 and Gal-3 were run using RStudio/RMarkdown. P-value of significance was set at <0.05. Suitability of the models were then evaluated by comparing the Akaike information criterion (AIC) value to other tested models.

### Results

ASC and IL-18 are Elevated in the Serum of Patients with NASH

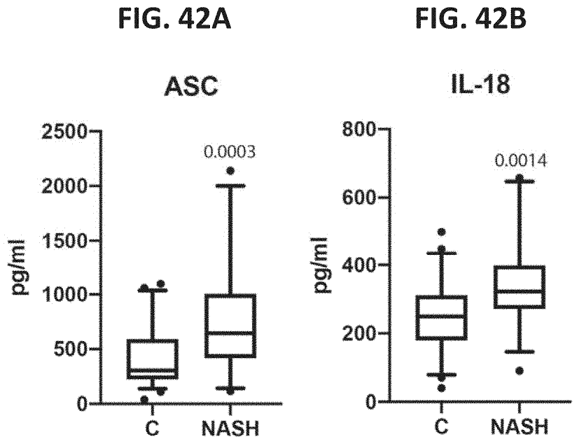

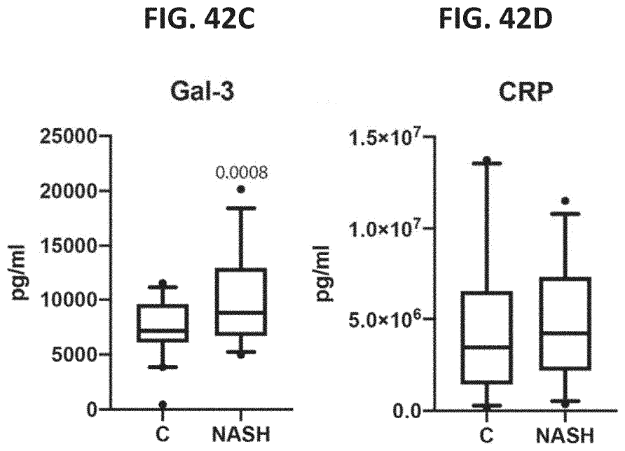

Serum samples from patients with NASH and aged-matched healthy donors were analyzed for the protein expression levels of ASC (FIG. 42A), IL-18 (FIG. 42B), Galectin-3 (Gal-3) (FIG. 42C) and C-Reaction Protein (CRP) (FIG. 42D). ASC and IL-18 proteins were significantly higher in the NASH group when compared to the control group, similar to Gal-3, a galectin known to play a role in pathophysiology of hepatic fibrosis from various chronic liver diseases. This suggests that ASC and IL-18 may play a role in the pathology of NASH.

### ASC as a Prominent Biomarker of NASH

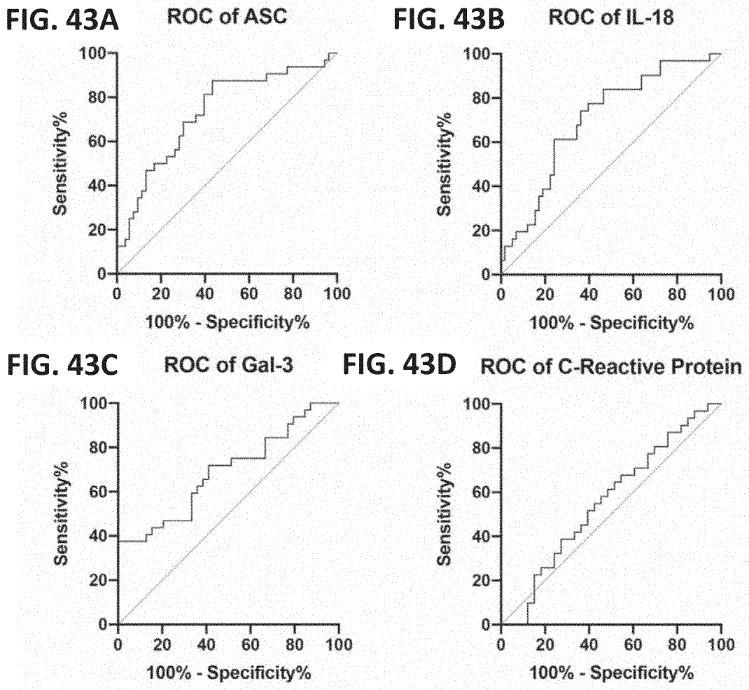

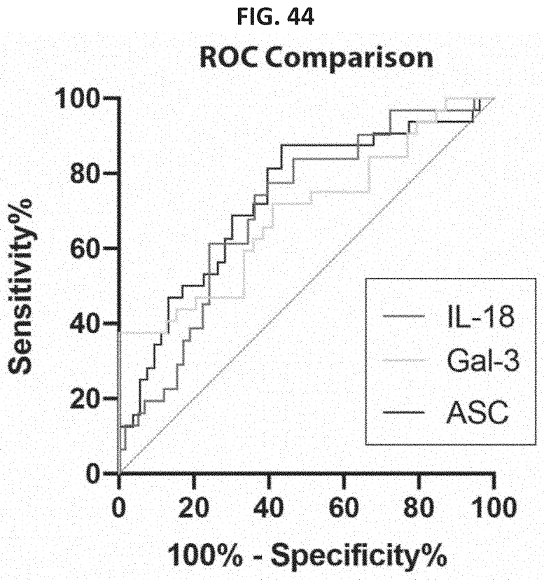

To determine if inflammasome signaling proteins may be used as biomarkers of NASH, the area under the curve (AUC) was calculated for ASC (FIG. 43A), IL-18 (FIG. 43B), Gal-3 (FIG. 43C) and CRP (FIG. 43D). Of the proteins that were analyzed, ASC had the highest AUC of 0.7317 (p=0.0004). IL-18 had an AUC of 0.7036 (p=0.0016) (Table 32). Moreover, ASC had a cut-off point of 394.9 pg/ml with 81% sensitivity and 60% specificity (Table 33; FIG. 44). Comparatively, the cut-off point for IL-18 was >269.2 with a sensitivity of 77% and a specificity of 60% (Table 33; FIG. 44).

TABLE 32
ROC analysis results for inflammasome signaling proteins in
serum in patients with NASH vs Control.

| Biomarker | AUC | Std. Error | 95% C.I. | p-value |
|---|---|---|---|---|
| ASC | 0.7317 | 0.05725 | 0.6195 to 0.8439 | 0.0004 |
| IL-18 | 0.7036 | 0.05671 | 0.5924 to 0.8147 | 0.0016 |
| Galectin-3 | 0.6891 | 0.06416 | 0.5633 to 0.8149 | 0.0064 |
| CRP | 0.5572 | 0.07247 | 0.4151 to 0.6992 | 04319 |

TABLE 33
Cut-off point analyses for inflammasome signaling
proteins in serum of NASH patients.

| | Cut-off | | | | | | |
|---|---|---|---|---|---|---|---|
| | point | Sensitivity | Specificity | PPV | NPV | Likelihood | Accuracy |
| Biomarker | (pg/ml) | (%) | (%) | (%) | (%) | Ratio | (%) |
| ASC | >394.9 | 81 | 60 | 55 | 84 | 2.051 | 68 |
| IL-18 | >269.2 | 77 | 60 | 51 | 83 | 1.952 | 66 |
| Galectin-3 | >7,120 | 75 | 49 | 55 | 70 | 1.463 | 61 |
| CRP | >2,895,004 | 68 | 42 | 52 | 58 | 1.177 | 55 |

TABLE 34
Results of Binomial Logistic Regression Modeling

| | Exponentiated | | | | |
|---|---|---|---|---|---|
| Model | Estimate | Accuracy | AUC | AIC | p-value |
| ASC + | 1.003646582 | 78% | 85% | 68.951 | 0.0262 |
| IL-18 + | 1.005912319 | | | | 0.11557 |
| Gal-3 | 1.000214045 | | | | 0.10643 |
| ASC | 1.0026012 | 71% | 72% | 102.03 | 0.000944 |
| ASC+ | 1.00184318 | 68% | 76% | 96.813 | 0.026752 |
| IL-18 | 1.00485550 | | | | .067626 |
| Gal-3 | 1.00028235 | 62% | 72% | 90.032 | 0.00310 |
| IL-18 | 1.00672453 | 64% | 70% | 108.82 | 0.003434 |
| ASC + | 1.00475151 | 75% | 82% | 73.036 | 0.00111 |
| Gal-3 | 1.00018294 | | | | 0.13440 |
| IL-18 | 1.009849176 | 70% | 83% | 76.818 | 0.001888 |
| Gal-3 | 1.000327311 | | | | 0.003965 |

### Conclusions

In this study, evidence that the inflammasome proteins ASC and IL-18 could be used as inflammatory biomarkers of NASH has been presented. Accordingly, in comparison to age-matched healthy donors, ASC and IL-18 were significantly higher in the serum of NASH patients. In addition, the AUC value for ASC (AUC: 0.7317) provides argument for ASC being a strong biomarker in AMD.

ASC and IL-18 are useful individually, in combination, or with a platform of other proteins (e.g., Gal-3 and/or CRP), for the diagnosis and prognosis of NASH. Moreover, logistic regression analyses suggest that ASC and IL-18, individually, significantly contribute to the pathology of NASH.

### NUMBERED EMBODIMENTS OF THE DISCLOSURE

Other subject matter contemplated by the present disclosure is set out in the following numbered embodiments:

1. A method of evaluating a patient suspected of having multiple sclerosis (MS), the method comprising: measuring the level of at least one inflammasome protein in a biological sample obtained from the patient; determining the presence or absence of a protein signature associated with MS, wherein the protein signature comprises an elevated level of the at least one inflammasome protein; and selecting the patient as having MS if the patient exhibits the presence of the protein signature.

2. The method of embodiment 1, wherein the patient is presenting with clinical symptoms consistent with MS.

3. The method of embodiment 1 or 2, wherein the MS is relapsing-remitting MS (RRMS), secondary-progressive MS (SPMS), primary-progressive MS (PPMS), or progressive-relapsing MS (PRMS).

4. The method of any one of the above embodiments, wherein the biological sample obtained from the patient is cerebrospinal fluid (CSF), CNS microdialysate, saliva, serum, plasma, urine or serum-derived extracellular vesicles (EVs).

5. The method of any one of the above embodiments, wherein the level of the at least one inflammasome protein in the protein signature is measured by an immunoassay utilizing one or more antibodies directed against the at least one inflammasome protein in the protein signature.

6. The method of any one of the above embodiments, wherein the at least one inflammasome protein is interleukin 18 (IL-18), IL-1beta, apoptosis-associated speck-like protein containing a caspase recruitment domain (ASC), caspase-1, or combinations thereof.

7. The method of any of the above embodiments, wherein the at least one inflammasome protein comprises each of caspase-1, IL-18, IL-1beta and ASC.

8. The method of any one of embodiments 1-6, wherein the at least one inflammasome protein comprises ASC.

9. The method of any one of embodiments 5-8, wherein the antibody binds to the PYRIN-PAAD-DAPIN domain (PYD), C-terminal caspase-recruitment domain (CARD) domain or a portion of the PYD or CARD domain of the ASC protein.

10. The method of any one of the above embodiments, wherein the level of the at least one inflammasome protein in the protein signature is enhanced relative to the level of the at least one inflammasome protein in a biological sample obtained from a control.

11. The method of embodiment 10, wherein the biological sample obtained from the control is cerebrospinal fluid (CSF), CNS microdialysate, saliva, serum, plasma, urine or serum-derived extracellular vesicles (EVs).

12. The method of embodiment 10 or 11, wherein the control is a healthy individual, wherein the healthy individual is an individual not presenting with clinical symptoms consistent with MS.

13. The method of any one of embodiments 10-12, wherein the at least one inflammasome protein comprises ASC, wherein the level of ASC is at least 50% higher than the level of ASC in the biological sample obtained from a control.

14. The method of any one of embodiments 1-9, wherein the level of the at least one inflammasome protein in the protein signature is enhanced relative to a pre-determined reference value or range of reference values.

15. The method of embodiment 14, wherein the biological sample obtained from patient is serum and the patient is selected as having MS with a sensitivity of at least 80%, 85%, 90%, 95%, 99% or 100% and a specificity of at least 90%.

16. The method of embodiment 14 or 15, wherein the biological sample is serum and the patient is selected as having MS with a specificity of at least 80%, 85%, 90%, 95%, 99% or 100%.

17. The method of embodiment 14, wherein the biological sample is serum and the patient is selected as having MS with a sensitivity of at least 90% and a specificity of at least 80%.

18. The method of any one of embodiments 14-17, wherein the at least one inflammasome protein comprises ASC.

19. The method of embodiment 18, wherein a cut-off value for determining the sensitivity, specificity or both is selected from Table 7.

20. The method of any one of embodiments 15-17, wherein the sensitivity and/or sensitivity is determined using the area under curve (AUC) from receiver operator characteristic (ROC) curves with confidence intervals of 95%.

21. A method of evaluating a patient suspected of having suffered a stroke, the method comprising: measuring the level of at least one inflammasome protein in a biological sample obtained from the patient; determining the presence or absence of a protein signature associated with stroke or a stroke-related injury, wherein the protein signature comprises an elevated level of the at least one inflammasome protein; and selecting the patient as having suffered from a stroke if the patient exhibits the presence of the protein signature.

22. The method of embodiment 21, wherein the patient is presenting with clinical symptoms consistent with stroke, wherein the stroke is ischemic stroke, transient ischemic stroke or hemorrhagic stroke.

23. The method of embodiment 21 or 22, wherein the biological sample obtained from the patient is cerebrospinal fluid (CSF), CNS microdialysate, saliva, serum, plasma, urine or serum-derived extracellular vesicles (EVs).

24. The method of any one of embodiments 21-23, wherein the level of the at least one inflammasome protein in the protein signature is measured by an immunoassay utilizing one or more antibodies directed against the at least one inflammasome protein in the protein signature.

25. The method of any one of embodiments 21-24, wherein the at least one inflammasome protein is interleukin 18 (IL-18), IL-1beta, apoptosis-associated speck-like protein containing a caspase recruitment domain (ASC), caspase-1, or combinations thereof.

26. The method of any of embodiments 21-25, wherein the at least one inflammasome protein comprises each of caspase-1, IL-18, IL-1beta and ASC.

27. The method of any one of embodiments 21-25, wherein the at least one inflammasome protein comprises ASC.

28. The method of any one of embodiments 25-27, wherein the antibody binds to the PYRIN-PAAD-DAPIN domain (PYD), C-terminal caspase-recruitment domain (CARD) domain or a portion of the PYD or CARD domain of the ASC protein.

29. The method of any one of embodiments 21-28, wherein the level of the at least one inflammasome protein in the protein signature is enhanced relative to the level of the at least one inflammasome protein in a biological sample obtained from a control.

30. The method of embodiment 29, wherein the biological sample obtained from the control is cerebrospinal fluid (CSF), CNS microdialysate, saliva, serum, plasma, urine or serum-derived extracellular vesicles (EVs).

31. The method of embodiment 29 or 30, wherein the control is a healthy individual, wherein the healthy individual is an individual not presenting with clinical symptoms consistent with MS.

32. The method of any one of embodiments 29-31, wherein the at least one inflammasome protein comprises ASC, wherein the level of ASC in a serum sample obtained from the subject is at least 70% higher than the level of ASC in a serum sample obtained from a control.

33. The method of any one of embodiments 29-31, wherein the at least one inflammasome protein comprises ASC, wherein the level of ASC in a serum-derived EV sample obtained from the subject is at least 110% higher than the level of ASC in a serum-derived EV sample obtained from a control.

34. The method of any one of embodiments 21-28, wherein the level of the at least one inflammasome protein in the protein signature is enhanced relative to a pre-determined reference value or range of reference values.

35. The method of embodiment 34, wherein the biological sample obtained from patient is serum and the patient is selected as having suffered a stroke with a sensitivity of at least 80%, 85%, 90%, 95%, 99% or 100% and a specificity of at least 90%.

36. The method of embodiment 34 or 35, wherein the biological sample is serum and the patient is selected as having suffered a stroke with a specificity of at least 80%, 85%, 90%, 95%, 99% or 100%.

37. The method of embodiment 34, wherein the biological sample is serum and the patient is selected as having suffered a stroke with a sensitivity of at least 100% and a specificity of at least 95%.

38. The method of any one of embodiments 35-37, wherein the at least one inflammasome protein comprises ASC.

39. The method of embodiment 38, wherein a cut-off value for determining the sensitivity, specificity or both is selected from Table 8.

40. The method of embodiment 34, wherein the biological sample obtained from patient is serum-derived EVs and the patient is selected as having suffered a stroke with a sensitivity of at least 80%, 85%, 90%, 95%, 99% or 100% and a specificity of at least 90%.

41. The method of embodiment 34 or 40, wherein the biological sample is serum-derived EVs and the patient is selected as having suffered a stroke with a specificity of at least 80%, 85%, 90%, 95%, 99% or 100%.

42. The method of embodiment 34, wherein the biological sample is serum-derived EVs and the patient is selected as having suffered a stroke with a sensitivity of at least 100% and a specificity of at least 100%.

43. The method of any one of embodiments 40-42, wherein the at least one inflammasome protein comprises ASC.

44. The method of embodiment 43, wherein a cut-off value for determining the sensitivity, specificity or both is selected from Table 9.

45. The method of any one of embodiments 35-37 or 40-42, wherein the sensitivity and/or sensitivity is determined using the area under curve (AUC) from receiver operator characteristic (ROC) curves with confidence intervals of 95%.

46. A method of treating a patient diagnosed with multiple sclerosis (MS), the method comprising administering a standard of care treatment for MS to the patient, wherein the diagnosis of MS was made by detecting an elevated level of at least one inflammasome protein in a biological sample obtained from the patient.

47. The method of embodiment 46, wherein the MS is relapsing-remitting MS (RRMS), secondary-progressive MS (SPMS), primary-progressive MS (PPMS), or progressive-relapsing MS (PRMS).

48. The method of embodiment 46 or 47, wherein the standard of care treatment is selected from therapies directed towards modifying disease outcome, managing relapses, managing symptoms or any combination thereof.

49. The method of embodiment 48, wherein the therapies directed toward modifying disease outcome are selected from beta-interferons, glatiramer acetate, fingolimod, teriflunomide, dimethyl fumarate, mitoxanthrone, ocrelizumab, alemtuzumab, daclizumab and natalizumab.

50. A method of treating a patient diagnosed with stroke or a stroke related injury, the method comprising administering a standard of care treatment for stroke or stroke-related injury to the patient, wherein the diagnosis of stroke or stroke-related injury was made by detecting an elevated level of at least one inflammasome protein in a biological sample obtained from the patient.

51. The method of embodiment 50, wherein the stroke is ischemic stroke, transient ischemic stroke or hemorrhagic stroke.

52. The method of embodiment 50 or 51, wherein the stroke is ischemic stroke or transient ischemic stroke and the standard of care treatment is selected from tissue plasminogen activator (tPA), antiplatelet medicine, anticoagulants, a carotid artery angioplasty, carotid endarterectomy, intra-arterial thrombolysis and mechanical clot removal in cerebral ischemia (MERCI) or a combination thereof.

53. The method of embodiment 50 or 51, wherein the stroke is hemorrhagic stroke and the standard of care treatment is an aneurysm clipping, coil embolization or arteriovenous malformation (AVM) repair.

54. The method of any one of embodiments 46-53, wherein the elevated level of the at least one inflammasome protein is measured by an immunoassay utilizing one or more antibodies directed against the at least one inflammasome protein.

55. The method of any one of embodiments 46-54, wherein the level of the at least one inflammasome protein is enhanced relative to the level of the at least one inflammasome protein in a control sample.

56. The method of any one of embodiments 46-54, wherein the level of the at least one inflammasome protein is enhanced relative to a pre-determined reference value or range of reference values.

57. The method of any one of embodiments 46-56, wherein the at least one inflammasome protein is interleukin 18 (IL-18), apoptosis-associated speck-like protein containing a caspase recruitment domain (ASC), caspase-1, or combinations thereof.

58. The method of embodiment 56 or 57, wherein the at least one inflammasome protein is caspase-1, IL-18, and ASC.

59. The method of embodiment 56 or 57, wherein the at least one inflammasome protein is ASC.

60. The method of embodiment 59, wherein the antibody binds to the PYRIN-PAAD-DAPIN domain (PYD), C-terminal caspase-recruitment domain (CARD) domain or a portion of the PYD or CARD domain of the ASC protein.

61. The method of any one of embodiments 46-60, wherein the biological sample is cerebrospinal fluid (CSF), CNS microdialysate, saliva, serum, plasma, urine or serum-derived extracellular vesicles (EVs).

62. A method of evaluating a patient suspected of having traumatic brain injury (TBI), the method comprising: measuring the level of at least one inflammasome protein in a biological sample obtained from the patient; determining the presence or absence of a protein signature associated with TBI, wherein the protein signature comprises an elevated level of the at least one inflammasome protein; and selecting the patient as having TBI if the patient exhibits the presence of the protein signature.

63. The method of embodiment 62, wherein the patient is presenting with clinical symptoms consistent with TBI.

64. The method of embodiment 62 or 63, wherein the biological sample obtained from the patient is cerebrospinal fluid (CSF), CNS microdialysate, saliva, serum, plasma, urine or serum-derived extracellular vehicles (EVs).

65. The method of any one of embodiments 62-64, wherein the level of the at least one inflammasome protein in the protein signature is measured by an immunoassay utilizing one or more antibodies directed against the at least one inflammasome protein in the protein signature.

66. The method of any one of embodiments 62-65, wherein the at least one inflammasome protein is interleukin 18 (IL-18), IL-1β, apoptosis-associated speck-like protein containing a caspase recruitment domain (ASC), caspase-1, or combinations thereof.

67. The method of any one of embodiments 61-66, wherein the at least one inflammasome protein comprises caspase-1.

The method of any one of embodiments 65-67, wherein the at least one inflammasome protein comprises caspase-1, wherein the level of caspase-1 is at least 50% higher than the level of caspase-1 in the biological sample obtained from a control.

68. The method of any one of embodiments 61-66, wherein the at least one inflammasome protein comprises ASC.

69. The method of any one of embodiments 66 or 68, wherein the antibody binds to the PYRIN-PAAD-DAPIN domain (PYD), C-terminal caspase-recruitment domain (CARD) domain or a portion of the PYD or CARD domain of the ASC protein.

70. The method of any one of embodiments 62-69, wherein the level of the at least one inflammasome protein in the protein signature is enhanced relative to the level of the at least one inflammasome protein in a biological sample obtained from a control.

71. The method of embodiment 70, wherein the at least one inflammasome protein comprises caspase-1, wherein the level of caspase-1 is at least 50% higher than the level of caspase-1 in the biological sample obtained from the control.

72. The method of embodiment 70, wherein the at least one inflammasome protein comprises ASC, wherein the level of ASC is at least 50% higher than the level of ASC in the biological sample obtained from the control.

73. The method of any one of embodiments 70-72, wherein the biological sample obtained from the control is cerebrospinal fluid (CSF), CNS microdialysate, saliva, serum, plasma, urine or serum-derived extracellular vesicles (EVs).

74. The method of any one of embodiments 70-73, wherein the control is a healthy individual, wherein the healthy individual is an individual not presenting with clinical symptoms consistent with TBI.

75. The method of any one of embodiments 62-69, wherein the level of the at least one inflammasome protein in the protein signature is enhanced relative to a pre-determined reference value or range of reference values.

76. The method of embodiment 75, wherein the biological sample obtained from patient is serum and the patient is selected as having TBI with a sensitivity of at least 80%, 85%, 90%, 95%, 99% or 100% and a specificity of at least 90%.

77. The method of embodiment 75 or 76, wherein the biological sample is serum and the patient is selected as having TBI with a specificity of at least 80%, 85%, 90%, 95%, 99% or 100%.

78. The method of embodiment 75, wherein the biological sample is serum and the patient is selected as having TBI with a sensitivity of at least 90% and a specificity of at least 80%.

79. The method of any one of embodiments 76-76, wherein the sensitivity and/or sensitivity is determined using the area under curve (AUC) from receiver operator characteristic (ROC) curves with confidence intervals of 95%.

80. The method of any one of embodiments 75-79, wherein the at least one inflammasome protein comprises ASC.

81. The method of embodiment 79, wherein a cut-off value for determining the sensitivity, specificity or both is selected from Tables 11B, 12B, 14A, 16, 17 or 19.

82. The method of any one of embodiments 75-79, wherein the at least one inflammasome protein comprises caspase-1.

83. The method of embodiment 82, wherein a cut-off value for determining the sensitivity, specificity or both is selected from Tables 11A or 15.

84. A method of evaluating a patient suspected of having a brain injury, the method comprising: measuring the level of at least one inflammasome protein in a biological sample obtained from the patient; determining the presence or absence of a protein signature associated with brain injury, wherein the protein signature comprises an elevated level of the at least one inflammasome protein; and selecting the patient as having brain injury if the patient exhibits the presence of the protein signature.

85. The method of embodiment 84, wherein the patient is presenting with clinical symptoms consistent with brain injury.

86. The method of embodiment 84 or 85, wherein the biological sample obtained from the patient is cerebrospinal fluid (CSF), CNS microdialysate, saliva, serum, plasma, urine or serum-derived extracellular vehicles (EVs).

87. The method of any one of embodiments 84-86, wherein the level of the at least one inflammasome protein in the protein signature is measured by an immunoassay utilizing one or more antibodies directed against the at least one inflammasome protein in the protein signature.

88. The method of any one of embodiments 84-87, wherein the at least one inflammasome protein is interleukin 18 (IL-18), IL-1β, apoptosis-associated speck-like protein containing a caspase recruitment domain (ASC), caspase-1, or combinations thereof.

89. The method of any one of embodiments 84-88, wherein the at least one inflammasome protein comprises ASC.

90. The method of embodiment 88 or 89, wherein the antibody binds to the PYRIN-PAAD-DAPIN domain (PYD), C-terminal caspase-recruitment domain (CARD) domain or a portion of the PYD or CARD domain of the ASC protein.

91. The method of any of embodiments 84-88, wherein the at least one inflammasome protein comprises caspase-1.

92. The method of any one of embodiments 84-91, wherein the level of the at least one inflammasome protein in the protein signature is enhanced relative to the level of the at least one inflammasome protein in a biological sample obtained from a control.

93. The method of embodiment 92, wherein the at least one inflammasome protein comprises ASC, wherein the level of ASC is at least 50% higher than the level of ASC in the biological sample obtained from the control.

94. The method of embodiment 92, wherein the at least one inflammasome protein comprises caspase-1, wherein the level of caspase-1 is at least 50% higher than the level of caspase-1 in the biological sample obtained from the control.

95. The method of any one of embodiments 92-94, wherein the biological sample obtained from the control is cerebrospinal fluid (CSF), CNS microdialysate, saliva, serum, plasma, urine or serum-derived extracellular vesicles (EVs).

96. The method of any one of embodiments 92-95, wherein the control is a healthy individual, wherein the healthy individual is an individual not presenting with clinical symptoms consistent with brain injury.

97. The method of any one of embodiments 84-96, wherein the brain injury is selected from a traumatic brain injury, stroke, mild cognitive impairment or multiple sclerosis.

98. The method of any one of embodiments 84-91, wherein the level of the at least one inflammasome protein in the protein signature is enhanced relative to a pre-determined reference value or range of reference values.

99. The method of embodiment 98, wherein the brain injury is traumatic brain injury (TBI).

100. The method of embodiment 99, wherein the biological sample obtained from patient is serum and the patient is selected as having TBI with a sensitivity of at least 80%, 85%, 90%, 95%, 99% or 100% and a specificity of at least 90%.

101. The method of embodiment 98 or 99, wherein the biological sample is serum and the patient is selected as having TBI with a specificity of at least 80%, 85%, 90%, 95%, 99% or 100%.

102. The method of embodiment 99, wherein the biological sample is serum and the patient is selected as having TBI with a sensitivity of at least 90% and a specificity of at least 80%.

103. The method of any one of embodiments 100-102, wherein the sensitivity and/or sensitivity is determined using the area under curve (AUC) from receiver operator characteristic (ROC) curves with confidence intervals of 95%.

104. The method of any one of embodiments 99-103, wherein the at least one inflammasome protein comprises ASC.

105. The method of embodiment 104, wherein a cut-off value for determining the sensitivity, specificity or both is selected from Tables 11B, 12B, 14A, 16, 17 or 19.

106. The method of any one of embodiments 99-103, wherein the at least one inflammasome protein comprises caspase-1.

107. The method of embodiment 106, wherein a cut-off value for determining the sensitivity, specificity or both is selected from Tables 11A or 15.

108. The method of embodiment 98, wherein the brain injury is multiple sclerosis (MS).

109. The method of embodiment 108, wherein the biological sample obtained from patient is serum and the patient is selected as having MS with a sensitivity of at least 80%, 85%, 90%, 95%, 99% or 100% and a specificity of at least 90%.

110. The method of embodiment 108 or 109, wherein the biological sample is serum and the patient is selected as having MS with a specificity of at least 80%, 85%, 90%, 95%, 99% or 100%.

111. The method of embodiment 108, wherein the biological sample is serum and the patient is selected as having MS with a sensitivity of at least 90% and a specificity of at least 80%.

112. The method of any one of embodiments 108-111, wherein the at least one inflammasome protein comprises ASC.

113. The method of embodiment 112, wherein a cut-off value for determining the sensitivity, specificity or both is selected from Table 7.

114. The method of any one of embodiments 109-113, wherein the sensitivity and/or sensitivity is determined using the area under curve (AUC) from receiver operator characteristic (ROC) curves with confidence intervals of 95%.

115. The method of embodiment 98, wherein the brain injury is stroke.

116. The method of embodiment 115, wherein the biological sample obtained from patient is serum and the patient is selected as having suffered a stroke with a sensitivity of at least 80%, 85%, 90%, 95%, 99% or 100% and a specificity of at least 90%.

117. The method of embodiment 115 or 116, wherein the biological sample is serum and the patient is selected as having suffered a stroke with a specificity of at least 80%, 85%, 90%, 95%, 99% or 100%.

118. The method of embodiment 115, wherein the biological sample is serum and the patient is selected as having suffered a stroke with a sensitivity of at least 100% and a specificity of at least 95%.

119. The method of any one of embodiments 116-118, wherein the at least one inflammasome protein comprises ASC.

120. The method of embodiment 119, wherein a cut-off value for determining the sensitivity, specificity or both is selected from Table 8.

121. The method of embodiment 115, wherein the biological sample obtained from patient is serum-derived EVs and the patient is selected as having suffered a stroke with a sensitivity of at least 80%, 85%, 90%, 95%, 99% or 100% and a specificity of at least 90%.

122. The method of embodiment 115 or 121, wherein the biological sample is serum-derived EVs and the patient is selected as having suffered a stroke with a specificity of at least 80%, 85%, 90%, 95%, 99% or 100%.

123. The method of embodiment 115, wherein the biological sample is serum-derived EVs and the patient is selected as having suffered a stroke with a sensitivity of at least 100% and a specificity of at least 100%.

124. The method of any one of embodiments 121-123, wherein the at least one inflammasome protein comprises ASC.

125. The method of embodiment 124, wherein a cut-off value for determining the sensitivity, specificity or both is selected from Table 9.

126. The method of any one of embodiments 116-118 or 121-123, wherein the sensitivity and/or sensitivity is determined using the area under curve (AUC) from receiver operator characteristic (ROC) curves with confidence intervals of 95%.

Other subject matter contemplated by the present disclosure that is related to mild cognitive impairment (MCI), Alzheimer's disease (AD), age-related macular degeneration (AMD) or inflammaging is set out in the following numbered embodiments:

1. A method of evaluating a patient suspected of having mild cognitive impairment (MCI), the method comprising: measuring an expression level of at least one inflammasome protein in a biological sample obtained from the patient; comparing the expression level of the at least one inflammasome protein in the biological sample to an expression level of one or more control MCI biomarkers; and selecting the patient as having MCI if the expression level of the at least one inflammasome protein in the biological sample is similar to the expression level of the one or more control MCI biomarkers.

2. The method of embodiment 1, wherein the expression level of the at least one inflammasome protein is similar to the expression level of the one or more control MCI biomarkers if the expression level or a parameter representative of the expression level of the at least one inflammasome protein is within 10%, 9%, 8%, 7%, 6%, 5%, 4%, 3%, 2% or 1% of the expression level or a parameter representative of the expression level of the one or more control MCI biomarkers.

3. The method of embodiment 1 or 2, wherein the expression level of the one or more control MCI biomarkers is measured in the biological sample obtained from the patient.

4. The method of embodiment 1 or 2, wherein the expression level of the one or more control MCI biomarkers is measured in a biological sample obtained from an individual previously diagnosed with MCI.

5. The method of embodiment 4, wherein the biological sample obtained from the individual previously diagnosed with MCI is a same type of biological sample obtained from the patient suspected of suffering from MCI.

6. The method of any one of embodiments 1-5, wherein the expression level of the at least one inflammasome protein and the expression level of the one or more control MCI biomarkers are enhanced relative to the expression level of the at least one inflammasome protein and the expression level of the one or more control MCI biomarkers in a biological sample obtained from a control.

7. The method of embodiment 6, wherein the biological sample obtained from the control is a same type of biological sample obtained from the patient suspected of suffering from MCI.

8. The method of embodiment 6 or 7, wherein the control is a healthy individual, wherein the healthy individual is an individual not presenting with clinical symptoms consistent with MCI.

9. The method of any one of embodiments 1-5, wherein the expression level of the at least one inflammasome protein and the expression level of the one or more control MCI biomarkers are enhanced relative to a pre-determined reference value or range of reference values for the at least one inflammasome protein and the one or more control MCI biomarkers.

10. The method of any one of embodiments 6-9, wherein the parameter representative of the expression level of the at least one inflammasome protein and the parameter representative of the expression level of the one or more control MCI biomarkers is an area under curve (AUC).

11. The method of any one of the above embodiments, wherein the patient is presenting with clinical symptoms consistent with MCI.

12. The method of any one of embodiments 1-11, wherein the biological sample obtained from the patient suspected of suffering from MCI is cerebrospinal fluid (CSF), CNS microdialysate, saliva, serum, plasma, urine or serum-derived extracellular vesicles (EVs).

13. The method of any one of embodiments 1-12, wherein the expression level of the at least one inflammasome protein and/or the one or more control MCI biomarkers is measured by an immunoassay utilizing one or more antibodies directed against the at least one inflammasome protein and/or the one or more control MCI biomarkers.

14. The method of any one of embodiments 1-13, wherein the at least one inflammasome protein is interleukin 18 (IL-18), IL-1β, apoptosis-associated speck-like protein containing a caspase recruitment domain (ASC), caspase-1, or combinations thereof.

15. The method of any one of embodiments 1-14, wherein the at least one inflammasome protein comprises ASC.

16. The method of any one of embodiments 1-14, wherein the at least one inflammasome protein comprises IL-18.

17. The method of any one of embodiments 1-16, wherein the one or more control MCI biomarkers are neurofilament light polypeptide (NFL), soluble APP-alpha (sAPPα) and/or soluble APP-beta (sAPPβ).

18. The method of embodiment 10, wherein the at least one inflammasome protein is ASC and the one or more control MCI biomarkers is soluble APP-alpha (sAPPα), wherein the AUC for ASC is 0.974 and the AUC for sAPP-alpha is 0.9687.

19. The method of embodiment 10, wherein the at least one inflammasome protein is ASC and the one or more control MCI biomarkers is soluble APP-beta (sAPPβ), wherein the AUC for ASC is 0.974 and the AUC for sAPP-beta is 0.9068.

20. The method of embodiment 10, wherein the at least one inflammasome protein is ASC and the one or more control MCI biomarkers is neurofilament light polypeptide (NFL) wherein the AUC for ASC is 0.974 and the AUC for NFL is 0.7734.

21. The method of any one of embodiments 1-20, wherein the biological sample obtained from the patient is serum and the patient is selected as having MCI with a sensitivity of at least 70%, 75%, 80%, 85%, 90%, 95%, 99% or 100% and a specificity of at least 55%.

22. The method of any one of embodiments 1-21, wherein the biological sample obtained from the patient is serum and the patient is selected as having MCI with a sensitivity of at least 70%, 75%, 80%, 85%, 90%, 95%, 99% or 100%.

23. The method of any one of embodiments 1-22, wherein the biological sample obtained from the patient is serum and the patient is selected as having MCI with a sensitivity of at least 70% and a specificity of at least 55%.

24. The method of any one of embodiments 21-23, wherein the specificity and/or sensitivity is determined using receiver operator characteristic (ROC) curves with confidence intervals of 95%.

25. The method of any one of embodiments 1-24, wherein said method further comprises assessing the presence of one or more symptoms associated with MCI in order to select the patient as having MCI.

26. The method of embodiment 25, wherein the one or more symptoms associated with MCI are forgetfulness, lack of focus, anxiety, difficulty making decisions, difficulty understanding instructions, difficulty planning, trouble navigating familiar environments, impulsivity, or questionable judgment as well as judging the time or sequence of steps needed to complete a complex task or visual perception.

27. A method of evaluating a patient suspected of having Alzheimer's Disease (AD), the method comprising: measuring an expression level of at least one inflammasome protein in a biological sample obtained from the patient; comparing the expression level of the at least one inflammasome protein in the biological sample to an expression level of one or more control AD biomarkers; and selecting the patient as having AD if the expression level of the at least one inflammasome protein in the biological sample is similar to the expression level of the one or more control AD biomarkers.

28. The method of embodiment 27, wherein the expression level of the at least one inflammasome protein is similar to the expression level of the one or more control AD biomarkers if the expression level or a parameter representative of the expression level of the at least one inflammasome protein is within 10%, 9%, 8%, 7%, 6%, 5%, 4%, 3%, 2% or 1% of the expression level or a parameter representative of the expression level of the one or more control AD biomarkers.

29. The method of embodiment 27 or 28, wherein the expression level of the one or more control AD biomarkers is measured in the biological sample obtained from the patient.

30. The method of embodiment 27 or 28, wherein the expression level of the one or more control AD biomarkers is measured in a biological sample obtained from an individual previously diagnosed with AD.

31. The method of embodiment 30, wherein the biological sample obtained from the individual previously diagnosed with AD is a same type of biological sample obtained from the patient suspected of suffering from AD.

32. The method of any one of embodiments 27-31, wherein the expression level of the at least one inflammasome protein and the expression level of the one or more control AD biomarkers are enhanced relative to the expression level of the at least one inflammasome protein and the expression level of the one or more control AD biomarkers in a biological sample obtained from a control.

33. The method of embodiment 32, wherein the biological sample obtained from the control is a same type of biological sample obtained from the patient suspected of suffering from AD.

34. The method of embodiment 32 or 33, wherein the control is a healthy individual, wherein the healthy individual is an individual not presenting with clinical symptoms consistent with AD.

35. The method of any one of embodiments 27-31, wherein the expression level of the at least one inflammasome protein and the expression level of the one or more control AD biomarkers are enhanced relative to a pre-determined reference value or range of reference values for the at least one inflammasome protein and the one or more control AD biomarkers.

36. The method of any one of embodiments 32-35, wherein the parameter representative of the expression level of the at least one inflammasome protein and the parameter representative of the expression level of the one or more control AD biomarkers is an area under curve (AUC).

37. The method of any one of embodiments 27-36, wherein the patient is presenting with clinical symptoms consistent with AD.

38. The method of any one of embodiments 27-37, wherein the biological sample obtained from the patient suspected of suffering from AD is cerebrospinal fluid (CSF), CNS microdialysate, saliva, serum, plasma, urine or serum-derived extracellular vesicles (EVs).

39. The method of any one of embodiments 27-38, wherein the expression level of the at least one inflammasome protein and/or the one or more control AD biomarkers is measured by an immunoassay utilizing one or more antibodies directed against the at least one inflammasome protein and/or the one or more control AD biomarkers.

40. The method of any one of embodiments 27-39, wherein the at least one inflammasome protein is interleukin 18 (IL-18), IL-1β, apoptosis-associated speck-like protein containing a caspase recruitment domain (ASC), caspase-1, or combinations thereof.

41. The method of any one of embodiments 27-40, wherein the at least one inflammasome protein comprises ASC.

42. The method of any one of embodiments 27-40, wherein the at least one inflammasome protein comprises IL-18.

43. The method of any one of embodiments 27-42, wherein the one or more control AD biomarkers are neurofilament light polypeptide (NFL), soluble APP-alpha (sAPPα) and/or soluble APP-beta (sAPPβ).

44. The method of embodiment 36, wherein the at least one inflammasome protein is ASC and the one or more control AD biomarkers is soluble APP-alpha (sAPPα), wherein the AUC for ASC is 0.833 and the AUC for sAPPα is 0.956.

45. The method of embodiment 36, wherein the at least one inflammasome protein is ASC and the one or more control AD biomarkers is soluble APPβ (sAPPβ), wherein the AUC for ASC is 0.833 and the AUC for sAPPβ is 0.919.

46. The method of embodiment 36, wherein the at least one inflammasome protein is ASC and the one or more control AD biomarkers is neurofilament light polypeptide (NFL), wherein the AUC for ASC is 0.833 and the AUC for NFL is 0.717.

47. The method of any one of embodiments 27-46, wherein the biological sample obtained from the patient is serum and the patient is selected as having AD with a sensitivity of at least 70%, 75%, 80%, 85%, 90%, 95%, 99% or 100% and a specificity of at least 55%.

48. The method of any one of embodiments 27-47, wherein the biological sample obtained from the patient is serum and the patient is selected as having AD with a sensitivity of at least 70%, 75%, 80%, 85%, 90%, 95%, 99% or 100%.

49. The method of any one of embodiments 27-48, wherein the biological sample obtained from the patient is serum and the patient is selected as having AD with a sensitivity of at least 70% and a specificity of at least 55%.

50. The method of any one of embodiments 47-49, wherein the specificity and/or sensitivity is determined using receiver operator characteristic (ROC) curves with confidence intervals of 95%.

51. The method of any one of embodiments 27-50, wherein said method further comprises assessing the presence of one or more symptoms associated with AD in order to select the patient as having AD.

52. The method of embodiment 51, wherein the one or more symptoms associated with AD are forgetfulness, lack of focus, anxiety, feeling anxious or overwhelmed when making decisions, difficulty understanding instructions or planning things, trouble navigating familiar environments, difficulty performing tasks, forgetting material that was just read, losing or misplacing a valuable object, difficulty with organization, confusion with time or place, trouble controlling bladder or bowels, personality or behavioral changes such as changes in mood or personality; changes in sleep patterns, difficulty communicating such as problems with words in speaking or writing, vulnerability to infections, impulsivity, or questionable judgment, trouble understanding visual images and spatial relationships, misplacing things and losing the ability to retrace steps, decreased or poor judgement, withdrawal from work or social activities.

53. The method of any one of embodiments 32-35, wherein the parameter representative of the expression level of the at least one inflammasome protein and the parameter representative of the expression level of the one or more control MCI biomarkers is a cut-off value.

54. The method of embodiment 55, wherein the at least one inflammasome protein is ASC and the cut-off value is above 264.9 pg/ml and below 560 pg/ml.

55. The method of any one of embodiments 32-35, wherein the parameter representative of the expression level of the at least one inflammasome protein and the parameter representative of the expression level of the one or more control MCI biomarkers is a cut-off value.

56. The method of embodiment 55, wherein the at least one inflammasome protein is ASC and the cut-off value is above 560 pg/ml.

57. A method of determining whether a patient is suffering from mild cognitive impairment (MCI) or Alzheimer's Disease (AD), the method comprising: measuring an expression level of at least one inflammasome protein in a biological sample obtained from the patient; comparing the expression level of the at least one inflammasome protein in the biological sample to a pre-determined reference value or range of reference values for the at least one inflammasome protein; and selecting the patient as having AD if the expression level of the at least one inflammasome protein is within the predetermined range of reference values or MCI if the expression level is above a pre-determined reference value.

58. The method of embodiment 57, wherein the at least one inflammasome protein is ASC.

58. The method of embodiment 58, wherein the predetermined range of reference values is between 264.9 pg/ml and 560 pg/ml.

59. The method of embodiment 58 or 59, wherein the pre-determined reference value is above 560 pg/ml.

60. A method of evaluating a patient suspected of age-related macular degeneration (AMD), the method comprising: measuring an expression level of at least one inflammasome protein in a biological sample obtained from the patient; determining the presence or absence of a protein signature associated with AMD, wherein the protein signature comprises an elevated expression level of the at least one inflammasome protein; and selecting the patient as having AMD if the patient exhibits the presence of the protein signature.

61. The method of embodiment 60, wherein the biological sample obtained from the patient is cerebrospinal fluid (CSF), CNS microdialysate, saliva, serum, plasma, urine or serum-derived extracellular vesicles (EVs).

62. The method of embodiment 60 or 61, wherein the level of the at least one inflammasome protein in the protein signature is measured by an immunoassay utilizing one or more antibodies directed against the at least one inflammasome protein in the protein signature.

63. The method of any one of embodiments 60-62, wherein the level of the at least one inflammasome protein in the protein signature is enhanced relative to the level of the at least one inflammasome protein in a biological sample obtained from a control.

64. The method of embodiment 63, wherein the biological sample obtained from the control is cerebrospinal fluid (CSF), CNS microdialysate, saliva, serum, plasma, urine or serum-derived extracellular vesicles (EVs).

65. The method of embodiment 63, wherein the control is a healthy individual not exhibiting the clinical symptoms of AMD.

66. The method of any one of embodiments 60-65, wherein the at least one inflammasome protein is interleukin 18 (IL-18), IL-1β, apoptosis-associated speck-like protein containing a caspase recruitment domain (ASC), caspase-1, or combinations thereof.

67. The method of any one of embodiments 60-66, wherein the at least one inflammasome protein comprises ASC, and wherein the AUC for ASC is 0.9823.

68. The method of any one of embodiments 60-66, wherein the at least one inflammasome protein comprises IL-18, and wherein the AUC for IL-18 is 0.7286.

69. The method of any one of embodiments 60-68, wherein the biological sample obtained from the patient is serum and the patient is selected as having AMD with a sensitivity of at least 70%, 75%, 80%, 85%, 90%, 95%, 99%, or 100%.

70. The method of any one of embodiments 60-69, wherein the biological sample obtained from the patient is serum and the patient is selected as having AMD with a sensitivity of at least 70%, 75%, 80%, 85%, 90%, 95%, 99%, or 100% and a specificity of at least 55%.

71. The method of any one of embodiments 69-70, wherein the specificity and/or sensitivity is determined using receiver operator characteristic (ROC) curves with confidence intervals of 95%.

72. The method of any one of embodiments 60-71, wherein said method further comprises assessing the presence of one or more symptoms associated with AMD in order to select the patient having AMD.

73. The method of embodiment 72, wherein the one or more symptoms associated with AMD are blurred vision, “fuzzy vision, seeing straight lines as wavy or distorted, seeing blurry areas on a printed page, difficulty reading or seeing details in low light levels, extra sensitivity to glare, dark or blurry areas in the center of vision, whiteout in the center of vision, or a change in the perception of color.

74. The method of any one of embodiments 60-73, wherein the parameter representative of the expression level of the at least one inflammasome protein is a cut-off value.

75. The method of embodiment 74, wherein the at least one inflammasome protein is ASC, and the cut-off value is above 365.6 pg/mL.

76. The method of embodiment 74, wherein the at least one inflammasome protein is IL-18, and the cut-off value is above 242.4 pg/mL.

77. A method of treating inflammaging in a subject, the method comprises administering to the subject a therapeutically effective amount of a monoclonal antibody or an antibody fragment thereof of that binds specifically to ASC, wherein the antibody or the antibody fragment comprises a heavy chain variable (VH) region and a light chain variable (VL) region, wherein the VH region amino acid sequence comprises HCDR1 of SEQ ID NO: 6, HCDR2 of SEQ ID NO: 7 and HCDR3 of SEQ ID NO: 8, or a variant thereof having at least one amino acid substitution in HCDR1, HCDR2, and/or HCDR3; and wherein the VL region amino acid sequence comprises LCDR1 of SEQ ID NO: 12, LCDR2 of SEQ ID NO: 13 and LCDR3 of SEQ ID NO: 14, or a variant thereof having at least one amino acid substitution in LCDR1, LCDR2, and/or LCDR3, thereby treating inflammaging in the subject.

78. The method of embodiment 77, wherein the VH region amino acid sequence of the monoclonal antibody or the antibody fragment thereof comprises SEQ ID NO: 18, 19, 20, 21, 22, or an amino acid sequence that is at least 95%, 96%, 97%, 98% or 99% identical to the amino acid sequence of SEQ ID NO: 18, 19, 20, 21 or 22; and wherein the VL region amino acid sequence of the monoclonal antibody or the antibody fragment thereof comprises SEQ ID NO: 28, 29, 30, 31, or an amino acid sequence that is at least 95%, 96%, 97%, 98% or 99% identical to the amino acid sequence of SEQ ID NO: 28, 29, 30 or 31.

79. The method of embodiment 77, wherein the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28.

80. The method of embodiment 77, wherein the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and wherein the VL region amino acid sequence of the monoclonal antibody or the antibody fragment thereof comprises SEQ ID NO: 29 or an amino acid sequence that is at least 95%, 96%, 97%, 98% or 99% identical to the amino acid sequence of SEQ ID NO: 29.

81. The method of embodiment 77, wherein the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30.

82. The method of embodiment 77, wherein the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31.

83. The method of embodiment 77, wherein the VH region amino acid sequence of the monoclonal antibody or the antibody fragment thereof comprises SEQ ID NO: 19, or an amino acid sequence that is at least 95%, 96%, 97%, 98% or 99% identical to the amino acid sequence of SEQ ID NO: 19; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28.

84. The method of embodiment 77, wherein the VH region amino acid sequence of the monoclonal antibody or the antibody fragment thereof comprises SEQ ID NO: 19, or an amino acid sequence that is at least 95%, 96%, 97%, 98% or 99% identical to the amino acid sequence of SEQ ID NO: 19; and wherein the VL region amino acid sequence comprises SEQ ID NO: 29 or an amino acid sequence that is at least 95%, 96%, 97%, 98% or 99% identical to the amino acid sequence of SEQ ID NO: 29.

85. The method of embodiment 77, wherein the VH region amino acid sequence of the monoclonal antibody or the antibody fragment thereof comprises SEQ ID NO: 19, or an amino acid sequence that is at least 95%, 96%, 97%, 98% or 99% identical to the amino acid sequence of SEQ ID NO: 19; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30.

86. The method of embodiment 77, wherein the VH region amino acid sequence of the monoclonal antibody or the antibody fragment thereof comprises SEQ ID NO: 19, or an amino acid sequence that is at least 95%, 96%, 97%, 98% or 99% identical to the amino acid sequence of SEQ ID NO: 19; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31.

87. The method of embodiment 77, wherein the VH region amino acid sequence of the monoclonal antibody or the antibody fragment thereof comprises SEQ ID NO: 20, or an amino acid sequence that is at least 95%, 96%, 97%, 98% or 99% identical to the amino acid sequence of SEQ ID NO: 20; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28.

88. The method of embodiment 77, wherein the VH region amino acid sequence comprises SEQ ID NO: 20, or an amino acid sequence that is at least 95%, 96%, 97%, 98% or 99% identical to the amino acid sequence of SEQ ID NO: 20; and wherein the VL region amino acid sequence comprises SEQ ID NO: 29 or an amino acid sequence that is at least 95%, 96%, 97%, 98% or 99% identical to the amino acid sequence of SEQ ID NO: 29.

89. The method of embodiment 77, wherein the VH region amino acid sequence of the monoclonal antibody or the antibody fragment thereof comprises SEQ ID NO: 20, or an amino acid sequence that is at least 95%, 96%, 97%, 98% or 99% identical to the amino acid sequence of SEQ ID NO: 20; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30.

90. The method of embodiment 77, wherein the VH region amino acid sequence of the monoclonal antibody or the antibody fragment thereof comprises SEQ ID NO: 20, or an amino acid sequence that is at least 95%, 96%, 97%, 98% or 99% identical to the amino acid sequence of SEQ ID NO: 20; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31.

91. The method of embodiment 77, wherein the VH region amino acid sequence of the monoclonal antibody or the antibody fragment thereof comprises SEQ ID NO: 21, or an amino acid sequence that is at least 95%, 96%, 97%, 98% or 99% identical to the amino acid sequence of SEQ ID NO: 21; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28.

92. The method of embodiment 77, wherein the VH region amino acid sequence comprises SEQ ID NO: 21, or an amino acid sequence that is at least 95%, 96%, 97%, 98% or 99% identical to the amino acid sequence of SEQ ID NO: 21; and wherein the VL region amino acid sequence comprises SEQ ID NO: 29 or an amino acid sequence that is at least 95%, 96%, 97%, 98% or 99% identical to the amino acid sequence of SEQ ID NO: 29.

93. The method of embodiment 77, wherein the VH region amino acid sequence of the monoclonal antibody or the antibody fragment thereof comprises SEQ ID NO: 21, or an amino acid sequence that is at least 95%, 96%, 97%, 98% or 99% identical to the amino acid sequence of SEQ ID NO: 21; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30.

94. The method of embodiment 77, wherein the VH region amino acid sequence of the monoclonal antibody or the antibody fragment thereof comprises SEQ ID NO: 21, or an amino acid sequence that is at least 95%, 96%, 97%, 98% or 99% identical to the amino acid sequence of SEQ ID NO: 21; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31.

95. The method of embodiment 77, wherein the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and wherein the VL region amino acid sequence of the monoclonal antibody or the antibody fragment thereof comprises SEQ ID NO: 28 or an amino acid sequence that is at least 95%, 96%, 97%, 98% or 99% identical to the amino acid sequence of SEQ ID NO: 28.

96. The method of embodiment 77, wherein the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and wherein the VL region amino acid sequence of the monoclonal antibody or the antibody fragment thereof comprises SEQ ID NO: 29 or an amino acid sequence that is at least 95%, 96%, 97%, 98% or 99% identical to the amino acid sequence of SEQ ID NO: 29.

97. The method of embodiment 77, wherein the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30.

98. The method of embodiment 77, wherein the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31.

99. The method of any one of embodiments 77-98, wherein the ASC is human ASC protein.

100. The method of any one of embodiments 77-99, wherein the antibody fragment is an Fab, an F(ab′)₂, an Fab′, an scFv, a single domain antibody, a diabody or a single chain camelid antibody.

101. The method of any one of embodiments 77-100, wherein the monoclonal antibody or the antibody fragment thereof is human, humanized or chimeric.

102. The method of any one of embodiments 77-101, wherein the administering the monoclonal antibody or the antibody fragment thereof reduces levels of at least inflammatory cytokine.

103. The method of any one of embodiments 77-102, wherein the administration of the monoclonal antibody or the antibody fragment thereof results in inhibition of inflammasome activation in the subject.

104. The method of any one of embodiments 77-103, wherein the administration of the monoclonal antibody or the antibody fragment thereof results in a reduction in the activity of ASC as compared to a control.

105. The method of embodiment 104, wherein the control is an untreated subject.

106. The method of any one of embodiments 77-105, wherein the administration is intracerebroventricularly, intraperitoneally, intravenously or by inhalation.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

In addition, the following particular applications are incorporated herein by reference: U.S. application Ser. No. 16/026,482 (now U.S. Pat. No. 10,703,811 issued on Jul. 7, 2020) filed on Jul. 3, 2018; PCT/US2019/040635 (WO 2020/010273 A1) filed on Jul. 3, 2019; and PCT/US2018/051899 (WO 2019/060516) filed on Sep. 20, 2018.

1-58. (canceled)

58-106. (canceled)

107. A method of determining whether a patient is suffering from a condition selected from the group consisting of mild cognitive impairment (MCI), Alzheimer's Disease (AD), and age-related macular degeneration (AMD), the method comprising:
measuring an expression level of at least one inflammasome protein in a biological sample obtained from the patient;
comparing the expression level of the at least one inflammasome protein in the biological sample to a pre-determined reference value or range of reference values for the at least one inflammasome protein; and
selecting the patient as having AD if the expression level of the at least one inflammasome protein is within the predetermined range of reference values, selecting the patient as having MCI if the expression level is above a pre-determined reference value, or selecting the patient as having AMD if the patient exhibits the presence of the protein signature.

108. The method of claim 106, wherein the at least one inflammasome protein is interleukin 18 (IL-18), IL-1β, apoptosis-associated speck-like protein containing a caspase recruitment domain (ASC), caspase-1, or combinations thereof.

109. The method of claim 108, wherein at least one inflammasome protein is ASC and the predetermined range of reference values is between 264.9 pg/ml and 560 pg/ml.

110. The method of claim 107, wherein the biological sample obtained from the patient is cerebrospinal fluid (CSF), CNS microdialysate, saliva, serum, plasma, urine or serum-derived extracellular vesicles (EVs).

111. The method of claim 107, wherein the level of the at least one inflammasome protein in the protein signature is measured by an immunoassay utilizing one or more antibodies directed against the at least one inflammasome protein in the protein signature.

112. The method of claim 107, wherein the control is a healthy individual not exhibiting the clinical symptoms of MCI, AD, or AMD.

113. The method of claim 108, wherein the at least one inflammasome protein comprises ASC, and wherein the AUC for ASC is 0.9823.

114. The method of claim 108, wherein the at least one inflammasome protein comprises IL-18, and wherein the AUC for is 0.7286.

115. The method of claim 107, wherein the biological sample obtained from the patient is serum and the patient is selected as having MCI, AD, or AMD with a sensitivity of at least 70%, 75%, 80%, 85%, 90%, 95%, 99%, or 100% and a specificity of at least 55%.

116. The method of claim 108, wherein the at least one inflammasome protein is ASC, wherein a parameter representative of the expression level of ASC is a cut-off value above 365.6 pg/mL.

117. The method of claim 108, wherein the at least one inflammasome protein is IL-18, wherein a parameter representative of the expression level of IL-18 is a cut-off value above 242.4 pg/mL.

118. A method of treating inflammaging in a subject, the method comprising:
administering to the subject a therapeutically effective amount of a monoclonal antibody or an antibody fragment thereof of that binds specifically to ASC, wherein the antibody or the antibody fragment comprises a heavy chain variable (VH) region and a light chain variable (VL) region,
wherein the VH region amino acid sequence comprises HCDR1 of SEQ ID NO: 6, HCDR2 of SEQ ID NO: 7 and HCDR3 of SEQ ID NO: 8, or a variant thereof having at least one amino acid substitution in HCDR1, HCDR2, and/or HCDR3; and
wherein the VL region amino acid sequence comprises LCDR1 of SEQ ID NO: 12, LCDR2 of SEQ ID NO: 13 and LCDR3 of SEQ ID NO: 14, or a variant thereof having at least one amino acid substitution in LCDR1, LCDR2, and/or LCDR3, thereby treating inflammaging in the subject.

119. The method of claim 118, wherein
the VH region amino acid sequence of the monoclonal antibody or the antibody fragment thereof comprises SEQ ID NO: 18, 19, 20, 21, 22, or an amino acid sequence that is at least 95%, 96%, 97%, 98% or 99% identical to the amino acid sequence of SEQ ID NO: 18, 19, 20, 21 or 22; and
wherein the VL region amino acid sequence of the monoclonal antibody or the antibody fragment thereof comprises SEQ ID NO: 28, 29, 30, 31, or an amino acid sequence that is at least 95%, 96%, 97%, 98% or 99% identical to the amino acid sequence of SEQ ID NO: 28, 29, 30 or 31.

120. The method of claim 118, wherein
the VH region amino acid sequence of the monoclonal antibody or the antibody fragment thereof comprises SEQ ID NO: 18, or an amino acid sequence that is at least 95%, 96%, 97%, 98% or 99% identical to the amino acid sequence of SEQ ID NO: 18; and
wherein the VL region amino acid sequence of the monoclonal antibody or the antibody fragment thereof comprises SEQ ID NO: 28, 29, 30, 31, or an amino acid sequence that is at least 95%, 96%, 97%, 98% or 99% identical to the amino acid sequence of SEQ ID NO: 28, 29, 30, or 31.

121. The method of claim 118, wherein
the VH region amino acid sequence of the monoclonal antibody or the antibody fragment thereof is selected from SEQ ID NO: 19, or an amino acid sequence that is at least 95%, 96%, 97%, 98% or 99% identical to the amino acid sequence of SEQ ID NO: 19; and
wherein the VL region amino acid sequence of the monoclonal antibody or the antibody fragment thereof is selected from SEQ ID NO: 28, 29, 30, 31, or an amino acid sequence that is at least 95%, 96%, 97%, 98% or 99% identical to the amino acid sequence of SEQ ID NO: 28, 29, 30, or 31.

122. The method of claim 118, wherein
the VH region amino acid sequence of the monoclonal antibody or the antibody fragment thereof comprises SEQ ID NO: 20, or an amino acid sequence that is at least 95%, 96%, 97%, 98% or 99% identical to the amino acid sequence of SEQ ID NO: 20; and
wherein the VL region amino acid sequence of the monoclonal antibody or the antibody fragment thereof comprises SEQ ID NO: 28, 29, 30, 31, or an amino acid sequence that is at least 96%, 97%, 98% or 99% identical to the amino acid sequence of SEQ ID NO: 28, 29, 30, or 31.

123. The method of claim 118, wherein
the VH region amino acid sequence of the monoclonal antibody or the antibody fragment thereof comprises SEQ ID NO: 21, or an amino acid sequence that is at least 95%, 96%, 97%, 98% or 99% identical to the amino acid sequence of SEQ ID NO: 21; and
wherein the VL region amino acid sequence of the monoclonal antibody or the antibody fragment thereof comprises SEQ ID NO: 28, 29, 30, 31, or an amino acid sequence that is at least 95%, 96%, 97%, 98% or 99% identical to the amino acid sequence of SEQ ID NO: 28, 29, 30, or 31.

124. The method of claim 118, wherein
the VH region amino acid sequence of the monoclonal antibody or the antibody fragment thereof comprises SEQ ID NO: 22, or an amino acid sequence that is at least 95%, 96%, 97%, 98% or 99% identical to the amino acid sequence of SEQ ID NO: 22; and
wherein the VL region amino acid sequence of the monoclonal antibody or the antibody fragment thereof comprises SEQ ID NO: 28, 29, 30, 31, or an amino acid sequence that is at least 95%, 96%, 97%, 98% or 99% identical to the amino acid sequence of SEQ ID NO: 28, 29, 30; or 31.

125. The method of claim 118, wherein
the VH region amino acid sequence of the monoclonal antibody or the antibody fragment thereof comprises SEQ ID NO: 18, 19, 20, 21 22, or an amino acid sequence that is at least 95%, 96%, 97%, 98% or 99% identical to the amino acid sequence of SEQ ID NO: 18, 19, 20, 21 or 22; and
wherein the VL region amino acid sequence comprises SEQ ID NO: 28 or an amino acid sequence that is at least 95%, 96%, 97%, 98% or 99% identical to the amino acid sequence of SEQ ID NO: 28.

126. The method of claim 118, wherein
the VH region amino acid sequence of the monoclonal antibody or the antibody fragment thereof comprises SEQ ID NO: 18, 19, 20, 21 22, or an amino acid sequence that is at least 95%, 96%, 97%, 98% or 99% identical to the amino acid sequence of SEQ ID NO: 18, 19, 20, 21 or 22; and
wherein the VL region amino acid sequence of the monoclonal antibody or the antibody fragment thereof comprises SEQ ID NO: 29 or an amino acid sequence that is at least 95%, 96%, 97%, 98% or 99% identical to the amino acid sequence of SEQ ID NO: 29.

127. The method of claim 118, wherein the VH region amino acid sequence of the monoclonal antibody or the antibody fragment thereof comprises SEQ ID NO: 18, 19, 20, 21 22, or an amino acid sequence that is at least 95%, 96%, 97%, 98% or 99% identical to the amino acid sequence of SEQ ID NO: 18, 19, 20, 21 or 22; and
wherein the VL region amino acid sequence of the monoclonal antibody or the antibody fragment thereof comprises SEQ ID NO: 30 or an amino acid sequence that is at least 95%, 96%, 97%, 98% or 99% identical to the amino acid sequence of SEQ ID NO: 30.

128. The method of claim 118, wherein
the VH region amino acid sequence of the monoclonal antibody or the antibody fragment thereof comprises SEQ ID NO: 18, 19, 20, 21 22, or an amino acid sequence that is at least 95%, 96%, 97%, 98% or 99% identical to the amino acid sequence of SEQ ID NO: 18, 19, 20, 21 or 22; and
wherein the VL region amino acid sequence of the monoclonal antibody or the antibody fragment thereof comprises SEQ ID NO: 31 or an amino acid sequence that is at least 95%, 96%, 97%, 98% or 99% identical to the amino acid sequence of SEQ ID NO: 31.

129. The method of claim 118, wherein the ASC is human ASC protein.

130. The method of claim 118, wherein the antibody fragment is an Fab, an F(ab′)₂, an Fab′, an scFv, a single domain antibody, a diabody or a single chain camelid antibody.

131. The method of claim 118, wherein the monoclonal antibody or the antibody fragment thereof is human, humanized or chimeric.

132. The method of claim 118, wherein the administering the monoclonal antibody or the antibody fragment thereof reduces levels of at least inflammatory cytokine.

133. The method of claim 118, wherein the administration of the monoclonal antibody or the antibody fragment thereof results in inhibition of inflammasome activation in the subject.

134. The method of claim 118, wherein the administration of the monoclonal antibody or the antibody fragment thereof results in a reduction in the activity of ASC as compared to an untreated subject as a control.

135. The method of claim 118, wherein the administration is intracerebroventricularly, intraperitoneally, intravenously or by inhalation.


SEQUENCE LISTING (41 sequences)

SEQ ID NO: 1 - protein, 16 aa, Rattus norvegicus
ALRQTQPYLVTDLEQS

SEQ ID NO: 2 - protein, 14 aa, Homo sapiens
RESQSYLVEDLERS

SEQ ID NO: 3 - protein, 19 aa, Homo sapiens
CEYYTEIREREREKSEKGR

SEQ ID NO: 4 - protein, 15 aa, Rattus norvegicus
MEESQSKEESNTEGC

SEQ ID NO: 5 - protein, 21 aa, Homo sapiens
KKFKLKLLSVPLREGYGRIPR

SEQ ID NO: 6 - protein, 7 aa, Artificial Sequence
  Heavy Chain (H) CDR1
TSGMGVS

SEQ ID NO: 7 - protein, 16 aa, Artificial Sequence
  Heavy Chain (H) CDR2
HIYWDDDKRYNPSLKS

SEQ ID NO: 8 - protein, 11 aa, Artificial Sequence
  Heavy Chain (H) CDR3
STPIVANAMDY

SEQ ID NO: 9 - DNA, 21 bp, Artificial Sequence
  Heavy Chain( H)  CDR1
actagtggaatgggtgtgagc

SEQ ID NO: 10 - DNA, 48 bp, Artificial Sequence
  Heavy Chain (H) CDR2
cacatttattgggatgatgataagcgctacaacccatctctgaagagc

SEQ ID NO: 11 - DNA, 33 bp, Artificial Sequence
  Heavy Chain (H) CDR3
agcacccccatcgtggccaacgccatggactac

SEQ ID NO: 12 - protein, 15 aa, Artificial Sequence
  Light (Kappa) (L) Chain CDR1
KASQSVDYDGDSYMN

SEQ ID NO: 13 - protein, 7 aa, Artificial Sequence
  Light (Kappa) (L) Chain CDR2
AASNLES

SEQ ID NO: 14 - protein, 9 aa, Artificial Sequence
  Light (Kappa) (L) Chain CDR3
QQSNEDPYT

SEQ ID NO: 15 - DNA, 45 bp, Artificial Sequence
  Light (Kappa) (L) Chain CDR1
aaggccagccagagtgttgactacgacggcgacagttacatgaat

SEQ ID NO: 16 - DNA, 21 bp, Artificial Sequence
  Light (Kappa) (L) Chain CDR2
gccgcatctaacctggaatcc

SEQ ID NO: 17 - DNA, 27 bp, Artificial Sequence
  Light (Kappa) (L) Chain CDR3
cagcaatctaatgaggacccttacact

SEQ ID NO: 18 - protein, 121 aa, Artificial Sequence
  Variable Heavy (VH) 1 Chain
QVTLKESGPAIVKPTQTLTLTCSFSGFSLSTSGMGVSWIRQPSGKGLEWLAHIYWDDDKRYNPSLKSRLTISKDSSKNQVVLKITSVDPVDTATYSCARSTPIVANAMDYWGQGTSVTVSS

SEQ ID NO: 19 - protein, 121 aa, Artificial Sequence
  Variable Heavy (VH) 2 Chain
QVTLKESGPALVKPTQTLTLTCSFSGFSLSTSGMGVSWIRQPAGKGLEWLAHIYWDDDKRYNPSLKSRLTISKDSSKNQVVLTMTNMDPVDTATYSCARSTPIVANAMDYWGQGTLVTVSS

SEQ ID NO: 20 - protein, 121 aa, Artificial Sequence
  Variable Heavy (VH) 3 Chain
QVTLKESGPALVKPTQTLTLTCSFSGFSLSTSGMGVSWIRQPAGKGLEWLAHIYWDDDKRYNPSLKSRLTISKDSSKNQVVLTMTNMDPVDTATYYCARSTPIVANAMDYWGQGTLVTVSS

SEQ ID NO: 21 - protein, 121 aa, Artificial Sequence
  Variable Heavy (VH) 4 Chain
QVTLKESGPALVKPTQTLTLTCTFSGFSLSTSGMGVSWIRQPAGKGLEWLAHIYWDDDKRYNPSLKSRLTISKDTSKNQVVLTMTNMDPVDTATYYCARSTPIVANAMDYWGQGTLVTVSS

SEQ ID NO: 22 - protein, 121 aa, Artificial Sequence
  Variable Heavy (VH) Chimeric (O) Chain
QVTLKESGPGILQPSQTLSLTCSFSGFSLSTSGMGVSWIRQPSGKGLEWLAHIYWDDDKRYNPSLKSRLTISKDSSSNQVFLKITSVDTADTATYSCARSTPIVANAMDYWGQGTSVTVSS

SEQ ID NO: 23 - DNA, 363 bp, Artificial Sequence
  Variable Heavy (VH) 1 Chain
caggtcaccttgaaggagtctggtcctgccatcgtgaaacccacacagaccctcacgctgacctgcagcttctctgggttctcactcagcactagtggaatgggtgtgagctggatccgtcagccctcaggaaagggcctggagtggcttgcacacatttattgggatgatgataagcgctacaacccatctctgaagagcaggctcaccatctccaaggacagctccaaaaaccaggtggtccttaaaatcaccagcgtggaccctgtggacacagccacatattcctgtgcacggagcacccccatcgtggccaacgccatggactactggggccaaggaaccagcgtcaccgtctcctca

SEQ ID NO: 24 - DNA, 363 bp, Artificial Sequence
  Variable Heavy (VH) 2 Chain
caggtcaccttgaaggagtctggtcctgccctggtgaaacccacacagaccctcacgctgacctgcagcttctctgggttctcactcagcactagtggaatgggtgtgagctggatccgtcagcccgccggaaagggcctggagtggcttgcacacatttattgggatgatgataagcgctacaacccatctctgaagagcaggctcaccatctccaaggacagctccaaaaaccaggtggtccttacaatgaccaacatggaccctgtggacacagccacatattcctgtgcacggagcacccccatcgtggccaacgccatggactactggggccaaggaaccctggtcaccgtctcctca

SEQ ID NO: 25 - DNA, 363 bp, Artificial Sequence
  Variable Heavy (VH) 3 Chain
caggtcaccttgaaggagtctggtcctgccctggtgaaacccacacagaccctcacgctgacctgcagcttctctgggttctcactcagcactagtggaatgggtgtgagctggatccgtcagcccgccggaaagggcctggagtggcttgcacacatttattgggatgatgataagcgctacaacccatctctgaagagcaggctcaccatctccaaggacagctccaaaaaccaggtggtccttacaatgaccaacatggaccctgtggacacagccacatattactgtgcacggagcacccccatcgtggccaacgccatggactactggggccaaggaaccctggtcaccgtctcctca

SEQ ID NO: 26 - DNA, 363 bp, Artificial Sequence
  Variable Heavy (VH) 4 Chain
caggtcaccttgaaggagtctggtcctgccctggtgaaacccacacagaccctcacgctgacctgcaccttctctgggttctcactcagcactagtggaatgggtgtgagctggatccgtcagcccgccggaaagggcctggagtggcttgcacacatttattgggatgatgataagcgctacaacccatctctgaagagcaggctcaccatctccaaggacacctccaaaaaccaggtggtccttacaatgaccaacatggaccctgtggacacagccacatattactgtgcacggagcacccccatcgtggccaacgccatggactactggggccaaggaaccctggtcaccgtctcctca

SEQ ID NO: 27 - DNA, 363 bp, Artificial Sequence
  Variable Heavy (VH) Chimeric (O) Chain
caggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggattcctccagcaaccaggtcttcctcaagatcaccagtgtggacactgcagatactgccacatactcctgtgctcgaagtactccgattgtagctaatgctatggactactggggtcaaggaacctcagtcaccgtctcctca

SEQ ID NO: 28 - protein, 111 aa, Artificial Sequence
  Variable Kappa Light (VL) 1 Chain
DIVLTQSPDSLAVSLGERATINCKASQSVDYDGDSYMNWYQQKPGQPPKLLIYAASNLESGIPARFSGSGSGTDFTLTISSLQEEDVATYYCQQSNEDPYTFGQGTKLEIK

SEQ ID NO: 29 - protein, 111 aa, Artificial Sequence
  Variable Kappa Light (VL) 2 Chain
DIVLTQSPDSLAVSLGERATINCKASQSVDYDGDSYMNWYQQKPGQPPKLLIYAASNLESGIPARFSGSGSGTDFTLTISSLQPEDVATYYCQQSNEDPYTFGQGTKLEIK

SEQ ID NO: 30 - protein, 111 aa, Artificial Sequence
  Variable Kappa Light (VL) 3 Chain
DIVMTQSPDSLAVSLGERATINCKASQSVDYDGDSYMNWYQQKPGQPPKLLIYAASNLESGIPARFSGSGSGTDFTLTISSLQPEDVATYYCQQSNEDPYTFGQGTKLEIK

SEQ ID NO: 31 - protein, 111 aa, Artificial Sequence
  Variable Kappa Light (VL) Chimeric (O) Chain
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 32 - DNA, 333 bp, Artificial Sequence
  Variable Kappa Light (VL) 1 Chain
gacatcgtgctgacccagtctccagactccctggctgtgtctctgggcgagagggccaccatcaactgcaaggccagccagagtgttgactacgacggcgacagttacatgaattggtaccagcagaaaccaggacagcctcctaagctgctcatttacgccgcatctaacctggaatccggcatccctgcccgattcagtggcagcgggtctgggacagatttcactctcaccatcagcagcctgcaggaggaagatgtggcaacttattactgtcagcaatctaatgaggacccttacacttttggccaggggaccaagctggagatcaaa

SEQ ID NO: 33 - DNA, 333 bp, Artificial Sequence
  Variable Kappa Light (VL) 2 Chain
gacatcgtgctgacccagtctccagactccctggctgtgtctctgggcgagagggccaccatcaactgcaaggccagccagagtgttgactacgacggcgacagttacatgaattggtaccagcagaaaccaggacagcctcctaagctgctcatttacgccgcatctaacctggaatccggcatccctgcccgattcagtggcagcgggtctgggacagatttcactctcaccatcagcagcctgcagcctgaagatgtggcaacttattactgtcagcaatctaatgaggacccttacacttttggccaggggaccaagctggagatcaaa

SEQ ID NO: 34 - DNA, 333 bp, Artificial Sequence
  Variable Kappa Light (VL) 3 Chain
gacatcgtgatgacccagtctccagactccctggctgtgtctctgggcgagagggccaccatcaactgcaaggccagccagagtgttgactacgacggcgacagttacatgaattggtaccagcagaaaccaggacagcctcctaagctgctcatttacgccgcatctaacctggaatccggcatccctgcccgattcagtggcagcgggtctgggacagatttcactctcaccatcagcagcctgcagcctgaagatgtggcaacttattactgtcagcaatctaatgaggacccttacacttttggccaggggaccaagctggagatcaaa

SEQ ID NO: 35 - DNA, 333 bp, Artificial Sequence
  Variable Kappa Light  (VL) Chimeric (O) Chain
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctggcatcccagccaggtttagtggcagtgggtctgggacagacttcaccctcaacatccatcctgtggaggaggaggatgctgcaacctattactgtcagcaaagtaatgaggacccgtacacgttcggaggggggaccaagctggaaataaaa

SEQ ID NO: 36 - protein, 753 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 37 - protein, 670 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQK

SEQ ID NO: 38 - protein, 654 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKM

SEQ ID NO: 39 - protein, 42 aa, Homo sapiens [sequence omitted]

SEQ ID NO: 40 - protein, 40 aa, Homo sapiens
DAEFRHDSGYEVHHQKLVFFAEDVGSNKGAIIGLMVGGVV

SEQ ID NO: 41 - protein, 542 aa, Homo sapiens
SSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD